(19) United States
(12) Patent Application Publication
(10) Pub. No.: US 2022/0016102 A1
(43) Pub. Date: Jan. 20, 2022

(54) ER PROTEIN REGULATORS AND USE THEREOF

(71) Applicant: ShanghaiTech University, ShanghaiShanghai (CN)

(72) Inventors: Xiaobao Yang, Shanghai (CN); Biao Jiang, Shanghai (CN); Renhong Sun, Shanghai (CN); Chaowei Ren, Shanghai (CN); Ning Sun, Shanghai (CN); Xing Qiu, Shanghai (CN)

(73) Assignee: ShanghaiTech University, Shanghai (CN)

(21) Appl. No.: 17/296,165

(22) PCT Filed: Nov. 20, 2019
(86) PCT No.: PCT/CN2019/119766

§ 371 Date: May 21, 2021

(30) Foreign Application Priority Data
Nov. 21, 2018 (CN) 201811390393.6

(51) Int. Cl. A61K 31/454; C07D 401/04; C07D 417/12; A61K 31/427; A61P 35/00

(52) U.S. Cl. CPC A61K 31/454; C07D 401/04; A61P 35/00; A61K 31/427; C07D 417/12

(57) ABSTRACT

The present disclosure relates to an ER protein regulator compound represented by formula (I) and use thereof. LIN in the compound represented by formula (I) is a linker; ULM is a small-molecule ligand of VHL or CRBN protease having a ubiquitylation function; and group X is CH₂, O or NH, and group X is covalently linked to ULM by means of the linker LIN. The designed and synthesized compounds of the present disclosure have wide pharmacological activity, has the function of regulating ER protein and inhibiting the activity of tumors, and can be used for preventing and/or treating diseases and disorders associated with estrogen receptors, or related tumor treatment.

### TECHNICAL FIELD

The present disclosure relates to compounds of formula (I) and use thereof, especially use for preventing and/or treating diseases or disorders associated with estrogen receptors (ERs) or for anti-tumor.

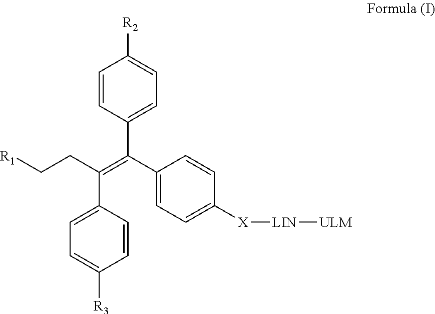

### BACKGROUND

Breast cancer is one of the most common malignant tumors in women worldwide, and the incidence of breast cancer worldwide has increased since the late 1970s. According to data released by the National Cancer Center, in 2014, there were about 278,900 new cases of female breast cancer in the country, accounting for 16.51% of the incidence of female malignant tumors, ranking first in the incidence of female malignant tumors. In breast tissue, the binding of estrogen to estrogen receptor will stimulate the estrogen receptor signaling pathway, thereby affecting the proliferation, differentiation and apoptosis of the breast cells. When this pathway is abnormal, it can cause an imbalance in related gene expression, excessive proliferation of breast cancer cells, and at the same time, apoptosis in breast cancer cells to be blocked, thereby inducing breast cancer.

The estrogen receptor (ER) is a member of the nuclear receptor superfamily, a steroid hormone protein, which can bind to its ligand, estrogen, to stimulate the estrogen receptor signaling pathway, act as a transcription factor activated by the ligand and participate in the up-regulation and down-regulation of related gene expression. The estrogen receptor is mainly located in the nucleus. When it binds to estrogen, the estrogen receptor dimerizes and binds to the estrogen response element (ERE) on the target gene through its DNA binding domain (DBD) to recruit related synergistic activating factors. These activating factors have histone acetyltransferase activity, and can acetylate histones, activate chromatin structure, increase the recruitment of RNA polymerase near the promoter, and regulate the transcription of downstream genes. Due to the large number of downstream genes and the expression of estrogen receptors in many cell types, effective regulation of estrogen receptors is of great significance for the prevention or treatment of estrogen-dependent diseases.

17-estradiol (E2) is the natural hormone of the estrogen receptor and the most active estrogen. It plays a very important role in target tissues such as reproductive organs, bones, cardiovascular and nervous systems. The reduction of estrogen production in postmenopausal women can cause diseases such as osteoporosis, atherosclerosis, and depression and the like. However, excessive estrogen content can stimulate breast cancer, uterine cancer and endometriosis. The estrogen receptor includes two subtypes, ERα and ERβ. These two subtypes have only 53% of the same amino acid sequence in the ligand binding region, therefore they have both the same ligand and their respective different ligands. They are widely expressed in different tissue types. ERα is present in breast cancer cells, endometrium, ovarian stromal cells and hypothalamus, while ERβ is expressed in tissues such as brain, bone, heart and endothelial cells. Therefore, the development of selective estrogen receptor ligands is expected to suppress the pathogenicity of estrogen on the one hand, while retaining its beneficial functions on the other hand.

For estrogen-dependent breast cancer, it is possible to inhibit the proliferation of tumor cells by blocking the production of estrogen or preventing the binding of estrogen to receptors. In the process of receptor-ligand binding, anti-estrogen drugs can compete with ER to block downstream signaling pathways to achieve therapeutic effects. Representative anti-estrogen drugs include toremifene and tamoxifen. Toremifene is a non-steroidal anti-estrogen drug with similar structure to estrogen, including two isomers: (Z)-isomer having anti-estrogen-activity and (E)-isomer having weak estrogen activity, wherein (Z)-isomer can compete with estrogen in the cell for binding to the corresponding receptor ER, so that the corresponding estrogen and estrogen receptor signaling pathways are blocked, and cancer cells cannot complete normal replication and transcription, which affects their normal proliferation. When the drug binds to the receptor to form a drug-receptor complex, the recycling of the receptor is blocked due to the uneasy dissociation of the complex, but the ER on the tumor surface still exists and can be activated by other pathways, and thus there will be drug resistance. Such drugs usually show partial agonism in other tissues and cells, so the estrogen-mediated activity is not completely blocked, and called selective estrogen receptor modulators (SERMs).

Therefore, there is an urgent need to adopt a new drug development pattern to develop new ER ligands, so that on the one hand, it can maintain the selectivity of SERMs for estrogen receptor binding, and on the other hand, it can regulate the expression level of ER protein.

### SUMMARY OF THE INVENTION

The protein degradation targeted drug (Proteolysis Targeting Drug, PROTAD) developed by the protein degradation technology platform provides the possibility for the development of this desirable drugs.

The ubiquitin-mediated protein degradation pathway is responsible for the selective degradation of most proteins in eukaryotic cells, and plays a role in cleaning up useless or harmful proteins in cells. The protein degradation technology platform makes use of this natural protein degradation pathway in the cell: through a specially designed bispecific protein regulator, the pathogenic target protein is ubiquitinated, and the pathway is activated for targeted degradation of the target protein. The PROTAD molecule contains the target protein ligand and the E3 ubiquitin ligase ligand, which are connected by a linker and can bind to the target protein and E3 ubiquitin ligase at the same time, so that the target protein that does not have natural ubiquitination conditions can be ubiquitinated, and then recognized and degraded by the proteasome. Compared with traditional small-molecule drug design, this new drug action mode only requires small-molecule drugs to temporarily bind to the target protein, and label the target protein as “needs to be cleaned up”, and thus a low drug dose can meet the requirements. These drugs can be recycled, and play a role only at nanomolar concentration in many cases, thus greatly reducing the risk of off-target effects and drug resistance. If toremifene-like SERMs are used as estrogen receptor ligands, this action mode can both retain its selective specificity, and will not have problem of partial agonism caused by large dosage when it is used as a common estrogen receptor regulator, thereby avoiding possible side effects. The PROTAD molecule thus designed is a potential desired drug for us that can treat diseases or disorders related to estrogen receptors (especially breast cancer) while having ER protein binding selectivity and regulating ER protein effects.

Therefore, in one aspect, the present disclosure provides a compound of formula (I):

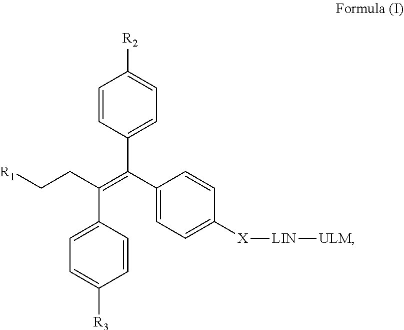

or salts, enantiomers, stereoisomers, solvates, or polymorphs thereof, wherein X, R₁, R₂, R₃, groups LW, and ULM and all substituents are as defined in the detailed description of the invention.

The present disclosure also provides a pharmaceutical composition comprising the compound of formula (I) or a pharmaceutically acceptable salt, enantiomers, stereoisomers, solvates, or polymorphs thereof, and at least one pharmaceutically acceptable carrier.

The present disclosure also provides a compound of formula (I), or a pharmaceutically acceptable salt, enantiomers, stereoisomers, solvates, or polymorphs thereof for use as a medicament:

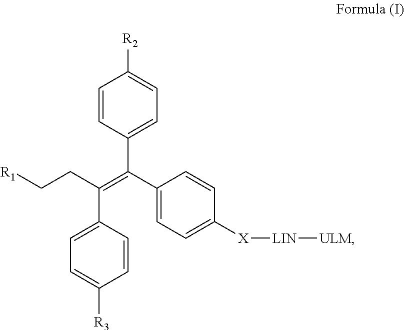

wherein X, R₁, R₂, R₃, groups LIN, and ULM and all substituents are as defined in the detailed description of the invention.

The present disclosure also provides the compound of formula (I), or a pharmaceutically acceptable salt, enantiomers, stereoisomers, solvates, or polymorphs thereof for use in the prevention and/or treatment of diseases or disorders associated with estrogen receptor.

The present disclosure further provides the use of the compound of formula (I), or a pharmaceutically acceptable salt, enantiomers, stereoisomers, solvates, or polymorphs thereof for manufacturing a medicament for preventing and/or treating diseases or disorders associated with estrogen receptor.

The present disclosure also provides a method for treating or preventing diseases or disorders associated with estrogen receptor, comprising administering to a subject in need a therapeutically effective amount of the compound of formula (I), or a pharmaceutically acceptable salt, enantiomers, stereoisomers, solvates, or polymorphs thereof, or the pharmaceutical composition.

### BRIEF DESCRIPTION OF DRAWINGS

FIGS. 1(A)-(O) show western blotting detection of the level of intracellular ER protein to characterize the regulatory effect of the corresponding ER protein regulators (also known as PROTAD small molecule) on the ER protein in the breast cancer cell line T47D.

FIGS. 2(A)-(F) show western blotting detection of the level of intracellular ER protein to characterize the regulatory effect of the corresponding ER protein regulators (also known as PROTAD small molecule) on the ER protein in the breast cancer cell line MCF-7.

FIG. 3 shows a growth inhibition experiment of the ER protein regulators according to the present invention in the breast cancer cell line MCF-7.

### DETAILED DESCRIPTION OF THE INVENTION

In one aspect, the present disclosure provides embodiment (1) which relates to a compound of formula (I):

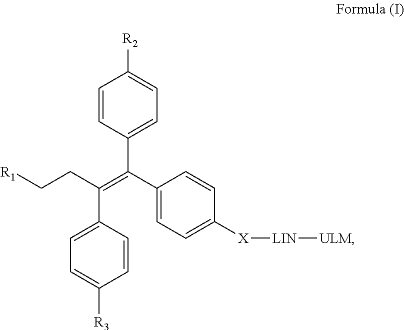

or salts, enantiomers, stereoisomers, solvates, or polymorphs thereof, wherein X is covalently bonded to ULM through a linking group LIN;

wherein R₁ represents halogen, R₂ represents H, halogen, or OH, and R₃ represents H, halogen, or OH; or R₁ represents H, and R₂ and R₃ are both halogen or OH;

X represents CH₂, O, or NH;

LIN is a linking group and represents -alkylene- (especially —C₁₋₆₀ alkylene-, preferably —C₁₋₅₀ alkylene-, more preferably —C₁₋₄₀ alkylene-, and most preferably —C₁₋₃₀ alkylene-), wherein the alkylene group is a linear or branched alkylene group optionally interrupted one or more times by one or more groups selected from the group consisting of O, CO, CON(R₄), N(R₅)CO, N(R₆), alkynylene, alkenylene, cycloalkylene, arylene, heterocyclylene, heteroarylene, or any combination thereof, wherein the linear or branched alkylene group is optionally substituted with one or more substituents; and R₄, R₅, and R₆ are each independently selected from the group consisting of H and C₁₋₃ alkyl,

ULM is a small-molecule ligand of VHL or CRBN protease having a ubiquitylation function.

Herein, LIN represents -alkylene-, wherein any one of the two ends of the -alkylene- can be connected to the group X, and the other end can be connected to ULM.

Embodiment (2) relates to the compound of formula (I) or salts, enantiomers, stereoisomers, solvates, or polymorphs thereof according to embodiment (1), wherein, R₁ represents halogen; R₂ represents H, halogen, or OH; R₃ represents H; and X represents O.

Embodiment (3) relates to the compound of formula (I) or salts, enantiomers, stereoisomers, solvates, or polymorphs thereof according to embodiment (1), wherein, R₁ represents halogen; R₂ represents H, halogen, or OH; R₃ represents halogen; and X represents O.

Embodiment (4) relates to the compound of formula (I) or salts, enantiomers, stereoisomers, solvates, or polymorphs thereof according to embodiment (1), wherein, R₁ represents halogen; R₂ represents H, halogen, or OH; R₃ represents OH; and X represents O.

Embodiment (5) relates to the compound of formula (I) or salts, enantiomers, stereoisomers, solvates, or polymorphs thereof according to embodiment (1), wherein, R₁ represents halogen; R₂ represents H; R₃ represents H, halogen, or OH; and X represents O.

Embodiment (6) relates to the compound of formula (I) or salts, enantiomers, stereoisomers, solvates, or polymorphs thereof according to embodiment (1), wherein, R₁ represents halogen; R₂ represents halogen; R₃ represents H, halogen, or OH; and X represents O.

Embodiment (7) relates to the compound of formula (I) or salts, enantiomers, stereoisomers, solvates, or polymorphs thereof according to embodiment (1), wherein, R₁ represents halogen; R₂ represents OH; R₃ represents H, halogen, or OH; and X represents O.

Embodiment (8) relates to the compound of formula (I) or salts, enantiomers, stereoisomers, solvates, or polymorphs thereof according to embodiment (1), wherein, R₁ represents halogen; R₂ and R₃ both represent H; and X represents O.

Embodiment (9) relates to the compound of formula (I) or salts, enantiomers, stereoisomers, solvates, or polymorphs thereof according to embodiment (1), wherein, R₁ represents halogen; R₂ represents OH; R₃ represents H; and X represents O.

Embodiment (10) relates to the compound of formula (I) or salts, enantiomers, stereoisomers, solvates, or polymorphs thereof according to embodiment (1), wherein, R₁ represents halogen; R₂ represents H; R₃ represents OH; and X represents O.

Embodiment (11) relates to the compound of formula (I) or salts, enantiomers, stereoisomers, solvates, or polymorphs thereof according to embodiment (1), wherein, R₁ represents halogen; R₂ and R₃ both represent OH; and X represents O.

Embodiment (12) relates to the compound of formula (I) or salts, enantiomers, stereoisomers, solvates, or polymorphs thereof according to embodiment (1), wherein, R₁ represents halogen; R₂ and R₃ both represent halogen; and X represents O.

Embodiment (13) relates to the compound of formula (I) or salts, enantiomers, stereoisomers, solvates, or polymorphs thereof according to embodiment (1), wherein, R₁ represents halogen; R₂ represents H; R₃ represents halogen; and X represents O.

Embodiment (14) relates to the compound of formula (I) or salts, enantiomers, stereoisomers, solvates, or polymorphs thereof according to embodiment (1), wherein, R₁ represents halogen; R₂ represents halogen; R₃ represents H; and X represents O.

Embodiment (15) relates to the compound of formula (I) or salts, enantiomers, stereoisomers, solvates, or polymorphs thereof according to embodiment (1), wherein, R₁ represents H; R₂ and R₃ both represent OH; and X represents O.

Embodiment (16) relates to the compound of formula (I) or salts, enantiomers, stereoisomers, solvates, or polymorphs thereof according to embodiment (1), wherein, R₁ represents H; R₂ and R₃ both represent halogen; and X represents O.

Embodiment (17) relates to the compound of formula (I) or salts, enantiomers, stereoisomers, solvates, or polymorphs thereof according to embodiment (1), wherein, R₁ represents halogen; R₂ represents H, halogen, or OH; R₃ represents H, halogen, or OH; and X represents O.

Embodiment (18) relates to the compound of formula (I) or salts, enantiomers, stereoisomers, solvates, or polymorphs thereof according to embodiment (1), wherein, R₁ represents halogen; R₂ represents H, halogen, or OH; R₃ represents H, halogen, or OH; and X represents CH₂.

Embodiment (19) relates to the compound of formula (I) or salts, enantiomers, stereoisomers, solvates, or polymorphs thereof according to embodiment (1), wherein, R₁ represents halogen; R₂ represents H, halogen, or OH; R₃ represents H, halogen, or OH; and X represents NH.

Embodiment (20) relates to the compound of formula (I) or salts, enantiomers, stereoisomers, solvates, or polymorphs thereof according to any one of embodiments (1) to (19), wherein, the ULM can represent the following structure of formula (II):

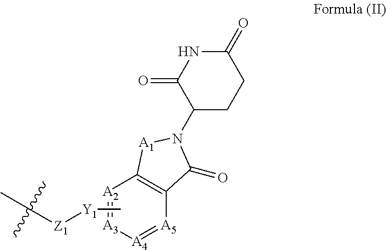

wherein A₁ represents CH₂ or CO; A₂, A₃, A₄, and A₅ are the same or different and each independently represent CH or N, wherein A₂, A₃, A₄, and A₅ are not N at the same time; Y₁ represents CH₂, NH, or O; and Z₁ represents CO or Z₁ is absent.

Embodiment (21) relates to the compound of formula (I) or salts, enantiomers, stereoisomers, solvates, or polymorphs thereof according to embodiment (20), wherein, one or two of A₂, A₃, A₄, and A₅ is/are N.

Embodiment (22) relates to the compound of formula (I) or salts, enantiomers, stereoisomers, solvates, or polymorphs thereof according to embodiment (20), wherein, A₂, A₃, A₄, and A₅ are all CH.

Embodiment (23) relates to the compound of formula (I) or salts, enantiomers, stereoisomers, solvates, or polymorphs thereof according to any one of embodiments (1) to (19), wherein, the ULM can represent the following structure of formula (III):

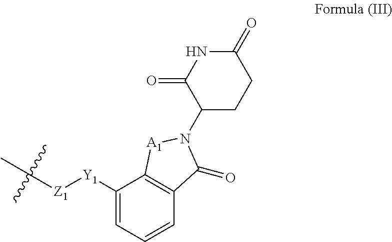

wherein A₁ represents CH₂ or CO; Y₁ represents CH₂, NH, or O; and Z₁ represents CO or Z₁ is absent.

Embodiment (24) relates to the compound of formula (I) or salts, enantiomers, stereoisomers, solvates, or polymorphs thereof according to embodiment (23), wherein, A₁ represents CH₂; Y₁ represents CH₂, NH, or O; and Z₁ represents CO.

Embodiment (25) relates to the compound of formula (I) or salts, enantiomers, stereoisomers, solvates, or polymorphs thereof according to embodiment (23), wherein, A₁ represents CH₂; Y₁ represents CH₂, NH, or O; and Z₁ is absent.

Embodiment (26) relates to the compound of formula (I) or salts, enantiomers, stereoisomers, solvates, or polymorphs thereof according to embodiment (23), wherein, A₁ represents CO; Y₁ represents CH₂, NH, or O; and Z₁ represents CO.

Embodiment (27) relates to the compound of formula (I) or salts, enantiomers, stereoisomers, solvates, or polymorphs thereof according to embodiment (23), wherein, A₁ represents CO; Y₁ represents CH₂, NH, or O; and Z₁ is absent.

Embodiment (28) relates to the compound of formula (I) or salts, enantiomers, stereoisomers, solvates, or polymorphs thereof according to any one of embodiments (1) to (19), wherein, the ULM can represent the following structure of formula (IV):

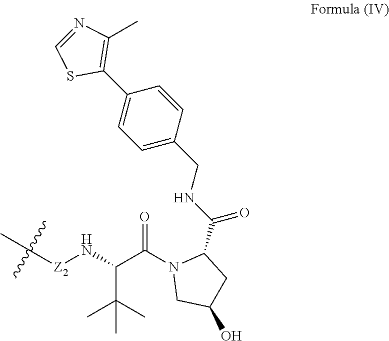

wherein Z₂ represents CO or Z₂ is absent.

Embodiment (29) relates to the compound of formula (I) or salts, enantiomers, stereoisomers, solvates, or polymorphs thereof according to any one of embodiments (1) to (28), wherein, the LIN represents:

a linear or branched C₁-C₃₀ alkylene chain; —(CH₂)ₙ₁—(O(CH₂)ₙ₂)ₘ₁—; —(CH₂)ₙ₁—(O(CH₂)ₙ₂)ₘ₁—O—(CH₂)ₙ₃—; —(CR₇R₈)ₙ₁—(O(CR₉R₁₀)ₙ₂)ₘ₁—; —(CR₁₁R₁₂)ₙ₁—(O(CR₁₃R₁₄)ₙ₂)ₘ₁—O—(CR₁₅R₁₆)ₙ₃—; —(CH₂)ₙ₁—N(R₆)—(CH₂)ₙ₂—; —(CH₂)ₙ₁—N(R₅)CO—(CH₂)ₙ₂—; —(CH₂)ₙ₁—(N(R₅)CO—(CH₂)ₙ₂)ₘ₁—; —(CH₂)ₙ₁—N(R₅)CO—(CH₂)ₙ₂—(O(CH₂)ₙ₃)ₘ₁—; —(CH₂)ₙ₁—(N(R₅)CO—(CH₂)ₙ₂)ₘ₁—O—(CH₂)ₙ₃—; —(CH₂)ₙ₁—N(R₅)CO—(CH₂)ₙ₂—(O(CH₂)ₙ₃)ₘ₁—O(CH₂)ₙ₄—; —(CH₂)ₙ₁-piperazinylene-(CH₂)ₙ₂—; —(CH₂)ₙ₁—N(R₅)CO—(CH₂)ₙ₂-piperazinylene-(CH₂)ₙ₃—; —(CH₂)ₙ₁—(N(R₅)CO—(CH₂)ₙ₂)ₘ₁-piperazinylene-(CH₂)ₙ₃—; —(CH₂)ₙ₁—piperazinylene-CO—(CH₂)ₙ₂—(O(CH₂)ₙ₃)ₘ₁—; —(CH₂)ₙ₁-piperazinylene-CO—(CH₂)ₙ₂—(O(CH₂)ₙ₃)ₘ₁—O(CH₂)ₙ₄—; —(CH₂)ₙ₁-piperazinylene-CO—(CH₂)ₙ₂—; —(CH₂)ₙ₁-phenylene-(CH₂)ₙ₂—; —(CH₂)ₙ₁—N(R₅)CO—(CH₂)ₙ₂-phenylene-(CH₂)ₙ₃—; —(CH₂)ₙ₁—(N(R₅)CO—(CH₂)ₙ₂)ₘ₁-phenylene-(CH₂)ₙ₃—; a linear or branched alkylene chain (especially C₁₋₆₀ alkylene chain) interrupted one or more times by one or more (especially 1-15, preferably 1-10, more preferably 1-5, and most preferably 1-3) selected from the group consisting of CO, alkynylene, alkenylene, cycloalkylene, arylene, heterocyclylene, or heteroarylene, or any combination thereof; or —(CH₂)ₙ₁—(O(CH₂)ₙ₂)ₘ₁— in which backbone carbon chain is interrupted one or more times by one or more (especially 1-15, preferably 1-10, more preferably 1-5, and most preferably 1-3) selected from the group consisting of CO, arylene, heterocyclylene, heteroarylene, or any combination thereof;

wherein,

R₅ and R₆ are each independently selected from the group consisting of H and C₁₋₃ alkyl;

R₇, R₈, R₉, R₁₀, R₁₁, R₁₂, R₁₃, R₁₄, R₁₅, and R₁₆ each independently represent H, linear or branched C₁₋₁₀ alkyl or C₃-C₁₀cycloalkyl, wherein in the same group LIN, R₇, R₈, R₉, and R₁₀ are not H at the same time; or R₁₁, R₁₂, R₁₃, R₁₄, R₁₅, and R₁₆ are not H at the same time; and

n1, n2, n3, n4, and ml each independently represent an integer of 1, 2, 3, 4, 5, 6, 7, 8, 9, 10, 11, 12, 13, 14, 15, 16 , 17, 18, 19, or 20.

Embodiment (30) relates to the compound of formula (I) or salts, enantiomers, stereoisomers, solvates, or polymorphs thereof according to any one of embodiments (1) to (29), wherein, the LIN represents:

—(CH₂)₂O(CH₂)₂O(CH₂)₂—;

—CH₂O(CH₂)₂₀CH₂—;

—CH₂₀(CH₂)₂O(CH₂)₂—;

—(CH₂)₃O(CH₂)₂—;

—(CH₂)₃O(CH₂)₂O(CH₂)₂—;

—(CH₂)₃O(CH₂)₃—;

—(CH₂)₂O(CH₂)₂—;

—(CH₂)₂O(CH₂)₂OCH₂—;

—(CH₂)₂O(CH₂)₂O(CH₂)₃—;

—(CH₂)₂O(CH₂)₂O(CH₂)₂O(CH₂)₂—;

—(CH₂)₂O(CH₂)₂O(CH₂)₂O(CH₂)₃—;

—(CH₂)₅O(CH₂)₂O(CH₂)₂O(CH₂)₅—;

—(CH₂)₅O(CH₂)₂O(CH₂)₂O(CH₂)₆—;

—(CH₂)₂O(CH₂)₂O(CH₂)₂O(CH₂)₂O(CH₂)₂—;

—(CH₂)₂O(CH₂)₂O(CH₂)₂O(CH₂)₂O(CH₂)₃—;

—(CH₂)₂O(CH₂)₂O(CH₂)₂O(CH₂)₂O(CH₂)₂O(CH₂)₂—;

—(CH₂)₃O(CH₂)₂O(CH₂)₂O(CH₂)₂O(CH₂)₂O(CH₂)₂—; or

—(CH₂)₃O(CH₂)₂O(CH₂)₂O(CH₂)₂O(CH₂)₂O(CH₂)₃—.

Embodiment (31) relates to the compound of formula (I) or salts, enantiomers, stereoisomers, solvates, or polymorphs thereof according to any one of embodiments (1) to (29), wherein, the LIN represents:

—CH₂—; —(CH₂)₂—; —(CH₂)₃—; —(CH₂)₄—; —(CH₂)₅—; —(CH₂)₆—; —(CH₂)₇—; —(CH₂)₈—; —(CH₂)₉—; —(CH₂)₁₀—; —(CH₂)₁₁—; —(CH₂)₁₂—; —(CH₂)₁₃—; —(CH₂)₁₄—; —(CH₂)₁₅—; —(CH₂)₁₆—; —(CH₂)₁₇—; —(CH₂)₁₈—; —(CH₂)₁₉—; or —(CH₂)₂₀.

Embodiment (32) relates to the compound of formula (I) or salts, enantiomers, stereoisomers, solvates, or polymorphs thereof according to any one of embodiments (1) to (31), wherein, the substituent(s) is/are selected from the group consisting of hydroxyl, amino, mercapto, halogen or combination thereof.

Embodiment (33) relates to the compound of formula (I) or salts, enantiomers, stereoisomers, solvates, or polymorphs thereof according to any one of embodiments (1) to (32), wherein the LIN is a linear or branched C₁-C₃oalkylene group substituted by one or more substituents selected from the group consisting of hydroxyl, amino, mercapto, halogen, or combination thereof.

Embodiment (34) relates to the compound of formula (I) or salts, enantiomers, stereoisomers, solvates, or polymorphs thereof according to any one of embodiments (1) to (29), wherein the LIN represents: —(CH₂)₁—NH—(CH₂)₁—; —(CH₂)₂—NH—(CH₂)₁—; —(CH₂)₂—NH—(CH₂)₂—; —(CH₂)₂—NH—(CH₂)₃—; —(CH₂)₂—NH—(CH₂)₄—; —(CH₂)₂—NH—(CH₂)₅—; —(CH₂)₂—NH—(CH₂)₆—; —(CH₂)₂—NH—(CH₂)₇—; —(CH₂)₂—NH—(CH₂)₈—; —(CH₂)₂—NH—(CH₂)₉—; —(CH₂)₂—NH—(CH₂)₁₀—; —(CH₂)₂—NH—(CH₂)₁₁—; —(CH₂)₂—NH—(CH₂)₁₂—; —(CH₂)₁—N(CH₃)—(CH₂)₈—; —(CH₂)₂—N(CH₃)—(CH₂)₁—; —(CH₂)₂—N(CH₃)—(CH₂)₂—; —(CH₂)₂—N(CH₃)—(CH₂)₃—; —(CH₂)₂—N(CH₃)—(CH₂)₄—; —(CH₂)₂—N(CH₃)—(CH₂)₅—; —(CH₂)₂—N(CH₃)—(CH₂)₆—; —(CH₂)₂—N(CH₃)—(CH₂)₇—; —(CH₂)₂—N(CH₃)—(CH₂)₈—; —(CH₂)₂—N(CH₃)—(CH₂)₉—; —(CH₂)₂—N(CH₃)—(CH₂)₁₀—; —(CH₂)₂—N(CH₃)—(CH₂)₁₁—; or —(CH₂)₂—N(CH₃)—(CH₂)₁₂—.

Embodiment (35) relates to the compound of formula (I) or salts, enantiomers, stereoisomers, solvates, or polymorphs thereof according to any one of embodiments (1) to (29), wherein the LIN represents: —(CH₂)₂—NHCO—CH₂—; —(CH₂)₂—NHCO—(CH₂)₂—; —(CH₂)₂—NHCO—(CH₂)₃—; —(CH₂)₂—NHCO—(CH₂)₄—; —(CH₂)₂—NHCO—(CH₂)₅—; —(CH₂)₂—NHCO—(CH₂)₆—; —(CH₂)₂—NHCO—(CH₂)₇—; —(CH₂)₂—NHCO—(CH₂)₈—; —(CH₂)₂—NHCO—(CH₂)₉—; —(CH₂)₂—NHCO—(CH₂)₁₀—; —(CH₂)₂—NHCO—(CH₂)₁₁—; —(CH₂)₂—NHCO—(CH₂)₁₂—; —(CH₂)₂—NHCO—(CH₂)₁₃—; —(CH₂)₂—NHCO—(CH₂)₁₄—; —(CH₂)₂—NHCO—(CH₂)₁₅—; —(CH₂)₂—N(CH₃)CO—CH₂—; —(CH₂)₂—N(CH₃)CO—(CH₂)₂—; —(CH₂)₂—N(CH₃)CO—(CH₂)₃—; —(CH₂)₂—N(CH₃)CO—(CH₂)₄—; —(CH₂)₂—N(CH₃)CO—(CH₂)₅—; —(CH₂)₂—N(CH₃)CO—(CH₂)₆—; —(CH₂)₂—N(CH₃)CO—(CH₂)₇—; —(CH₂)₂—N(CH₃)CCO—(CH₂)₈—; —(CH₂)₂—N(CH₃)CO—(CH₂)₉—; —(CH₂)₂—N(CH₃)CO—(CH₂)₁₀—; —(CH₂)₂—N(CH₃)CO—(CH₂)₁₁—; —(CH₂)₂—N(CH₃)CO—(CH₂)₁₂—; —(CH₂)₂—N(CH₃)CO—(CH₂)₁₃—; —(CH₂)₂—N(CH₃)CO—(CH₂)₁₄—; or —(CH₂)₂—N(CH₃)CO—(CH₂)₁₅—.

Embodiment (36) relates to the compound of formula (I) or salts, enantiomers, stereoisomers, solvates, or polymorphs thereof according to any one of embodiments (1) to (29), wherein the LIN represents: —(CH₂)₂—NHCO—(CH₂)₂—O(CH₂)₂—; —(CH₂)₂—NHCO—(CH₂)₂—(O(CH₂)₂)₂—; —(CH₂)₂—NHCO—(CH₂)₂—(O(CH₂)₂)₃—; —(CH₂)₂—NHCO—(CH₂)₂—(O(CH₂)₂)₄—; —(CH₂)₂—NHCO—(CH₂)₂—(O(CH₂)₂)₅—; —(CH₂)₂—NHCO—(CH₂)₂—(O(CH₂)₂)₆—; —(CH₂)₂—NHCO—(CH₂)₂—(O(CH₂)₂)₇—; —(CH₂)₂—NHCO—(CH₂)₂—(O(CH₂)₂)₈—; —(CH₂)₂—NHCO—(CH₂)₂—(O(CH₂)₂)₉—; —(CH₂)₂—NHCO—(CH₂)₂—(O(CH₂)₂)₁₀—; —(CH₂)₂—N(CH₃)CO—(CH₂)₂—O(CH₂)₂—; —(CH₂)₂—N(CH₃)CO—(CH₂)₂—(O(CH₂)₂)₂—; —(CH₂)₂—N(CH₃)CO—(CH₂)₂^{—(O(CH}₂)₂)₃—; —(CH₂)₂—N(CH₃)CO—(CH₂)₂—(O(CH₂)₂)₄—; —(CH₂)₂—N(CH₃)CO—(CH₂)₂—(O(CH₂)₂)₅—; —(CH₂)₂—N(CH₃)CO—(CH₂)₂—(O(CH₂)₂)₆—; —(CH₂)₂—N(CH₃)CO—(CH₂)₂—(O(CH₂)₂)₇—; —(CH₂)₂—N(CH₃)CO—(CH₂)₂—(O(CH₂)₂)₈—; —(CH₂)₂—N(CH₃)CO—(CH₂)₂—(O(CH₂)₂)₀—; or —(CH₂)₂—N(CH₃)CO—(CH₂)₂—(O(CH₂)₂)₁₀—.

Embodiment (37) relates to the compound of formula (I) or salts, enantiomers, stereoisomers, solvates, or polymorphs thereof according to any one of embodiments (1) to (29), wherein the LIN represents: —(CH₂)₂—NHCO—CH₂—O(CH₂)₂—OCH₂—; —(CH₂)₂—NHCO—(CH₂)₂—O(CH₂)₂—OCH₂—; —(CH₂)₂—NHCO—(CH₂)₂—(O(CH₂)₂)₂—OCH₂—; —(CH₂)₂—NHCO—(CH₂)₂—(O(CH₂)₂)₂—O(CH₂)₃—; —(CH₂)₂—N(CH₃)CO—CH₂—O(CH₂)₂—OCH₂—; —(CH₂)₂—N(CH₃)CO—CH₂—(O(CH₂)₂)₂—OCH₂—; —(CH₂)₂—N(CH₃)CO—CH₂—(O(CH₂)₂)₃—OCH₂—; or —(CH₂)₂—N(CH₃)CO—CH₂—(O(CH₂)₂)₂—O(CH₂)₃—.

Embodiment (38) relates to the compound of formula (I) or salts, enantiomers, stereoisomers, solvates, or polymorphs thereof according to any one of embodiments (1) to (29), wherein the LIN represents: —(CH₂)₂—NHCO—(CH₂)₂-piperazinylene-CH₂—; —(CH₂)₂—NHCO—(CH₂)₂-piperazinylene-(CH₂)₃—; —(CH₂)₂—NHCO—(CH₂)₃-piperazinylene-(CH₂)₂—; —(CH₂)₂—NHCO—(CH₂)₃-piperazinylene-(CH₂)₃—; —(CH₂)₂—NHCO—CH₂-piperazinylene-(CH₂)₂—; —(CH₂)₂—N(CH₃)CO—(CH₂)₂-piperazinylene-(CH₂)₂—; —(CH₂)₂—N(CH₃)CO—CH₂-piperazinylene-(CH₂)₂—; —(CH₂)₂—N(CH₃)CO—(CH₂)₂-piperazinylene-(CH₂)₃—; —(CH₂)₂—N(CH₃)CO—(CH₂)₃-piperazinylene-(CH₂)₂—; —(CH₂)₂—N(CH₃)CO—(CH₂)₃-piperazinylene-(CH₂)₃—; —(CH₂)₂—(NHCO—(CH₂)₂)₂-piperazinylene-CH₂—; —(CH₂)₂—(NHCO—(CH₂)₂)₂-piperazinylene-(CH₂)₂—; —(CH₂)₂—(NHCO—(CH₂)₂)₂-piperazinylene-(CH₂)₃—; —(CH₂)₂—(NHCO—(CH₂)₂)₂-piperazinylene-(CH₂)₄—; —(CH₂)₂—(NHCO—(CH₂)₂)₃-piperazinylene-(CH₂)₂—; —(CH₂)₂—(N(CH₃)CO—(CH₂)₂)₂-piperazinylene-CH₂—; —(CH₂)₂—(N(CH₃)CO—(CH₂)₂)₂-piperazinylene-(CH₂)₂—; —(CH₂)₂—(N(CH₃)CO—(CH₂)₂)₂-piperazinylene-(CH₂)₃—; —(CH₂)₂—(N(CH₃)CO—(CH₂)₂)₂-piperazinylene-(CH₂)₄—; or —(CH₂)₂—(N(CH₃)CO—(CH₂)₂)₃-piperazinylene-(CH₂)₂—.

Embodiment (39) relates to the compound of formula (I) or salts, enantiomers, stereoisomers, solvates, or polymorphs thereof according to any one of embodiments (1) to (29), wherein the LIN represents —(CH₂)ₙ₁-piperazinylene-(CH₂)ₙ₂-, wherein n1 and n2 each independently represent an interger of 1, 2, 3, 4, 5, 6, 7, 8, 9, 10, 11, 12, 13, 14, 15, 16 , 17, 18, 19, or 20.

Embodiment (40) relates to the compound of formula (I) or salts, enantiomers, stereoisomers, solvates, or polymorphs thereof according to embodiment (39), wherein the LIN represents -CH₂-piperazinylene-CH₂—; —CH₂-piperazinylene-(CH₂)₂—; —CH₂-piperazinylene-(CH₂)₃—; —CH₂-piperazinylene-(CH₂)₄—; —CH₂-piperazinylene-(CH₂)₅—; —(CH₂)₂-piperazinylene-CH₂—; —(CH₂)₂-piperazinylene-(CH₂)₂—; —(CH₂)₂-piperazinylene-(CH₂)₃—; —(CH₂)₂-piperazinylene-(CH₂)₄—; —(CH₂)₂-piperazinylene-(CH₂)₅—; —(CH₂)₂-piperazinylene-(CH₂)₆—; —(CH₂)₂-piperazinylene-(CH₂)₇—; —(CH₂)₂-piperazinylene-(CH₂)₈—; —(CH₂)₂-piperazinylene-(CH₂)₉—; or —(CH₂)₂-piperazinylene-(CH₂)₁₀—.

Embodiment (41) relates to the compound of formula (I) or salts, enantiomers, stereoisomers, solvates, or polymorphs thereof according to any one of embodiments (1) to (29), wherein the LIN is —(CH₂)ₙ₁-piperazinylene-CO—(CH₂)ₙ₂-(O(CH₂)ₙ₃)ₘ₁—, wherein n1, n2, n3, and m1 each independently represent an integer of 1, 2, 3, 4, 5, 6, 7, 8, 9, 10, 11, 12, 13, 14, 15, 16 , 17, 18, 19, or 20.

Embodiment (42) relates to the compound of formula (I) or salts, enantiomers, stereoisomers, solvates, or polymorphs thereof according to embodiment (41), wherein the LIN is —(CH₂)₂—piperazinylene-CO—CH₂—O(CH₂)₂—, —(CH₂)₂-piperazinylene-CO—CH₂—OCH₂—, —(CH₂)₂-piperazinylene-CO—CH₂—O(CH₂)₂—OCH₂—, —(CH₂)₂-piperazinylene-CO—(CH₂)₂—O(CH₂)₂—, —(CH₂)₂-piperazinylene-CO—(CH₂)₂—(O(CH₂)₂)₂—, —(CH₂)₂-piperazinylene-CO—(CH₂)₂—(O(CH₂)₂)₃—, —(CH₂)₂-piperazinylene-CO—(CH₂)₂—(O(CH₂)₂)₄—, —(CH₂)₂-piperazinylene-CO—(CH₂)₂—(O(CH₂)₂)₅—, —(CH₂)₂-piperazinylene-CO—(CH₂)₂—(O(CH₂)₂)₆—, —(CH₂)₂-piperazinylene-CO—(CH₂)₂—(O(CH₂)₂)₇—, —(CH₂)₂-piperazinylene-CO—(CH₂)₂—(O(CH₂)₂)₈—, —(CH₂)₂-piperazinylene-CO—(CH₂)₂—(O(CH₂)₂)₉—, or —(CH₂)₂-piperazinylene-CO—(CH₂)₂—(O(CH₂)₂)₁₀—.

Embodiment (43) relates to the compound of formula (I) or salts, enantiomers, stereoisomers, solvates, or polymorphs thereof according to any one of embodiments (1) to (29), wherein the LIN is —(CH₂)ₙ₁-piperazinylene-CO—(CH₂)ₙ₂—, wherein n1 and n2 each independently represent an integer of 1, 2, 3, 4, 5, 6, 7, 8, 9, 10, 11, 12, 13, 14, 15, 16 , 17, 18, 19, or 20.

Embodiment (44) relates to the compound of formula (I) or salts, enantiomers, stereoisomers, solvates, or polymorphs thereof according to embodiment (43), wherein the LIN is —(CH₂)₂-piperazinylene-CO—CH₂—, —(CH₂)₂-piperazinylene-CO—(CH₂)₂—, —(CH₂)₂-piperazinylene-CO—(CH₂)₃—, —(CH₂)₂-piperazinylene-CO—(CH₂)₄—, —(CH₂)₂-piperazinylene-CO—(CH₂)₅—, —(CH₂)₂-piperazinylene-CO—(CH₂)₆—, —(CH₂)₂-piperazinylene-CO—(CH₂)₇—, —(CH₂)₂-piperazinylene-CO—(CH₂)₈—, —(CH₂)₂-piperazinylene-CO—(CH₂)₉—, or —(CH₂)₂-piperazinylene-CO—(CH₂)₁₀—.

Embodiment (45) relates to the compound of formula (I) or salts, enantiomers, stereoisomers, solvates, or polymorphs thereof according to any one of embodiments (1) to (29), wherein the LIN is —(CH₂)ₙ₁-phenylene-(CH₂)ₙ₂—, wherein n1 and n2 each independently represent an integer of 1, 2, 3, 4, 5, 6, 7, 8, 9, 10, 11, 12, 13, 14, 15, 16 , 17, 18, 19, or 20.

Embodiment (46) relates to the compound of formula (I) or salts, enantiomers, stereoisomers, solvates, or polymorphs thereof according to embodiment (45), wherein the LIN is —CH₂-phenylene-CH₂—, —(CH₂)₂-phenylene-(CH₂)₂—, —(CH₂)₂-phenylene-(CH₂)₃—, —(CH₂)₂-phenylene-(CH₂)₄—, —(CH₂)₂-phenylene- (CH₂)₅—, —(CH₂)₃-phenylene-(CH₂)₂—, —(CH₂)₄-phenylene-(CH₂)₂—, or —(CH₂)₄-phenylene-(CH₂)₃—.

Embodiment (47) relates to the compound of formula (I) or salts, enantiomers, stereoisomers, solvates, or polymorphs thereof according to any one of embodiments (1) to (29), wherein the LIN is —(CH₂)₂—NHCO—(CH₂)₂-phenylene-(CH₂)₂—, —(CH₂)₂—NHCO—CH₂-phenylene-(CH₂)₂—, —(CH₂)₂—NHCO—(CH₂)₃-phenylene-(CH₂)₂—, —(CH₂)₂—NHCO—(CH₂)₂-phenylene-(CH₂)₃—, —(CH₂)₂—N(CH₃)CO—(CH₂)₂-phenylene- (CH₂)₂—, —(CH₂)₂—N(CH₃)CO—(CH₂)₃-phenylene-(CH₂)₂—, —(CH₂)₂—N(CH₃)CO—(CH₂)₂-phenylene-(CH₂)₃—, —(CH₂)₂—(NHCO—(CH₂)₂)₂-phenylene-(CH₂)₂—, —(CH₂)₂—(NHCO—(CH₂)₂)₂-phenylene-(CH₂)₃—, —(CH₂)₂—(N(CH₃)CO—(CH₂)₂)₂-phenylene-(CH₂)₂—, or —(CH₂)₂—(N(CH₃)CO—(CH₂)₂)₃-phenylene-(CH₂)₂—.

Embodiment (48) relates to the compound of formula (I) or salts, enantiomers, stereoisomers, solvates, or polymorphs thereof according to embodiment (1), wherein

R₁ represents halogen, R₂ and R₃ represent H, and X represents O;

ULM represents the following structure of formula (IV):

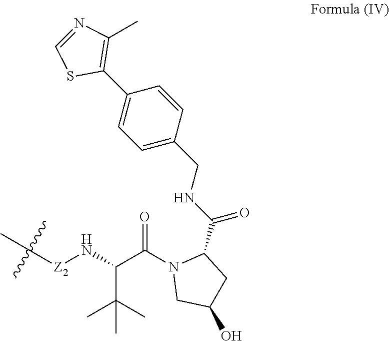

wherein Z₂ represents CO or Z₂ is absent; and
LIN represents alkylene, wherein
   the alkylene group is a linear or branched alkylene group interrupted one or more times by one or more groups selected from the group consisting of O, CON(R₄), N(R₅)CO, or any combination thereof, wherein the linear or branched alkylene group is optionally substituted with one or more substituents selected from the group consisting of hydroxyl, amino, mercapto, and halogen; and R₄ and R₅ are each independently selected from the group consisting of H and C₁₋₃ alkyl.

Embodiment (49) relates to the compound of formula (I) or salts, enantiomers, stereoisomers, solvates, or polymorphs thereof according to embodiment (48), wherein

LIN represents —(CH₂)ₙ₁—N(R₅)CO—(CH₂)ₙ₂—, or —(CH₂)ₙ₁—N(R₅)CO—(CH₂)ₙ₂—(O(CH₂)ₙ₃)ₘ₁—, wherein the hydrogen atom(s) on the carbon atom(s) of backbone carbon chain of the LIN group is/are optionally replaced by one or more substituents selected from the group consisting of hydroxyl, amino, mercapto, and halogen; R₅ is selected from the group consisting of H and C₁₋₃ alkyl; and n1, n2, n3, and ml each independently represent an integer of 1, 2, 3, 4, 5, 6, 7, 8, 9, 10, 11, 12, 13, 14, 15, 16 , 17, 18, 19, or 20.

Embodiment (50) relates to the compound of formula (I) or salts, enantiomers, stereoisomers, solvates, or polymorphs thereof according to embodiments (48) or (49), wherein LIN represents:

—(CH₂)₂—N(CH₃)CO—(CH₂)₂—O(CH₂)₂—; —(CH₂)₂—N(CH₃)CO—(CH₂)₂—(O(CH₂)₂)₂—; —(CH₂)₂—N(CH₃)CO—(CH₂)₂—(O(CH₂)₂)₃—; —(CH₂)₂—N(CH₃)CO—(CH₂)₂—(O(CH₂)₂)₄—; —(CH₂)₂—N(CH₃)CO—(CH₂)₂—(O(CH₂)₂)₅—; —(CH₂)₂—N(CH₃)CO—(CH₂)₂—(O(CH₂)₂)₆—; —(CH₂)₂—N(CH₃)CO—(CH₂)₂—(O(CH₂)₂)₇—; —(CH₂)₂—N(CH₃)CO—(CH₂)₂—(O(CH₂)₂)₈—; —(CH₂)₂—N(CH₃)CO—(CH₂)₂—(O(CH₂)₂)₉—; or —(CH₂)₂—N(CH₃)CO—(CH₂)₂—(O(CH₂)₂)₁₀—.

Embodiment (51) relates to the compound of formula (I) or salts, enantiomers, stereoisomers, solvates, or polymorphs thereof according to embodiments (48) or (49), wherein LIN represents:

—(CH₂)₂—N(CH₃)CO—CH₂—; —(CH₂)₂—N(CH₃)CO—(CH₂)₂—; —(CH₂)₂—N(CH₃)CO—(CH₂)₃—; —(CH₂)₂—N(CH₃)CO—(CH₂)₄—; —(CH₂)₂—N(CH₃)CO—(CH₂)₅—; —(CH₂)₂—N(CH₃)CO—(CH₂)₆—; —(CH₂)₂—N(CH₃)CO—(CH₂)₇—; —(CH₂)₂—N(CH₃)CO—(CH₂)₈—; —(CH₂)₂—N(CH₃)CO—(CH₂)₉—; —(CH₂)₂—N(CH₃)CO—(CH₂)₁₀; —(CH₂)₂—N(CH₃)CO—(CH₂)₁₁—; —(CH₂)₂—N(CH₃)CO—(CH₂)₁₂—; —(CH₂)²—N(CH₃)CO—(CH₂)₁₃—; —(CH₂)₂—N(CH₃)CO—(CH₂)₁₄—; or —(CH₂)₂—N(CH₃)CO—(CH₂)₁₅—.

Embodiment (52) relates to the compound of formula (I) or salts, enantiomers, stereoisomers, solvates, or polymorphs thereof according to embodiment (1), wherein

R₁ represents halogen, R₂ and R₃ represent H, and X represents O;

ULM represents the following structure of formula (II):

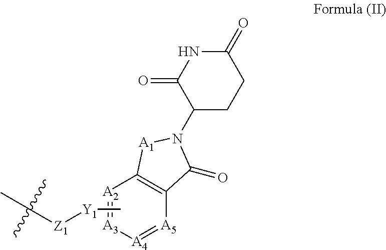

wherein Y₁ represents CH₂, NH, or O; Z₁ represents CO or Z₁ is absent; A₁ represents CH₂ or CO; A₂, A₃, A₄, and A₅ are the same or different and each independently represent CH or N, provided that A₂, A₃, A₄, and A₅ are not N at the same time; and

LIN represents alkylene, wherein
the alkylene group is a linear or branched alkylene group interrupted one or more times by one or more groups selected from the group consisting of CON(R₄), N(R₅)CO, heterocyclylene, heteroarylene, or any combination thereof, wherein the linear or branched alkylene group is optionally substituted with one or more substituents selected from the group consisting of hydroxyl, amino, mercapto, and halogen; R₄ and R₅ are each independently selected from the group consisting of H and C₁₋₃ alkyl; and the heterocyclylene and heteroarylene group are optionally substituted with the substituent(s) selected from the group consisting of C₁₋₃ alkyl, C₁₋₃ alkoxy, cyano, trifluoromethyl, heterocyclyl, halogen, amino, or hydroxyl.

Embodiment (53) relates to the compound of formula (I) or salts, enantiomers, stereoisomers, solvates, or polymorphs thereof according to embodiment (52), wherein LIN is —(CH₂)ₙ₁—N(R₅)CO—(CH₂)ₙ₂-piperazinylene-(CH₂)ₙ₃—, wherein the hydrogen atom(s) on the carbon atom(s) of backbone carbon chain of the LIN group is/are optionally replaced by one or more substituents selected from the group consisting of hydroxyl, amino, mercapto, and halogen; R₅ is selected from the group consisting of H and C₁₋₃ alkyl; and n1, n2, and n3 each independently represent an integer of 1, 2, 3, 4, 5, 6, 7, 8, 9, 10, 11, 12, 13, 14, 15, 16 , 17, 18, 19, or 20, and wherein said piperazinylene group is optionally substituted with a substituent(s) selected from the group consisting of C₁₋₃ alkyl, C₁₋₃ alkoxy, cyano, trifluoromethyl, heterocyclyl, halogen, amino, or hydroxyl.

Embodiment (54) relates to the compound of formula (I) or salts, enantiomers, stereoisomers, solvates, or polymorphs thereof according to embodiment (52), wherein, the ULM represents the following structure of formula (III):

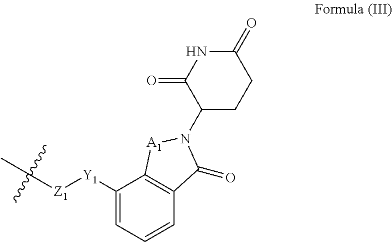

wherein A₁ represents CH₂ or CO; Y₁ represents NH; and Z₁ represents CO or Z₁ is absent.

Embodiment (55) relates to the compound of formula (I) or salts, enantiomers, stereoisomers, solvates, or polymorphs thereof according to any one of embodiments (52) to (54), wherein LIN represents:

—(CH₂)₂—N(CH₃)CO—(CH₂)₂-piperazinylene-(CH₂)₂—; —(CH₂)₂—N(CH₃)CO—CH₂-piperazinylene-(CH₂)₂—; —(CH₂)₂—N(CH₃)CO—(CH₂)₂-piperazinylene-(CH₂)₃—; —(CH₂)₂—N(CH₃)CO—(CH₂)₃-piperazinylene-(CH₂)₂—; or —(CH₂)₂—N(CH₃)CO—(CH₂)₃-piperazinylene-(CH₂)₃—.

Embodiment (56) relates to the compound of formula (I) or salts, enantiomers, stereoisomers, solvates, or polymorphs thereof according to embodiment (1), wherein

R₁ represents halogen, R₂ represents OH, R₃ represents H, and X represents O;

ULM represents the following structure of formula (II):

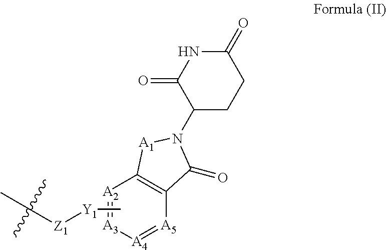

wherein Y₁ represents CH₂, NH, or O; Z₁ represents CO or Z₁ is absent; A₁ represents CH₂ or CO; A₂, A₃, A₄, and A₅ are the same or different and each independently represent CH or N, provided that A₂, A₃, A₄, and A₅ are not N at the same time; and

LIN represents alkylene, wherein
the alkylene group is a linear or branched alkylene group interrupted one or more times by the group(s) selected from the group consisting of CON(R₄), N(R₅)CO, or any combination thereof, wherein the linear or branched alkylene group is optionally substituted with one or more substituents selected from the group consisting of hydroxyl, amino, mercapto, and halogen; R₄ and R₅ are each independently selected from the group consisting of H and C₁₋₃ alkyl.

Embodiment (57) relates to the compound of formula (I) or salts, enantiomers, stereoisomers, solvates, or polymorphs thereof according to embodiment (56), wherein LIN represents —(CH₂)ₙ₁—N(R₅)CO—(CH₂)ₙ₂—, wherein the hydrogen atom(s) on the carbon atom(s) of backbone carbon chain of the LIN group is/are optionally replaced by one or more substituents selected from the group consisting of hydroxyl, amino, mercapto, and halogen; R₅ is selected from the group consisting of H and C₁₋₃ alkyl; and n1 and n2 each independently represent an integer of 1, 2, 3, 4, 5, 6, 7, 8, 9, 10, 11, 12, 13, 14, 15, 16 , 17, 18, 19, or 20.

Embodiment (58) relates to the compound of formula (I) or salts, enantiomers, stereoisomers, solvates, or polymorphs thereof according to embodiment (56), wherein, the ULM represents the following structure of formula (III):

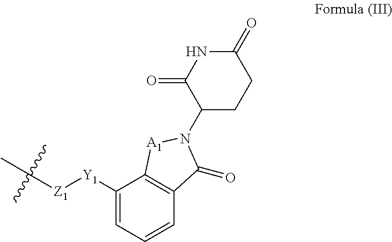

wherein A₁ represents CH₂ or CO; Y₁ represents NH; and Z₁ represents CO or Z₁ is absent.

Embodiment (59) relates to the compound of formula (I) or salts, enantiomers, stereoisomers, solvates, or polymorphs thereof according to any one of embodiments (56) to (58), wherein LIN represents:

—(CH₂)₂—NHCO—CH₂—; —(CH₂)₂—NHCO—(CH₂)₂—; —(CH₂)₂—NHCO—(CH₂)₃—; —(CH₂)₂—NHCO—(CH₂)₄—; —(CH₂)₂—NHCO—(CH₂)₅—; —(CH₂)₂—NHCO—(CH₂)₆—; —(CH₂)₂—NHCO—(CH₂)₇—; —(CH₂)₂—NHCO—(CH₂)₈—; —(CH₂)₂—NHCO—(CH₂)₉—; —(CH₂)₂—NHCO—(CH₂)₁₀—; —(CH₂)₂—NHCO—(CH₂)₁₁—; —(CH₂)₂—NHCO—(CH₂)₁₂—; —(CH₂)₂—NHCO—(CH₂)₁₃—; —(CH₂)₂—NHCO—(CH₂)₁₄—; or —(CH₂)₂—NHCO—(CH₂)₁₅—.

Embodiment (60) relates to the compound of formula (I) or salts, enantiomers, stereoisomers, solvates, or polymorphs thereof according to embodiment (1), wherein

R₁ represents halogen, R₂ represents OH, R₃ represents H, and X represents O;

ULM represents the following structure of formula (IV):

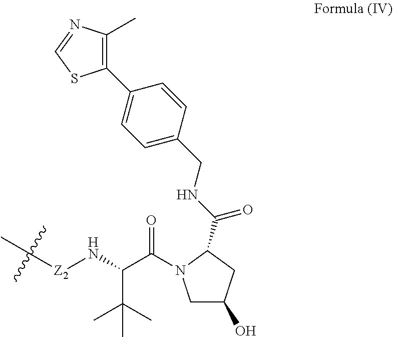

wherein Z₂ represents CO or Z₂ is absent; and

LIN represents alkylene, wherein
the alkylene group is a linear or branched alkylene group interrupted one or more times by the group(s) selected from the group consisting of O, CO, CON(R₄), N(R₅)CO, heterocyclylene, heteroarylene, or any combination thereof, wherein the linear or branched alkylene group is optionally substituted with one or more substituents selected from the group consisting of hydroxyl, amino, mercapto, and halogen; R₄ and R₅ are each independently selected from the group consisting of H and C₁₋₃ alkyl; and the heterocyclylene and heteroarylene group are optionally substituted with the substituent(s) selected from the group consisting of C₁₋₃ alkyl, C₁₋₃ alkoxy, cyano, trifluoromethyl, heterocyclyl, halogen, amino, or hydroxyl.

Embodiment (61) relates to the compound of formula (I) or salts, enantiomers, stereoisomers, solvates, or polymorphs thereof according to embodiment (60), wherein:
LIN represents —(CH₂)ₙ₁—N(R₅)CO—(CH₂)ₙ₂—(O(CH₂)ₙ₃)ₘ₁—, —(CH₂)ₙ₁—N(R₅)CO—(CH₂)ₙ₂—(O(CH₂)ₙ₃)ₘ₁—O(CH₂)ₙ₄—, —(CH₂)ₙ₁—N(R₅)CO—(CH₂)ₙ₂—, —(CH₂)ₙ₁—N(R₅)CO—(CH₂)ₙ₂-piperazinylene-(CH₂)ₙ₃—, —(CH₂)ₙ₁-piperazinylene-CO—(CH₂)ₙ₂—, or —(CH₂)ₙ₁-piperazinylene-CO—(CH₂)ₙ₂—(O(CH₂)ₙ₃)ₘ₁—, wherein the hydrogen atom(s) on the carbon atom(s) of backbone carbon chain of the LIN group is/are optionally replaced by one or more substituents selected from the group consisting of hydroxyl, amino, mercapto, and halogen; R₅ is selected from the group consisting of H and C₁₋₃ alkyl; and n1, n2, n3, n4, and ml each independently represent an integer of 1, 2, 3, 4, 5, 6, 7, 8, 9, 10, 11, 12, 13, 14, 15, 16 , 17, 18, 19, or 20, and wherein said piperazinylene group is optionally substituted with the substituent(s) selected from the group consisting of C₁₋₃ alkyl, C₁₋₃ alkoxy, cyano, trifluoromethyl, heterocyclyl, halogen, amino, or hydroxyl.

Embodiment (62) relates to the compound of formula (I) or salts, enantiomers, stereoisomers, solvates, or polymorphs thereof according to embodiments (60) or (61), wherein LIN represents:

—(CH₂)₂—NHCO—CH₂—O(CH₂)₂—OCH₂—; —(CH₂)₂—NHCO—(CH₂)₂—O(CH₂)₂—OCH₂—; —(CH₂)₂—NHCO—(CH₂)₂—(O(CH₂)₂)₂—OCH₂—; —(CH₂)₂—NHCO—(CH₂)₂—(O(CH₂)₂)₂—O(CH₂)₃—; —(CH₂)₂—N(CH₃)CO—CH₂—O(CH₂)₂—OCH₂—; —(CH₂)₂—N(CH₃)CO—CH₂—(O(CH₂)₂)₂—OCH₂—; —(CH₂)₂—N(CH₃)CO—CH₂—(O(CH₂)₂)₃—OCH₂—; —(CH₂)₂—N(CH₃)CO—CH₂—(O(CH₂)₂)₂—O(CH₂)₃—; —(CH₂)₂—NHCO—(CH₂)₂—O(CH₂)₂—; —(CH₂)₂—NHCO—(CH₂)₂—(O(CH₂)₂)₂—; —(CH₂)₂—NHCO—(CH₂)₂—(O(CH₂)₂)₃—; —(CH₂)₂—NHCO—(CH₂)₂—(O(CH₂)₂)₄—; —(CH₂)₂—NHCO—(CH₂)₂—(O(CH₂)₂)₅—; —(CH₂)₂—NHCO—(CH₂)₂—(O(CH₂)₂)₆—; —(CH₂)₂—NHCO—(CH₂)₂—(O(CH₂)₂)₇—; —(CH₂)₂—NHCO—(CH₂)₂—(O(CH₂)₂)₈—; —(CH₂)₂—NHCO—(CH₂)₂—(O(CH₂)₂)₉—; or —(CH₂)₂—NHCO—(CH₂)₂—(O(CH₂)₂)₁₀—.

Embodiment (63) relates to the compound of formula (I) or salts, enantiomers, stereoisomers, solvates, or polymorphs thereof according to embodiments (60) or (61), wherein LIN represents:

—(CH₂)₂—NHCO—CH₂—; —(CH₂)₂—NHCO—(CH₂)₂—; —(CH₂)₂—NHCO—(CH₂)₃—; —(CH₂)₂—NHCO—(CH₂)₄—; —(CH₂)₂—NHCO—(CH₂)₅—; —(CH₂)₂—NHCO—(CH₂)₆—; —(CH₂)₂—NHCO—(CH₂)₇—; —(CH₂)₂—NHCO—(CH₂)₈—; —(CH₂)₂—NHCO—(CH₂)₉—; —(CH₂)₂—NHCO—(CH₂)₁₀—; —(CH₂)₂—NHCO—(CH₂)₁₁—; —(CH₂)₂—NHCO—(CH₂)₁₂—; —(CH₂)₂—NHCO—(CH₂)₁₃—; —(CH₂)₂—NHCO—(CH₂)₁₄—; —(CH₂)₂—NHCO—(CH₂)₁₅—; —(CH₂)₂—NHCO—(CH₂)₁₆—; —(CH₂)₂—NHCO—(CH₂)₁₇—; —(CH₂)₂—NHCO—(CH₂)₁₈—; —(CH₂)₂—NHCO—(CH₂)₁₉—; or —(CH₂)₂—NHCO—(CH₂)₂₉—.

Embodiment (64) relates to the compound of formula (I) or salts, enantiomers, stereoisomers, solvates, or polymorphs thereof according to embodiments (60) or (61), wherein LIN represents:

—(CH₂)₂—NHCO—(CH₂)₂-piperazinylene-(CH₂)₂—; —(CH₂)₂—NHCO—(CH₂)₂-piperazinylene-(CH₂)₃—; —(CH₂)₂—NHCO—(CH₂)₃-piperazinylene-(CH₂)₂—; —(CH₂)₂—NHCO—(CH₂)₃-piperazinylene-(CH₂)₃—; or —(CH₂)₂—NHCO—CH₂-piperazinylene-(CH₂)₂—.

Embodiment (65) relates to the compound of formula (I) or salts, enantiomers, stereoisomers, solvates, or polymorphs thereof according to embodiments (60) or (61), wherein LIN represents:

—(CH₂)₂-piperazinylene-CO—CH₂—O(CH₂)₂—; —(CH₂)₂-piperazinylene-CO—CH₂—OCH₂—; —(CH₂)₂-piperazinylene-CO—CH₂—O(CH₂)₂—OCH₂—; —(CH₂)₂-piperazinylene-CO—(CH₂)₂—O(CH₂)₂—; —(CH₂)₂—piperazinylene-CO—(CH₂)₂—(O(CH₂)₂)₂—; —(CH₂)₂-piperazinylene-CO—(CH₂)₂—(O(CH₂)₂)₃—; —(CH₂)₂-piperazinylene- CO—(CH₂)₂—(O(CH₂)₂)₄—; —(CH₂)₂-piperazinylene-CO—(CH₂)₂—(O(CH₂)₂)₅—; —(CH₂)₂-piperazinylene-CO—(CH₂)₂—(O(CH₂)₂)₆—; —(CH₂)₂-piperazinylene-CO—(CH₂)₂—(O(CH₂)₂)₇—; —(CH₂)₂-piperazinylene-CO—(CH₂)₂—(O(CH₂)₂)₈—; —(CH₂)₂-piperazinylene-CO—(CH₂)₂—(O(CH₂)₂)₉—; or —(CH₂)₂-piperazinylene-CO—(CH₂)₂—(O(CH₂)₂)₁₀—.

Embodiment (66) relates to the compound of formula (I) or salts, enantiomers, stereoisomers, solvates, or polymorphs thereof according to embodiments (60) or (61), wherein LIN represents:

—(CH₂)₂-piperazinylene-CO—CH₂—; —(CH₂)₂-piperazinylene-CO—(CH₂)₂—; —(CH₂)₂-piperazinylene-CO—(CH₂)₃—; —(CH₂)₂-piperazinylene-CO—(CH₂)₄—; —(CH₂)₂-piperazinylene-CO—(CH₂)₅—; —(CH₂)₂-piperazinylene-CO—(CH₂)₆—; —(CH₂)₂-piperazinylene-CO—(CH₂)₇—; —(CH₂)₂-piperazinylene-CO—(CH₂)₈—; —(CH₂)₂-piperazinylene-CO—(CH₂)₉—; —(CH₂)₂-piperazinylene-CO—(CH₂)₁₀—; —(CH₂)₂-piperazinylene-CO—(CH₂)₁₁—; —(CH₂)₂-piperazinylene-CO—(CH₂)₁₂—; —(CH₂)₂-piperazinylene-CO—(CH₂)₁₃—; —(CH₂)₂-piperazinylene-CO—(CH₂)₁₄—; or —(CH₂)₂-piperazinylene-CO—(CH₂)₁₅—.

Embodiment (67) relates to the compound of formula (I) or salts, enantiomers, stereoisomers, solvates, or polymorphs thereof according to embodiment (1), wherein

R₁ represents H, R₂ and R₃ each independently represent OH, and X represents O;

ULM represents the following structure of formula (IV):

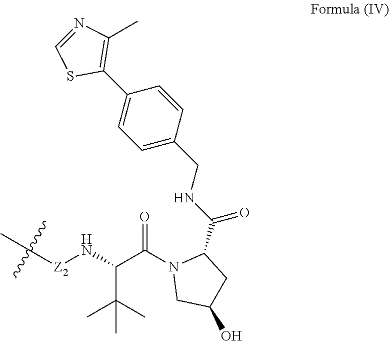

wherein Z₂ represents CO or Z₂ is absent; and
LIN represents alkylene, wherein

the alkylene group is a linear or branched alkylene group interrupted one or more times by the group(s) selected from the group consisting of O, CON(R₄), N(R₅)CO, or any combination thereof, wherein the linear or branched alkylene group is optionally substituted with one or more substituents selected from the group consisting of hydroxyl, amino, mercapto, and halogen; and R₄ and R₅ are each independently selected from the group consisting of H and C₁₋₃ alkyl.

Embodiment (68) relates to the compound of formula (I) or salts, enantiomers, stereoisomers, solvates, or polymorphs thereof according to embodiment (67), wherein:

LIN represents —(CH₂)ₙ₁—N(R₅)CO—(CH₂)ₙ₂—(O(CH₂)ₙ₃)ₘ₁—, or —(CH₂)ₙ₁—N(R₅)CO—(CH₂)ₙ₂—, wherein the hydrogen atom(s) on the carbon atom(s) of backbone carbon chain of the LIN group is/are optionally replaced by one or more substituents selected from the group consisting of hydroxyl, amino, mercapto, and halogen; R₅ is selected from the group consisting of H and C₁₋₃ alkyl; and n1, n2, n3, and ml each independently represent an integer of 1, 2, 3, 4, 5, 6, 7, 8, 9, 10, 11, 12, 13, 14, 15, 16 , 17, 18, 19, or 20.

Embodiment (69) relates to the compound of formula (I) or salts, enantiomers, stereoisomers, solvates, or polymorphs thereof according to embodiments (67) or (68), wherein LIN represents:

—(CH₂)₂—NHCO—(CH₂)₂—O(CH₂)₂—; —(CH₂)₂—NHCO—(CH₂)₂—(O(CH₂)₂)₂—; —(CH₂)₂—NHCO—(CH₂)₂—(O(CH₂)₂)₃—; —(CH₂)₂—NHCO—(CH₂)₂—(O(CH₂)₂)₄—; —(CH₂)₂—NHCO—(CH₂)₂—(O(CH₂)₂)s—; —(CH₂)₂—NHCO—(CH₂)₂—(O(CH₂)₂)₆—; —(CH₂)₂—NHCO—(CH₂)₂—(O(CH₂)₂)₇—; —(CH₂)₂—NHCO—(CH₂)₂—(O(CH₂)₂)₈—; —(CH₂)₂—NHCO—(CH₂)₂—(O(CH₂)₂)₉—; or —(CH₂)₂—NHCO—(CH₂)₂—(O(CH₂)₂)₁₀—.

Embodiment (70) relates to the compound of formula (I) or salts, enantiomers, stereoisomers, solvates, or polymorphs thereof according to embodiments (67) or (68), wherein LIN represents:

—(CH₂)₂—NHCO—CH₂—; —(CH₂)₂—NHCO—(CH₂)₂—; —(CH₂)₂—NHCO—(CH₂)₃—; —(CH₂)₂—NHCO—(CH₂)₄—; —(CH₂)₂—NHCO—(CH₂)₅—; —(CH₂)₂—NHCO—(CH₂)₆—; —(CH₂)₂—NHCO—(CH₂)₇—; —(CH₂)₂—NHCO—(CH₂)₈—; —(CH₂)₂—NHCO—(CH₂)₉—; —(CH₂)₂—NHCO—(CH₂)₁₀—; —(CH₂)₂—NHCO—(CH₂)₁₁—; —(CH₂)₂—NHCO—(CH₂)₁₂—; —(CH₂)₂—NHCO—(CH₂)₁₃—; —(CH₂)₂—NHCO—(CH₂)₁₄—; or —(CH₂)₂—NHCO—(CH₂)₁₅—.

Embodiment (71) relates to the compound of formula (I) or salts, enantiomers, stereoisomers, solvates, or polymorphs thereof according to embodiment (1), wherein

R₁ represents H, R₂ and R₃ each independently represent OH, and X represents O;

ULM represents the following structure of formula (II):

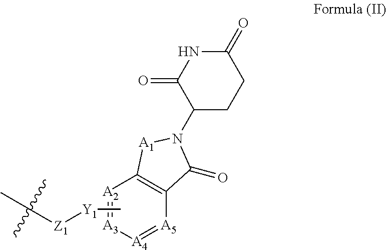

wherein Y₁ represents CH₂, NH, or O; Z₁ represents CO or Z₁ is absent; A₁ represents CH₂ or CO; A₂, A₃, A₄, and A₅ are the same or different and each independently represent CH or N, provided that A₂, A₃, A₄, and A₅ are not N at the same time; and

LIN represents alkylene, wherein
the alkylene group is a linear or branched alkylene group interrupted one or more times by one or more groups selected from the group consisting of CON(R₄), N(R₅)CO, heterocyclylene, heteroarylene, or any combination thereof, wherein the linear or branched alkylene group is optionally substituted with one or more substituents selected from the group consisting of hydroxyl, amino, mercapto, and halogen; R₄ and R₅ are each independently selected from the group consisting of H and C₁₋₃ alkyl; and the heterocyclylene and heteroarylene group are optionally substituted with the substituent(s) selected from the group consisting of C₁₋₃ alkyl, C₁₋₃ alkoxy, cyano, trifluoromethyl, heterocyclyl, halogen, amino, or hydroxyl.

Embodiment (72) relates to the compound of formula (I) or salts, enantiomers, stereoisomers, solvates, or polymorphs thereof according to embodiment (71), wherein LIN is —(CH₂)ₙ₁—N(R₅)CO—(CH₂)ₙ₂—piperazinylene-(CH₂)ₙ₃—, wherein the hydrogen atom(s) on the carbon atom(s) of backbone carbon chain of the LIN group is/are optionally replaced by one or more substituents selected from the group consisting of hydroxyl, amino, mercapto, and halogen; R₅ is selected from the group consisting of H and C₁₋₃ alkyl; and nl, n2, and n3 each independently represent an integer of 1, 2, 3, 4, 5, 6, 7, 8, 9, 10, 11, 12, 13, 14, 15, 16 , 17, 18, 19, or 20, and wherein said piperazinylene group is optionally substituted with the substituent(s) selected from the group consisting of C₁₋₃ alkyl, C₁₋₃ alkoxy, cyano, trifluoromethyl, heterocyclyl, halogen, amino, or hydroxyl.

Embodiment (73) relates to the compound of formula (I) or salts, enantiomers, stereoisomers, solvates, or polymorphs thereof according to embodiment (71), wherein, the ULM represents the following structure of formula (III):

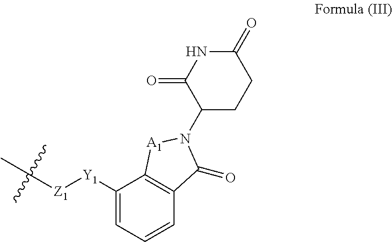

wherein A₁ represents CH₂ or CO; Y₁ represents NH; and Z₁ is absent.

Embodiment (74) relates to the compound of formula (I) or salts, enantiomers, stereoisomers, solvates, or polymorphs thereof according to any one of embodiments (71) to (73), wherein LIN represents:

—(CH₂)₂—NHCO—(CH₂)₂-piperazinylene-(CH₂)₂—; —(CH₂)₂—NHCO—(CH₂)₂-piperazinylene-(CH₂)₃—; —(CH₂)₂—NHCO—(CH₂)₃-piperazinylene-(CH₂)₂—; —(CH₂)₂—NHCO—(CH₂)₃-piperazinylene-(CH₂)₃—; or —(CH₂)₂—NHCO—CH₂-piperazinylene-(CH₂)₂—.

In the general formulas of LIN containing “-phenylene-” in each of the foregoing embodiments, the two chemical moieties of LIN interrupted by “-phenylene-” can attach to the benzene ring of “-phenylene-” in an ortho-, meta- or para-arrangement; and the benzene ring can optionally be substituted by an additional third, fourth, fifth or sixth substituent which are selected from the group consisting of C₁-C₃ alkyl, hydroxyl, amino, mercapto, halogen, C₁₋₃ alkoxy, C₁₋₃ alkylamino, C₁₋₃ haloalkyl, cyano or a combination thereof. In the general formulas of LIN containing “-piperazinylene-” in each of the foregoing embodiments, the two chemical moieties of LIN interrupted by “-piperazinylene-” can respectively attach to two nitrogen atoms of piperazine; and the piperazinylene ring can optionally be substituted by an additional third, fourth, fifth or sixth substituent which are selected from the group consisting of C₁-C₃ alkyl, hydroxyl, amino, mercapto, halogen, C₁₋₃ alkoxy, C₁₋₃ alkylamino, C₁₋₃ haloalkyl, cyano or a combination thereof. In the general formulas of LIN containing cycloalkylene, arylene, heterocyclylene or heteroarylene in each of the foregoing embodiments, the two chemical moieties of LIN interrupted by cycloalkylene, arylene, heterocyclylene or heteroarylene can attach to said cycloalkylene ring, arylene ring, heterocyclylene ring or heteroarylene ring, respectively, in an ortho-, meta- or para-arrangement, wherein said cycloalkylene ring, arylene ring, heterocyclylene ring or heteroarylene ring can optionally be substituted by one or more additional substituents selected from the group consisting of C₁-C₃alkyl, hydroxyl, amino, mercapto, halogen, C₁-C₃alkoxy, C₁-C₃alkylamino, C₁-C₃haloalkyl, cyano or a combination thereof.

Particularly preferred are the following compounds of formula (I) of the present disclosure and salts (especially pharmaceutically acceptable salts), enantiomers, stereoisomers, solvates, or polymorphs thereof in Table 1:

TABLE 1
The compounds of the present invention

| Compound | |
|---|---|
| No. | The compound's name in English |
| SIAIS180001 | (Z)-N-(2-(4-(4-chloro-1,2-diphenylbut-1-en-1-yl)phenoxy)ethyl)-3-(2-((2-(2,6- |
| | dioxopiperidin-3-yl)-1,3-dioxoisoindolin-4-yl)amino)ethoxy)-N- |
| | methylpropanamide |
| SIAIS180002 | (Z)-N-(2-(4-(4-chloro-1,2-diphenylbut-1-en-1-yl)phenoxy)ethyl)-3-(2-(2-((2-(2,6- |
| | dioxopiperidin-3-yl)-1,3-dioxoisoindolin-4-yl)amino)ethoxy)ethoxy)-N- |
| | methylpropanamide |
| SIAIS180004 | (Z)-N-(2-(4-(4-chloro-1,2-diphenylbut-1-en-1-yl)phenoxy)ethyl)-3-(2-(2-(2-((2- |
| | (2,6-dioxopiperidin-3-yl)-1,3-dioxoisoindolin-4-yl)amino)ethoxy)ethoxy)ethoxy)- |
| | N-methylpropanamide |
| SIAIS180006 | (Z)-N-(2-(4-(4-chloro-1,2-diphenylbut-1-en-1-yl)phenoxy)ethyl)-1-((2-(2,6- |
| | dioxopiperidin-3-yl)-1,3-dioxoisoindolin-4-yl)amino)-N-methyl-3,6,9,12- |
| | tetraoxapentadecan-15-amide |
| SIAIS180007 | (Z)-N-(2-(4-(4-chloro-1,2-diphenylbut-1-en-1-yl)phenoxy)ethyl)-1-((2-(2,6- |
| | dioxopiperidin-3-yl)-1,3-dioxoisoindolin-4-yl)amino)-N-methyl-3,6,9,12,15- |
| | pentaoxaoctadecan-18-amide |
| SIAIS180008 | (Z)-N-(2-(4-(4-chloro-1,2-diphenylbut-1-en-1-yl)phenoxy)ethyl)-2-((2-(2,6- |
| | dioxopiperidin-3-yl)-1,3-dioxoisoindolin-4-yl)amino)-N-methylacetamide |
| SIAIS180009 | (Z)-N-(2-(4-(4-chloro-1,2-diphenylbut-1-en-1-yl)phenoxy)ethyl)-3-((2-(2,6- |
| | dioxopiperidin-3-yl)-1,3-dioxoisoindolin-4-yl)amino)-N-methylpropanamide |
| SIAIS180010 | (Z)-N-(2-(4-(4-chloro-1,2-diphenylbut-1-en-1-yl)phenoxy)ethyl)-4-((2-(2,6- |
| | dioxopiperidin-3-yl)-1,3-dioxoisoindolin-4-yl)amino)-N-methylbutanamide |
| SIAIS180011 | (Z)-N-(2-(4-(4-chloro-1,2-diphenylbut-1-en-1-yl)phenoxy)ethyl)-5-((2-(2,6- |
| | dioxopiperidin-3-yl)-1,3-dioxoisoindolin-4-yl)amino)-N-methylpentanamide |
| SIAIS180012 | (Z)-N-(2-(4-(4-chloro-1,2-diphenylbut-1-en-1-yl)phenoxy)ethyl)-6-((2-(2,6- |
| | dioxopiperidin-3-yl)-1,3-dioxoisoindolin-4-yl)amino)-N-methylhexanamide |
| SIAIS180013 | (Z)-N-(2-(4-(4-chloro-1,2-diphenylbut-1-en-1-yl)phenoxy)ethyl)-7-((2-(2,6- |
| | dioxopiperidin-3-yl)-1,3-dioxoisoindolin-4-yl)amino)-N-methylheptanamide |
| | (Z)-N-(2-(4-(4-chloro-1,2-diphenylbut-1-en-1-yl)phenoxy)ethyl)-3-(3-((2-(2,6- |
| | dioxopiperidin-3-yl)-1-oxoisoindolin-4-yl)amino)-3-oxopropoxy)-N- |
| | methylpropanamide |
| | (Z)-N-(2-(4-(4-chloro-1,2-diphenylbut-1-en-1-yl)phenoxy)ethyl)-3-(2-(3-((2-(2,6- |
| | dioxopiperidin-3-yl)-1-oxoisoindolin-4-yl)amino)-3-oxopropoxy)ethoxy)-N- |
| | methylpropanamide |
| | (Z)-N-(2-(4-(4-chloro-1,2-diphenylbut-1-en-1-yl)phenoxy)ethyl)-3-(2-(2-(3-((2- |
| | (2,6-dioxopiperidin-3-yl)-1-oxoisoindolin-4-yl)amino)-3- |
| | oxopropoxy)ethoxy)ethoxy)-N-methylpropanamide |
| | (Z)-N1-(2-(4-(4-chloro-1,2-diphenylbut-1-en-1-yl)phenoxy)ethyl)-N16-(2-(2,6- |
| | dioxopiperidin-3-yl)-1-oxoisoindolin-4-yl)-N1-methyl-4,7,10,13- |
| | tetraoxahexadecanediamide |
| | (Z)-N1-(2-(4-(4-chloro-1,2-diphenylbut-1-en-1-yl)phenoxy)ethyl)-N19-(2-(2,6- |
| | dioxopiperidin-3-yl)-1-oxoisoindolin-4-yl)-N1-methyl-4,7,10,13,16- |
| | pentaoxanonadecanediamide |
| | (Z)-N-(2-(4-(4-chloro-1,2-diphenylbut-1-en-1-yl)phenoxy)ethyl)-3-(2-((2-(2,6- |
| | dioxopiperidin-3-yl)-1-oxoisoindolin-4-yl)amino)ethoxy)-N-methylpropanamide |
| | (Z)-N-(2-(4-(4-chloro-1,2-diphenylbut-1-en-1-yl)phenoxy)ethyl)-3-(2-(2-((2-(2,6- |
| | dioxopiperidin-3-yl)-1-oxoisoindolin-4-yl)amino)ethoxy)ethoxy)-N- |
| | methylpropanamide |
| | (Z)-N-(2-(4-(4-chloro-1,2-diphenylbut-1-en-1-yl)phenoxy)ethyl)-3-(2-(2-(2-((2- |
| | (2,6-dioxopiperidin-3-yl)-1-oxoisoindolin-4-yl)amino)ethoxy)ethoxy)ethoxy)-N- |
| | methylpropanamide |
| | (Z)-N-(2-(4-(4-chloro-1,2-diphenylbut-1-en-1-yl)phenoxy)ethyl)-1-((2-(2,6- |
| | dioxopiperidin-3-yl)-1-oxoisoindolin-4-yl)amino)-N-methyl-3,6,9,12- |
| | tetraoxapentadecan-15-amide |
| | (Z)-N-(2-(4-(4-chloro-1,2-diphenylbut-1-en-1-yl)phenoxy)ethyl)-1-((2-(2,6- |
| | dioxopiperidin-3-yl)-1-oxoisoindolin-4-yl)amino)-N-methyl-3,6,9,12,15- |
| | pentaoxaoctadecan-18-amide |
| SIAIS180090 | (Z)-N1-(2-(4-(4-chloro-1,2-diphenylbut-1-en-1-yl)phenoxy)ethyl)-N3-(2-(2,6- |
| | dioxopiperidin-3-yl)-1-oxoisoindolin-4-yl)-N1-methylmalonamide |
| SIAIS180091 | (Z)-N1-(2-(4-(4-chloro-1,2-diphenylbut-1-en-1-yl)phenoxy)ethyl)-N4-(2-(2,6- |
| | dioxopiperidin-3-yl)-1-oxoisoindolin-4-yl)-N1-methylsuccinamide |
| SIAIS180092 | (Z)-N1-(2-(4-(4-chloro-1,2-diphenylbut-1-en-1-yl)phenoxy)ethyl)-N5-(2-(2,6- |
| | dioxopiperidin-3-yl)-1-oxoisoindolin-4-yl)-N1-methylglutaramide |
| SIAIS180093 | (Z)-N1-(2-(4-(4-chloro-1,2-diphenylbut-1-en-1-yl)phenoxy)ethyl)-N6-(2-(2,6- |
| | dioxopiperidin-3-yl)-1-oxoisoindolin-4-yl)-N1-methyladipamide |
| SIAIS180094 | (Z)-N1-(2-(4-(4-chloro-1,2-diphenylbut-1-en-1-yl)phenoxy)ethyl)-N7-(2-(2,6- |
| | dioxopiperidin-3-yl)-1-oxoisoindolin-4-yl)-N1-methylheptanediamide |
| | (Z)-N1-(2-(4-(4-chloro-1,2-diphenylbut-1-en-1-yl)phenoxy)ethyl)-N8-(2-(2,6- |
| | dioxopiperidin-3-yl)-1-oxoisoindolin-4-yl)-N1-methyloctanediamide |
| | (Z)-N-(2-(4-(4-chloro-1,2-diphenylbut-1-en-1-yl)phenoxy)ethyl)-3-((2-(2,6- |
| | dioxopiperidin-3-yl)-1-oxoisoindolin-4-yl)amino)-N-methylpropanamide |
| | (Z)-N-(2-(4-(4-chloro-1,2-diphenylbut-1-en-1-yl)phenoxy)ethyl)-4-((2-(2,6- |
| | dioxopiperidin-3-yl)-1-oxoisoindolin-4-yl)amino)-N-methylbutanamide |
| | (Z)-N-(2-(4-(4-chloro-1,2-diphenylbut-1-en-1-yl)phenoxy)ethyl)-5-((2-(2,6- |
| | dioxopiperidin-3-yl)-1-oxoisoindolin-4-yl)amino)-N-methylpentanamide |
| | (Z)-N-(2-(4-(4-chloro-1,2-diphenylbut-1-en-1-yl)phenoxy)ethyl)-6-((2-(2,6- |
| | dioxopiperidin-3-yl)-1-oxoisoindolin-4-yl)amino)-N-methylhexanamide |
| | (Z)-N-(2-(4-(4-chloro-1,2-diphenylbut-1-en-1-yl)phenoxy)ethyl)-7-((2-(2,6- |
| | dioxopiperidin-3-yl)-1-oxoisoindolin-4-yl)amino)-N-methylheptanamide |
| | (Z)-N-(2-(4-(4-chloro-1,2-diphenylbut-1-en-1-yl)phenoxy)ethyl)-8-((2-(2,6- |
| | dioxopiperidin-3-yl)-1-oxoisoindolin-4-yl)amino)-N-methyloctanamide |
| SIAIS180039 | (2S,4R)-1-((S)-2-(tert-butyl)-14-(4-((Z)-4-chloro-1,2-diphenylbut-1-en-1- |
| | yl)phenoxy)-12-methyl-4,11-dioxo-6,9-dioxa-3,12-diazatetradecanoyl)-4-hydroxy- |
| | N-(4-(4-methylthiazol-5-yl)benzyl)pyrrolidine-2-carboxamide |
| SIAIS180023 | (2S,4R)-1-((S)-2-(tert-butyl)-16-(4-((Z)-4-chloro-1,2-diphenylbut-1-en-1- |
| | yl)phenoxy)-14-methyl-4,13-dioxo-7,10-dioxa-3,14-diazahexadecanoyl)-4- |
| | hydroxy-N-(4-(4-methylthiazol-5-yl)benzyl)pyrrolidine-2-carboxamide |
| SIAIS180024 | (2S,4R)-1-((S)-2-(tert-butyl)-19-(4-((Z)-4-chloro-1,2-diphenylbut-1-en-1- |
| | yl)phenoxy)-17-methyl-4,16-dioxo-7,10,13-trioxa-3,17-diazanonadecanoyl)-4- |
| | hydroxy-N-(4-(4-methylthiazol-5-yl)benzyl)pyrrolidine-2-carboxamide |
| SIAIS180025 | N1-(2-(4-((Z)-4-chloro-1,2-diphenylbut-1-en-1-yl)phenoxy)ethyl)-N16-((S)-1- |
| | ((2S,4R)-4-hydroxy-2-((4-(4-methylthiazol-5-yl)benzyl)carbamoyl)pyrrolidin-1- |
| | yl)-3,3-dimethyl-1-oxobutan-2-yl)-N1-methyl-4,7,10,13- |
| | tetraoxahexadecanediamide |
| SIAIS180022 | N1-(2-(4-((Z)-4-chloro-1,2-diphenylbut-1-en-1-yl)phenoxy)ethyl)-N19-((S)-1- |
| | ((2S,4R)-4-hydroxy-2-((4-(4-methylthiazol-5-yl)benzyl)carbamoyl)pyrrolidin-1- |
| | yl)-3,3-dimethyl-1-oxobutan-2-yl)-N1-methyl-4,7,10,13,16- |
| | pentaoxanonadecanediamide |
| SIAIS180026 | N1-(2-(4-((Z)-4-chloro-1,2-diphenylbut-1-en-1-yl)phenoxy)ethyl)-N4-((S)-1- |
| | ((2S,4R)-4-hydroxy-2-((4-(4-methylthiazol-5-yl)benzyl)carbamoyl)pyrrolidin-1- |
| | yl)-3,3-dimethyl-1-oxobutan-2-yl)-N1-methylsuccinamide |
| SIAIS180027 | N1-(2-(4-((Z)-4-chloro-1,2-diphenylbut-1-en-1-yl)phenoxy)ethyl)-N5-((S)-1- |
| | ((2S,4R)-4-hydroxy-2-((4-(4-methylthiazol-5-yl)benzyl)carbamoyl)pyrrolidin-1- |
| | yl)-3,3-dimethyl-1-oxobutan-2-yl)-N1-methylglutaramide |
| SIAIS180028 | N1-(2-(4-((Z)-4-chloro-1,2-diphenylbut-1-en-1-yl)phenoxy)ethyl)-N6-((S)-1- |
| | ((2S,4R)-4-hydroxy-2-((4-(4-methylthiazol-5-yl)benzyl)carbamoyl)pyrrolidin-1- |
| | yl)-3,3-dimethyl-1-oxobutan-2-yl)-N1-methyladipamide |
| SIAIS180029 | N1-(2-(4-((Z)-4-chloro-1,2-diphenylbut-1-en-1-yl)phenoxy)ethyl)-N7-((S)-1- |
| | ((2S,4R)-4-hydroxy-2-((4-(4-methylthiazol-5-yl)benzyl)carbamoyl)pyrrolidin-1- |
| | yl)-3,3-dimethyl-1-oxobutan-2-yl)-N1-methylheptanediamide |
| SIAIS180033 | N1-(2-(4-((Z)-4-chloro-1,2-diphenylbut-1-en-1-yl)phenoxy)ethyl)-N8-((S)-1- |
| | ((2S,4R)-4-hydroxy-2-((4-(4-methylthiazol-5-yl)benzyl)carbamoyl)pyrrolidin-1- |
| | yl)-3,3-dimethyl-1-oxobutan-2-yl)-N1-methyloctanediamide |
| SIAIS180035 | N1-(2-(4-((Z)-4-chloro-1,2-diphenylbut-1-en-1-yl)phenoxy)ethyl)-N9-((S)-1- |
| | ((2S,4R)-4-hydroxy-2-((4-(4-methylthiazol-5-yl)benzyl)carbamoyl)pyrrolidin-1- |
| | yl)-3,3-dimethyl-1-oxobutan-2-yl)-N1-methylnonanediamide |
| SIAIS180036 | N1-(2-(4-((Z)-4-chloro-1,2-diphenylbut-1-en-1-yl)phenoxy)ethyl)-N10-((S)-1- |
| | ((2S,4R)-4-hydroxy-2-((4-(4-methylthiazol-5-yl)benzyl)carbamoyl)pyrrolidin-1- |
| | yl)-3,3-dimethyl-1-oxobutan-2-yl)-N1-methyldecanediamide |
| SIAIS208105 | (2S,4R)-1-((S)-2-(3-(4-(3-((2-(4-((Z)-4-chloro-1,2-diphenylbut-1-en-1- |
| | yl)phenoxy)ethyl)(methyl)amino)-3-oxopropyl)piperazin-1-yl)propanamido)-3,3- |
| | dimethylbutanoyl)-4-hydroxy-N-(4-(4-methylthiazol-5-yl)benzyl)pyrrolidine-2- |
| | carboxamide |
| SIAIS208125 | (2S,4R)-1-((S)-2-(3-(4-(3-((2-(4-((Z)-4-chloro-1,2-diphenylbut-1-en-1- |
| | yl)phenoxy)ethyl)(methyl)amino)-3-oxopropyl)phenyl)propanamido)-3,3- |
| | dimethylbutanoyl)-4-hydroxy-N-(4-(4-methylthiazol-5-yl)benzyl)pyrrolidine-2- |
| | carboxamide |
| SIAIS208135 | (Z)-N-(2-(4-(4-chloro-1,2-diphenylbut-1-en-1-yl)phenoxy)ethyl)-3-(4-(2-((2-(2,6- |
| | dioxopiperidin-3-yl)-1,3-dioxoisoindolin-4-yl)amino)ethyl)piperazin-1-yl)-N- |
| | methylpropanamide |
| | (Z)-N-(2-(4-(4-chloro-1,2-diphenylbut-1-en-1-yl)phenoxy)ethyl)-3-(4-(2-((2-(2,6- |
| | dioxopiperidin-3-yl)-1,3-dioxoisoindolin-4-yl)amino)ethyl)phenyl)-N- |
| | methylpropanamide |
| | (2S,4R)-1-((S)-2-(7-((2-(4-((Z)-4-chloro-1,2-diphenylbut-1-en-1- |
| | yl)phenoxy)ethyl)(methyl)amino)heptanamido)-3,3-dimethylbutanoyl)-4-hydroxy- |
| | N-(4-(4-methylthiazol-5-yl)benzyl)pyrrolidine-2-carboxamide |
| | (2S,4R)-1-((S)-2-((7-((2-(4-((Z)-4-chloro-1,2-diphenylbut-1-en-1- |
| | yl)phenoxy)ethyl)(methyl)amino)-7-oxoheptyl)amino)-3,3-dimethylbutanoyl)-4- |
| | hydroxy-N-(4-(4-methylthiazol-5-yl)benzyl)pyrrolidine-2-carboxamide |
| | (2S,4R)-1-((S)-2-((7-((2-(4-((Z)-4-chloro-1,2-diphenylbut-1-en-1- |
| | yl)phenoxy)ethyl)(methyl)amino)heptyl)amino)-3,3-dimethylbutanoyl)-4-hydroxy- |
| | N-(4-(4-methylthiazol-5-yl)benzyl)pyrrolidine-2-carboxamide |
| | (Z)-4-((2-(3-(4-(2-(4-(4-chloro-1,2-diphenylbut-1-en-1- |
| | yl)phenoxy)ethyl)piperazin-1-yl)-3-oxopropoxy)ethyl)amino)-2-(2,6- |
| | dioxopiperidin-3-yl)isoindoline-1,3-dione |
| | (Z)-4-((2-(2-(3-(4-(2-(4-(4-chloro-1,2-diphenylbut-1-en-1- |
| | yl)phenoxy)ethyl)piperazin-1-yl)-3-oxopropoxy)ethoxy)ethyl)amino)-2-(2,6- |
| | dioxopiperidin-3-yl)isoindoline-1,3-dione |
| | (Z)-4-((2-(2-(2-(3-(4-(2-(4-(4-chloro-1,2-diphenylbut-1-en-1- |
| | yl)phenoxy)ethyl)piperazin-1-yl)-3-oxopropoxy)ethoxy)ethoxy)ethyl)amino)-2- |
| | (2,6-dioxopiperidin-3-yl)isoindoline-1,3-dione |
| | (Z)-4-((15-(4-(2-(4-(4-chloro-1,2-diphenylbut-1-en-1-yl)phenoxy)ethyl)piperazin- |
| | 1-yl)-15-oxo-3,6,9,12-tetraoxapentadecyl)amino)-2-(2,6-dioxopiperidin-3- |
| | yl)isoindoline-1,3-dione |
| | (Z)-4-((18-(4-(2-(4-(4-chloro-1,2-diphenylbut-1-en-1-yl)phenoxy)ethyl)piperazin- |
| | 1-yl)-18-oxo-3,6,9,12,15-pentaoxaoctadecyl)amino)-2-(2,6-dioxopiperidin-3- |
| | yl)isoindoline-1,3-dione |
| | (Z)-4-((2-(4-(2-(4-(4-chloro-1,2-diphenylbut-1-en-1-yl)phenoxy)ethyl)piperazin-1- |
| | yl)-2-oxoethyl)amino)-2-(2,6-dioxopiperidin-3-yl)isoindoline-1,3-dione |
| | (Z)-4-((3-(4-(2-(4-(4-chloro-1,2-diphenylbut-1-en-1-yl)phenoxy)ethyl)piperazin-1- |
| | yl)-3-oxopropyl)amino)-2-(2,6-dioxopiperidin-3-yl)isoindoline-1,3-dione |
| | (Z)-4-((4-(4-(2-(4-(4-chloro-1,2-diphenylbut-1-en-1-yl)phenoxy)ethyl)piperazin-1- |
| | yl)-4-oxobutyl)amino)-2-(2,6-dioxopiperidin-3-yl)isoindoline-1,3-dione |
| | (Z)-4-((5-(4-(2-(4-(4-chloro-1,2-diphenylbut-1-en-1-yl)phenoxy)ethyl)piperazin-1- |
| | yl)-5-oxopentyl)amino)-2-(2,6-dioxopiperidin-3-yl)isoindoline-1,3-dione |
| | (Z)-4-((6-(4-(2-(4-(4-chloro-1,2-diphenylbut-1-en-1-yl)phenoxy)ethyl)piperazin-1- |
| | yl)-6-oxohexyl)amino)-2-(2,6-dioxopiperidin-3-yl)isoindoline-1,3-dione |
| | (Z)-4-((7-(4-(2-(4-(4-chloro-1,2-diphenylbut-1-en-1-yl)phenoxy)ethyl)piperazin-1- |
| | yl)-7-oxoheptyl)amino)-2-(2,6-dioxopiperidin-3-yl)isoindoline-1,3-dione |
| | (Z)-3-(3-(4-(2-(4-(4-chloro-1,2-diphenylbut-1-en-1-yl)phenoxy)ethyl)piperazin-1- |
| | yl)-3-oxopropoxy)-N-(2-(2,6-dioxopiperidin-3-yl)-1-oxoisoindolin-4- |
| | yl)propanamide |
| | (Z)-3-(2-(3-(4-(2-(4-(4-chloro-1,2-diphenylbut-1-en-1-yl)phenoxy)ethyl)piperazin- |
| | 1-yl)-3-oxopropoxy)ethoxy)-N-(2-(2,6-dioxopiperidin-3-yl)-1-oxoisoindolin-4- |
| | yl)propanamide |
| | (Z)-3-(2-(2-(3-(4-(2-(4-(4-chloro-1,2-diphenylbut-1-en-1- |
| | yl)phenoxy)ethyl)piperazin-1-yl)-3-oxopropoxy)ethoxy)ethoxy)-N-(2-(2,6- |
| | dioxopiperidin-3-yl)-1-oxoisoindolin-4-yl)propanamide |
| | (Z)-16-(4-(2-(4-(4-chloro-1,2-diphenylbut-1-en-1-yl)phenoxy)ethyl)piperazin-1- |
| | yl)-N-(2-(2,6-dioxopiperidin-3-yl)-1-oxoisoindolin-4-yl)-16-oxo-4,7,10,13- |
| | tetraoxahexadecanamide |
| | (Z)-19-(4-(2-(4-(4-chloro-1,2-diphenylbut-1-en-1-yl)phenoxy)ethyl)piperazin-1- |
| | yl)-N-(2-(2,6-dioxopiperidin-3-yl)-1-oxoisoindolin-4-yl)-19-oxo-4,7,10,13,16- |
| | pentaoxanonadecanamide |
| | (Z)-3-(4-((2-(3-(4-(2-(4-(4-chloro-1,2-diphenylbut-1-en-1- |
| | yl)phenoxy)ethyl)piperazin-1-yl)-3-oxopropoxy)ethyl)amino)-1-oxoisoindolin-2- |
| | yl)piperidine-2,6-dione |
| | (Z)-3-(4-((2-(2-(3-(4-(2-(4-(4-chloro-1,2-diphenylbut-1-en-1- |
| | yl)phenoxy)ethyl)piperazin-1-yl)-3-oxopropoxy)ethoxy)ethyl)amino)-1- |
| | oxoisoindolin-2-yl)piperidine-2,6-dione |
| | (Z)-3-(4-((2-(2-(2-(3-(4-(2-(4-(4-chloro-1,2-diphenylbut-1-en-1- |
| | yl)phenoxy)ethyl)piperazin-1-yl)-3-oxopropoxy)ethoxy)ethoxy)ethyl)amino)-1- |
| | oxoisoindolin-2-yl)piperidine-2,6-dione |
| | (Z)-3-(4-((15-(4-(2-(4-(4-chloro-1,2-diphenylbut-1-en-1- |
| | yl)phenoxy)ethyl)piperazin-1-yl)-15-oxo-3,6,9,12-tetraoxapentadecyl)amino)-1- |
| | oxoisoindolin-2-yl)piperidine-2,6-dione |
| | (Z)-3-(4-((18-(4-(2-(4-(4-chloro-1,2-diphenylbut-1-en-1- |
| | yl)phenoxy)ethyl)piperazin-1-yl)-18-oxo-3,6,9,12,15-pentaoxaoctadecyl)amino)-1- |
| | oxoisoindolin-2-yl)piperidine-2,6-dione |
| | (Z)-3-(4-(2-(4-(4-chloro-1,2-diphenylbut-1-en-1-yl)phenoxy)ethyl)piperazin-1-yl)- |
| | N-(2-(2,6-dioxopiperidin-3-yl)-1-oxoisoindolin-4-yl)-3-oxopropanamide |
| | (Z)-4-(4-(2-(4-(4-chloro-1,2-diphenylbut-1-en-1-yl)phenoxy)ethyl)piperazin-1-yl)- |
| | N-(2-(2,6-dioxopiperidin-3-yl)-1-oxoisoindolin-4-yl)-4-oxobutanamide |
| | (Z)-5-(4-(2-(4-(4-chloro-1,2-diphenylbut-1-en-1-yl)phenoxy)ethyl)piperazin-1-yl)- |
| | N-(2-(2,6-dioxopiperidin-3-yl)-1-oxoisoindolin-4-yl)-5-oxopentanamide |
| | (Z)-6-(4-(2-(4-(4-chloro-1,2-diphenylbut-1-en-1-yl)phenoxy)ethyl)piperazin-1-yl)- |
| | N-(2-(2,6-dioxopiperidin-3-yl)-1-oxoisoindolin-4-yl)-6-oxohexanamide |
| | (Z)-7-(4-(2-(4-(4-chloro-1,2-diphenylbut-1-en-1-yl)phenoxy)ethyl)piperazin-1-yl)- |
| | N-(2-(2,6-dioxopiperidin-3-yl)-1-oxoisoindolin-4-yl)-7-oxoheptanamide |
| | (Z)-3-(4-((2-(4-(2-(4-(4-chloro-1,2-diphenylbut-1-en-1- |
| | yl)phenoxy)ethyl)piperazin-1-yl)-2-oxoethyl)amino)-1-oxoisoindolin-2- |
| | yl)piperidine-2,6-dione |
| | (Z)-3-(4-((3-(4-(2-(4-(4-chloro-1,2-diphenylbut-1-en-1- |
| | yl)phenoxy)ethyl)piperazin-1-yl)-3-oxopropyl)amino)-1-oxoisoindolin-2- |
| | yl)piperidine-2,6-dione |
| | (Z)-3-(4-((4-(4-(2-(4-(4-chloro-1,2-diphenylbut-1-en-1- |
| | yl)phenoxy)ethyl)piperazin-1-yl)-4-oxobutyl)amino)-1-oxoisoindolin-2- |
| | yl)piperidine-2,6-dione |
| | (Z)-3-(4-((5-(4-(2-(4-(4-chloro-1,2-diphenylbut-1-en-1- |
| | yl)phenoxy)ethyl)piperazin-1-yl)-5-oxopentyl)amino)-1-oxoisoindolin-2- |
| | yl)piperidine-2,6-dione |
| | (Z)-3-(4-((6-(4-(2-(4-(4-chloro-1,2-diphenylbut-1-en-1- |
| | yl)phenoxy)ethyl)piperazin-1-yl)-6-oxohexyl)amino)-1-oxoisoindolin-2- |
| | yl)piperidine-2,6-dione |
| | (Z)-3-(4-((7-(4-(2-(4-(4-chloro-1,2-diphenylbut-1-en-1- |
| | yl)phenoxy)ethyl)piperazin-1-yl)-7-oxoheptyl)amino)-1-oxoisoindolin-2- |
| | yl)piperidine-2,6-dione |
| | (2S,4R)-1-((S)-2-(2-(2-(2-(4-(2-(4-((Z)-4-chloro-1,2-diphenylbut-1-en-1- |
| | yl)phenoxy)ethyl)piperazin-1-yl)-2-oxoethoxy)ethoxy)acetamido)-3,3- |
| | dimethylbutanoyl)-4-hydroxy-N-(4-(4-methylthiazol-5-yl)benzyl)pyrrolidine-2- |
| | carboxamide |
| | (2S,4R)-1-((S)-2-(3-(2-(3-(4-(2-(4-((Z)-4-chloro-1,2-diphenylbut-1-en-1- |
| | yl)phenoxy)ethyl)piperazin-1-yl)-3-oxopropoxy)ethoxy)propanamido)-3,3- |
| | dimethylbutanoyl)-4-hydroxy-N-(4-(4-methylthiazol-5-yl)benzyl)pyrrolidine-2- |
| | carboxamide |
| | (2S,4R)-1-((S)-2-(tert-butyl)-16-(4-(2-(4-((Z)-4-chloro-1,2-diphenylbut-1-en-1- |
| | yl)phenoxy)ethyl)piperazin-1-yl)-4,16-dioxo-7,10,13-trioxa-3-azahexadecanoyl)-4- |
| | hydroxy-N-(4-(4-methylthiazol-5-yl)benzyl)pyrrolidine-2-carboxamide |
| | (2S,4R)-1-((S)-2-(tert-butyl)-19-(4-(2-(4-((Z)-4-chloro-1,2-diphenylbut-1-en-1- |
| | yl)phenoxy)ethyl)piperazin-1-yl)-4,19-dioxo-7,10,13,16-tetraoxa-3- |
| | azanonadecanoyl)-4-hydroxy-N-(4-(4-methylthiazol-5-yl)benzyl)pyrrolidine-2- |
| | carboxamide |
| | (2S,4R)-1-((S)-2-(tert-butyl)-22-(4-(2-(4-((Z)-4-chloro-1,2-diphenylbut-1-en-1- |
| | yl)phenoxy)ethyl)piperazin-1-yl)-4,22-dioxo-7,10,13,16,19-pentaoxa-3- |
| | azadocosanoyl)-4-hydroxy-N-(4-(4-methylthiazol-5-yl)benzyl)pyrrolidine-2- |
| | carboxamide |
| | (2S,4R)-1-((S)-2-(4-(4-(2-(4-((Z)-4-chloro-1,2-diphenylbut-1-en-1- |
| | yl)phenoxy)ethyl)piperazin-1-yl)-4-oxobutanamido)-3,3-dimethylbutanoyl)-4- |
| | hydroxy-N-(4-(4-methylthiazol-5-yl)benzyl)pyrrolidine-2-carboxamide |
| | (2S,4R)-1-((S)-2-(5-(4-(2-(4-((Z)-4-chloro-1,2-diphenylbut-1-en-1- |
| | yl)phenoxy)ethyl)piperazin-1-yl)-5-oxopentanamido)-3,3-dimethylbutanoyl)-4- |
| | hydroxy-N-(4-(4-methylthiazol-5-yl)benzyl)pyrrolidine-2-carboxamide |
| | (2S,4R)-1-((S)-2-(6-(4-(2-(4-((Z)-4-chloro-1,2-diphenylbut-1-en-1- |
| | yl)phenoxy)ethyl)piperazin-1-yl)-6-oxohexanamido)-3,3-dimethylbutanoyl)-4- |
| | hydroxy-N-(4-(4-methylthiazol-5-yl)benzyl)pyrrolidine-2-carboxamide |
| | (2S,4R)-1-((S)-2-(7-(4-(2-(4-((Z)-4-chloro-1,2-diphenylbut-1-en-1- |
| | yl)phenoxy)ethyl)piperazin-1-yl)-7-oxoheptanamido)-3,3-dimethylbutanoyl)-4- |
| | hydroxy-N-(4-(4-methylthiazol-5-yl)benzyl)pyrrolidine-2-carboxamide |
| | (2S,4R)-1-((S)-2-(8-(4-(2-(4-((Z)-4-chloro-1,2-diphenylbut-1-en-1- |
| | yl)phenoxy)ethyl)piperazin-1-yl)-8-oxooctanamido)-3,3-dimethylbutanoyl)-4- |
| | hydroxy-N-(4-(4-methylthiazol-5-yl)benzyl)pyrrolidine-2-carboxamide |
| | (2S,4R)-1-((S)-2-(9-(4-(2-(4-((Z)-4-chloro-1,2-diphenylbut-1-en-1- |
| | yl)phenoxy)ethyl)piperazin-1-yl)-9-oxononanamido)-3,3-dimethylbutanoyl)-4- |
| | hydroxy-N-(4-(4-methylthiazol-5-yl)benzyl)pyrrolidine-2-carboxamide |
| | (2S,4R)-1-((S)-2-(10-(4-(2-(4-((Z)-4-chloro-1,2-diphenylbut-1-en-1- |
| | yl)phenoxy)ethyl)piperazin-1-yl)-10-oxodecanamido)-3,3-dimethylbutanoyl)-4- |
| | hydroxy-N-(4-(4-methylthiazol-5-yl)benzyl)pyrrolidine-2-carboxamide |
| | (2S,4R)-1-((S)-2-(7-(4-(2-(4-((Z)-4-chloro-1,2-diphenylbut-1-en-1- |
| | yl)phenoxy)ethyl)piperazin-1-yl)heptanamido)-3,3-dimethylbutanoyl)-4-hydroxy- |
| | N-(4-(4-methylthiazol-5-yl)benzyl)pyrrolidine-2-carboxamide |
| | (2S,4R)-1-((S)-2-((7-(4-(2-(4-((Z)-4-chloro-1,2-diphenylbut-1-en-1- |
| | yl)phenoxy)ethyl)piperazin-1-yl)-7-oxoheptyl)amino)-3,3-dimethylbutanoyl)-4- |
| | hydroxy-N-(4-(4-methylthiazol-5-yl)benzyl)pyrrolidine-2-carboxamide |
| | (2S,4R)-1-((S)-2-((7-(4-(2-(4-((Z)-4-chloro-1,2-diphenylbut-1-en-1- |
| | yl)phenoxy)ethyl)piperazin-1-yl)heptyl)amino)-3,3-dimethylbutanoyl)-4-hydroxy- |
| | N-(4-(4-methylthiazol-5-yl)benzyl)pyrrolidine-2-carboxamide |
| SIAIS208138 | (N-(2-(4-(4-chloro-1-(4-hydroxyphenyl)-2-phenylbut-1-en-1-yl)phenoxy)ethyl)-3- |
| | (2-((2-(2,6-dioxopiperidin-3-yl)-1,3-dioxoisoindolin-4- |
| | yl)amino)ethoxy)propanamide |
| SIAIS208139 | N-(2-(4-(4-chloro-1-(4-hydroxyphenyl)-2-phenylbut-1-en-1-yl)phenoxy)ethyl)-3- |
| | (2-(2-((2-(2,6-dioxopiperidin-3-yl)-1,3-dioxoisoindolin-4- |
| | yl)amino)ethoxy)ethoxy)propanamide |
| SIAIS208140 | N-(2-(4-(4-chloro-1-(4-hydroxyphenvl)-2-phenylbut-1-en-1-yl)phenoxy)ethyl)-3- |
| | (2-(2-(2-((2-(2,6-dioxopiperidin-3-yl)-1,3-dioxoisoindolin-4- |
| | yl)amino)ethoxy)ethoxy)ethoxy)propanamide |
| SIAIS208141 | N-(2-(4-(4-chloro-1-(4-hydroxyphenyl)-2-phenylbut-1-en-1-yl)phenoxy)ethyl)-1- |
| | ((2-(2,6-dioxopiperidin-3-yl)-1,3-dioxoisoindolin-4-yl)amino)-3,6,9,12- |
| | tetraoxapentadecan-15-amide |
| | N-(2-(4-(4-chloro-1-(4-hydroxyphenyl)-2-phenylbut-1-en-1-yl)phenoxy)ethyl)-1- |
| | ((2-(2,6-dioxopiperidin-3-yl)-1,3-dioxoisoindolin-4-yl)amino)-3,6,9,12,15- |
| | pentaoxaoctadecan-18-amide |
| SIAIS208142 | N-(2-(4-(4-chloro-1-(4-hydroxyphenyl)-2-phenylbut-1-en-1-yl)phenoxy)ethyl)-2- |
| | ((2-(2,6-dioxopiperidin-3-yl)-1,3-dioxoisoindolin-4-yl)amino)acetamide |
| SIAIS208143 | N-(2-(4-(4-chloro-1-(4-hydroxyphenyl)-2-phenylbut-1-en-1-yl)phenoxy)ethyl)-3- |
| | ((2-(2,6-dioxopiperidin-3-yl)-1,3-dioxoisoindolin-4-yl)amino)propanamide |
| | N-(2-(4-(4-chloro-1-(4-hydroxyphenyl)-2-phenylbut-1-en-1-yl)phenoxy)ethyl)-4- |
| | ((2-(2,6-dioxopiperidin-3-yl)-1,3-dioxoisoindolin-4-yl)amino)butanamide |
| SIAIS208144 | N-(2-(4-(4-chloro-1-(4-hydroxyphenyl)-2-phenylbut-1-en-1-yl)phenoxy)ethyl)-5- |
| | ((2-(2,6-dioxopiperidin-3-yl)-1,3-dioxoisoindolin-4-yl)amino)pentanamide |
| | N-(2-(4-(4-chloro-1-(4-hydroxyphenyl)-2-phenylbut-1-en-1-yl)phenoxy)ethyl)-6- |
| | ((2-(2,6-dioxopiperidin-3-yl)-1,3-dioxoisoindolin-4-yl)amino)hexanamide |
| SIAIS208145 | N-(2-(4-(4-chloro-1-(4-hydroxyphenyl)-2-phenylbut-1-en-1-yl)phenoxy)ethyl)-7- |
| | ((2-(2,6-dioxopiperidin-3-yl)-1,3-dioxoisoindolin-4-yl)amino)heptanamide |
| | N-(2-(4-(4-chloro-1-(4-hydroxyphenyl)-2-phenylbut-1-en-1-yl)phenoxy)ethyl)-3- |
| | (3-((2-(2,6-dioxopiperidin-3-yl)-1-oxoisoindolin-4-yl)amino)-3- |
| | oxopropoxy)propanamide |
| | N-(2-(4-(4-chloro-1-(4-hydroxyphenyl)-2-phenylbut-1-en-1-yl)phenoxy)ethyl)-3- |
| | (2-(3-((2-(2,6-dioxopiperidin-3-yl)-1-oxoisoindolin-4-yl)amino)-3- |
| | oxopropoxy)ethoxy)propanamide |
| | N-(2-(4-(4-chloro-1-(4-hydroxyphenyl)-2-phenylbut-1-en-1-yl)phenoxy)ethyl)-3- |
| | (2-(2-(3-((2-(2,6-dioxopiperidin-3-yl)-1-oxoisoindolin-4-yl)amino)-3- |
| | oxopropoxy)ethoxy)ethoxy)propanamide |
| | N1-(2-(4-(4-chloro-1-(4-hydroxyphenyl)-2-phenylbut-1-en-1-yl)phenoxy)ethyl)- |
| | N16-(2-(2,6-dioxopiperidin-3-yl)-1-oxoisoindolin-4-yl)-4,7,10,13- |
| | tetraoxahexadecanediamide |
| | N1-(2-(4-(4-chloro-1-(4-hydroxyphenyl)-2-phenylbut-1-en-1-yl)phenoxy)ethyl)- |
| | N19-(2-(2,6-dioxopiperidin-3-yl)-1-oxoisoindolin-4-yl)-4,7,10,13,16- |
| | pentaoxanonadecanediamide |
| | N-(2-(4-(4-chloro-1-(4-hydroxyphenyl)-2-phenylbut-1-en-1-yl)phenoxy)ethyl)-3- |
| | (2-((2-(2,6-dioxopiperidin-3-yl)-1-oxoisoindolin-4-yl)amino)ethoxy)propanamide |
| | N-(2-(4-(4-chloro-1-(4-hydroxyphenyl)-2-phenylbut-1-en-1-yl)phenoxy)ethyl)-3- |
| | (2-(2-((2-(2,6-dioxopiperidin-3-yl)-1-oxoisoindolin-4- |
| | yl)amino)ethoxy)ethoxy)propanamide |
| | N-(2-(4-(4-chloro-1-(4-hydroxyphenyl)-2-phenylbut-1-en-1-yl)phenoxy)ethyl)-3- |
| | (2-(2-(2-((2-(2,6-dioxopiperidin-3-yl)-1-oxoisoindolin-4- |
| | yl)amino)ethoxy)ethoxy)ethoxy)propanamide |
| | N-(2-(4-(4-chloro-1-(4-hydroxyphenyl)-2-phenylbut-1-en-1-yl)phenoxy)ethyl)-1- |
| | ((2-(2,6-dioxopiperidin-3-yl)-1-oxoisoindolin-4-yl)amino)-3,6,9,12- |
| | tetraoxapentadecan-15-amide |
| | N-(2-(4-(4-chloro-1-(4-hydroxyphenyl)-2-phenylbut-1-en-1-yl)phenoxy)ethyl)-1- |
| | ((2-(2,6-dioxopiperidin-3-yl)-1-oxoisoindolin-4-yl)amino)-3,6,9,12,15- |
| | pentaoxaoctadecan-18-amide |
| | N1-(2-(4-(4-chloro-1-(4-hydroxyphenyl)-2-phenylbut-1-en-1-yl)phenoxy)ethyl)- |
| | N3-(2-(2,6-dioxopiperidin-3-yl)-1-oxoisoindolin-4-yl)malonamide |
| | N1-(2-(4-(4-chloro-1-(4-hydroxyphenyl)-2-phenylbut-1-en-1-yl)phenoxy)ethyl)- |
| | N4-(2-(2,6-dioxopiperidin-3-yl)-1-oxoisoindolin-4-yl)succinamide |
| | N1-(2-(4-(4-chloro-1-(4-hydroxyphenyl)-2-phenylbut-1-en-1-yl)phenoxy)ethyl)- |
| | N5-(2-(2,6-dioxopiperidin-3-yl)-1-oxoisoindolin-4-yl)glutaramide |
| | N1-(2-(4-(4-chloro-1-(4-hydroxyphenyl)-2-phenylbut-1-en-1-yl)phenoxy)ethyl)- |
| | N6-(2-(2,6-dioxopiperidin-3-yl)-1-oxoisoindolin-4-yl)adipamide |
| | N1-(2-(4-(4-chloro-1-(4-hydroxyphenyl)-2-phenylbut-1-en-1-yl)phenoxy)ethyl)- |
| | N7-(2-(2,6-dioxopiperidin-3-yl)-1-oxoisoindolin-4-yl)heptanediamide |
| | N1-(2-(4-(4-chloro-1-(4-hydroxyphenyl)-2-phenylbut-1-en-1-yl)phenoxy)ethyl)- |
| | N8-(2-(2,6-dioxopiperidin-3-yl)-1-oxoisoindolin-4-yl)octanediamide |
| SIAIS251029 | N-(2-(4-(4-chloro-1-(4-hydroxyphenyl)-2-phenylbut-1-en-1-yl)phenoxy)ethyl)-2- |
| | ((2-(2,6-dioxopiperidin-3-yl)-1-oxoisoindolin-4-yl)amino)acetamide |
| | N-(2-(4-(4-chloro-1-(4-hydroxyphenyl)-2-phenylbut-1-en-1-yl)phenoxy)ethyl)-3- |
| | ((2-(2,6-dioxopiperidin-3-yl)-1-oxoisoindolin-4-yl)amino)propanamide |
| SIAIS251030 | N-(2-(4-(4-chloro-1-(4-hydroxyphenyl)-2-phenylbut-1-en-1-yl)phenoxy)ethyl)-4- |
| | ((2-(2,6-dioxopiperidin-3-yl)-1-oxoisoindolin-4-yl)amino)butanamide |
| SIAIS251031 | N-(2-(4-(4-chloro-1-(4-hydroxyphenyl)-2-phenylbut-1-en-1-yl)phenoxy)ethyl)-5- |
| | ((2-(2,6-dioxopiperidin-3-yl)-1-oxoisoindolin-4-yl)amino)pentanamide |
| SIAIS251032 | N-(2-(4-(4-chloro-1-(4-hydroxyphenyl)-2-phenylbut-1-en-1-yl)phenoxy)ethyl)-6- |
| | ((2-(2,6-dioxopiperidin-3-yl)-1-oxoisoindolin-4-yl)amino)hexanamide |
| SIAIS251033 | N-(2-(4-(4-chloro-1-(4-hydroxyphenyl)-2-phenylbut-1-en-1-yl)phenoxy)ethyl)-7- |
| | ((2-(2,6-dioxopiperidin-3-yl)-1-oxoisoindolin-4-yl)amino)heptanamide |
| | N-(2-(4-(4-chloro-1-(4-hydroxyphenyl)-2-phenylbut-1-en-1-yl)phenoxy)ethyl)-8- |
| | ((2-(2,6-dioxopiperidin-3-yl)-1-oxoisoindolin-4-yl)amino)octanamide |
| SIAIS208041 | (2S,4R)-1-((S)-2-(tert-butyl)-14-(4-(4-chloro-1-(4-hydroxyphenyl)-2-phenylbut-1- |
| | en-1-yl)phenoxy)-4,11-dioxo-6,9-dioxa-3,12-diazatetradecanoyl)-4-hydroxy-N-(4- |
| | (4-methylthiazol-5-yl)benzyl)pyrrolidine-2-carboxamide |
| SIAIS208017 | (2S,4R)-1-((S)-2-(tert-butyl)-16-(4-(4-chloro-1-(4-hydroxyphenyl)-2-phenylbut-1- |
| | en-1-yl)phenoxy)-4,13-dioxo-7,10-dioxa-3,14-diazahexadecanoyl)-4-hydroxy-N- |
| | (4-(4-methylthiazol-5-yl)benzyl)pyrrolidine-2-carboxamide |
| SIAIS208018 | (2S,4R)-1-((S)-2-(tert-butyl)-19-(4-(4-chloro-1-(4-hydroxyphenyl)-2-phenylbut-1- |
| | en-1-yl)phenoxy)-4,16-dioxo-7,10,13-trioxa-3,17-diazanonadecanoyl)-4-hydroxy- |
| | N-(4-(4-methylthiazol-5-yl)benzyl)pyrrolidine-2-carboxamide |
| SIAIS208019 | N1-(2-(4-(4-chloro-1-(4-hydroxyphenyl)-2-phenylbut-1-en-1-yl)phenoxy)ethyl)- |
| | N16-((S)-1-((2S,4R)-4-hydroxy-2-((4-(4-methylthiazol-5- |
| | yl)benzyl)carbamoyl)pyrrolidin-1-yl)-3,3-dimethyl-1-oxobutan-2-yl)-4,7,10,13- |
| | tetraoxahexadecanediamide |
| SIAIS208045 | N1-(2-(4-(4-chloro-1-(4-hydroxyphenyl)-2-phenylbut-1-en-1-yl)phenoxy)ethyl)- |
| | N19-((S)-1-((2S,4R)-4-hydroxy-2-((4-(4-methylthiazol-5- |
| | yl)benzyl)carbamoyl)pyrrolidin-1-yl)-3,3-dimethyl-1-oxobutan-2-yl)-4,7,10,13,16- |
| | pentaoxanonadecanediamide |
| SIAIS208020 | N1-(2-(4-(4-chloro-1-(4-hydroxyphenyl)-2-phenylbut-1-en-1-yl)phenoxy)ethyl)- |
| | N4-((S)-1-((2S,4R)-4-hydroxy-2-((4-(4-methylthiazol-5- |
| | yl)benzyl)carbamoyl)pyrrolidin-1-yl)-3,3-dimethyl-1-oxobutan-2-yl)succinamide |
| SIAIS208031 | N1-(2-(4-(4-chloro-1-(4-hydroxyphenyl)-2-phenylbut-1-en-1-yl)phenoxy)ethyl)- |
| | N5-((S)-1-((2S,4R)-4-hydroxy-2-((4-(4-methylthiazol-5- |
| | yl)benzyl)carbamoyl)pyrrolidin-1-yl)-3,3-dimethyl-1-oxobutan-2-yl)glutaramide |
| SIAIS208032 | N1-(2-(4-(4-chloro-1-(4-hydroxyphenyl)-2-phenylbut-1-en-1-yl)phenoxy)ethyl)- |
| | N6-((S)-1-((2S,4R)-4-hydroxy-2-((4-(4-methylthiazol-5- |
| | yl)benzyl)carbamoyl)pyrrolidin-1-yl)-3,3-dimethyl-1-oxobutan-2-yl)adipamide |
| SIAIS208033 | N1-(2-(4-(4-chloro-1-(4-hydroxyphenyl)-2-phenylbut-1-en-1-yl)phenoxy)ethyl)- |
| | N7-((S)-1-((2S,4R)-4-hydroxy-2-((4-(4-methylthiazol-5- |
| | yl)benzyl)carbamoyl)pyrrolidin-1-yl)-3,3-dimethyl-1-oxobutan-2- |
| | yl)heptanediamide |
| SIAIS208034 | N1-(2-(4-(4-chloro-1-(4-hydroxyphenyl)-2-phenylbut-1-en-1-yl)phenoxy)ethyl)- |
| | N8-((S)-1-((2S,4R)-4-hydroxy-2-((4-(4-methylthiazol-5- |
| | yl)benzyl)carbamoyl)pyrrolidin-1-yl)-3,3-dimethyl-1-oxobutan-2-yl)octanediamide |
| SIAIS208035 | N1-(2-(4-(4-chloro-1-(4-hydroxyphenyl)-2-phenylbut-1-en-1-yl)phenoxy)ethyl)- |
| | N9-((S)-1-((2S,4R)-4-hydroxy-2-((4-(4-methylthiazol-5- |
| | yl)benzyl)carbamoyl)pyrrolidin-1-yl)-3,3-dimethyl-1-oxobutan-2- |
| | yl)nonanediamide |
| SIAIS208036 | N1-(2-(4-((Z)-4-chloro-1-(4-hydroxyphenyl)-2-phenylbut-1-en-1- |
| | yl)phenoxy)ethyl)-N10-((S)-1-((2S,4R)-4-hydroxy-2-((4-(4-methylthiazol-5- |
| | yl)benzyl)carbamoyl)pyrrolidin-1-yl)-3,3-dimethyl-1-oxobutan-2-yl)decanediamide |
| SIAIS208037 | N1-(2-(4-(4-chloro-1-(4-hydroxyphenyl)-2-phenylbut-1-en-1-yl)phenoxy)ethyl)- |
| | N11-((S)-1-((2S,4R)-4-hydroxy-2-((4-(4-methylthiazol-5- |
| | yl)benzyl)carbamoyl)pyrrolidin-1-yl)-3,3-dimethyl-1-oxobutan-2- |
| | yl)undecanediamide |
| SIAIS208038 | N1-(2-(4-(4-chloro-1-(4-hydroxyphenyl)-2-phenylbut-1-en-1-yl)phenoxy)ethyl)- |
| | N14-((S)-1-((2S,4R)-4-hydroxy-2-((4-(4-methylthiazol-5- |
| | yl)benzyl)carbamoyl)pyrrolidin-1-yl)-3,3-dimethyl-1-oxobutan-2- |
| | yl)tetradecanediamide |
| SIAIS208039 | N1-(2-(4-(4-chloro-1-(4-hydroxyphenyl)-2-phenylbut-1-en-1-yl)phenoxy)ethyl)- |
| | N16-((S)-1-((2S,4R)-4-hydroxy-2-((4-(4-methylthiazol-5- |
| | yl)benzyl)carbamoyl)pyrrolidin-1-yl)-3,3-dimethyl-1-oxobutan-2- |
| | yl)hexadecanediamide |
| SIAIS208107 | (2S,4R)-1-((S)-2-(3-(4-(3-((2-(4-(4-chloro-1-(4-hydroxyphenyl)-2-phenylbut-1-en- |
| | 1-yl)phenoxy)ethyl)amino)-3-oxopropyl)piperazin-1-yl)propanamido)-3,3- |
| | dimethylbutanoyl)-4-hydroxy-N-(4-(4-methylthiazol-5-yl)benzyl)pyrrolidine-2- |
| | carboxamide |
| SIAIS208127 | (2S,4R)-1-((S)-2-(3-(4-(3-((2-(4-(4-chloro-1-(4-hydroxyphenyl)-2-phenylbut-1-en- |
| | 1-yl)phenoxy)ethyl)amino)-3-oxopropyl)phenyl)propanamido)-3,3- |
| | dimethylbutanoyl)-4-hydroxy-N-(4-(4-methylthiazol-5-yl)benzyl)pyrrolidine-2- |
| | carboxamide |
| SIAIS208137 | N-(2-(4-(4-chloro-1-(4-hydroxyphenyl)-2-phenylbut-1-en-1-yl)phenoxy)ethyl)-3- |
| | (4-(2-((2-(2,6-dioxopiperidin-3-yl)-1,3-dioxoisoindolin-4- |
| | yl)amino)ethyl)piperazin-1-yl)propanamide |
| | N-(2-(4-(4-chloro-1-(4-hydroxyphenyl)-2-phenylbut-1-en-1-yl)phenoxy)ethyl)-3- |
| | (4-(2-((2-(2,6-dioxopiperidin-3-yl)-1,3-dioxoisoindolin-4- |
| | yl)amino)ethyl)phenyl)propanamide |
| | (2S,4R)-1-((S)-2-(7-((2-(4-(4-chloro-1-(4-hydroxyphenyl)-2-phenylbut-1-en-1- |
| | yl)phenoxy)ethyl)amino)heptanamido)-3,3-dimethylbutanoyl)-4-hydroxy-N-(4-(4- |
| | methylthiazol-5-yl)benzyl)pyrrolidine-2-carboxamide |
| | (2S,4R)-1-((S)-2-((7-((2-(4-(4-chloro-1-(4-hydroxyphenyl)-2-phenylbut-1-en-1- |
| | yl)phenoxy)ethyl)amino)-7-oxoheptyl)amino)-3,3-dimethylbutanoyl)-4-hydroxy- |
| | N-(4-(4-methylthiazol-5-yl)benzyl)pyrrolidine-2-carboxamide |
| | (2S,4R)-1-((S)-2-((7-((2-(4-(4-chloro-1-(4-hydroxyphenyl)-2-phenylbut-1-en-1- |
| | yl)phenoxy)ethyl)amino)heptyl)amino)-3,3-dimethylbutanoyl)-4-hydroxy-N-(4-(4- |
| | methylthiazol-5-yl)benzyl)pyrrolidine-2-carboxamide |
| | 4-((2-(3-(4-(2-(4-(4-chloro-1-(4-hydroxyphenyl)-2-phenylbut-1-en-1- |
| | yl)phenoxy)ethyl)piperazin-1-yl)-3-oxopropoxy)ethyl)amino)-2-(2,6- |
| | dioxopiperidin-3-yl)isoindoline-1,3-dione |
| | 4-((2-(2-(3-(4-(2-(4-(4-chloro-1-(4-hydroxyphenyl)-2-phenylbut-1-en-1- |
| | yl)phenoxy)ethyl)piperazin-1-yl)-3-oxopropoxy)ethoxy)ethyl)amino)-2-(2,6- |
| | dioxopiperidin-3-yl)isoindoline-1,3-dione |
| | 4-((2-(2-(2-(3-(4-(2-(4-(4-chloro-1-(4-hydroxyphenyl)-2-phenylbut-1-en-1- |
| | yl)phenoxy)ethyl)piperazin-1-yl)-3-oxopropoxy)ethoxy)ethoxy)ethyl)amino)-2- |
| | (2,6-dioxopiperidin-3-yl)isoindoline-1,3-dione |
| | 4-((15-(4-(2-(4-(4-chloro-1-(4-hydroxyphenyl)-2-phenylbut-1-en-1- |
| | yl)phenoxy)ethyl)piperazin-1-yl)-15-oxo-3,6,9,12-tetraoxapentadecyl)amino)-2- |
| | (2,6-dioxopiperidin-3-yl)isoindoline-1,3-dione |
| | 4-((18-(4-(2-(4-(4-chloro-1-(4-hydroxyphenyl)-2-phenylbut-1-en-1- |
| | yl)phenoxy)ethyl)piperazin-1-yl)-18-oxo-3,6,9,12,15-pentaoxaoctadecyl)amino)-2- |
| | (2,6-dioxopiperidin-3-yl)isoindoline-1,3-dione |
| | 4-((2-(4-(2-(4-(4-chloro-1-(4-hydroxyphenyl)-2-phenylbut-1-en-1- |
| | yl)phenoxy)ethyl)piperazin-1-yl)-2-oxoethyl)amino)-2-(2,6-dioxopiperidin-3- |
| | yl)isoindoline-1,3-dione |
| | 4-((3-(4-(2-(4-(4-chloro-1-(4-hydroxyphenyl)-2-phenylbut-1-en-1- |
| | yl)phenoxy)ethyl)piperazin-1-yl)-3-oxopropyl)amino)-2-(2,6-dioxopiperidin-3- |
| | yl)isoindoline-1,3-dione |
| | 4-((4-(4-(2-(4-(4-chloro-1-(4-hydroxyphenyl)-2-phenylbut-1-en-1- |
| | yl)phenoxy)ethyl)piperazin-1-yl)-4-oxobutyl)amino)-2-(2,6-dioxopiperidin-3- |
| | yl)isoindoline-1,3-dione |
| | 4-((5-(4-(2-(4-(4-chloro-1-(4-hydroxyphenyl)-2-phenylbut-1-en-1- |
| | yl)phenoxy)ethyl)piperazin-1-yl)-5-oxopentyl)amino)-2-(2,6-dioxopiperidin-3- |
| | yl)isoindoline-1,3-dione |
| | 4-((6-(4-(2-(4-(4-chloro-1-(4-hydroxyphenyl)-2-phenylbut-1-en-1- |
| | yl)phenoxy)ethyl)piperazin-1-yl)-6-oxohexyl)amino)-2-(2,6-dioxopiperidin-3- |
| | yl)isoindoline-1,3-dione |
| | 4-((7-(4-(2-(4-(4-chloro-1-(4-hydroxyphenyl)-2-phenylbut-1-en-1- |
| | yl)phenoxy)ethyl)piperazin-1-yl)-7-oxoheptyl)amino)-2-(2,6-dioxopiperidin-3- |
| | yl)isoindoline-1,3-dione |
| | 3-(3-(4-(2-(4-(4-chloro-1-(4-hydroxyphenyl)-2-phenylbut-1-en-1- |
| | yl)phenoxy)ethyl)piperazin-1-yl)-3-oxopropoxy)-N-(2-(2,6-dioxopiperidin-3-yl)-1- |
| | oxoisoindolin-4-yl)propanamide |
| | 3-(2-(3-(4-(2-(4-(4-chloro-1-(4-hydroxyphenyl)-2-phenylbut-1-en-1- |
| | yl)phenoxy)ethyl)piperazin-1-yl)-3-oxopropoxy)ethoxy)-N-(2-(2,6-dioxopiperidin- |
| | 3-yl)-1-oxoisoindolin-4-yl)propanamide |
| | 3-(2-(2-(3-(4-(2-(4-(4-chloro-1-(4-hydroxyphenyl)-2-phenylbut-1-en-1- |
| | yl)phenoxy)ethyl)piperazin-1-yl)-3-oxopropoxy)ethoxy)ethoxy)-N-(2-(2,6- |
| | dioxopiperidin-3-yl)-1-oxoisoindolin-4-yl)propanamide |
| | 16-(4-(2-(4-(4-chloro-1-(4-hydroxyphenyl)-2-phenylbut-1-en-1- |
| | yl)phenoxy)ethyl)piperazin-1-yl)-N-(2-(2,6-dioxopiperidin-3-yl)-1-oxoisoindolin- |
| | 4-yl)-16-oxo-4,7,10,13-tetraoxahexadecanamide |
| | 19-(4-(2-(4-(4-chloro-1-(4-hydroxyphenyl)-2-phenylbut-1-en-1- |
| | yl)phenoxy)ethyl)piperazin-1-yl)-N-(2-(2,6-dioxopiperidin-3-yl)-1-oxoisoindolin- |
| | 4-yl)-19-oxo-4,7,10,13,16-pentaoxanonadecanamide |
| | 3-(4-((2-(3-(4-(2-(4-(4-chloro-1-(4-hydroxyphenyl)-2-phenylbut-1-en-1- |
| | yl)phenoxy)ethyl)piperazin-1-yl)-3-oxopropoxy)ethyl)amino)-1-oxoisoindolin-2- |
| | yl)piperidine-2,6-dione |
| | 3-(4-((2-(2-(3-(4-(2-(4-(4-chloro-1-(4-hydroxyphenyl)-2-phenylbut-1-en-1- |
| | yl)phenoxy)ethyl)piperazin-1-yl)-3-oxopropoxy)ethoxy)ethyl)amino)-1- |
| | oxoisoindolin-2-yl)piperidine-2,6-dione |
| | 3-(4-((2-(2-(2-(3-(4-(2-(4-(4-chloro-1-(4-hydroxyphenyl)-2-phenylbut-1-en-1- |
| | yl)phenoxy)ethyl)piperazin-1-yl)-3-oxopropoxy)ethoxy)ethoxy)ethyl)amino)-1- |
| | oxoisoindolin-2-yl)piperidine-2,6-dione |
| | 3-(4-((15-(4-(2-(4-(4-chloro-1-(4-hydroxyphenyl)-2-phenylbut-1-en-1- |
| | yl)phenoxy)ethyl)piperazin-1-yl)-15-oxo-3,6,9,12-tetraoxapentadecyl)amino)-1- |
| | oxoisoindolin-2-yl)piperidine-2,6-dione |
| | 3-(4-((18-(4-(2-(4-(4-chloro-1-(4-hydroxyphenyl)-2-phenylbut-1-en-1- |
| | yl)phenoxy)ethyl)piperazin-1-yl)-18-oxo-3,6,9,12,15-pentaoxaoctadecyl)amino)-1- |
| | oxoisoindolin-2-yl)piperidine-2,6-dione |
| | 3-(4-(2-(4-(4-chloro-1-(4-hydroxyphenyl)-2-phenylbut-1-en-1- |
| | yl)phenoxy)ethyl)piperazin-1-yl)-N-(2-(2,6-dioxopiperidin-3-yl)-1-oxoisoindolin- |
| | 4-yl)-3-oxopropanamide |
| | 4-(4-(2-(4-(4-chloro-1-(4-hydroxyphenyl)-2-phenylbut-1-en-1- |
| | yl)phenoxy)ethyl)piperazin-1-yl)-N-(2-(2,6-dioxopiperidin-3-yl)-1-oxoisoindolin- |
| | 4-yl)-4-oxobutanamide |
| | 5-(4-(2-(4-(4-chloro-1-(4-hydroxyphenyl)-2-phenylbut-1-en-1- |
| | yl)phenoxy)ethyl)piperazin-1-yl)-N-(2-(2,6-dioxopiperidin-3-yl)-1-oxoisoindolin- |
| | 4-yl)-5-oxopentanamide |
| | 6-(4-(2-(4-(4-chloro-1-(4-hydroxyphenyl)-2-phenylbut-1-en-1- |
| | yl)phenoxy)ethyl)piperazin-1-yl)-N-(2-(2,6-dioxopiperidin-3-yl)-1-oxoisoindolin- |
| | 4-yl)-6-oxohexanamide |
| | 7-(4-(2-(4-(4-chloro-1-(4-hydroxyphenyl)-2-phenylbut-1-en-1- |
| | yl)phenoxy)ethyl)piperazin-1-yl)-N-(2-(2,6-dioxopiperidin-3-yl)-1-oxoisoindolin- |
| | 4-yl)-7-oxoheptanamide |
| | 3-(4-((2-(4-(2-(4-(4-chloro-1-(4-hydroxyphenyl)-2-phenylbut-1-en-1- |
| | yl)phenoxy)ethyl)piperazin-1-yl)-2-oxoethyl)amino)-1-oxoisoindolin-2- |
| | yl)piperidine-2,6-dione |
| | 3-(4-((3-(4-(2-(4-(4-chloro-1-(4-hydroxyphenyl)-2-phenylbut-1-en-1- |
| | yl)phenoxy)ethyl)piperazin-1-yl)-3-oxopropyl)amino)-1-oxoisoindolin-2- |
| | yl)piperidine-2,6-dione |
| | 3-(4-((4-(4-(2-(4-(4-chloro-1-(4-hydroxyphenyl)-2-phenylbut-1-en-1- |
| | yl)phenoxy)ethyl)piperazin-1-yl)-4-oxobutyl)amino)-1-oxoisoindolin-2- |
| | yl)piperidine-2,6-dione |
| | 3-(4-((5-(4-(2-(4-(4-chloro-1-(4-hydroxyphenyl)-2-phenylbut-1-en-1- |
| | yl)phenoxy)ethyl)piperazin-1-yl)-5-oxopentyl)amino)-1-oxoisoindolin-2- |
| | yl)piperidine-2,6-dione |
| | 3-(4-((6-(4-(2-(4-(4-chloro-1-(4-hydroxyphenyl)-2-phenylbut-1-en-1- |
| | yl)phenoxy)ethyl)piperazin-1-yl)-6-oxohexyl)amino)-1-oxoisoindolin-2- |
| | yl)piperidine-2,6-dione |
| | 3-(4-((7-(4-(2-(4-(4-chloro-1-(4-hydroxyphenyl)-2-phenylbut-1-en-1- |
| | yl)phenoxy)ethyl)piperazin-1-yl)-7-oxoheptyl)amino)-1-oxoisoindolin-2- |
| | yl)piperidine-2,6-dione |
| SIAIS251048 | (2S,4R)-1-((S)-2-(2-(2-(4-(2-(4-(4-chloro-1-(4-hydroxyphenyl)-2-phenylbut-1-en- |
| | 1-yl)phenoxy)ethyl)piperazin-1-yl)-2-oxoethoxy)acetamido)-3,3- |
| | dimethylbutanoyl)-4-hydroxy-N-(4-(4-methylthiazol-5-yl)benzyl)pyrrolidine-2- |
| | carboxamide |
| | (2S,4R)-1-((S)-2-(2-(2-(2-(4-(2-(4-(4-chloro-1-(4-hydroxyphenyl)-2-phenylbut-1- |
| | en-1-yl)phenoxy)ethyl)piperazin-1-yl)-2-oxoethoxy)ethoxy)acetamido)-3,3- |
| | dimethylbutanoyl)-4-hydroxy-N-(4-(4-methylthiazol-5-yl)benzyl)pyrrolidine-2- |
| | carboxamide |
| SIAIS251049 | (2S,4R)-1-((S)-2-(3-(2-(3-(4-(2-(4-(4-chloro-1-(4-hydroxyphenyl)-2-phenylbut-1- |
| | en-1-yl)phenoxy)ethyl)piperazin-1-yl)-3-oxopropoxy)ethoxy)propanamido)-3,3- |
| | dimethylbutanoyl)-4-hydroxy-N-(4-(4-methylthiazol-5-yl)benzyl)pyrrolidine-2- |
| | carboxamide |
| SIAIS251050 | (2S,4R)-1-((S)-2-(tert-butyl)-16-(4-(2-(4-(4-chloro-1-(4-hydroxyphenyl)-2- |
| | phenylbut-1-en-1-yl)phenoxy)ethyl)piperazin-1-yl)-4,16-dioxo-7,10,13-trioxa-3- |
| | azahexadecanoyl)-4-hydroxy-N-(4-(4-methylthiazol-5-yl)benzyl)pyrrolidine-2- |
| | carboxamide |
| SIAIS251051 | (2S,4R)-1-((S)-2-(tert-butyl)-19-(4-(2-(4-(4-chloro-1-(4-hydroxyphenyl)-2- |
| | phenylbut-1-en-1-yl)phenoxy)ethyl)piperazin-1-yl)-4,19-dioxo-7,10,13,16- |
| | tetraoxa-3-azanonadecanoyl)-4-hydroxy-N-(4-(4-methylthiazol-5- |
| | yl)benzyl)pyrrolidine-2-carboxamide |
| | (2S,4R)-1-((S)-2-(tert-butyl)-22-(4-(2-(4-(4-chloro-1-(4-hydroxyphenyl)-2- |
| | phenylbut-1-en-1-yl)phenoxy)ethyl)piperazin-1-yl)-4,22-dioxo-7,10,13,16,19- |
| | pentaoxa-3-azadocosanoyl)-4-hydroxy-N-(4-(4-methylthiazol-5- |
| | yl)benzyl)pyrrolidine-2-carboxamide |
| SIAIS251041 | (2S,4R)-1-((S)-2-(4-(4-(2-(4-(4-chloro-1-(4-hydroxyphenyl)-2-phenylbut-1-en-1- |
| | yl)phenoxy)ethyl)piperazin-1-yl)-4-oxobutanamido)-3,3-dimethylbutanoyl)-4- |
| | hydroxy-N-(4-(4-methylthiazol-5-yl)benzyl)pyrrolidine-2-carboxamide |
| SIAIS251042 | (2S,4R)-1-((S)-2-(5-(4-(2-(4-(4-chloro-1-(4-hydroxyphenyl)-2-phenylbut-1-en-1- |
| | yl)phenoxy)ethyl)piperazin-1-yl)-5-oxopentanamido)-3,3-dimethylbutanoyl)-4- |
| | hydroxy-N-(4-(4-methylthiazol-5-yl)benzyl)pyrrolidine-2-carboxamide |
| SIAIS251043 | (2S,4R)-1-((S)-2-(6-(4-(2-(4-(4-chloro-1-(4-hydroxyphenyl)-2-phenylbut-1-en-1- |
| | yl)phenoxy)ethyl)piperazin-1-yl)-6-oxohexanamido)-3,3-dimethylbutanoyl)-4- |
| | hydroxy-N-(4-(4-methylthiazol-5-yl)benzyl)pyrrolidine-2-carboxamide |
| | (2S,4R)-1-((S)-2-(7-(4-(2-(4-(4-chloro-1-(4-hydroxyphenyl)-2-phenylbut-1-en-1- |
| | yl)phenoxy)ethyl)piperazin-1-yl)-7-oxoheptanamido)-3,3-dimethylbutanoyl)-4- |
| | hydroxy-N-(4-(4-methylthiazol-5-yl)benzyl)pyrrolidine-2-carboxamide |
| SIAIS251045 | (2S,4R)-1-((S)-2-(8-(4-(2-(4-(4-chloro-1-(4-hydroxyphenyl)-2-phenylbut-1-en-1- |
| | yl)phenoxy)ethyl)piperazin-1-yl)-8-oxooctanamido)-3,3-dimethylbutanoyl)-4- |
| | hydroxy-N-(4-(4-methylthiazol-5-yl)benzyl)pyrrolidine-2-carboxamide |
| SIAIS251046 | (2S,4R)-1-((S)-2-(9-(4-(2-(4-(4-chloro-1-(4-hydroxyphenyl)-2-phenylbut-1-en-1- |
| | yl)phenoxy)ethyl)piperazin-1-yl)-9-oxononanamido)-3,3-dimethylbutanoyl)-4- |
| | hydroxy-N-(4-(4-methylthiazol-5-yl)benzyl)pyrrolidine-2-carboxamide |
| SIAIS251047 | (2S,4R)-1-((S)-2-(10-(4-(2-(4-(4-chloro-1-(4-hydroxyphenyl)-2-phenylbut-1-en-1- |
| | yl)phenoxy)ethyl)piperazin-1-yl)-10-oxodecanamido)-3,3-dimethylbutanoyl)-4- |
| | hydroxy-N-(4-(4-methylthiazol-5-yl)benzyl)pyrrolidine-2-carboxamide |
| | (2S,4R)-1-((S)-2-(7-(4-(2-(4-(4-chloro-1-(4-hydroxyphenyl)-2-phenylbut-1-en-1- |
| | yl)phenoxy)ethyl)piperazin-1-yl)heptanamido)-3,3-dimethylbutanoyl)-4-hydroxy- |
| | N-(4-(4-methylthiazol-5-yl)benzyl)pyrrolidine-2-carboxamide |
| | (2S,4R)-1-((S)-2-((7-(4-(2-(4-(4-chloro-1-(4-hydroxyphenyl)-2-phenylbut-1-en-1- |
| | yl)phenoxy)ethyl)piperazin-1-yl)-7-oxoheptyl)amino)-3,3-dimethylbutanoyl)-4- |
| | hydroxy-N-(4-(4-methylthiazol-5-yl)benzyl)pyrrolidine-2-carboxamide |
| | (2S,4R)-1-((S)-2-((7-(4-(2-(4-(4-chloro-1-(4-hydroxyphenyl)-2-phenylbut-1-en-1- |
| | yl)phenoxy)ethyl)piperazin-1-yl)heptyl)amino)-3,3-dimethylbutanoyl)-4-hydroxy- |
| | N-(4-(4-methylthiazol-5-yl)benzyl)pyrrolidine-2-carboxamide |
| | N-(2-(4-(4-chloro-1,2-bis(4-hydroxyphenyl)but-1-en-1-yl)phenoxy)ethyl)-3-(2-((2- |
| | (2,6-dioxopiperidin-3-yl)-1,3-dioxoisoindolin-4-yl)amino)ethoxy)propanamide |
| | N-(2-(4-(4-chloro-1,2-bis(4-hydroxyphenyl)but-1-en-1-yl)phenoxy)ethyl)-3-(2-(2- |
| | ((2-(2,6-dioxopiperidin-3-yl)-1,3-dioxoisoindolin-4- |
| | yl)amino)ethoxy)ethoxy)propanamide |
| | N-(2-(4-(4-chloro-1,2-bis(4-hydroxyphenyl)but-1-en-1-yl)phenoxy)ethyl)-3-(2-(2- |
| | (2-((2-(2,6-dioxopiperidin-3-yl)-1,3-dioxoisoindolin-4- |
| | yl)amino)ethoxy)ethoxy)ethoxy)propanamide |
| | N-(2-(4-(4-chloro-1,2-bis(4-hydroxyphenyl)but-1-en-1-yl)phenoxy)ethyl)-1-((2- |
| | (2,6-dioxopiperidin-3-yl)-1,3-dioxoisoindolin-4-yl)amino)-3,6,9,12- |
| | tetraoxapentadecan-15-amide |
| | N-(2-(4-(4-chloro-1,2-bis(4-hydroxyphenyl)but-1-en-1-yl)phenoxy)ethyl)-1-((2- |
| | (2,6-dioxopiperidin-3-yl)-1,3-dioxoisoindolin-4-yl)amino)-3,6,9,12,15- |
| | pentaoxaoctadecan-18-amide |
| | N-(2-(4-(4-chloro-1,2-bis(4-hydroxyphenyl)but-1-en-1-yl)phenoxy)ethyl)-2-((2- |
| | (2,6-dioxopiperidin-3-yl)-1,3-dioxoisoindolin-4-yl)amino)acetamide |
| | N-(2-(4-(4-chloro-1,2-bis(4-hydroxyphenyl)but-1-en-1-yl)phenoxy)ethyl)-3-((2- |
| | (2,6-dioxopiperidin-3-yl)-1,3-dioxoisoindolin-4-yl)amino)propanamide |
| | N-(2-(4-(4-chloro-1,2-bis(4-hydroxyphenyl)but-1-en-1-yl)phenoxy)ethyl)-4-((2- |
| | (2,6-dioxopiperidin-3-yl)-1,3-dioxoisoindolin-4-yl)amino)butanamide |
| | N-(2-(4-(4-chloro-1,2-bis(4-hydroxyphenyl)but-1-en-1-yl)phenoxy)ethyl)-5-((2- |
| | (2,6-dioxopiperidin-3-yl)-1,3-dioxoisoindolin-4-yl)amino)pentanamide |
| | N-(2-(4-(4-chloro-1,2-bis(4-hydroxyphenyl)but-1-en-1-yl)phenoxy)ethyl)-6-((2- |
| | (2,6-dioxopiperidin-3-yl)-1,3-dioxoisoindolin-4-yl)amino)hexanamide |
| | N-(2-(4-(4-chloro-1,2-bis(4-hydroxyphenyl)but-1-en-1-yl)phenoxy)ethyl)-7-((2- |
| | (2,6-dioxopiperidin-3-yl)-1,3-dioxoisoindolin-4-yl)amino)heptanamide |
| | N-(2-(4-(4-chloro-1,2-bis(4-hydroxyphenyl)but-1-en-1-yl)phenoxy)ethyl)-3-(3-((2- |
| | (2,6-dioxopiperidin-3-yl)-1-oxoisoindolin-4-yl)amino)-3-oxopropoxy)propanamide |
| | N-(2-(4-(4-chloro-1,2-bis(4-hydroxyphenyl)but-1-en-1-yl)phenoxy)ethyl)-3-(2-(3- |
| | ((2-(2,6-dioxopiperidin-3-yl)-1-oxoisoindolin-4-yl)amino)-3- |
| | oxopropoxy)ethoxy)propanamide |
| | N-(2-(4-(4-chloro-1,2-bis(4-hydroxyphenyl)but-1-en-1-yl)phenoxy)ethyl)-3-(2-(2- |
| | (3-((2-(2,6-dioxopiperidin-3-yl)-1-oxoisoindolin-4-yl)amino)-3- |
| | oxopropoxy)ethoxy)ethoxy)propanamide |
| | N1-(2-(4-(4-chloro-1,2-bis(4-hydroxyphenyl)but-1-en-1-yl)phenoxy)ethyl)-N16-(2- |
| | (2,6-dioxopiperidin-3-yl)-1-oxoisoindolin-4-yl)-4,7,10,13- |
| | tetraoxahexadecanediamide |
| | N1-(2-(4-(4-chloro-1,2-bis(4-hydroxyphenyl)but-1-en-1-yl)phenoxy)ethyl)-N19-(2- |
| | (2,6-dioxopiperidin-3-yl)-1-oxoisoindolin-4-yl)-4,7,10,13,16- |
| | pentaoxanonadecanediamide |
| | N-(2-(4-(4-chloro-1,2-bis(4-hydroxyphenyl)but-1-en-1-yl)phenoxy)ethyl)-3-(2-((2- |
| | (2,6-dioxopiperidin-3-yl)-1-oxoisoindolin-4-yl)amino)ethoxy)propanamide |
| | N-(2-(4-(4-chloro-1,2-bis(4-hydroxyphenyl)but-1-en-1-yl)phenoxy)ethyl)-3-(2-(2- |
| | ((2-(2,6-dioxopiperidin-3-yl)-1-oxoisoindolin-4- |
| | yl)amino)ethoxy)ethoxy)propanamide |
| | N-(2-(4-(4-chloro-1,2-bis(4-hydroxyphenyl)but-1-en-1-yl)phenoxy)ethyl)-3-(2-(2- |
| | (2-((2-(2,6-dioxopiperidin-3-yl)-1-oxoisoindolin-4- |
| | yl)amino)ethoxy)ethoxy)ethoxy)propanamide |
| | N-(2-(4-(4-chloro-1,2-bis(4-hydroxyphenyl)but-1-en-1-yl)phenoxy)ethyl)-1-((2- |
| | (2,6-dioxopiperidin-3-yl)-1-oxoisoindolin-4-yl)amino)-3,6,9,12- |
| | tetraoxapentadecan-15-amide |
| | N-(2-(4-(4-chloro-1,2-bis(4-hydroxyphenyl)but-1-en-1-yl)phenoxy)ethyl)-1-((2- |
| | (2,6-dioxopiperidin-3-yl)-1-oxoisoindolin-4-yl)amino)-3,6,9,12,15- |
| | pentaoxaoctadecan-18-amide |
| | N1-(2-(4-(4-chloro-1,2-bis(4-hydroxyphenyl)but-1-en-1-yl)phenoxy)ethyl)-N3-(2- |
| | (2,6-dioxopiperidin-3-yl)-1-oxoisoindolin-4-yl)malonamide |
| | N1-(2-(4-(4-chloro-1,2-bis(4-hydroxyphenyl)but-1-en-1-yl)phenoxy)ethyl)-N4-(2- |
| | (2,6-dioxopiperidin-3-yl)-1-oxoisoindolin-4-yl)succinamide |
| | N1-(2-(4-(4-chloro-1,2-bis(4-hydroxyphenyl)but-1-en-1-yl)phenoxy)ethyl)-N5-(2- |
| | (2,6-dioxopiperidin-3-yl)-1-oxoisoindolin-4-yl)glutaramide |
| | N1-(2-(4-(4-chloro-1,2-bis(4-hydroxyphenyl)but-1-en-1-yl)phenoxy)ethyl)-N6-(2- |
| | (2,6-dioxopiperidin-3-yl)-1-oxoisoindolin-4-yl)adipamide |
| | N1-(2-(4-(4-chloro-1,2-bis(4-hydroxyphenyl)but-1-en-1-yl)phenoxy)ethyl)-N7-(2- |
| | (2,6-dioxopiperidin-3-yl)-1-oxoisoindolin-4-yl)heptanediamide |
| | N1-(2-(4-(4-chloro-1,2-bis(4-hydroxyphenyl)but-1-en-1-yl)phenoxy)ethyl)-N8-(2- |
| | (2,6-dioxopiperidin-3-yl)-1-oxoisoindolin-4-yl)octanediamide |
| | N-(2-(4-(4-chloro-1,2-bis(4-hydroxyphenyl)but-1-en-1-yl)phenoxy)ethyl)-3-((2- |
| | (2,6-dioxopiperidin-3-yl)-1-oxoisoindolin-4-yl)amino)propanamide |
| | N-(2-(4-(4-chloro-1,2-bis(4-hydroxyphenyl)but-1-en-1-yl)phenoxy)ethyl)-4-((2- |
| | (2,6-dioxopiperidin-3-yl)-1-oxoisoindolin-4-yl)amino)butanamide |
| | N-(2-(4-(4-chloro-1,2-bis(4-hydroxyphenyl)but-1-en-1-yl)phenoxy)ethyl)-5-((2- |
| | (2,6-dioxopiperidin-3-yl)-1-oxoisoindolin-4-yl)amino)pentanamide |
| | N-(2-(4-(4-chloro-1,2-bis(4-hydroxyphenyl)but-1-en-1-yl)phenoxy)ethyl)-6-((2- |
| | (2,6-dioxopiperidin-3-yl)-1-oxoisoindolin-4-yl)amino)hexanamide |
| | N-(2-(4-(4-chloro-1,2-bis(4-hydroxyphenyl)but-1-en-1-yl)phenoxy)ethyl)-7-((2- |
| | (2,6-dioxopiperidin-3-yl)-1-oxoisoindolin-4-yl)amino)heptanamide |
| | N-(2-(4-(4-chloro-1,2-bis(4-hydroxyphenyl)but-1-en-1-yl)phenoxy)ethyl)-8-((2- |
| | (2,6-dioxopiperidin-3-yl)-1-oxoisoindolin-4-yl)amino)octanamide |
| | (2S,4R)-1-((S)-2-(tert-butyl)-14-(4-(4-chloro-1,2-bis(4-hydroxyphenyl)but-1-en-1- |
| | yl)phenoxy)-4,11-dioxo-6,9-dioxa-3,12-diazatetradecanoyl)-4-hydroxy-N-(4-(4- |
| | methylthiazol-5-yl)benzyl)pyrrolidine-2-carboxamide |
| | (2S,4R)-1-((S)-2-(tert-butyl)-16-(4-(4-chloro-1,2-bis(4-hydroxyphenyl)but-1-en-1- |
| | yl)phenoxy)-4,13-dioxo-7,10-dioxa-3,14-diazahexadecanoyl)-4-hydroxy-N-(4-(4- |
| | methylthiazol-5-yl)benzyl)pyrrolidine-2-carboxamide |
| SIAIS307146 | (2S,4R)-1-((S)-2-(tert-butyl)-19-(4-(4-chloro-1,2-bis(4-hydroxyphenyl)but-1-en-1- |
| | yl)phenoxy)-4,16-dioxo-7,10,13-trioxa-3,17-diazanonadecanoyl)-4-hydroxy-N-(4- |
| | (4-methylthiazol-5-yl)benzyl)pyrrolidine-2-carboxamide |
| SIAIS307147 | N1-(2-(4-(4-chloro-1,2-bis(4-hydroxyphenyl)but-1-en-1-yl)phenoxy)ethyl)-N16- |
| | ((S)-1-((2S,4R)-4-hydroxy-2-((4-(4-methylthiazol-5- |
| | yl)benzyl)carbamoyl)pyrrolidin-1-yl)-3,3-dimethyl-1-oxobutan-2-yl)-4,7,10,13- |
| | tetraoxahexadecanediamide |
| SIAIS307148 | N1-(2-(4-(4-chloro-1,2-bis(4-hydroxyphenyl)but-1-en-1-yl)phenoxy)ethyl)-N19- |
| | ((S)-1-((2S,4R)-4-hydroxy-2-((4-(4-methylthiazol-5- |
| | yl)benzyl)carbamoyl)pyrrolidin-1-yl)-3,3-dimethyl-1-oxobutan-2-yl)-4,7,10,13,16- |
| | pentaoxanonadecanediamide |
| | N1-(2-(4-(4-chloro-1,2-bis(4-hydroxyphenyl)but-1-en-1-yl)phenoxy)ethyl)-N4- |
| | ((S)-1-((2S,4R)-4-hydroxy-2-((4-(4-methylthiazol-5- |
| | yl)benzyl)carbamoyl)pyrrolidin-1-yl)-3,3-dimethyl-1-oxobutan-2-yl)succinamide |
| SIAIS307149 | N1-(2-(4-(4-chloro-1,2-bis(4-hydroxyphenyl)but-1-en-1-yl)phenoxy)ethyl)-N5- |
| | ((S)-1-((2S,4R)-4-hydroxy-2-((4-(4-methylthiazol-5- |
| | yl)benzyl)carbamoyl)pyrrolidin-1-yl)-3,3-dimethyl-1-oxobutan-2-yl)glutaramide |
| SIAIS307150 | N1-(2-(4-(4-chloro-1,2-bis(4-hydroxyphenyl)but-1-en-1-yl)phenoxy)ethyl)-N6- |
| | ((S)-1-((2S,4R)-4-hydroxy-2-((4-(4-methylthiazol-5- |
| | yl)benzyl)carbamoyl)pyrrolidin-1-yl)-3,3-dimethyl-1-oxobutan-2-yl)adipamide |
| SIAIS307151 | N1-(2-(4-(4-chloro-1,2-bis(4-hydroxyphenyl)but-1-en-1-yl)phenoxy)ethyl)-N7- |
| | ((S)-1-((2S,4R)-4-hydroxy-2-((4-(4-methylthiazol-5- |
| | yl)benzyl)carbamoyl)pyrrolidin-1-yl)-3,3-dimethyl-1-oxobutan-2- |
| | yl)heptanediamide |
| SIAIS307152 | N1-(2-(4-(4-chloro-1,2-bis(4-hydroxyphenyl)but-1-en-1-yl)phenoxy)ethyl)-N8- |
| | ((S)-1-((2S,4R)-4-hydroxy-2-((4-(4-methylthiazol-5- |
| | yl)benzyl)carbamoyl)pyrrolidin-1-yl)-3,3-dimethyl-1-oxobutan-2-yl)octanediamide |
| SIAIS307153 | N1-(2-(4-(4-chloro-1,2-bis(4-hydroxyphenyl)but-1-en-1-yl)phenoxy)ethyl)-N9- |
| | ((S)-1-((2S,4R)-4-hydroxy-2-((4-(4-methylthiazol-5- |
| | yl)benzyl)carbamoyl)pyrrolidin-1-yl)-3,3-dimethyl-1-oxobutan-2- |
| | yl)nonanediamide |
| SIAIS307154 | N1-(2-(4-(4-chloro-1,2-bis(4-hydroxyphenyl)but-1-en-1-yl)phenoxy)ethyl)-N10- |
| | ((S)-1-((2S,4R)-4-hydroxy-2-((4-(4-methylthiazol-5- |
| | yl)benzyl)carbamoyl)pyrrolidin-1-yl)-3,3-dimethyl-1-oxobutan-2-yl)decanediamide |
| SIAIS307155 | N1-(2-(4-(4-chloro-1,2-bis(4-hydroxyphenyl)but-1-en-1-yl)phenoxy)ethyl)-N11- |
| | ((S)-1-((2S,4R)-4-hydroxy-2-((4-(4-methylthiazol-5- |
| | yl)benzyl)carbamoyl)pyrrolidin-1-yl)-3,3-dimethyl-1-oxobutan-2- |
| | yl)undecanediamide |
| | N1-(2-(4-(4-chloro-1,2-bis(4-hydroxyphenyl)but-1-en-1-yl)phenoxy)ethyl)-N14- |
| | ((S)-1-((2S,4R)-4-hydroxy-2-((4-(4-methylthiazol-5- |
| | yl)benzyl)carbamoyl)pyrrolidin-1-yl)-3,3-dimethyl-1-oxobutan-2- |
| | yl)tetradecanediamide |
| | N1-(2-(4-(4-chloro-1,2-bis(4-hydroxyphenyl)but-1-en-1-yl)phenoxy)ethyl)-N16- |
| | ((S)-1-((2S,4R)-4-hydroxy-2-((4-(4-methylthiazol-5- |
| | yl)benzyl)carbamoyl)pyrrolidin-1-yl)-3,3-dimethyl-1-oxobutan-2- |
| | yl)hexadecanediamide |
| | (2S,4R)-1-((S)-2-(3-(4-(3-((2-(4-(4-chloro-1,2-bis(4-hydroxyphenyl)but-1-en-1- |
| | yl)phenoxy)ethyl)amino)-3-oxopropyl)piperazin-1-yl)propanamido)-3,3- |
| | dimethylbutanoyl)-4-hydroxy-N-(4-(4-methylthiazol-5-yl)benzyl)pyrrolidine-2- |
| | carboxamide |
| | (2S,4R)-1-((S)-2-(3-(4-(3-((2-(4-(4-chloro-1,2-bis(4-hydroxyphenyl)but-1-en-1- |
| | yl)phenoxy)ethyl)amino)-3-oxopropyl)phenyl)propanamido)-3,3- |
| | dimethylbutanoyl)-4-hydroxy-N-(4-(4-methylthiazol-5-yl)benzyl)pyrrolidine-2- |
| | carboxamide |
| | N-(2-(4-(4-chloro-1,2-bis(4-hydroxyphenyl)but-1-en-1-yl)phenoxy)ethyl)-3-(4-(2- |
| | ((2-(2,6-dioxopiperidin-3-yl)-1,3-dioxoisoindolin-4-yl)amino)ethyl)piperazin-1- |
| | yl)propanamide |
| | N-(2-(4-(4-chloro-1,2-bis(4-hydroxyphenyl)but-1-en-1-yl)phenoxy)ethyl)-3-(4-(2- |
| | ((2-(2,6-dioxopiperidin-3-yl)-1,3-dioxoisoindolin-4- |
| | yl)amino)ethyl)phenyl)propanamide |
| | (2S,4R)-1-((S)-2-(7-((2-(4-(4-chloro-1,2-bis(4-hydroxyphenyl)but-1-en-1- |
| | yl)phenoxy)ethyl)amino)heptanamido)-3,3-dimethylbutanoyl)-4-hydroxy-N-(4-(4- |
| | methylthiazol-5-yl)benzyl)pyrrolidine-2-carboxamide |
| | (2S,4R)-1-((S)-2-((7-((2-(4-(4-chloro-1,2-bis(4-hydroxyphenyl)but-1-en-1- |
| | yl)phenoxy)ethyl)amino)-7-oxoheptyl)amino)-3,3-dimethylbutanoyl)-4-hydroxy- |
| | N-(4-(4-methylthiazol-5-yl)benzyl)pyrrolidine-2-carboxamide |
| | (2S,4R)-1-((S)-2-((7-((2-(4-(4-chloro-1,2-bis(4-hydroxyphenyl)but-1-en-1- |
| | yl)phenoxy)ethyl)amino)heptyl)amino)-3,3-dimethylbutanoyl)-4-hydroxy-N-(4-(4- |
| | methylthiazol-5-yl)benzyl)pyrrolidine-2-carboxamide |
| | 4-((2-(3-(4-(2-(4-(4-chloro-1,2-bis(4-hydroxyphenyl)but-1-en-1- |
| | yl)phenoxy)ethyl)piperazin-1-yl)-3-oxopropoxy)ethyl)amino)-2-(2,6- |
| | dioxopiperidin-3-yl)isoindoline-1,3-dione |
| | 4-((2-(2-(3-(4-(2-(4-(4-chloro-1,2-bis(4-hydroxyphenyl)but-1-en-1- |
| | yl)phenoxy)ethyl)piperazin-1-yl)-3-oxopropoxy)ethoxy)ethyl)amino)-2-(2,6- |
| | dioxopiperidin-3-yl)isoindoline-1,3-dione |
| | 4-((2-(2-(2-(3-(4-(2-(4-(4-chloro-1,2-bis(4-hydroxyphenyl)but-1-en-1- |
| | yl)phenoxy)ethyl)piperazin-1-yl)-3-oxopropoxy)ethoxy)ethoxy)ethyl)amino)-2- |
| | (2,6-dioxopiperidin-3-yl)isoindoline-1,3-dione |
| | 4-((15-(4-(2-(4-(4-chloro-1,2-bis(4-hydroxyphenyl)but-1-en-1- |
| | yl)phenoxy)ethyl)piperazin-1-yl)-15-oxo-3,6,9,12-tetraoxapentadecyl)amino)-2- |
| | (2,6-dioxopiperidin-3-yl)isoindoline-1,3-dione |
| | 4-((18-(4-(2-(4-(4-chloro-1,2-bis(4-hydroxyphenyl)but-1-en-1- |
| | yl)phenoxy)ethyl)piperazin-1-yl)-18-oxo-3,6,9,12,15-pentaoxaoctadecyl)amino)-2- |
| | (2,6-dioxopiperidin-3-yl)isoindoline-1,3-dione |
| | 4-((2-(4-(2-(4-(4-chloro-1,2-bis(4-hydroxyphenyl)but-1-en-1- |
| | yl)phenoxy)ethyl)piperazin-1-yl)-2-oxoethyl)amino)-2-(2,6-dioxopiperidin-3- |
| | yl)isoindoline-1,3-dione |
| | 4-((3-(4-(2-(4-(4-chloro-1,2-bis(4-hydroxyphenyl)but-1-en-1- |
| | yl)phenoxy)ethyl)piperazin-1-yl)-3-oxopropyl)amino)-2-(2,6-dioxopiperidin-3- |
| | yl)isoindoline-1,3-dione |
| | 4-((4-(4-(2-(4-(4-chloro-1,2-bis(4-hydroxyphenyl)but-1-en-1- |
| | yl)phenoxy)ethyl)piperazin-1-yl)-4-oxobutyl)amino)-2-(2,6-dioxopiperidin-3- |
| | yl)isoindoline-1,3-dione |
| | 4-((5-(4-(2-(4-(4-chloro-1,2-bis(4-hydroxyphenyl)but-1-en-1- |
| | yl)phenoxy)ethyl)piperazin-1-yl)-5-oxopentyl)amino)-2-(2,6-dioxopiperidin-3- |
| | yl)isoindoline-1,3-dione |
| | 4-((6-(4-(2-(4-(4-chloro-1,2-bis(4-hydroxyphenyl)but-1-en-1- |
| | yl)phenoxy)ethyl)piperazin-1-yl)-6-oxohexyl)amino)-2-(2,6-dioxopiperidin-3- |
| | yl)isoindoline-1,3-dione |
| | 4-((7-(4-(2-(4-(4-chloro-1,2-bis(4-hydroxyphenyl)but-1-en-1- |
| | yl)phenoxy)ethyl)piperazin-1-yl)-7-oxoheptyl)amino)-2-(2,6-dioxopiperidin-3- |
| | yl)isoindoline-1,3-dione |
| | 3-(4-((2-(3-(4-(2-(4-(4-chloro-1,2-bis(4-hydroxyphenyl)but-1-en-1- |
| | yl)phenoxy)ethyl)piperazin-1-yl)-3-oxopropoxy)ethyl)amino)-1-oxoisoindolin-2- |
| | yl)piperidine-2,6-dione |
| | 3-(4-((2-(2-(3-(4-(2-(4-(4-chloro-1,2-bis(4-hydroxyphenyl)but-1-en-1- |
| | yl)phenoxy)ethyl)piperazin-1-yl)-3-oxopropoxy)ethoxy)ethyl)amino)-1- |
| | oxoisoindolin-2-yl)piperidine-2,6-dione |
| | 3-(4-((2-(2-(2-(3-(4-(2-(4-(4-chloro-1,2-bis(4-hydroxyphenyl)but-1-en-1- |
| | yl)phenoxy)ethyl)piperazin-1-yl)-3-oxopropoxy)ethoxy)ethoxy)ethyl)amino)-1- |
| | oxoisoindolin-2-yl)piperidine-2,6-dione |
| | 3-(4-((15-(4-(2-(4-(4-chloro-1,2-bis(4-hydroxyphenyl)but-1-en-1- |
| | yl)phenoxy)ethyl)piperazin-1-yl)-15-oxo-3,6,9,12-tetraoxapentadecyl)amino)-1- |
| | oxoisoindolin-2-yl)piperidine-2,6-dione |
| | 3-(4-((18-(4-(2-(4-(4-chloro-1,2-bis(4-hydroxyphenyl)but-1-en-1- |
| | yl)phenoxy)ethyl)piperazin-1-yl)-18-oxo-3,6,9,12,15-pentaoxaoctadecyl)amino)-1- |
| | oxoisoindolin-2-yl)piperidine-2,6-dione |
| | 3-(4-((2-(4-(2-(4-(4-chloro-1,2-bis(4-hydroxyphenyl)but-1-en-1- |
| | yl)phenoxy)ethyl)piperazin-1-yl)-2-oxoethyl)amino)-1-oxoisoindolin-2- |
| | yl)piperidine-2,6-dione |
| | 3-(4-((3-(4-(2-(4-(4-chloro-1,2-bis(4-hydroxyphenyl)but-1-en-1- |
| | yl)phenoxy)ethyl)piperazin-1-yl)-3-oxopropyl)amino)-1-oxoisoindolin-2- |
| | yl)piperidine-2,6-dione |
| | 3-(4-((4-(4-(2-(4-(4-chloro-1,2-bis(4-hydroxyphenyl)but-1-en-1- |
| | yl)phenoxy)ethyl)piperazin-1-yl)-4-oxobutyl)amino)-1-oxoisoindolin-2- |
| | yl)piperidine-2,6-dione |
| | 3-(4-((5-(4-(2-(4-(4-chloro-1,2-bis(4-hydroxyphenyl)but-1-en-1- |
| | yl)phenoxy)ethyl)piperazin-1-yl)-5-oxopentyl)amino)-1-oxoisoindolin-2- |
| | yl)piperidine-2,6-dione |
| | 3-(4-((6-(4-(2-(4-(4-chloro-1,2-bis(4-hydroxyphenyl)but-1-en-1- |
| | yl)phenoxy)ethyl)piperazin-1-yl)-6-oxohexyl)amino)-1-oxoisoindolin-2- |
| | yl)piperidine-2,6-dione |
| | 3-(4-((7-(4-(2-(4-(4-chloro-1,2-bis(4-hydroxyphenyl)but-1-en-1- |
| | yl)phenoxy)ethyl)piperazin-1-yl)-7-oxoheptyl)amino)-1-oxoisoindolin-2- |
| | yl)piperidine-2,6-dione |
| | (2S,4R)-1-((S)-2-(2-(2-(2-(4-(2-(4-(4-chloro-1,2-bis(4-hydroxyphenyl)but-1-en-1- |
| | yl)phenoxy)ethyl)piperazin-1-yl)-2-oxoethoxy)ethoxy)acetamido)-3,3- |
| | dimethylbutanoyl)-4-hydroxy-N-(4-(4-methylthiazol-5-yl)benzyl)pyrrolidine-2- |
| | carboxamide |
| | (2S,4R)-1-((S)-2-(3-(2-(3-(4-(2-(4-(4-chloro-1,2-bis(4-hydroxyphenyl)but-1-en-1- |
| | yl)phenoxy)ethyl)piperazin-1-yl)-3-oxopropoxy)ethoxy)propanamido)-3,3- |
| | dimethylbutanoyl)-4-hydroxy-N-(4-(4-methylthiazol-5-yl)benzyl)pyrrolidine-2- |
| | carboxamide |
| | (2S,4R)-1-((S)-2-(tert-butyl)-16-(4-(2-(4-(4-chloro-1,2-bis(4-hydroxyphenyl)but- |
| | 1-en-1-yl)phenoxy)ethyl)piperazin-1-yl)-4,16-dioxo-7,10,13-trioxa-3- |
| | azahexadecanoyl)-4-hydroxy-N-(4-(4-methylthiazol-5-yl)benzyl)pyrrolidine-2- |
| | carboxamide |
| | (2S,4R)-1-((S)-2-(tert-butyl)-19-(4-(2-(4-(4-chloro-1,2-bis(4-hydroxyphenyl)but- |
| | 1-en-1-yl)phenoxy)ethyl)piperazin-1-yl)-4,19-dioxo-7,10,13,16-tetraoxa-3- |
| | azanonadecanoyl)-4-hydroxy-N-(4-(4-methylthiazol-5-yl)benzyl)pyrrolidine-2- |
| | carboxamide |
| | (2S,4R)-1-((S)-2-(tert-butyl)-22-(4-(2-(4-(4-chloro-1,2-bis(4-hydroxyphenyl)but- |
| | 1-en-1-yl)phenoxy)ethyl)piperazin-1-yl)-4,22-dioxo-7,10,13,16,19-pentaoxa-3- |
| | azadocosanoyl)-4-hydroxy-N-(4-(4-methylthiazol-5-yl)benzyl)pyrrolidine-2- |
| | carboxamide |
| | (2S,4R)-1-((S)-2-(4-(4-(2-(4-(4-chloro-1,2-bis(4-hydroxyphenyl)but-1-en-1- |
| | yl)phenoxy)ethyl)piperazin-1-yl)-4-oxobutanamido)-3,3-dimethylbutanoyl)-4- |
| | hydroxy-N-(4-(4-methylthiazol-5-yl)benzyl)pyrrolidine-2-carboxamide |
| | (2S,4R)-1-((S)-2-(5-(4-(2-(4-(4-chloro-1,2-bis(4-hydroxyphenyl)but-1-en-1- |
| | yl)phenoxy)ethyl)piperazin-1-yl)-5-oxopentanamido)-3,3-dimethylbutanoyl)-4- |
| | hydroxy-N-(4-(4-methylthiazol-5-yl)benzyl)pyrrolidine-2-carboxamide |
| | (2S,4R)-1-((S)-2-(6-(4-(2-(4-(4-chloro-1,2-bis(4-hydroxyphenyl)but-1-en-1- |
| | yl)phenoxy)ethyl)piperazin-1-yl)-6-oxohexanamido)-3,3-dimethylbutanoyl)-4- |
| | hydroxy-N-(4-(4-methylthiazol-5-yl)benzyl)pyrrolidine-2-carboxamide |
| | (2S,4R)-1-((S)-2-(7-(4-(2-(4-(4-chloro-1,2-bis(4-hydroxyphenyl)but-1-en-1- |
| | yl)phenoxy)ethyl)piperazin-1-yl)-7-oxoheptanamido)-3,3-dimethylbutanoyl)-4- |
| | hydroxy-N-(4-(4-methylthiazol-5-yl)benzyl)pyrrolidine-2-carboxamide |
| | (2S,4R)-1-((S)-2-(8-(4-(2-(4-(4-chloro-1,2-bis(4-hydroxyphenyl)but-1-en-1- |
| | yl)phenoxy)ethyl)piperazin-1-yl)-8-oxooctanamido)-3,3-dimethylbutanoyl)-4- |
| | hydroxy-N-(4-(4-methylthiazol-5-yl)benzyl)pyrrolidine-2-carboxamide |
| | (2S,4R)-1-((S)-2-(9-(4-(2-(4-(4-chloro-1,2-bis(4-hydroxyphenyl)but-1-en-1- |
| | yl)phenoxy)ethyl)piperazin-1-yl)-9-oxononanamido)-3,3-dimethylbutanoyl)-4- |
| | hydroxy-N-(4-(4-methylthiazol-5-yl)benzyl)pyrrolidine-2-carboxamide |
| | (2S,4R)-1-((S)-2-(10-(4-(2-(4-(4-chloro-1,2-bis(4-hydroxyphenyl)but-1-en-1- |
| | yl)phenoxy)ethyl)piperazin-1-yl)-10-oxodecanamido)-3,3-dimethylbutanoyl)-4- |
| | hydroxy-N-(4-(4-methylthiazol-5-yl)benzyl)pyrrolidine-2-carboxamide |
| | (2S,4R)-1-((S)-2-(7-(4-(2-(4-(4-chloro-1,2-bis(4-hydroxyphenyl)but-1-en-1- |
| | yl)phenoxy)ethyl)piperazin-1-yl)heptanamido)-3,3-dimethylbutanoyl)-4-hydroxy- |
| | N-(4-(4-methylthiazol-5-yl)benzyl)pyrrolidine-2-carboxamide |
| | (2S,4R)-1-((S)-2-((7-(4-(2-(4-(4-chloro-1,2-bis(4-hydroxyphenyl)but-1-en-1- |
| | yl)phenoxy)ethyl)piperazin-1-yl)-7-oxoheptyl)amino)-3,3-dimethylbutanoyl)-4- |
| | hydroxy-N-(4-(4-methylthiazol-5-yl)benzyl)pyrrolidine-2-carboxamide |
| | (2S,4R)-1-((S)-2-((7-(4-(2-(4-(4-chloro-1,2-bis(4-hydroxyphenyl)but-1-en-1- |
| | yl)phenoxy)ethyl)piperazin-1-yl)heptyl)amino)-3,3-dimethylbutanoyl)-4-hydroxy- |
| | N-(4-(4-methylthiazol-5-yl)benzyl)pyrrolidine-2-carboxamide |
| | N-(2-(4-(1,2-bis(4-hydroxyphenyl)but-1-en-1-yl)phenoxy)ethyl)-3-(2-((2-(2,6- |
| | dioxopiperidin-3-yl)-1,3-dioxoisoindolin-4-yl)amino)ethoxy)propanamide |
| | N-(2-(4-(1,2-bis(4-hydroxyphenyl)but-1-en-1-yl)phenoxy)ethyl)-3-(2-(2-((2-(2,6- |
| | dioxopiperidin-3-yl)-1,3-dioxoisoindolin-4-yl)amino)ethoxy)ethoxy)propanamide |
| | N-(2-(4-(1,2-bis(4-hydroxyphenyl)but-1-en-1-yl)phenoxy)ethyl)-3-(2-(2-(2-((2- |
| | (2,6-dioxopiperidin-3-yl)-1,3-dioxoisoindolin-4- |
| | yl)amino)ethoxy)ethoxy)ethoxy)propanamide |
| | N-(2-(4-(1,2-bis(4-hydroxyphenyl)but-1-en-1-yl)phenoxy)ethyl)-1-((2-(2,6- |
| | dioxopiperidin-3-yl)-1,3-dioxoisoindolin-4-yl)amino)-3,6,9,12-tetraoxapentadecan- |
| | 15-amide |
| | N-(2-(4-(1,2-bis(4-hydroxyphenyl)but-1-en-1-yl)phenoxy)ethyl)-1-((2-(2,6- |
| | dioxopiperidin-3-yl)-1,3-dioxoisoindolin-4-yl)amino)-3,6,9,12,15- |
| | pentaoxaoctadecan-18-amide |
| | N-(2-(4-(1,2-bis(4-hydroxyphenyl)but-1-en-1-yl)phenoxy)ethyl)-2-((2-(2,6- |
| | dioxopiperidin-3-yl)-1,3-dioxoisoindolin-4-yl)amino)acetamide |
| | N-(2-(4-(1,2-bis(4-hydroxyphenyl)but-1-en-1-yl)phenoxy)ethyl)-3-((2-(2,6- |
| | dioxopiperidin-3-yl)-1,3-dioxoisoindolin-4-yl)amino)propanamide |
| | N-(2-(4-(1,2-bis(4-hydroxyphenyl)but-1-en-1-yl)phenoxy)ethyl)-4-((2-(2,6- |
| | dioxopiperidin-3-yl)-1,3-dioxoisoindolin-4-yl)amino)butanamide |
| | N-(2-(4-(1,2-bis(4-hydroxyphenyl)but-1-en-1-yl)phenoxy)ethyl)-5-((2-(2,6- |
| | dioxopiperidin-3-yl)-1,3-dioxoisoindolin-4-yl)amino)pentanamide |
| | N-(2-(4-(1,2-bis(4-hydroxyphenyl)but-1-en-1-yl)phenoxy)ethyl)-6-((2-(2,6- |
| | dioxopiperidin-3-yl)-1,3-dioxoisoindolin-4-yl)amino)hexanamide |
| | N-(2-(4-(1,2-bis(4-hydroxyphenyl)but-1-en-1-yl)phenoxy)ethyl)-7-((2-(2,6- |
| | dioxopiperidin-3-yl)-1,3-dioxoisoindolin-4-yl)amino)heptanamide |
| | N-(2-(4-(1,2-bis(4-hydroxyphenyl)but-1-en-1-yl)phenoxy)ethyl)-3-(3-((2-(2,6- |
| | dioxopiperidin-3-yl)-1-oxoisoindolin-4-yl)amino)-3-oxopropoxy)propanamide |
| | N-(2-(4-(1,2-bis(4-hydroxyphenyl)but-1-en-1-yl)phenoxy)ethyl)-3-(2-(3-((2-(2,6- |
| | dioxopiperidin-3-yl)-1-oxoisoindolin-4-yl)amino)-3- |
| | oxopropoxy)ethoxy)propanamide |
| | N-(2-(4-(1,2-bis(4-hydroxyphenyl)but-1-en-1-yl)phenoxy)ethyl)-3-(2-(2-(3-((2- |
| | (2,6-dioxopiperidin-3-yl)-1-oxoisoindolin-4-yl)amino)-3- |
| | oxopropoxy)ethoxy)ethoxy)propanamide |
| | N1-(2-(4-(1,2-bis(4-hydroxyphenyl)but-1-en-1-yl)phenoxy)ethyl)-N16-(2-(2,6- |
| | dioxopiperidin-3-yl)-1-oxoisoindolin-4-yl)-4,7,10,13-tetraoxahexadecanediamide |
| | N1-(2-(4-(1,2-bis(4-hydroxyphenyl)but-1-en-1-yl)phenoxy)ethyl)-N19-(2-(2,6- |
| | dioxopiperidin-3-yl)-1-oxoisoindolin-4-yl)-4,7,10,13,16- |
| | pentaoxanonadecanediamide |
| | N-(2-(4-(1,2-bis(4-hydroxyphenyl)but-1-en-1-yl)phenoxy)ethyl)-3-(2-((2-(2,6- |
| | dioxopiperidin-3-yl)-1-oxoisoindolin-4-yl)amino)ethoxy)propanamide |
| | N-(2-(4-(1,2-bis(4-hydroxyphenyl)but-1-en-1-yl)phenoxy)ethyl)-3-(2-(2-((2-(2,6- |
| | dioxopiperidin-3-yl)-1-oxoisoindolin-4-yl)amino)ethoxy)ethoxy)propanamide |
| | N-(2-(4-(1,2-bis(4-hydroxyphenyl)but-1-en-1-yl)phenoxy)ethyl)-3-(2-(2-(2-((2- |
| | (2,6-dioxopiperidin-3-yl)-1-oxoisoindolin-4- |
| | yl)amino)ethoxy)ethoxy)ethoxy)propanamide |
| | N-(2-(4-(1,2-bis(4-hydroxyphenyl)but-1-en-1-yl)phenoxy)ethyl)-1-((2-(2,6- |
| | dioxopiperidin-3-yl)-1-oxoisoindolin-4-yl)amino)-3,6,9,12-tetraoxapentadecan-15- |
| | amide |
| | N-(2-(4-(1,2-bis(4-hydroxyphenyl)but-1-en-1-yl)phenoxy)ethyl)-1-((2-(2,6- |
| | dioxopiperidin-3-yl)-1-oxoisoindolin-4-yl)amino)-3,6,9,12,15-pentaoxaoctadecan- |
| | 18-amide |
| | N1-(2-(4-(1,2-bis(4-hydroxyphenyl)but-1-en-1-yl)phenoxy)ethyl)-N3-(2-(2,6- |
| | dioxopiperidin-3-yl)-1-oxoisoindolin-4-yl)malonamide |
| | N1-(2-(4-(1,2-bis(4-hydroxyphenyl)but-1-en-1-yl)phenoxy)ethyl)-N4-(2-(2,6- |
| | dioxopiperidin-3-yl)-1-oxoisoindolin-4-yl)succinamide |
| | N1-(2-(4-(1,2-bis(4-hydroxyphenyl)but-1-en-1-yl)phenoxy)ethyl)-N5-(2-(2,6- |
| | dioxopiperidin-3-yl)-1-oxoisoindolin-4-yl)glutaramide |
| | N1-(2-(4-(1,2-bis(4-hydroxyphenyl)but-1-en-1-yl)phenoxy)ethyl)-N6-(2-(2,6- |
| | dioxopiperidin-3-yl)-1-oxoisoindolin-4-yl)adipamide |
| | N1-(2-(4-(1,2-bis(4-hydroxyphenyl)but-1-en-1-yl)phenoxy)ethyl)-N7-(2-(2,6- |
| | dioxopiperidin-3-yl)-1-oxoisoindolin-4-yl)heptanediamide |
| | N1-(2-(4-(1,2-bis(4-hydroxyphenyl)but-1-en-1-yl)phenoxy)ethyl)-N8-(2-(2,6- |
| | dioxopiperidin-3-yl)-1-oxoisoindolin-4-yl)octanediamide |
| | N-(2-(4-(1,2-bis(4-hydroxyphenyl)but-1-en-1-yl)phenoxy)ethyl)-3-((2-(2,6- |
| | dioxopiperidin-3-yl)-1-oxoisoindolin-4-yl)amino)propanamide |
| | N-(2-(4-(1,2-bis(4-hydroxyphenyl)but-1-en-1-yl)phenoxy)ethyl)-4-((2-(2,6- |
| | dioxopiperidin-3-yl)-1-oxoisoindolin-4-yl)amino)butanamide |
| | N-(2-(4-(1,2-bis(4-hydroxyphenyl)but-1-en-1-yl)phenoxy)ethyl)-5-((2-(2,6- |
| | dioxopiperidin-3-yl)-1-oxoisoindolin-4-yl)amino)pentanamide |
| | N-(2-(4-(1,2-bis(4-hydroxyphenyl)but-1-en-1-yl)phenoxy)ethyl)-6-((2-(2,6- |
| | dioxopiperidin-3-yl)-1-oxoisoindolin-4-yl)amino)hexanamide |
| | N-(2-(4-(1,2-bis(4-hydroxyphenyl)but-1-en-1-yl)phenoxy)ethyl)-7-((2-(2,6- |
| | dioxopiperidin-3-yl)-1-oxoisoindolin-4-yl)amino)heptanamide |
| | N-(2-(4-(1,2-bis(4-hydroxyphenyl)but-1-en-1-yl)phenoxy)ethyl)-8-((2-(2,6- |
| | dioxopiperidin-3-yl)-1-oxoisoindolin-4-yl)amino)octanamide |
| | (2S,4R)-1-((S)-14-(4-(1,2-bis(4-hydroxyphenyl)but-1-en-1-yl)phenoxy)-2-(tert- |
| | butyl)-4,11-dioxo-6,9-dioxa-3,12-diazatetradecanoyl)-4-hydroxy-N-(4-(4- |
| | methylthiazol-5-yl)benzyl)pyrrolidine-2-carboxamide |
| SIAIS208173 | (2S,4R)-1-((S)-16-(4-(1,2-bis(4-hydroxyphenyl)but-1-en-1-yl)phenoxy)-2-(tert- |
| | butyl)-4,13-dioxo-7,10-dioxa-3,14-diazahexadecanoyl)-4-hydroxy-N-(4-(4- |
| | methylthiazol-5-yl)benzyl)pyrrolidine-2-carboxamide |
| SIAIS208174 | (2S,4R)-1-((S)-19-(4-(1,2-bis(4-hydroxyphenyl)but-1-en-1-yl)phenoxy)-2-(tert- |
| | butyl)-4,16-dioxo-7,10,13-trioxa-3,17-diazanonadecanoyl)-4-hydroxy-N-(4-(4- |
| | methylthiazol-5-yl)benzyl)pyrrolidine-2-carboxamide |
| | N1-(2-(4-(1,2-bis(4-hydroxyphenyl)but-1-en-1-yl)phenoxy)ethyl)-N16-((S)-1- |
| | ((2S,4R)-4-hydroxy-2-((4-(4-methylthiazol-5-yl)benzyl)carbamoyl)pyrrolidin-1- |
| | yl)-3,3-dimethyl-1-oxobutan-2-yl)-4,7,10,13-tetraoxahexadecanediamide |
| | N1-(2-(4-(1,2-bis(4-hydroxyphenyl)but-1-en-1-yl)phenoxy)ethyl)-N19-((S)-1- |
| | ((2S,4R)-4-hydroxy-2-((4-(4-methylthiazol-5-yl)benzyl)carbamoyl)pyrrolidin-1- |
| | yl)-3,3-dimethyl-1-oxobutan-2-yl)-4,7,10,13,16-pentaoxanonadecanediamide |
| | N1-(2-(4-(1,2-bis(4-hydroxyphenyl)but-1-en-1-yl)phenoxy)ethyl)-N4-((S)-1- |
| | ((2S,4R)-4-hydroxy-2-((4-(4-methylthiazol-5-yl)benzyl)carbamoyl)pyrrolidin-1- |
| | yl)-3,3-dimethyl-1-oxobutan-2-yl)succinamide |
| | Nl-(2-(4-(1,2-bis(4-hydroxyphenyl)but-1-en-1-yl)phenoxy)ethyl)-N5-((S)-1- |
| | ((2S,4R)-4-hydroxy-2-((4-(4-methylthiazol-5-yl)benzyl)carbamoyl)pyrrolidin-1- |
| | yl)-3,3-dimethyl-1-oxobutan-2-yl)glutaramide |
| | N1-(2-(4-(1,2-bis(4-hydroxyphenyl)but-1-en-1-yl)phenoxy)ethyl)-N6-((S)-1- |
| | ((2S,4R)-4-hydroxy-2-((4-(4-methylthiazol-5-yl)benzyl)carbamoyl)pyrrolidin-1- |
| | yl)-3,3-dimethyl-1-oxobutan-2-yl)adipamide |
| | N1-(2-(4-(1,2-bis(4-hydroxyphenyl)but-1-en-1-yl)phenoxy)ethyl)-N7-((S)-1- |
| | ((2S,4R)-4-hydroxy-2-((4-(4-methylthiazol-5-yl)benzyl)carbamoyl)pyrrolidin-1- |
| | yl)-3,3-dimethyl-1-oxobutan-2-yl)heptanediamide |
| | N1-(2-(4-(1,2-bis(4-hydroxyphenyl)but-1-en-1-yl)phenoxy)ethyl)-N8-((S)-1- |
| | ((2S,4R)-4-hydroxy-2-((4-(4-methylthiazol-5-yl)benzyl)carbamoyl)pyrrolidin-1- |
| | yl)-3,3-dimethyl-1-oxobutan-2-yl)octanediamide |
| SIAIS208167 | N1-(2-(4-(1,2-bis(4-hydroxyphenyl)but-1-en-1-yl)phenoxy)ethyl)-N9-((S)-1- |
| | ((2S,4R)-4-hydroxy-2-((4-(4-methylthiazol-5-yl)benzyl)carbamoyl)pyrrolidin-1- |
| | yl)-3,3-dimethyl-1-oxobutan-2-yl)nonanediamide |
| SIAIS208168 | N1-(2-(4-(1,2-bis(4-hydroxyphenyl)but-1-en-1-yl)phenoxy)ethyl)-N10-((S)-1- |
| | ((2S,4R)-4-hydroxy-2-((4-(4-methylthiazol-5-yl)benzyl)carbamoyl)pyrrolidin-1- |
| | yl)-3,3-dimethyl-1-oxobutan-2-yl)decanediamide |
| SIAIS208169 | N1-(2-(4-(1,2-bis(4-hydroxyphenyl)but-1-en-1-yl)phenoxy)ethyl)-N11-((S)-1- |
| | ((2S,4R)-4-hydroxy-2-((4-(4-methylthiazol-5-yl)benzyl)carbamoyl)pyrrolidin-1- |
| | yl)-3,3-dimethyl-1-oxobutan-2-yl)undecanediamide |
| | N1-(2-(4-(1,2-bis(4-hydroxyphenyl)but-1-en-1-yl)phenoxy)ethyl)-N14-((S)-1- |
| | ((2S,4R)-4-hydroxy-2-((4-(4-methylthiazol-5-yl)benzyl)carbamoyl)pyrrolidin-1- |
| | yl)-3,3-dimethyl-1-oxobutan-2-yl)tetradecanediamide |
| | N1-(2-(4-(1,2-bis(4-hydroxyphenyl)but-1-en-1-yl)phenoxy)ethyl)-N16-((S)-1- |
| | ((2S,4R)-4-hydroxy-2-((4-(4-methylthiazol-5-yl)benzyl)carbamoyl)pyrrolidin-1- |
| | yl)-3,3-dimethyl-1-oxobutan-2-yl)hexadecanediamide |
| | (2S,4R)-1-((S)-2-(3-(4-(3-((2-(4-(1,2-bis(4-hydroxyphenyl)but-1-en-1- |
| | yl)phenoxy)ethyl)amino)-3-oxopropyl)piperazin-1-yl)propanamido)-3,3- |
| | dimethylbutanoyl)-4-hydroxy-N-(4-(4-methylthiazol-5-yl)benzyl)pyrrolidine-2- |
| | carboxamide |
| | (2S,4R)-1-((S)-2-(3-(4-(3-((2-(4-(1,2-bis(4-hydroxyphenyl)but-1-en-1- |
| | yl)phenoxy)ethyl)amino)-3-oxopropyl)phenyl)propanamido)-3,3- |
| | dimethylbutanoyl)-4-hydroxy-N-(4-(4-methylthiazol-5-yl)benzyl)pyrrolidine-2- |
| | carboxamide |
| SIAIS208172 | N-(2-(4-(1,2-bis(4-hydroxyphenyl)but-1-en-1-yl)phenoxy)ethyl)-3-(4-(2-((2-(2,6- |
| | dioxopiperidin-3-yl)-1,3-dioxoisoindolin-4-yl)amino)ethyl)piperazin-1- |
| | yl)propanamide |
| | N-(2-(4-(1,2-bis(4-hydroxyphenyl)but-1-en-1-yl)phenoxy)ethyl)-3-(4-(2-((2-(2,6- |
| | dioxopiperidin-3-yl)-1,3-dioxoisoindolin-4-yl)amino)ethyl)phenyl)propanamide |
| | (2S,4R)-1-((S)-2-(7-((2-(4-(1,2-bis(4-hydroxyphenyl)but-1-en-1- |
| | yl)phenoxy)ethyl)amino)heptanamido)-3,3-dimethylbutanoyl)-4-hydroxy-N-(4-(4- |
| | methylthiazol-5-yl)benzyl)pyrrolidine-2-carboxamide |
| | (2S,4R)-1-((S)-2-((7-((2-(4-(1,2-bis(4-hydroxyphenyl)but-1-en-1- |
| | yl)phenoxy)ethyl)amino)-7-oxoheptyl)amino)-3,3-dimethylbutanoyl)-4-hydroxy- |
| | N-(4-(4-methylthiazol-5-yl)benzyl)pyrrolidine-2-carboxamide |
| | (2S,4R)-1-((S)-2-((7-((2-(4-(1,2-bis(4-hydroxyphenyl)but-1-en-1- |
| | yl)phenoxy)ethyl)amino)heptyl)amino)-3,3-dimethylbutanoyl)-4-hydroxy-N-(4-(4- |
| | methylthiazol-5-yl)benzyl)pyrrolidine-2-carboxamide |
| | 4-((2-(3-(4-(2-(4-(1,2-bis(4-hydroxyphenyl)but-1-en-1-yl)phenoxy)ethyl)piperazin- |
| | 1-yl)-3-oxopropoxy)ethyl)amino)-2-(2,6-dioxopiperidin-3-yl)isoindoline-1,3-dione |
| | 4-((2-(2-(3-(4-(2-(4-(1,2-bis(4-hydroxyphenyl)but-1-en-1- |
| | yl)phenoxy)ethyl)piperazin-1-yl)-3-oxopropoxy)ethoxy)ethyl)amino)-2-(2,6- |
| | dioxopiperidin-3-yl)isoindoline-1,3-dione |
| | 4-((2-(2-(2-(3-(4-(2-(4-(1,2-bis(4-hydroxyphenyl)but-1-en-1- |
| | yl)phenoxy)ethyl)piperazin-1-yl)-3-oxopropoxy)ethoxy)ethoxy)ethyl)amino)-2- |
| | (2,6-dioxopiperidin-3-yl)isoindoline-1,3-dione |
| | 4-((15-(4-(2-(4-(1,2-bis(4-hydroxyphenyl)but-1-en-1-yl)phenoxy)ethyl)piperazin- |
| | 1-yl)-15-oxo-3,6,9,12-tetraoxapentadecyl)amino)-2-(2,6-dioxopiperidin-3- |
| | yl)isoindoline-1,3-dione |
| | 4-((18-(4-(2-(4-(1,2-bis(4-hydroxyphenyl)but-1-en-1-yl)phenoxy)ethyl)piperazin- |
| | 1-yl)-18-oxo-3,6,9,12,15-pentaoxaoctadecyl)amino)-2-(2,6-dioxopiperidin-3- |
| | yl)isoindoline-1,3-dione |
| | 4-((2-(4-(2-(4-(1,2-bis(4-hydroxyphenyl)but-1-en-1-yl)phenoxy)ethyl)piperazin-1- |
| | yl)-2-oxoethyl)amino)-2-(2,6-dioxopiperidin-3-yl)isoindoline-1,3-dione |
| | 4-((3-(4-(2-(4-(1,2-bis(4-hydroxyphenyl)but-1-en-1-yl)phenoxy)ethyl)piperazin-1- |
| | yl)-3-oxopropyl)amino)-2-(2,6-dioxopiperidin-3-yl)isoindoline-1,3-dione |
| | 4-((4-(4-(2-(4-(1,2-bis(4-hydroxyphenyl)but-1-en-1-yl)phenoxy)ethyl)piperazin-1- |
| | yl)-4-oxobutyl)amino)-2-(2,6-dioxopiperidin-3-yl)isoindoline-1,3-dione |
| | 4-((5-(4-(2-(4-(1,2-bis(4-hydroxyphenyl)but-1-en-1-yl)phenoxy)ethyl)piperazin-1- |
| | yl)-5-oxopentyl)amino)-2-(2,6-dioxopiperidin-3-yl)isoindoline-1,3-dione |
| | 4-((6-(4-(2-(4-(1,2-bis(4-hydroxyphenyl)but-1-en-1-yl)phenoxy)ethyl)piperazin-1- |
| | yl)-6-oxohexyl)amino)-2-(2,6-dioxopiperidin-3-yl)isoindoline-1,3-dione |
| | 4-((7-(4-(2-(4-(1,2-bis(4-hydroxyphenyl)but-1-en-1-yl)phenoxy)ethyl)piperazin-1- |
| | yl)-7-oxoheptyl)amino)-2-(2,6-dioxopiperidin-3-yl)isoindoline-1,3-dione |
| | 3-(4-((2-(3-(4-(2-(4-(1,2-bis(4-hydroxyphenyl)but-1-en-1- |
| | yl)phenoxy)ethyl)piperazin-1-yl)-3-oxopropoxy)ethyl)amino)-1-oxoisoindolin-2- |
| | yl)piperidine-2,6-dione |
| | 3-(4-((2-(2-(3-(4-(2-(4-(1,2-bis(4-hydroxyphenyl)but-1-en-1- |
| | yl)phenoxy)ethyl)piperazin-1-yl)-3-oxopropoxy)ethoxy)ethyl)amino)-1- |
| | oxoisoindolin-2-yl)piperidine-2,6-dione |
| | 3-(4-((2-(2-(2-(3-(4-(2-(4-(1,2-bis(4-hydroxyphenyl)but-1-en-1- |
| | yl)phenoxy)ethyl)piperazin-1-yl)-3-oxopropoxy)ethoxy)ethoxy)ethyl)amino)-1- |
| | oxoisoindolin-2-yl)piperidine-2,6-dione |
| | 3-(4-((15-(4-(2-(4-(1,2-bis(4-hydroxyphenyl)but-1-en-1- |
| | yl)phenoxy)ethyl)piperazin-1-yl)-15-oxo-3,6,9,12-tetraoxapentadecyl)amino)-1- |
| | oxoisoindolin-2-yl)piperidine-2,6-dione |
| | 3-(4-((18-(4-(2-(4-(1,2-bis(4-hydroxyphenyl)but-1-en-1- |
| | yl)phenoxy)ethyl)piperazin-1-yl)-18-oxo-3,6,9,12,15-pentaoxaoctadecyl)amino)-1- |
| | oxoisoindolin-2-yl)piperidine-2,6-dione |
| | 3-(4-((2-(4-(2-(4-(1,2-bis(4-hydroxyphenyl)but-1-en-1-yl)phenoxy)ethyl)piperazin- |
| | 1-yl)-2-oxoethyl)amino)-1-oxoisoindolin-2-yl)piperidine-2,6-dione |
| | 3-(4-((3-(4-(2-(4-(1,2-bis(4-hydroxyphenyl)but-1-en-1-yl)phenoxy)ethyl)piperazin- |
| | 1-yl)-3-oxopropyl)amino)-1-oxoisoindolin-2-yl)piperidine-2,6-dione |
| | 3-(4-((4-(4-(2-(4-(1,2-bis(4-hydroxyphenyl)but-1-en-1-yl)phenoxy)ethyl)piperazin- |
| | 1-yl)-4-oxobutyl)amino)-1-oxoisoindolin-2-yl)piperidine-2,6-dione |
| | 3-(4-((5-(4-(2-(4-(1,2-bis(4-hydroxyphenyl)but-1-en-1-yl)phenoxy)ethyl)piperazin- |
| | 1-yl)-5-oxopentyl)amino)-1-oxoisoindolin-2-yl)piperidine-2,6-dione |
| | 3-(4-((6-(4-(2-(4-(1,2-bis(4-hydroxyphenyl)but-1-en-1-yl)phenoxy)ethyl)piperazin- |
| | 1-yl)-6-oxohexyl)amino)-1-oxoisoindolin-2-yl)piperidine-2,6-dione |
| | 3-(4-((7-(4-(2-(4-(1,2-bis(4-hydroxyphenyl)but-1-en-1-yl)phenoxy)ethyl)piperazin- |
| | 1-yl)-7-oxoheptyl)amino)-1-oxoisoindolin-2-yl)piperidine-2,6-dione |
| | (2S,4R)-1-((S)-2-(2-(2-(2-(4-(2-(4-(1,2-bis(4-hydroxyphenyl)but-1-en-1- |
| | yl)phenoxy)ethyl)piperazin-1-yl)-2-oxoethoxy)ethoxy)acetamido)-3,3- |
| | dimethylbutanoyl)-4-hydroxy-N-(4-(4-methylthiazol-5-yl)benzyl)pyrrolidine-2- |
| | carboxamide |
| | (2S,4R)-1-((S)-2-(3-(2-(3-(4-(2-(4-(1,2-bis(4-hydroxyphenyl)but-1-en-1- |
| | yl)phenoxy)ethyl)piperazin-1-yl)-3-oxopropoxy)ethoxy)propanamido)-3,3- |
| | dimethylbutanoyl)-4-hydroxy-N-(4-(4-methylthiazol-5-yl)benzyl)pyrrolidine-2- |
| | carboxamide |
| | (2S,4R)-1-((S)-16-(4-(2-(4-(1,2-bis(4-hydroxyphenyl)but-1-en-1- |
| | yl)phenoxy)ethyl)piperazin-1-yl)-2-(tert-butyl)-4,16-dioxo-7,10,13-trioxa-3- |
| | azahexadecanoyl)-4-hydroxy-N-(4-(4-methylthiazol-5-yl)benzyl)pyrrolidine-2- |
| | carboxamide |
| | (2S,4R)-1-((S)-19-(4-(2-(4-(1,2-bis(4-hydroxyphenyl)but-1-en-1- |
| | yl)phenoxy)ethyl)piperazin-1-yl)-2-(tert-butyl)-4,19-dioxo-7,10,13,16-tetraoxa-3- |
| | azanonadecanoyl)-4-hydroxy-N-(4-(4-methylthiazol-5-yl)benzyl)pyrrolidine-2- |
| | carboxamide |
| | (2S,4R)-1-((S)-22-(4-(2-(4-(1,2-bis(4-hydroxyphenyl)but-1-en-1- |
| | yl)phenoxy)ethyl)piperazin-1-yl)-2-(tert-butyl)-4,22-dioxo-7,10,13,16,19-pentaoxa- |
| | 3-azadocosanoyl)-4-hydroxy-N-(4-(4-methylthiazol-5-yl)benzyl)pyrrolidine-2- |
| | carboxamide |
| | (2S,4R)-1-((S)-2-(4-(4-(2-(4-(1,2-bis(4-hydroxyphenyl)but-1-en-1- |
| | yl)phenoxy)ethyl)piperazin-1-yl)-4-oxobutanamido)-3,3-dimethylbutanoyl)-4- |
| | hydroxy-N-(4-(4-methylthiazol-5-yl)benzyl)pyrrolidine-2-carboxamide |
| | (2S,4R)-1-((S)-2-(5-(4-(2-(4-(1,2-bis(4-hydroxyphenyl)but-1-en-1- |
| | yl)phenoxy)ethyl)piperazin-1-yl)-5-oxopentanamido)-3,3-dimethylbutanoyl)-4- |
| | hydroxy-N-(4-(4-methylthiazol-5-yl)benzyl)pyrrolidine-2-carboxamide |
| | (2S,4R)-1-((S)-2-(6-(4-(2-(4-(1,2-bis(4-hydroxyphenyl)but-1-en-1- |
| | yl)phenoxy)ethyl)piperazin-1-yl)-6-oxohexanamido)-3,3-dimethylbutanoyl)-4- |
| | hydroxy-N-(4-(4-methylthiazol-5-yl)benzyl)pyrrolidine-2-carboxamide |
| | (2S,4R)-1-((S)-2-(7-(4-(2-(4-(1,2-bis(4-hydroxyphenyl)but-1-en-1- |
| | yl)phenoxy)ethyl)piperazin-1-yl)-7-oxoheptanamido)-3,3-dimethylbutanoyl)-4- |
| | hydroxy-N-(4-(4-methylthiazol-5-yl)benzyl)pyrrolidine-2-carboxamide |
| | (2S,4R)-1-((S)-2-(8-(4-(2-(4-(1,2-bis(4-hydroxyphenyl)but-1-en-1- |
| | yl)phenoxy)ethyl)piperazin-1-yl)-8-oxooctanamido)-3,3-dimethylbutanoyl)-4- |
| | hydroxy-N-(4-(4-methylthiazol-5-yl)benzyl)pyrrolidine-2-carboxamide |
| | (2S,4R)-1-((S)-2-(9-(4-(2-(4-(1,2-bis(4-hydroxyphenyl)but-1-en-1- |
| | yl)phenoxy)ethyl)piperazin-1-yl)-9-oxononanamido)-3,3-dimethylbutanoyl)-4- |
| | hydroxy-N-(4-(4-methylthiazol-5-yl)benzyl)pyrrolidine-2-carboxamide |
| | (2S,4R)-1-((S)-2-(10-(4-(2-(4-(1,2-bis(4-hydroxyphenyl)but-1-en-1- |
| | yl)phenoxy)ethyl)piperazin-1-yl)-10-oxodecanamido)-3,3-dimethylbutanoyl)-4- |
| | hydroxy-N-(4-(4-methylthiazol-5-yl)benzyl)pyrrolidine-2-carboxamide |
| | (2S,4R)-1-((S)-2-(7-(4-(2-(4-(1,2-bis(4-hydroxyphenyl)but-1-en-1- |
| | yl)phenoxy)ethyl)piperazin-1-yl)heptanamido)-3,3-dimethylbutanoyl)-4-hydroxy- |
| | N-(4-(4-methylthiazol-5-yl)benzyl)pyrrolidine-2-carboxamide |
| | (2S,4R)-1-((S)-2-((7-(4-(2-(4-(1,2-bis(4-hydroxyphenyl)but-1-en-1- |
| | yl)phenoxy)ethyl)piperazin-1-yl)-7-oxoheptyl)amino)-3,3-dimethylbutanoyl)-4- |
| | hydroxy-N-(4-(4-methylthiazol-5-yl)benzyl)pyrrolidine-2-carboxamide |
| | (2S,4R)-1-((S)-2-((7-(4-(2-(4-(1,2-bis(4-hydroxyphenyl)but-1-en-1- |
| | yl)phenoxy)ethyl)piperazin-1-yl)heptyl)amino)-3,3-dimethylbutanoyl)-4-hydroxy- |
| | N-(4-(4-methylthiazol-5-yl)benzyl)pyrrolidine-2-carboxamide |

It is to be understood that the compound of formula (I) of the present disclosure may have a stereo configuration and can therefore exist in more than one stereoisomer form. The present disclosure also relates to compounds of formula (I) having a stereo configuration in pure or substantially pure isomeric form, e.g., greater than about 90% enantiomeric/diastereomeric excess (“ee”), such as greater than about 95% ee or 97% ee, or greater than about 99% ee, and mixtures thereof, including racemic mixtures. The purification of said isomers and the separation of said isomeric mixtures may be achieved by asymmetric synthesis (for example, by using chiral intermediates) and/or chiral resolution and the like.

In another aspect, the present disclosure also provides a pharmaceutical composition, including, as an active ingredient, the compound of formula (I) according to the present disclosure or a pharmaceutically acceptable salt, enantiomers, stereoisomers, solvates, or polymorphs thereof, and a pharmaceutically acceptable carrier.

In one embodiment, the pharmaceutical composition of the present disclosure further includes at least one additional therapeutic agent.

In one embodiment, said additional therapeutic agent is used for treating or preventing a cancer.

In one embodiment, the cancer includes, but is not limited to, breast cancer.

The pharmaceutical composition containing said active ingredient according to the present disclosure can be formulated into any suitable formulations such as sprays, patches, tablets, capsules, dragees, troches, powders, granules, powder injections, or liquid formulations (such as suspensions, solutions, emulsions or syrups) and the like, depending upon route of administration (including, but not limited to, nasal, inhalation, topical, oral, oral mucosa, rectal, intrapleural, intraperitoneal, vaginal, intramuscular, subcutaneous, transdermal, epidural, intrathecal and intravenous administration).

In another aspect of the present disclosure, the compound of formula (I) according to the present disclosure or a pharmaceutically acceptable salt, racemic mixtures, enantiomers, stereoisomers, solvates, or polymorphs thereof, is useful as a medicament.

In another aspect of the present disclosure, the compound of formula (I) according to the present disclosure or a pharmaceutical acceptable salt, racemic mixtures, enantiomers, stereoisomers, solvates, or polymorphs thereof, is useful for treating and/or preventing diseases or disorders associated with estrogen receptor.

In one embodiment, the diseases or disorders associated with estrogen receptor include, but are not limited to, estrogen-dependent diseases or disorders.

The estrogen-dependent diseases or disorders include, but are not limited to, cancer (especially cancers associated with estrogen receptor), osteoporosis, atherosclerosis, atrophic vaginitis, proliferative diseases, tumor metastasis, bipolar disorder, depression, and inducing ovulation in anovulatory infertile subject, etc.

In one embodiment, the cancer (especially the cancers associated with estrogen receptor) includes, but is not limited to, uterine cancer, breast cancer, ovarian tumor, malignant melanoma, etc.

In one embodiment, the breast cancer includes, but is not limited to, ER-positive breast cancer in menopausal women with CYP2D6 gene defect, lymph node-positive breast cancer, ductal carcinoma in situ of the breast, etc.

In another aspect, the present disclosure provides use of the compound of formula (I) according to the present disclosure or a pharmaceutical acceptable salt, racemic mixtures, enantiomers, stereoisomers, solvates, or polymorphs thereof for the manufacture of a medicament for treating and/or preventing diseases or disorders associated with estrogen receptor. In one embodiment, the diseases or disorders associated with estrogen receptor include, but are not limited to, estrogen-dependent diseases or disorders. The estrogen-dependent diseases or disorders include, but are not limited to, cancer (especially cancers associated with estrogen receptor), osteoporosis, atherosclerosis, atrophic vaginitis, proliferative diseases, tumor metastasis, bipolar disorder, depression, and inducing ovulation in anovulatory infertile subject, etc. In one embodiment, the cancer (especially the cancers associated with estrogen receptor) include, but are not limited to, uterine cancer, breast cancer, ovarian tumor, malignant melanoma, etc. In one embodiment, the breast cancer includes, but is not limited to, ER-positive breast cancer in menopausal women with CYP2D6 gene defect, lymph node-positive breast cancer, ductal carcinoma in situ of the breast, etc.

In another aspect, the present disclosure provides the method for treatment or prevention of diseases or disorders associated with estrogen receptor in a subject, comprising administering to the subject a therapeutically effective amount of the compound of fomular (I) accordign to the present disclosure or a pharmaceutically acceptable salt, racemic mixtures, enantiomers, stereoisomers, solvates, or polymorphs thereof, or the pharmaceutical composition accordign to the present disclosure.

In one embodiment of the method for treatment or prevention of diseases or disorders associated with estrogen receptor according to the present disclosure, the diseases or disorders associated with estrogen receptor include, but are not limited to, estrogen-dependent diseases or disorders. The estrogen-dependent diseases or disorders include, but are not limited to, cancer (especially cancers associated with estrogen receptor), osteoporosis, atherosclerosis, atrophic vaginitis, proliferative diseases, tumor metastasis, bipolar disorder, depression, and inducing ovulation in anovulatory infertile subject, etc. In one embodiment, the cancer (especially the cancers associated with estrogen receptor) include, but are not limited to, uterine cancer, breast cancer, ovarian tumor, malignant melanoma, etc. In one embodiment, the breast cancer includes, but is not limited to, ER-positive breast cancer in menopausal women with CYP2D6 gene defect, lymph node-positive breast cancer, ductal carcinoma in situ of the breast, etc.

In the method according to the present disclosure, the compound of formula (I) or a pharmaceutical acceptable salt, racemic mixtures, enantiomers, stereoisomers, solvates, or polymorphs thereof, or the pharmaceutical composition, can be administered to the subject via at least one of route of administration selected from the group consisting of nasal administration, inhalation administration, topical administration, oral administration, oral mucosal administration, rectal administration, intrapleural administration, intraperitoneal administration, vaginal administration, intramuscular administration, subcutaneous, transdermal, epidural, intrathecal, and intravenous administration.

### Definition

Unless otherwise specified, the following terms, phrases, and symbols used herein generally have the meanings as defined below.

In the present disclosure, the compounds of formula (I) of the present disclosure are also referred to as ER protein regulators or PROTAD (small) molecules, which can be used interchangeably.

In the present disclosure, the terms “LIN” and “linker” are used interchangeably, and both of them refer to the linker group of the compound of formula (I).

In the present disclosure, the terms “intermediate LM” refers to an intermediate compound which is used in the following scheme for synthesizing the target ER protein regulators of the present disclosure by reacting with toremifene derivatives or tamoxifen derivatives.

Herein, a bond interrupted by a wavy line shows the point of attachment of the radical depicted to remaining moieties of the molecule. For example, the group Z₁ depicted below

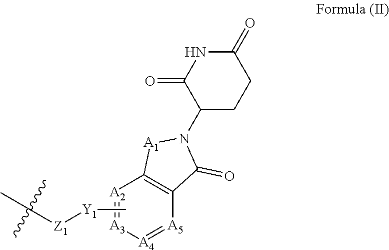

represents the group Z₁, which is connected to the group LIN of the compound of formula (I).

As used herein, the term “halogen atom” or “halogen” used alone or in combination refers to fluorine, chlorine, bromine or iodine, and is preferably F, Cl or Br.

As used herein, the term “alkyl” used alone or in combination refers to a linear or branched alkyl group. The term “(Cₓ-C_{y}) alkyl” or “C_{x-y} alkyl” (x and y are each an integer) refers to a linear or branched alkyl group containing from x to y carbon atoms. The term “C₁₋₁₀ alkyl” used alone or in combination in the present disclosure refers to a linear or branched alkyl group containing from 1 to 10 carbon atoms. The C₁₋₁₀ alkyl group of the present disclosure is preferably a C₁₋₉ alkyl group, more preferably C₁₋₈ alkyl group, still more preferably C₂₋₈ alkyl group, more preferably C₁₋₇ alkyl group, even more preferably C₁₋₆ alkyl, C₁₋₅ alkyl, or C₁₋₄ alkyl. Representative examples include, methyl, ethyl, n-propyl, isopropyl, n-butyl, isobutyl, sec-butyl, tert-butyl, pentyl, isopentyl, neopentyl, tert-pentyl, hexyl, heptyl, octyl, nonyl and decyl. The term “C₁₋₃ alkyl group” in the present disclosure refers to an alkyl group containing from 1 to 3 carbon atoms, and its representative examples include methyl, ethyl, n-propyl, and isopropyl. In the present disclosure, the “alkyl” is optionally substituted, and the substituent(s) of “alkyl” is/are preferably one or more selected from halogen, cyano, C₁₋₃ alkyl, C₁₋₃ alkoxy, trifluoromethyl, heterocyclyl, or a combination thereof.

As used herein, the term “alkylene” (which is used interchangeably with “alkylene chain”) used alone or in combination refers to a linear or branched divalent saturated hydrocarbon group composed of carbon and hydrogen atoms. The term “Cₓ-C_{y} alkylene” or “C_{x-y} alkylene” (x and y are each an integer) refers to a linear or branched alkylene group containing from x to y carbon atoms. The C₁-C₃₀ alkylene group in the present disclosure is preferably C₁-C₂₉ alkylene, C₁-C₂₈ alkylene, C₁⁻C₂₇ alkylene, C₁-C₂₆ alkylene, C₁-C₂₅ alkylene, C₁-C₂₄ alkylene, C₁-C₂₃ alkylene, C₁-C₂₂ alkylene, C₁-C₂₁ alkylene, C₁-C₂₀ alkylene, C₁-C₁₉ alkylene, C₁-C₁₉ alkylene, C₁-C₁₇ alkylene, C₁-C₁₆ alkylene, C₁-C₁₅ alkylene, C₁-C₁₄ alkylene, C₁-C₁₃ alkylene, C₁-C₁₂ alkylene, C₁-C₁₁ alkylene, C₁-C₁₀ alkylene , C₁-C₉ alkylene, C₁-C₈ alkylene, C ₁-C₇ alkylene, C₁-C₆ alkylene, Ci-05 alkylene, C₁-C₄ alkylene, C₁-C₃ alkylene, or C₁-C₂ alkylene. Representative examples include, but are not limited to, methylene, ethylene, propylene, isopropylidene, butylene, isobutylene, sec-butylene, tert-butylene, n-pentylene, isopentylene, neopentylene, tert-pentylene, hexylene, heptylene, octylene, nonylene, decylene, undecylene, dodecylene, tridecylene, tetradecylene, pentadecylene, hexadecylene, heptadecylene, octadecylene, nonadecylene, eicosylene, heneicosylene, docosylene, tricosylene, tetracosylene, pentacosylene, hexacosylene, heptacosylene, octacosylene, nonacosylene, and triacontylene.

As used herein, the term “aryl” used alone or in combination refers to a divalent aromatic hydrocarbon group containing from 5 to 14 carbon atoms and optionally one or more fused rings, such as phenyl group, naphthyl group or fluorenyl group. In the present disclosure, the “aryl” is optionally substituted. A substituted aryl group refers to an aryl group optionally substituted 1 to 3 times with a substituent(s), wherein the substituent is preferably selected from C₁₋₃ alkyl, cyano, C₁₋₃ alkoxy, trifluoromethyl, heterocyclyl, halogen, amino, or hydroxyl.

As used herein, the term “arylene” used alone or in combination refers to a divalent aromatic hydrocarbon group containing from 5 to 14 carbon atoms and optionally one or more fused rings, such as phenylene group, naphthylene group or fluorenylene group. In the present disclosure, the “arylene” is optionally substituted. A substituted arylene group refers to an arylene group optionally substituted 1 to 3 times with a substituent(s), wherein the substituent is selected from C₁₋₃ alkyl, C₁₋₃ alkoxy, halogen, amino, or hydroxyl.

As used herein, the term “alkoxy” used alone or in combination refers to a linear or branched alkoxy group having the formula of —O-alkyl. Preferably, the alkyl of the alkoxy may contain 1-10 carbon atoms. Representative examples of “alkoxy” include, but are not limited to, methoxy, ethoxy, propoxy, isopropoxy, n-butoxy, isobutoxy, tert-butoxy, pentoxy, 2-pentyloxy, isopentyloxy, neopentyloxy, hexyloxy, 2-hexyloxy, 3-hexyloxy, 3-methylpentyloxy, etc. The term “C₁-C₃ alkoxy” or “C₁₋₃ alkoxy” used alone or in combination refers to a linear or branched alkoxy group containing from 1 to 3 carbon atoms. Representative examples of C₁₋₃ alkoxy include, but are not limited to, methoxy, ethoxy, n-propoxy, and isopropoxy. Preferred are methoxy and ethoxy.

As used herein, the term “cycloalkyl” used alone or in combination refers to a saturated or partially unsaturated (i.e., containing one or more double bonds, but not having a fully conjugated π-electron system) monocyclic or bicyclic cyclic hydrocarbon radical having from 3 to 12 carbon atoms. The term “C₃-C₁₀ cycloalkyl” used alone or in combination refers to a saturated or partially unsaturated (i.e., containing one or more double bonds, but not having a fully conjugated π-electron system) monocyclic or bicyclic cyclic hydrocarbon radical having from 3 to 10 carbon atoms. Representative examples of cycloalkyl include, but are not limited to, cyclopropyl, cyclobutyl, cyclopentyl, cyclopentenyl, cyclohexyl, cyclohexenyl, cycloheptyl, cyclooctyl, decalinyl, octahydropentalenyl, octahydro-1H-indenyl, and Spiro-cycloalkyl. In the present disclosure, the “cycloalkyl” is optionally substituted, and the substituent(s) is/are preferably selected from halogen, cyano, C₁₋₃ alkyl, C₁₋₃ alkoxy, trifluoromethyl, heterocyclyl, and a combination thereof.

As used herein, the term “cycloalkylene”, used alone or in combination, refers to a saturated or partially unsaturated (e.g., containing one or more double bonds, but not having a fully conjugated π-electron system) divalent monocyclic or bicyclic cyclic hydrocarbon group having from 3 to 12 carbon atoms. Representative examples of cycloalkylene include, but are not limited to, cyclopropylene, cyclobutylene, cyclopentylene, cyclopentenylene, cyclohexylene, cyclohexenylene, cycloheptylene, cyclooctylene, decalinylene, octahydropentalenylene, octahydro-1H-indenylene, and Spiro-cycloalkylene.

As used herein, the term “heteroaryl” used alone or in combination refers to a 5- to 10-membered monocyclic or bicyclic aromatic ring group containing one or more (eg, from 1 to 6, or from 1 to 5, or from 1 to 4, or from 1 to 3) heteroatoms independently selected from the group consisting of oxygen, nitrogen and sulfur. Representative examples of such heteroaryl groups include, but are not limited to, furanyl, oxazolyl, isoxazolyl, oxadiazolyl, thienyl, thiazolyl, isothiazolyl, thiadiazolyl, pyrrolyl, imidazolyl, pyrazolyl, triazolyl, pyridyl, pyrimidinyl, pyridazinyl, pyrazinyl, indolyl, isoindolyl, benzofuranyl, isobenzofuranyl, benzothienyl, indazolyl, benzimidazolyl, benzoxazolyl, benzoisoxazolyl, benzothiazolyl, benzoisothiazolyl, benzotriazolyl, benzo[2,1,3]oxadiazolyl, benzo[2,1,3]thiadiazolyl, benzo[1,2,3]thiadiazolyl, quinolyl, isoquinolyl, naphthyridinyl, cinnolinyl, quinazolinyl, quinoxalinyl, phthalazinyl, pyrazolo[1,5-a]pyridyl, pyrazolo [1,5-a]pyrimidyl, imidazo [1,2-a]pyridyl, 1H-pyrrolo [3 ,2-b]pyridyl, 1H-pyrrolo [2,3-b]pyridyl, 4H-fluoro [3 ,2-b]pyrrolyl, pyrrolo [2,1-b] thiazolyl and imidazo [2,1-b]thiazolyl. Hetero aryl groups may be unsubstituted or substituted as explicitly defined. The substituted heteroaryl group refers to a heteroaryl group optionally substituted 1 to 3 times with a substituent(s) preferably selected from the group consisting of C₁₋₃ alkyl, C₁₋₃ alkoxy, cyano, trifluoromethyl, heterocyclyl, halogen, amino, or hydroxyl.

As used herein, the term “heteroarylene” used alone or in combination refers to a 5- to 10-membered monocyclic or bicyclic divalent aromatic ring group containing one or more (eg, from 1 to 6, or from 1 to 5, or from 1 to 4, or from 1 to 3) heteroatoms independently selected from the group consisting of oxygen, nitrogen, and sulfur. Representative examples of such heteroarylene groups include, but are not limited to, furanylene, oxazolylene, isoxazolylene, oxadiazolylene, thienylene, thiazolylene, isothiazolylene, thiadiazolylene, pyrrolylene, imidazolylene, pyrazolylene, triazolylene, pyridylene, pyrimidinylene, pyridazinylene, pyrazinylene, indolylene, isoindolylene, benzofuranylene, isobenzofuranylene, benzothienylene, indazolylene, benzimidazolylene, benzoxazolylene, benzoisoxazolylene, benzothiazolylene, benzoisothiazolylene, benzotriazolylene, benzo[2,1,3]oxadiazolylene, benzo[2,1,3]thiadiazolylene, benzo[1,2,3]thiadiazolylene, quinolylene, isoquinolylene, naphthyridinylene, cinnolinylene, quinazolinylene, quinoxalinylene, phthalazinylene, pyrazolo[1,5-a]pyridinylene, pyrazolo[1,5-a]pyrimidinylene, imidazo[1,2-a]pyridinylene, 1H-pyrrolo[3,2-b]pyridinylene, 1H-pyrrolo[2,3-b]pyridinylene, 4H-fluoro[3,2-b]pyrrolylene, pyrrolo[2,1-b]thiazolylene, and imidazo[2,1-b]thiazolylene. The heteroarylene group may be unsubstituted or substituted as explicitly defined.

As used herein, the term “heterocyclyl” used alone or in combination refers to a 3- to 12-membered saturated or partially unsaturated (i.e., containing one or more double bonds, but not having a fully conjugated i-electron system) monocyclic, bicyclic, or tricyclic cyclic hydrocarbon group containing one or more (e.g., from 1 to 5, or from 1 to 4) heteroatoms independently selected from the group consisting of sulfur, oxygen, and nitrogen. In some embodiments, “heterocyclyl” may preferably refer to a 3- to 6-membered saturated or partially unsaturated (i.e., containing one or more double bonds, but not having a fully conjugated n-electron system) monocyclic cyclic hydrocarbon group containing one or more heteroatoms independently selected from the group consisting of sulfur, oxygen, and nitrogen. Representative examples of the heterocyclyl include, but are not limited to, azetidinyl, oxetanyl, pyrrolidinyl, imidazolidinyl, pyrazolidyl, triazolyl, tetrahydrofuranyl, tetrahydropyranyl, tetrahydrothienyl, tetrahydrothiopyranyl, oxazolidinyl, thiazolidinyl, piperidinyl, piperazinyl, morpholinyl, thiomorpholinyl, and dioxacyclohexyl. The heterocyclyl may be unsubstituted or substituted as explicitly defined, and the substituent(s) of the heterocyclyl can be preferably selected from the group consisting of C₁₋₃ alkyl, C₁₋₃ alkoxy, cyano, trifluoromethyl, heterocyclyl, halogen, amino, or hydroxy.

As used herein, the term “heterocyclylene” used alone or in combination refers to a 3- to 12-membered saturated or partially unsaturated (i.e., having one or more double bonds, but not having a completely conjugated n-electron system) bivalent monocyclic, bicyclic, or tricyclic cyclic hydrocarbon group, containing one or more (e.g., from 1 to 5, or from 1 to 4) heteroatoms independently selected from the group consisting of sulfur, oxygen, and nitrogen. In some embodiments, “heterocyclylene” may preferably refer to a 3- to 6-membered saturated or partially unsaturated (i.e., containing one or more double bonds, but not having a fully conjugated π-electron system) bivalent monocyclic cyclic hydrocarbon group containing one or more heteroatoms independently selected from the group consisting of sulfur, oxygen, and nitrogen. Representative examples of the heterocyclylene group include, but are not limited to, azetidinylene, oxetanylene, pyrrolidinylene, imidazolidylene, pyrazolidylene, triazolylend, tetrahydrofuranylene, tetrahydropyranylene, tetrahydrothienylene, tetrahydrothiopyranylene, oxazolidinylene, thiazolidinylene, piperidinylene, piperazinylene, morpholinylene, thiomorpholinylene, and dioxacyclohexylene. The heterocyclylene group may be unsubstituted or substituted as explicitly defined. The substituent(s) of the heterocyclylene can be preferably selected from C₁₋₃ alkyl, C₁₋₃ alkoxy, cyano, trifluoromethyl, heterocyclyl, halogen, amino or hydroxyl.

As used herein, the term “alkynylene” used alone or in combination refers to a linear or branched divalent hydrocarbon group containing one or more carbon-carbon triple bonds and containing from 2 to 10 (preferably from 2 to 6, more preferably from 2 to 4) carbon atoms. Preferred examples of the alkynylene group include, but are not limited to, ethynylene, 1-propynylene, 1-butynylene, and 1,3-alkadiynylene.

As used herein, the term “alkynyl” used alone or in combination refers to a linear or branched hydrocarbon group containing one or more carbon-carbon triple bonds and containing from 2 to 10 (preferably from 2 to 6, more preferably from 2 to 4) carbon atoms. Preferred examples of the alkynyl group include, but are not limited to, ethynyl, 1-propynyl, 1-butynyl, and 1,3-alkadiynyl.

As used herein, the term “alkenylene” used alone or in combination refers to a linear or branched divalent hydrocarbon group containing one or more carbon-carbon double bonds and containing from 2 to 40 (more preferably from 2 to 35, from 2 to 30, from 2 to 25, from 2 to 20, from 2 to 15, from 2 to 10, from 2 to 6, or from 2 to 5, especialy preferably from 2 to 4 or from 2 to 3) carbon atoms. Preferred examples of the alkenylene group include, but are not limited to, vinylidene (e.g., —CH═CH—), 1-propenylene, allylidene, 1-butenylene, 2-butenylene, 3-butenylene, isobutenylene, pentenylene, pent-2,4-dienylene, 1-methyl-but-1-enylene , 2-methyl-but-1-enylene, 3-methyl-but-1-enylene, 1-methyl-but-2-enylene, 2-methyl-but-2-enylene, 3-methyl-but-2-enylene, 1-methyl-but-3-enylene, 2-methyl-but-3-enylene, 3-methyl-but-3-enylene, hexenylene, heptenylene, octenylene, oct-2-enylene, nonenylene, decenylene, dodec-2-enylene, isododecenylene, dodec-2-enylene, octadec-4-enylene, or 3,7,11,11-tetramethyl-2,6,10-undec atrienylene.

herein, the term “alkenyl” used alone or in combination refers to a linear or branched hydrocarbon group containing one or more carbon-carbon double bonds and containing from 2 to 40 (more preferably from 2 to 35, from 2 to 30, from 2 to 25, from 2 to 20, from 2 to 15, from 2 to 10, from 2 to 6, or from 2 to 5, especialy preferably from 2 to 4 or from 2 to 3) carbon atoms. Preferred examples of the alkenylene group include, but are not limited to, vinyl (e.g., CH₂═CH—), 1-propenyl, allyl, 1-butenyl, 2-butenyl, 3-butenyl, isobutenyl, pentenyl, pent-2,4-dienyl, 1-methyl-but-1-enyl, 2-methyl-but-1-enyl, 3-methyl-but-1-enyl, 1-methyl-but-2-enyl, 2-methyl-but-2-enyl, 3-methyl-but-2-enyl, 1-methyl-but-3-enyl, 2-methyl-but-3-enyl, 3-methyl-but-3-enyl, hexenyl, heptenyl, octenyl, oct-2-enyl, nonenyl, decenyl, dodec-2-enyl, isododecenyl, dodec-2-enyl, octadec-4-enyl, or 3,7,11,11-tetramethyl-2,6,10-undec atrienyl.

In the present disclosure, salts or pharmaceutically acceptable salts, and enantiomers, stereoisomers, solvates, polymorphs of the compound of formula (I) according to the disclosure are also encompassed within the scope of this present disclosure.

In all embodiments of the present disclosure, the salt or pharmaceutically acceptable salt of the compound of formula (I) refers to non-toxic inorganic or organic acid and/or base addition salts. Examples include sulfate, hydrochloride, citrate, maleate, sulfonate, citrate, lactate, tartrate, fumarate, phosphate, dihydrophosphate, pyrophosphate, metaphosphate, oxalate, malonate, benzoate, mandelate, succinate, glycolate, or p-toluenesulfonate, etc..

“Pharmaceutically acceptable carrier” refers to a pharmaceutically acceptable material, such as a filler, stabilizer, dispersant, suspending agent, diluent, excipient, thickener, solvent, or encapsulating material, with which the useful compounds according to the present disclosure are carried or transported into or administered to a patient so that they can perform their intended function. Generally, such constructs are carried or transported from one organ or part of the body to another organ or part of the body. The carrier is compatible with the other ingredients of the formulation, including the compounds useful in the present disclosure, and is not harmful to the patient, and the carrier must be “acceptable.” Some examples of materials that can be used as pharmaceutically acceptable carriers include, but are not limited to, sugars such as lactose, glucose, and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository wax; oils such as peanut oil, cottonseed oil, safflower oil, sesame oil, olive oil, corn oil and soybean oil; glycols such as propylene glycol; polyols such as glycerol, sorbitol, mannitol, and polyethylene glycol; esters such as ethyl oleate and ethyl laurate; agar; buffers such as magnesium hydroxide and aluminum hydroxide; surfactant phosphate buffer solution; and other common non-toxic compatible substances used in pharmaceutical preparations.

The term “treatment” or “treating” refers to the administration of the compound of formula I or a pharmaceutically acceptable salt thereof according to the present disclosure, or the pharmaceutical composition containing the compound of formula I or a pharmaceutically acceptable salt thereof as an active ingredient, to a subject to mitigate (alleviate) undesirable diseases or conditions, such as the development of cancer. The beneficial or desired clinical results of the present disclosure include, but are not limited to: alleviating symptoms, reducing the severity of the disease, stabilizing the state of the disease, slowing down or delaying the progression of the disease, improving or alleviating the condition, and alleviating the disease.

A “therapeutically effective amount” of a compound of the present disclosure depends on the age, sex, and weight of the patient, the patient's current medical condition, and the cancer progression of the patient being treated. Those skilled in the art will be able to determine a suitable dosage based on these and other factors.

The term “room temperature” used herein refers to the ambient temperature, such as a temperature of 20-30° C.

The compounds developed by the present disclosure belong to regulators targeting specific ER protein, which are composed of three parts: target protein anchoring element, recruitment elements (ULM) for protein degradation system (e.g., E3 ubiquitin ligase), and link unit (linker or LIN). The present disclosure selects SERMs capable of targeting ER proteins as anchoring elements, and an E3 ligase ligand is combined with SERMs through a linker to develop a degrader targeting ER protein. For ER(+) breast cancer, through the specific recognition of target ER proteins by SERMs, the activity of the target ER protein is inhibited, and at the same time, E3 ligase specifically ubiquitinates target ER protein to achieve degradation and elimination of the target ER protein, and finally can remove the target ER protein from tumor cells. Degrading and removing the ER protein completely and inhibiting the activity of residual ER protein can not only inhibit tumorigenesis and progression, but also potentially overcome resistance to targeted drugs. This research has been successfully applied to a degrading agent that targets specific proteins, providing a new treatment strategy for breast cancer patients in the context of precision medicine. In addition, the ER protein regulator designed and developed by the present disclosure has different regulatory effects in different tissue cells, and the correlation between different tumors and ER protein is also different, so it may also be used to treat estrogen-dependent tumors, such as cancer (including but not limited to breast cancer such as ER-positive breast cancer in menopausal women with CYP2D6 gene defect, lymph node-positive breast cancer, uterine cancer, ductal carcinoma in situ of the breast, ovarian tumor, malignant melanoma, etc.), osteoporosis, atherosclerosis, atrophic vaginitis, proliferative diseases, tumor metastasis, bipolar disorder, depression, inducing ovulation in anovulatory infertile subject and other diseases.

### EXAMPLES

In the following description, numerous specific details are set forth in order to provide a thorough understanding of the present disclosure. The present disclosure may be practiced without some or all of these specific details. In other cases, well-known process operations have not been described in detail in order not to unnecessarily obscure the present disclosure. Although the present disclosure will be described in conjunction with specific embodiments, it should be understood that this is not intended to limit the present disclosure to these embodiments.

The following abbreviations are used throughout the description and examples:
Ar Argon gas
DCM Dichloromethane
DIPEA N,N-diisopropylethylamine
DMF N,N-dimethylformamide
DMAP N,N-Dimethyl-4-pyridinamine
DMSO Dimethyl sulfoxide
equiv equivalent
EDCI 1-(3-Dimethylaminopropyl)-3-ethylcarbodiimide
ESI Electrospray ionization
EtOH Ethanol
EtONa Sodium ethoxide
HATU 2-(7-azabenzotriazol-1-yl)-N,N,N′,N′-tetramethyluronium
hexafluorophosphate
HOAc or AcOH acetic acid
HOAT 1-hydroxy-7-azobenzotriazole
HOB T 1-hydroxybenzotriazole
HPLC High performance liquid chromatography
HRMS High resolution mass spectrometry
LC-MS Liquid chromatography-mass spectrometry
LRMS Low resolution mass spectrometry
LC Liquid chromatography
NMP N-methylpyrrolidone
NMM N-methylmorpholine
¹H NMR Proton nuclear magnetic resonance
r.t. Room temperature
TEA Triethylamine
TFA Trifluoroacetate
THF Tetrahydrofuran
TLC Thin layer chromatography
TMS Trimethylsilyl
TsOH p-toluenesulfonic acid

In the present disclosure, the ¹H NMR spectrum is measured using a Bruker-500 MHz nuclear magnetic resonance instrument, and CD₃OD containing 0.1% TMS is used as a solvent, wherein the ¹H NMR spectrum uses CD₃OD (δ=3.31 ppm) as an internal standard; or CDCl₃ containing 0.1% TMS is used as the solvent, wherein the ¹H NMR spectrum uses CDCl₃ (δ=7.26 ppm) as the internal standard; or DMSO-d₆ containing 0.03% TMS as the solvent, wherein the ¹H NMR spectrum uses DMSO-d₆ (δ=2.50 ppm) as the internal standard; LCMS spectrum was measured on an AB Triple 4600 mass spectrometer, HPLC preparation was measured on a SHIMADZU LC-20AP type instrument, and HPLC purity was measured on a SHIMADZU LC-30AP or Waters 1525 type instrument. All reactions were performed in the air without special instructions; the reactions were followed by TLC or LC-MS.

Solvents and reagents are processed as follows:

The solvents used in the reaction such as DCM, DMF, anhydrous EtOH, and anhydrous Me0H and the like were purchased from Chinese Sinopharm Group. Preparative grade CH₃CN and deionized water are used in HPLC preparation. Both toremifene derivative A and tamoxifen derivative A were purchased commercially. Unless otherwise stated, other reagents, materials and medicines were purchased commercially and used directly.

General Synthesis Methods

General Synthetic Method for Intermediate LM (Pomalidomide PEG Linker):

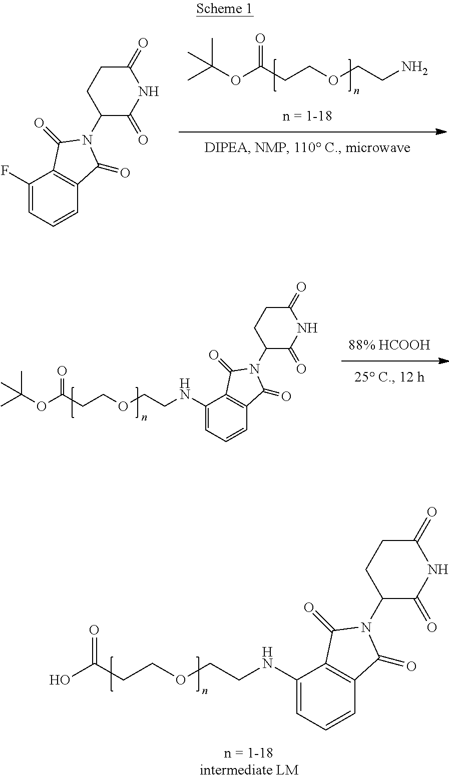

In step 1, a 30 mL microwave reaction tube was charged with 2-(2,6-dioxopiperidin-3-yl)-4-fluoroisoindolin-1,3-dione (5 mmol, 1 equiv), the corresponding amine (6 mmol, 1.2 equiv) and DIPEA (25 mmol, 5 equiv), followed by addition of NMP (8 mL). The reaction mixture was stirred at room temperature for 10 min, then slowly bubbled with argon gas, heated to 110° C. in a microwave reactor and stirred for 2 h. After cooling to r.t., the resulting mixture was poured into 90% NaCl aqueous solution, extracted with ethyl acetate (4×50 mL). The organic phases were combined, washed with water (2×30 mL) and saturated brine (50 mL), dried over anhydrous sodium sulfate and concentrated under reduced pressure to remove the solvent. The resulting residue was purified by silica gel chromatography (eluent(v/v): petroleum ether(PE)/ethyl acetate(EtOAc)=1:1) to give the desired tert-butyl ester intermediate. In step 2, this tert-butyl ester intermediate and 88% formic acid (20 mL) were sequentially added in a 50 mL of round-bottom flask, and the reaction mixture was stirred for 12h at room temperature. After removing the reaction solvent under reduced pressure, the resulting residue was treated by addition of water and freeze-drying to afford target compound.

GeneraL Synthetic Method for Intermediate LM (Pomalidomide Alkyl Carbon Chain Linker):

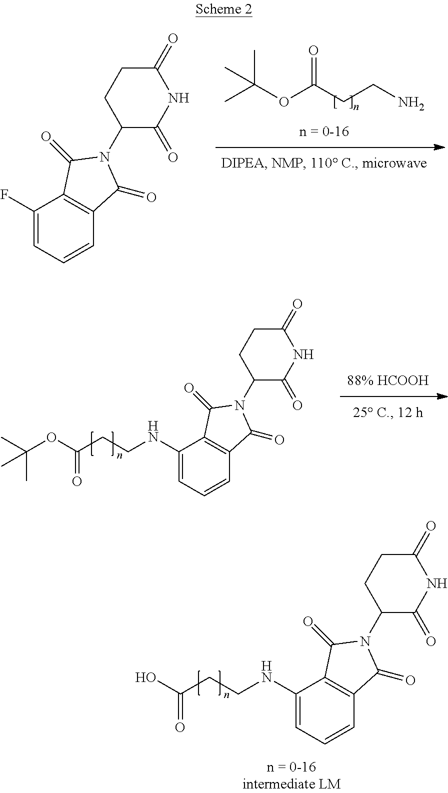

In step 1, a 30 mL microwave reaction tube was charged with 2-(2,6-dioxopiperidin-3-yl)-4-fluoroisoindolin-1,3-dione (7 mmol, 1 equiv), the corresponding amine (8.4 mmol, 1.2 equiv) and DIPEA (35 mmol, 5 equiv), followed by addition of NMP (8 mL). The reaction mixture was stirred at room temperature for 10 min, then slowly bubbled with argon gas, heated to 110° C. in a microwave reactor and stirred for 2 h. After cooling to r.t., the resulting mixture was poured into 90% NaCl aqueous solution, extracted with ethyl acetate (4×50 mL) . The organic phases were combined, washed with water (2×30 mL) and saturated brine (50 mL), dried over anhydrous sodium sulfate and concentrated under reduced pressure. The resulting residue was purified by silica gel chromatography (eluent(v/v): PE/EtOAc=1:1) to give the desired tert-butyl ester intermediate. In step 2, this tert-butyl ester intermediate and 88% formic acid (20 mL) were sequentially added in a 50 mL of round-bottom flask, and the reaction mixture was stirred at room temperature for 12h. After removing the reaction solvent under reduced pressure, the resulting residue was treated by addition of water and freeze-drying to afford target compound.

General Synthetic Method for Intermediate LM (VHL-1 PEG Linker):

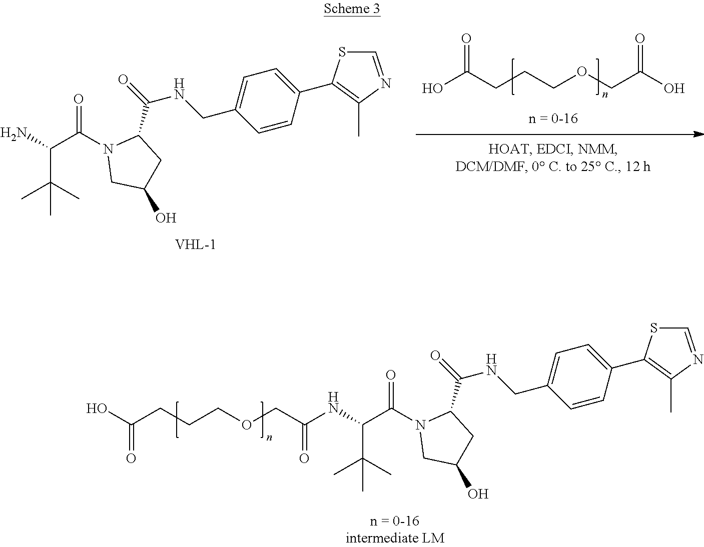

A 250 mL three-necked flask was charged with the corresponding diacid (5.0 mmol, 2.5 equiv), anhydrous DMF (10 mL) and anhydrous DCM (150 mL). The mixture was stirred and cooled with an ice bath, and then added NMM (10.0 mmol, 5 equiv), VHL-1 (2 mmol, 1 equiv), HOAT (2.4 mmol, 1.2 equiv) and EDCI (2.4 mmol, 1.2 equiv). After the addition, the resulting reaction mixture was stirred in the ice bath for 5 h, then warmed to room temperature and stirred overnight. After the reaction, the reaction mixture was quenched with deionized water (1 mL) and then concentrated under reduced pressure to remove DCM, and the residue was subjected to reverse-phase C18 column chromatography (eluent (v/v): acetonitrile/(water+0.1%TFA)=10%-100%) for separation and purification. The acetonitrile was removed by evaporation under reduced pressure, and the residue was lyophilized to yield the target product.

General Synthetic Method for Intermediate LM (VHL-1 Alkyl Carbon Chain Linker):

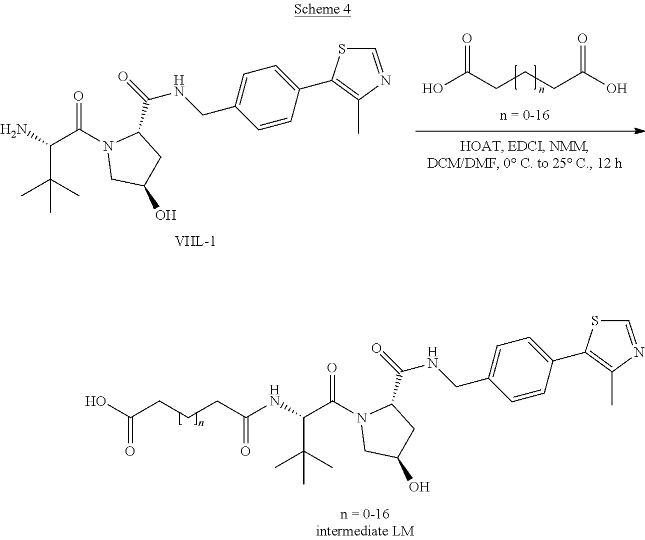

A 250 mL three-necked flask was charged with the corresponding diacid (5.0 mmol, 2.5 equiv), anhydrous DMF (10 mL) and anhydrous DCM (150 mL). The mixture was stirred and cooled with an ice bath, and then added NMM (10.0 mmol, 5 equiv), VHL-1 (2 mmol, 1 equiv), HOAT (2.4 mmol, 1.2 equiv) and EDCI (2.4 mmol, 1.2 equiv). After the addition, the resulting reaction mixture was stirred in the ice bath for 5 h, then warmed to room temperature and stirred overnight. After the reaction, the reaction mixture was quenched with deionized water (1 mL) and then concentrated under reduced pressure to remove DCM, and the residue was subjected to reverse-phase C₁₈ column chromatography (eluent (v/v): acetonitrile/(water+0.1%TFA)=10% -100%) for separation and purification. The acetonitrile was removed by evaporation under reduced pressure, and the residue was lyophilized to yield the target product.

General Synthesis Method for Intermediate LM (Lenalidomide Carbonyl Alkyl Carbon Chain Linker):

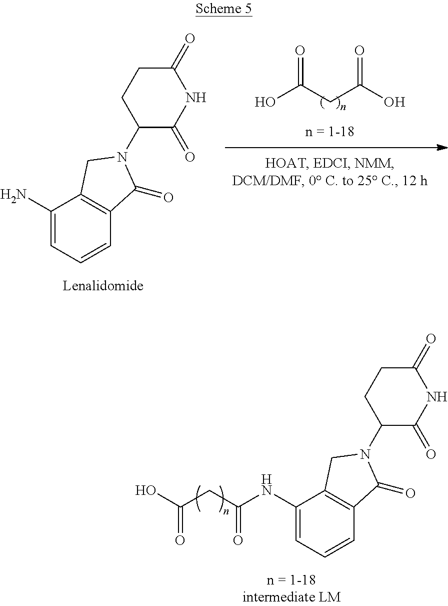

A 250 mL three-necked flask was charged with the corresponding diacid (5.0 mmol, 2.5 equiv) and subsequently anhydrous DMF (10 mL) and anhydrous DCM (150 mL). The mixture was stirred and cooled with an ice bath, and then added lenalidomide (2 mmol, 1 equiv), NMM (10.0 mmol, 5 equiv), HOAT (2.4 mmol, 1.2 equiv) and EDCI (2.4 mmol, 1.2 equiv). After the addition, the resulting reaction mixture was then warmed to room temperature and stirred overnight. After the reaction, the reaction mixture was quenched with deionized water (1 mL) and then concentrated under reduced pressure to remove DCM, and the residue was subjected to reverse-phase C₁₈ column chromatography (eluent (v/v) : acetonitrile/(water+0.1%TFA)=10% -100%) for separation and purification. The acetonitrile was removed by evaporation under reduced pressure, and the residue was lyophilized to yield the target product.

General Synthesis Method for Intermediate LM (Lenalidomide Alkyl Carbon Chain Linker):

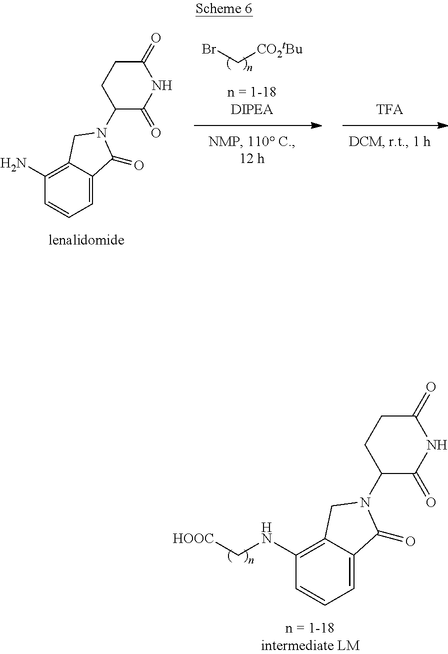

A single-neck flask was charged with lenalidomide (2 mmol, 1 equiv), NMP (10 mL) and corresponding tert-butyl bromide (2.4 mmol, 1.2 equiv) and DIPEA (6 mmol, 3 equiv). The mixture was stirred at 110° C. for 12 h. After cooling to room temperature, the resulting solution was subjected to reverse-phase C₁₈ column chromatography (eluent (v/v): acetonitrile/(water+0.1%TFA)=10% -100%) for separation and purification, to yield the desired tert-butyl ester intermediate. This intermediate was treated with TFA in DCM for 1 h at room temperature. After concentration under reduced pressure and freeze-drying, the desired product was obtained.

General Synthetic Method for ER Protein Regulator According to the Present Invention:

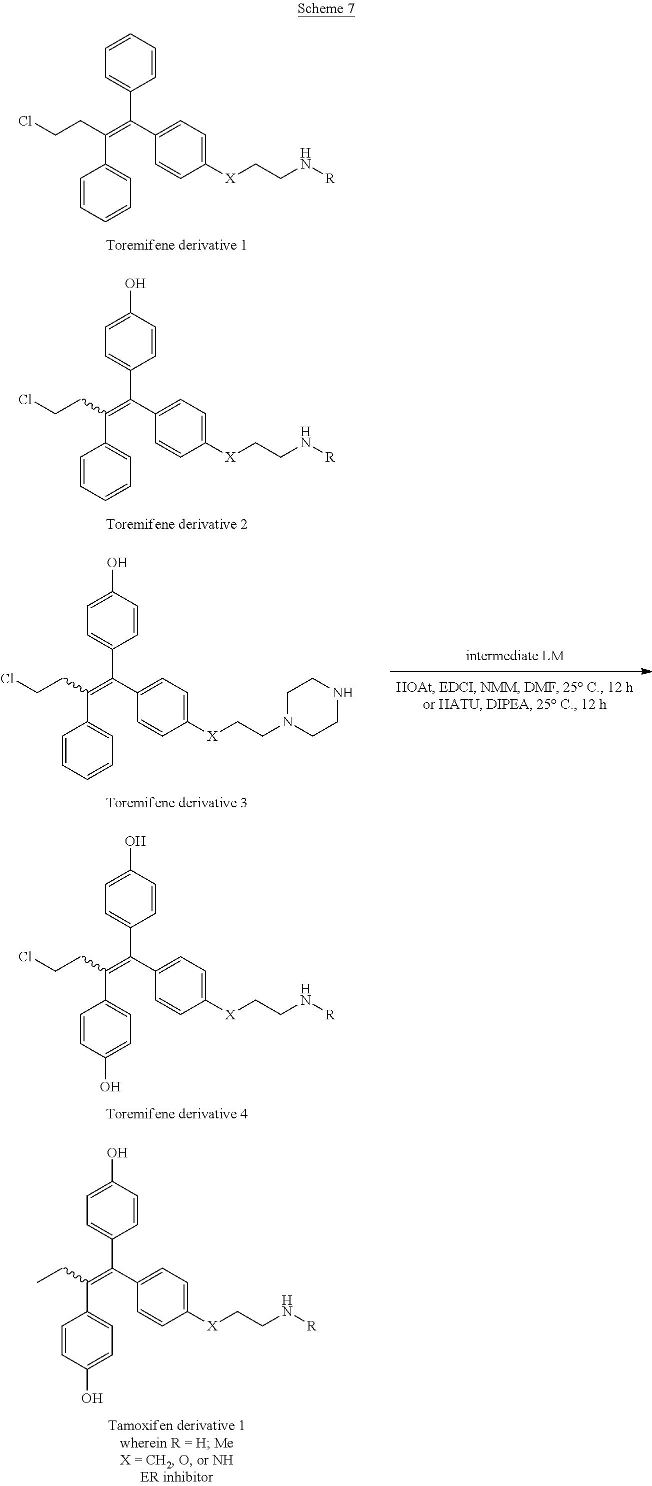

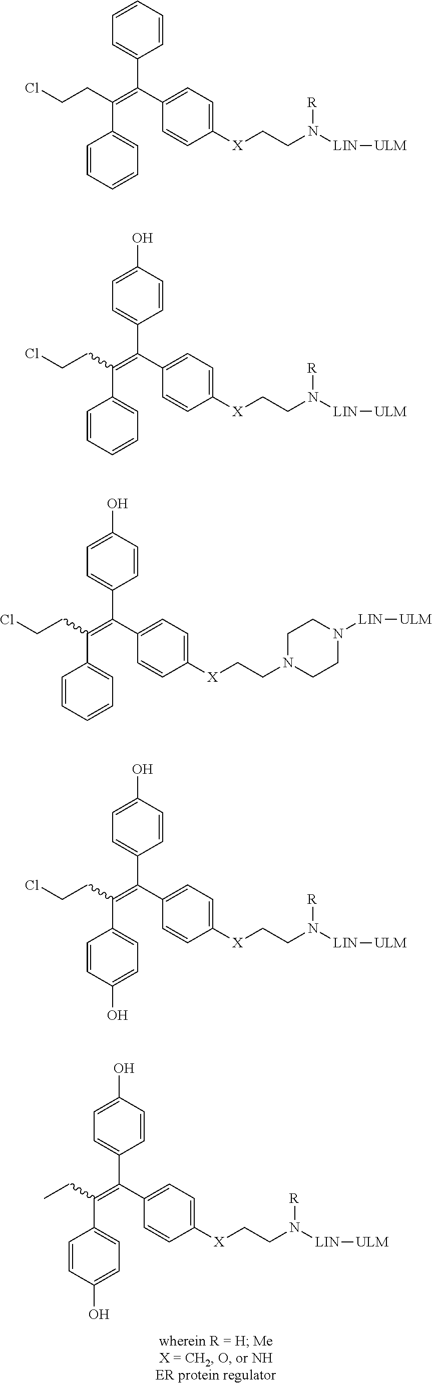

To a reaction flask were added corresponding selective ER inhibitor (1 equiv), corresponding intermediate LM (1 equiv), HOAt (2 equiv), EDCI (2 equiv), anhydrous DMF (2 mL) and NMM (5 equiv) sequentially at r.t.. The reaction mixture was stirred at room temperature overnight. After the reaction was complete as detected by LC-MS, the resulting solution was subjected to preparative HPLC (eluent (v/v): acetonitrile/(water+0.05%HCl)=10% -100%) for separation and purification. The acetonitrile was removed by rotary evaporation, and the residue was lyophilized to yield the target product.

### Intermediate Preparation Examples

### Intermediate Example 1

### Synthesis of Toremifene Derivative B

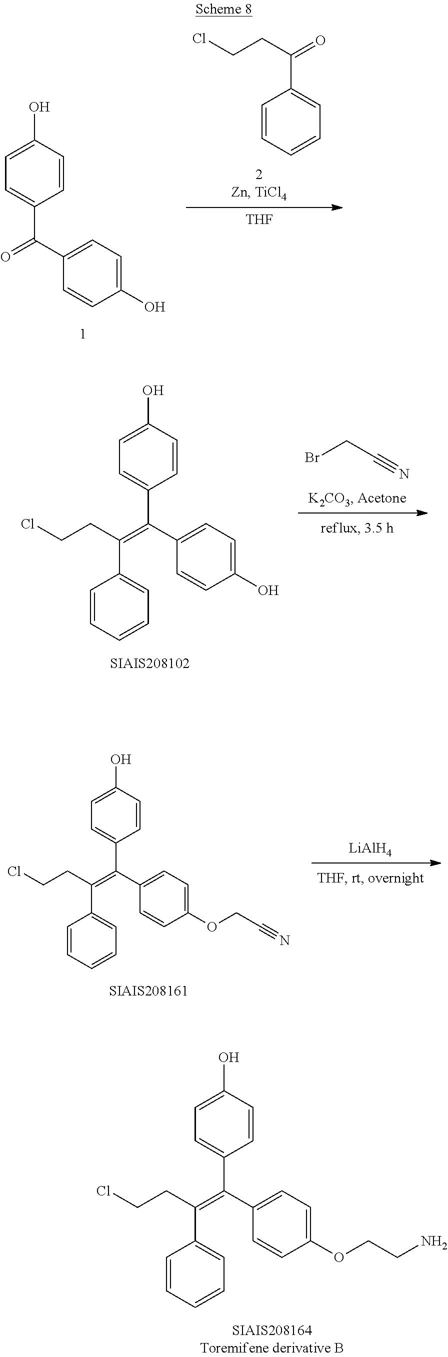

### Synthesis of 4,4′-(4-chloro-2-phenylbut-1-ene-1,1-diyl)biphenol (SIAIS 208102)

A dried three-necked flask equipped with a reflux condenser was charged with Zinc powder (6.5 g, 100 mmol), followed by evacuation and refilling with argon gas for three times, and then addition of THF (80 mL) under argon. TiCl₄ was added dropwise at 0° C. The mixture was warmed to r.t., and refluxed for 2 h. After the mixture was cooled to room temperature, a solution of compound 1 (2.14 g, 10 mmol) and compound 2 (5.1 g, 30 mmol) in THF (80 mL) was added, and refluxed in the dark for 3 h. After the reaction was complete, the resulting mixture was cooled down to r.t., and rotary evaporated to remove most of the solvent. The reaction was quenched with saturated ammonium chloride solution. The resulting mixture was extracted with ethyl acetate. The combined organic phases were washed sequentially with water and saturated brine, dried over anhydrous sodium sulfate, concentrated under reduced pressure, and the resulting residue was then separated and purified by silica gel column chromatography (the eluent is petroleum ether: ethyl acetate=2:1) to obtain a yellow solid product (3 g, 86% yield). ¹H NMR (500 MHz, CDCl₃) δ 7.21-7.10 (m, 7H), 6.84-6.81 (m, 2H), 6.75-6.72 (m, 2H), 6.49-6.46 (m, 2H), 4.99 (s, 1H), 4.73 (s, 1H), 3.45-3.36 (m, 2H), 2.99-2.91 (m, 2H). HRMS (ESI) m/z: calcd for C₂₂H₂₀ClO₂⁺ [M+H]⁺, 351.1146; found, 351.1138.

### Synthesis of 2-(4-(4-chloro-1-(4-hydroxyphenyl)-2-phenylbut-1-en-1-yl)phenoxy)acetonitrile (SIAIS208161)

A single-neck flask was sequentially charged with SIAIS208102 (1.5 g, 4.28 mmol), acetone (15 mL), K₂CO₃ (592 mg, 4.28 mmol), and bromoacetonitrile (257 mg, 2.14 mmol), followed by evacuation and refilling with argon gas for three times, and then heated to reflux under Ar gas for 3.5 h. After the reaction was complete, the resulting mixture was cooled down to room temperature, and rotary evaporated to remove the solvent. The resulting residue was separated and purified by silica gel column chromatography (the eluent is pure dichloromethane) to obtain a pale yellow liquid product (782 mg, 94% yield). ¹H NMR (500 MHz, CDCl₃) δ 7.31-7.27 (m, 1H), 7.21-7.18 (m, 2H), 7.17-7.14 (m, 2H), 7.13-7.10 (m, 2H), 7.00-6.97 (m, 1H), 6.86-6.83 (m, 2H), 6.75-6.70 (m, 1H), 6.65-6.61 (m, 1H), 6.51-6.47 (m, 1H), 4.95-4.70 (m, 1H), 4.81 (s, 1H), 4.70 (s, 1H), 4.64 (s, 1H), 3.45-3.39 (m, 2H), 2.97-2.91 (m, 2H). HRMS (ESI) m/z: calcd for C₂₄H₂₁ClNO₂⁺ [M+H]⁺, 390.1255; found, 390.1263.

### Synthesis of 4-(1-(4-(2-aminoethoxy)phenyl)-4-chloro-2-phenylbut-1-en-1-yl)phenol (SIAI5208164)

A single-neck flask was sequentially charged with SIAI5208161 (782 mg, 2 mmol) and THF (25 mL), followed by addtion in batches of LiAlH₄ at 0° C., and then evacuation and refilling with argon gas for three times. The mixture was stirred at room temperature under argon gas overnight. After the reaction was complete, the resulting mixture was quenched with saturated ammonium chloride solution, rotary evaporated to remove the solvent, washed with methanol, and filtered. The filtrate was concentrated under reduced pressure and then purified by reverse-phase C18 column chromatography (eluent is water (containing 0.05% HCl) and acetonitrile), to yield the desired product as a pale yellow solid (473 mg, 60% yield). ¹H NMR (500 MHz, DMSO) δ 9.68-9.17 (m, 1H), 8.12 (d, J=41.4 Hz, 3H), 7.24-7.18 (m, 3H), 7.16-7.12 (m, 3H), 7.06 (d, J=8.5 Hz, 1H), 7.00 (d, J=8.7 Hz, 1H), 6.77 (t, J=8.4 Hz, 2H), 6.65 (d, J=8.8 Hz, 1H), 6.61 (d, J=8.6 Hz, 1H), 6.42 (d, J=8.6 Hz, 1H), 4.20 (t, J=4.9 Hz, 1H), 4.03 (t, J=4.9 Hz, 1H), 3.43 (t, J=7.3 Hz, 2H), 3.23 (s, 1H), 3.12 (s, 1H), 2.93-2.83 (m, 2H). HRMS (ESI) m/z: calcd for C₂₄H₂₅ClNO₂⁺ [M+H]⁺, 394.1568; found, 394.1561.

### Intermediate Example 2

### Synthesis of Toremifene Derivative C

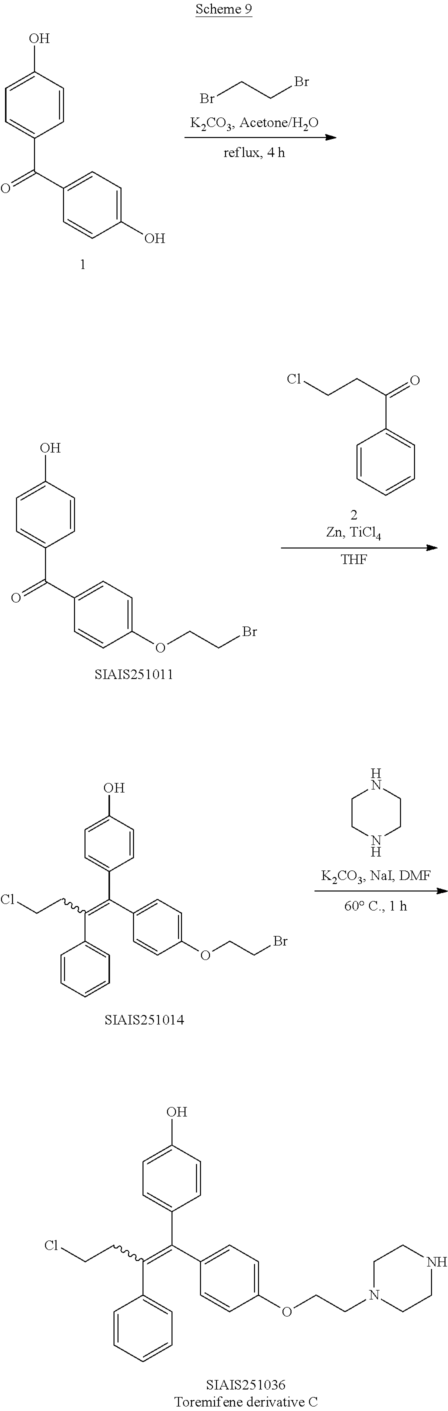

### Synthesis of (4-(2-bromoethoxy)phenyl)(4-hydroxyphenyl)methanone (SIAIS251011):

A single-neck flask equipped with a reflux condenser was sequentially charged with compound 1 (2.38 g, 11.1 mmol), Acetone/H₂O (30 mL/4 mL), dibromoethane (15 mL) and potassium carbonate (3.02 g, 21.8 mmol), followed by evacuation and refilling with argon gas for three times. The reaction mixture was heated to reflux under nitrogen gas for 4 h. After the reaction was complete, the resulting mixture was cooled down to r.t., and rotary evaporated to remove most of the solvent. The reaction was quenched with saturated ammonium chloride solution. The resulting mixture was extracted with ethyl acetate, and the combined organic phases were washed sequentially with water and saturated brine, dryed over anhydrous sodium sulfate, concentrated under reduced pressure. The resulting residue was then separated and purified by silica gel column chromatography (the eluent is petroleum ether: ethyl acetate=2:1) to obtain a white solid product (1.47 g, 41% yield). ¹H NMR (500 MHz, CDCl₃) δ 7.81-7.77 (m, 2H), 7.76-7.72 (m, 2H), 7.00-6.95 (m, 2H), 6.94-6.88 (m, 2H), 5.77 (s, 1H), 4.37 (t, J=6.2 Hz, 2H), 3.68 (t, J=6.2 Hz, 2H). HRMS (ESI) m/z: calcd for C₁₅H₁₄BrO₃⁺ [M+H]⁺, 321.0121; found, 321.0117.

### Synthesis of 4-(1-(4-(2-bromoethoxy)phenyl)-4-chloro-2-phenylbut-1-en-1-yl)phenol (SIAI5251014)

A dried three-necked flask equipped with a reflux condenser was charged with Zinc powder (2.32 g, 35.5 mmol), followed by evacuation and refilling with argon gas for three times, and then addition of THF (40 mL) under argon. TiCl₄ (3.37 g, 17.75 mmol) was added dropwise at 0° C. under argon. The mixture was warmed to r.t. and refluxed for 2 h. After the mixture was cooled to room temperature, a solution of intermediate SIAIS251011 (1.14 g, 3.55 mmol) and compound 2 (1.8 g, 10.65 mmol) in THF (40 mL) was added, and refluxed for 3 h in the dark. After the reaction was complete, the resulting mixture was cooled to R.T., and rotary evaporated to remove most of the solvent. The reaction was quenched with saturated ammonium chloride solution. The resulting mixture was extracted with ethyl acetate. The combined organic phases were washed sequentially with water and saturated brine, dryed over anhydrous sodium sulfate, concentrated under reduced pressure, and the resulting residue was then separated and purified by silica gel column chromatography (the eluent is petroleum ether:ethyl acetate=2:1) to obtain a yellow solid product (1.17 g, 72% yield). ¹H NMR (500 MHz, CDCl₃) δ 7.17 (m, 7H), 6.93-6.89 (m, 2H), 6.76-6.71 (m, 2H), 6.50-6.45 (m, 2H), 4.71 (s, 1H), 4.32 (t, J=6.3 Hz, 2H), 3.66 (t, J=6.3 Hz, 2H), 3.42 (t, J=7.5 Hz, 2H), 2.95 (td, J=7.4, 3.9 Hz, 2H). HRMS (ESI) m/z: calcd for C₂₄H₂₃BrClO₂⁺ [M+H]⁺, 457.0564; found, 457.0560.

### Synthesis of 4-(4-chloro-2-phenyl-1-(4-(2-(piperazin-1-yl)ethoxy)phenyl)but-1-en-1-yl)phenol (SIAI5251036)

A single-neck flask was sequentially charged with SIAIS251014 (1.3 g, 2.84 mmol), DMF (15 mL), potassium carbonate (1.18 g, 8.52 mmol) and sodium iodide (4.3 g, 28.4 mmol). The mixture was stirred at 60° C. for 1 h, and then cooled down to r.t., filtered, and washed with methanol. The filtrate was concentrated under reduced pressure and then purified by reverse-phase C18 column chromatography (eluent is water (containing 0.05% HCl) and acetonitrile), to yield the desired product as a white solid (520 mg, 40% yield). ¹H NMR (500 MHz, DMSO) δ 7.23-7.12 (m, 7H), 6.95 (dd, J=8.8, 2.5 Hz, 2H), 6.60 (d, J=8.5 Hz, 2H), 6.40 (d, J=8.6 Hz, 2H), 4.07 (t, J=5.8 Hz, 2H), 3.43 (t, J=7.3 Hz, 2H), 3.19-3.16 (m, 1H), 2.90-2.85 (m, 2H), 2.71-2.60 (m, 4H), 2.57-2.52 (m, 2H), 2.42-2.38 (m, 2H), 2.32-2.27 (m, 2H). HRMS (ESI) m/z: calcd for C₂₈H₃₂ClN₂O₂⁺ [M+H]⁺, 463.2147; found, 463.2142.

### Intermediate Example 3

3-(2-((2-(2,6-dioxopiperidin-3-yl)-1,3-dioxoisoindolin-4-yl)amino)ethoxy)propanoic acid (SIAIS151001) was prepared according to scheme 1, by using tert-butyl 3-(2-aminoethoxy)propionate as the starting material amine. The target product SIAIS151001 was obtained as yellow solid (1.0 g, 48% yield). ¹H NMR (500 MHz, DMSO) δ 12.17 (s, 1H), 11.09 (s, 1H), 7.57 (dd, J=8.5, 7.5 Hz, 1H), 7.13 (d, J=8.6 Hz, 1H), 7.04 (d, J=7.0 Hz, 1H), 6.59 (t, J=5.7 Hz, 1H), 5.05 (dd, J=12.8, 5.4 Hz, 1H), 3.65 (t, J=6.3 Hz, 2H), 3.59 (t, J=5.5 Hz, 2H), 3.46 (q, J=5.5 Hz, 2H), 2.91-2.83 (m, 1H), 2.61-2.52 (m, 2H), 2.46 (t, J=6.3 Hz, 2H), 2.05-2.00 (m, 1H); HRMS (ESI) m/z: calcd for C₁₈H₂₀N₃O₇⁺ [M+H]⁺, 390.1301; found, 390.1261.

### Intermediate Example 4

3-(2-(2-((2-(2,6-dioxopiperidin-3-yl)-1,3-dioxoisoindolin-4-yl)amino)ethoxy)ethoxy)propanoic acid (SIAIS151004) was prepared according to scheme 1, except that the starting material amine was tert-butyl 3-(2-(2-aminoethoxy)ethoxy)propanoate. The target product SIAIS151004 was obtained as yellow solid (0.95 g, 51% yield). ¹H NMR (500 MHz, DMSO) δ 11.09 (s, 1H), 7.58 (dd, J=8.0, 7.5 Hz, 1H), 7.14 (d, J=8.6 Hz, 1H), 7.04 (d, J=7.0 Hz, 1H), 6.60 (t, J=5.7 Hz, 1H), 5.05 (dd, J=12.8, 5.4 Hz, 1H), 3.62-3.58 (m, 4H), 3.56-3.54 (m, 2H), 3.52-3.49 (m, 2H), 3.46 (dd, J=11.1, 5.5 Hz, 2H), 2.92-2.84 (m, 1H), 2.66-2.51 (m, 2H), 2.42 (t, J=6.4 Hz, 2H), 2.06-1.98 (m, 1H); HRMS (ESI) m/z: calcd for C₂₀H₂₄N₃O₈⁺ [M+H]⁺, 434.1558; found, 434.1445.

### Intermediate Example 5

3-(2-(2-(2-((2-(2,6-dioxopiperidin-3-yl)-1,3-dioxoisoindolin-4-yl)amino)ethoxy)ethoxy)ethoxy)propanoic acid (SIAIS151005) was prepared according to scheme 1, except that the starting material amine was tert-butyl 3-(2-(2-(2-aminoethoxy)ethoxy)ethoxy)propanoate. The target product SIAIS151005 was obtained as yellow solid (0.95 g, 61% yield). ¹H NMR (500 MHz, DMSO) δ 11.09 (s, 1H), 7.58 (dd, J=8.0, 7.0 Hz, 1H), 7.15 (d, J=8.6 Hz, 1H), 7.04 (d, J=7.0 Hz, 1H), 6.61 (t, J=5.8 Hz, 1H), 5.05 (dd, J=12.8, 5.4 Hz, 1H), 3.63-3.48 (m, 14H), 2.92-2.83 (m, 1H), 2.64-2.52 (m, 2H), 2.18 (t, J=8.1 Hz, 2H), 2.07-1.99 (m, 1H). HRMS (ESI) m/z: calcd for C₂₂H₂₈N₃O₉⁺[ M+H]⁺, 478.1820; found, 478.1159.

### Intermediate Example 6

1-((2-(2,6-dioxopiperidin-3-yl)-1,3-dioxoisoindolin-4-yl)amino)-3,6,9,12-tetraoxapentadecan-15-oic acid (SIAIS151006) was prepared according to scheme 1, except that the starting material amine was tert-butyl 1-amino-3,6,9,12-tetraoxapentadecan-15-oate. The target product SIAIS151006 was obtained as yellow solid (0.87 g, 53% yield). ¹H NMR (500 MHz, DMSO) δ 11.09 (s, 1H), 7.58 (dd, J=8.5, 7.5 Hz, 1H), 7.15 (d, J=8.6 Hz, 1H), 7.04 (d, J=7.0 Hz, 1H), 6.60 (t, J=5.7 Hz, 1H), 5.05 (dd, J=12.8, 5.4 Hz, 1H), 3.63-3.48 (m, 18H), 2.92-2.84 (m, 1H), 2.63-2.52 (m, 2H), 2.41 (t, J=6.4 Hz, 2H), 2.07-1.98 (m, 1H). HRMS (ESI) m/z: calcd for C₂₄H₃₂N₃O₁₀⁺ [M+H]⁺, 522.2082; found, 522.2178.

### Intermediate Example 7

1-((2-(2,6-dioxopiperidin-3-yl)-1,3-dioxoisoindolin-4-yl)amino)-3,6,9,12,15-pentaoxaoctadecan-18-oic acid (SIAIS151007) was prepared according to scheme 1, except that the starting material amine was tert-butyl 1-amino-3,6,9,12,15-pentaoxaoctadecan-18-oate. The target product SIAIS151007 was obtained as yellow solid (0.8 g, 51% yield). ¹H NMR (500 MHz, DMSO) δ 11.09 (s, 1H), 7.58 (t, J=8.0 Hz, 1H), 7.14 (d, J=8.6 Hz, 1H), 7.04 (d, J=7.0 Hz, 1H), 6.60 (t, J=5.7 Hz, 1H), 5.05 (dd, J=12.8, 5.4 Hz, 1H), 3.63-3.54 (m, 8H), 3.54-3.48 (m, 12H), 3.30 (dd, J=7.0 Hz, 4H), 2.92-2.84 (m, 1H), 2.63-2.52 (m, 2H), 2.06-1.99 (m, 1H). HRMS (ESI) m/z: calcd for C₂₆H₃₆N₃O₁₁⁺ [M+H]⁺, 566.2344; found, 566.2679.

### Intermediate Example 8

(2-(2,6-dioxopiperidin-3-yl)-1,3-dioxoisoindolin-4-yl)glycine (SIAIS151025) was prepared according to scheme 2, except that the starting material amine was tert-butyl glycine. The target product SIAIS151025 was obtained as yellow solid (1.2 g, 48% yield). ¹H NMR (500 MHz, DMSO) δ 11.10 (s, 1H), 7.59 (dd, J=15.9, 8.5 Hz, 1H), 7.07 (d, J=7.0 Hz, 1H), 6.99 (d, J=8.6 Hz, 1H), 6.86 (t, J=5.7 Hz, 1H), 5.06 (dt, J=15.1, 7.6 Hz, 1H), 4.08 (d, J=5.7 Hz, 2H), 2.92-2.84 (m, 1H), 2.63-2.52 (m, 2H), 2.07-2.02 (m, 1H). HRMS (ESI) m/z: calcd for C₁₈H₂₀N₃O₆⁺ [M+H]⁺, 332.0877; found, 332.0720.

### Intermediate Example 9

3-((2-(2,6-dioxopiperidin-3-yl)-1,3-dioxoisoindolin-4-yl)amino)propanoic acid (SIAIS151026) was prepared according to scheme 2, except that the starting material amine was tert-butyl 3-amino-propanoate. The target product SIAIS151026 was obtained as yellow solid (0.93 g, 39% yield). ¹H NMR (500 MHz, DMSO) δ 11.09 (s, 1H), 7.59 (dd, J=8.0, 7.5 Hz, 1H), 7.15 (d, J=8.6 Hz, 1H), 7.04 (d, J=7.0 Hz, 1H), 6.67 (t, J=6.0 Hz, 1H), 5.05 (dd, J=12.8, 5.4 Hz, 1H), 3.53 (dd, J=12.6, 6.3 Hz, 2H), 2.92-2.84 (m, 1H), 2.65-2.53 (m, 4H), 2.08-1.98 (m, 1H). HRMS (ESI) m/z: calcd for C₁₆H₁₆N₃O₆⁺ [M+H⁺, 346.1034; found, 346.0868.

### Intermediate Example 10

4-((2-(2,6-dioxopiperidin-3-yl)-1,3-dioxoisoindolin-4-yl)amino)butanoic acid (SIAIS151019) was prepared according to scheme 2, except that the starting material amine was tert-butyl 4-amino-butanoate. The target product SIAIS151019 was obtained as yellow solid (0.8 g, 61% yield). ¹H NMR (500 MHz, DMSO) δ 12.14 (s, 1H), 11.09 (s, 1H), 7.58 (dd, J=8.4, 7.3 Hz, 1H), 7.13 (d, J=8.6 Hz, 1H), 7.02 (d, J=7.0 Hz, 1H), 6.65 (t, J=6.0 Hz, 1H), 5.05 (dd, J=12.8, 5.4 Hz, 1H), 3.32 (dd, J=13.7, 6.7 Hz, 2H), 2.94-2.82 (m, 1H), 2.66-2.51 (m, 2H), 2.30 (t, J=7.2 Hz, 2H), 2.05-2.00 (m, 1H), 1.82-1.75 (m, 2H). HRMS (ESI) m/z: calcd for C₁₇H₁₈N₃O₆⁺ [M+H⁺, 360.1190; found, 360.1223.

### Intermediate Example 11

4-((2-(2,6-dioxopiperidin-3-yl)-1,3-dioxoisoindolin-4-yl)amino)pentanoic acid (SIAIS151020) was prepared according to scheme 2, except that the starting material amine was tert-butyl 5-amino-pentanoate. The target product SIAIS151020 was obtained as yellow solid (0.9 g, 50% yield). ¹H NMR (500 MHz, DMSO) δ 12.05 (s, 1H), 11.11 (s, 1H), 7.57 (dd, J=8.3, 7.4 Hz, 1H), 7.09 (d, J=8.6 Hz, 1H), 7.02 (d, J=7.0 Hz, 1H), 6.56 (t, J=5.9 Hz, 1H), 5.05 (dd, J=12.7, 5.4 Hz, 1H), 3.32-3.28 (m, 2H), 2.94-2.82 (m, 1H), 2.62-2.51 (m, 2H), 2.27-2.25 (m, 2H), 2.06-1.99 (m, 1H), 1.62-1.53 (m, 4H). HRMS (ESI) m/z: calcd for C₁₈H₂₀N₃O₆⁺ [M+H]⁺, 374.1347; found, 374.1384.

### Intermediate Example 12

4-((2-(2,6-dioxopiperidin-3-yl)-1,3-dioxoisoindolin-4-yl)amino)hexanoic acid (SIAIS151027) was prepared according to scheme 2, except that the starting material amine was tert-butyl 6-amino-hexanoate. The target product SIAIS151027 was obtained as yellow solid (1.26 g, 61% yield). ¹H NMR (500 MHz, DMSO) δ 12.00 (s, 1H), 11.09 (s, 1H), 7.58 (dd, J=8.3, 7.4 Hz, 1H), 7.09 (d, J=8.6 Hz, 1H), 7.02 (d, J=7.0 Hz, 1H), 6.54 (t, J=5.9 Hz, 1H), 5.05 (dd, J=12.8, 5.4 Hz, 1H), 3.30-3.27 (m, 2H), 2.92-2.84 (m, 1H), 2.63-2.51 (m, 2H), 2.21 (t, J=7.5 Hz, 2H), 2.08-1.98 (m, 1H), 1.60-1.50 (m, 4H), 1.38-1.31 (m, 2H). HRMS (ESI) m/z: calcd for C₁₉H₂₂N₃O₆⁺ [M+H]⁺, 388.1503; found, 388.1119.

### Intermediate Example 13

7-((2-(2,6-dioxopiperidin-3-yl)-1,3-dioxoisoindolin-4-yl)amino)heptanoic acid (SIAIS151086) was prepared according to scheme 2, except that the starting material amine was tert-butyl 7-amino-heptanoate. The target product SIAIS151086 was obtained as yellow solid (1.3 g, 64% yield). ¹H NMR (500 MHz, DMSO) δ 12.04 (s, 1H), 11.09 (s, 1H), 7.58 (dd, J=8.3, 7.3 Hz, 1H), 7.09 (d, J=8.6 Hz, 1H), 7.02 (d, J=7.0 Hz, 1H), 6.53 (t, J=5.9 Hz, 1H), 5.05 (dd, J=12.7, 5.4 Hz, 1H), 3.28 (dd, J=13.4, 6.7 Hz, 2H), 2.94-2.82 (m, 1H), 2.65-2.51 (m, 2H), 2.19 (t, J=7.3 Hz, 2H), 2.05-2.00 (m, 1H), 1.60-1.53 (m, 2H), 1.53-1.46 (m, 2H), 1.37-1.28 (m, 4H). HRMS (ESI) m/z: calcd for C₂₀H₂₄N₃O₆⁺ [M+H]⁺, 402.1660; found, 402.1643.

### Intermediate Example 14

2-(2-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-2-oxoethoxy)acetic acid (SIAIS164112) was prepared according to scheme 3, except that the starting material diacid was 2,2′-oxydiacetic acid. The target product SIAIS164112 was obtained as white solid (0.3 g, 27% yield). ¹H NMR (500 MHz, MeOD) δ 9.09 (s, 1H), 7.50-7.43 (m, 4H), 4.70 (d, J=2.3 Hz, 1H), 4.60-4.50 (m, 3H), 4.37 (d, J=15.5 Hz, 1H), 4.27 (d, J=3.7 Hz, 1H), 4.22 (d, J=8.5 Hz, 1H), 4.14-4.10 (m, 2H), 3.90 (d, J=11.2 Hz, 1H), 3.81 (dd, J=11.0, 3.8 Hz, 1H), 2.50 (s, 3H), 2.25-2.21 (m, 1H), 2.12-2.06 (m, 1H), 1.05 (s, 9H). HRMS (ESI) m/z: calcd for C₂₆H₃₅N₄O₇S⁺ [M+H]⁺, 547.2221; found, 547.2118.

### Intermediate Example 15

2-(2-(2-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-2-oxoethoxy)ethoxy)acetic acid (SIAIS151010) was prepared according to scheme 3, except that the starting material diacid was 2,2′-(ethane-1,2-diylbis(oxy))diacetic acid. The target product SIAIS151010 was obtained as white solid (0.2 g, 23% yield). ¹H NMR (500 MHz, DMSO) δ 8.98 (s, 1H), 8.60 (t, J=5.9 Hz, 1H), 7.48 (d, J=9.5 Hz, 1H), 7.40 (s, 4H), 4.57 (d, J=9.6 Hz, 1H), 4.47-4.37 (m, 2H), 4.35 (s, 1H), 4.29-4.22 (m, 1H), 4.07 (d, J=12.5 Hz, 1H), 3.97 (s, 2H), 3.69-3.59 (m, 8H), 2.44 (s, 3H), 2.07-2.03 (m, 1H), 1.93-1.87 (m, 1H), 0.94 (s, 9H). HRMS (ESI) m/z: calcd for C₂₈H₃₉N₄O₈S⁺ [M+H]⁺, 591.2483; found, 591.2365.

### Intermediate Example 16

3-(2-(3-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-3-oxopropoxy)ethoxy)propanoic acid (SIAIS151002) was prepared according to scheme 3, except that the starting material diacid was 3,3′-(ethane-1,2-diylbis(oxy))dipropionic acid. The target product SIAIS151002 was obtained as white solid (0.53 g, 44% yield). ¹H NMR (500 MHz, DMSO) δ 12.17 (s, 1H), 8.99 (s, 1H), 8.57 (t, J=6.0 Hz, 1H), 7.92 (d, J=9.3 Hz, 1H), 7.41 (dd, J=18.5, 8.2 Hz, 4H), 4.55 (d, J=9.5 Hz, 1H), 4.46-4.40 (m, 2H), 4.36 (s, 1H), 4.23 (dd, J=15.8, 5.4 Hz, 1H), 3.69-3.56 (m, 7H), 3.49-3.46 (m, 4H), 2.58-2.53 (m, 1H), 2.47-2.42 (m, 2H), 2.45 (s, 3H), 2.39-2.32 (m, 1H), 2.06-2.01 (m, 1H), 1.95-1.88 (m, 1H), 0.94 (s, 9H). HRMS (ESI) m/z: calcd for C₃₀H₄₃N₄O₈S⁺ [M+H]⁺, 619.2796; found, 619.2973.

### Intermediate Example 17

(S)-15-((2S,4R)-4-hydroxy-2-((4-(4-methylthiazol-5-yl)benzyl)carbamoyl)pyrrolidin-1-carbonyl)-16,16-dimethyl-13-oxo-4,7,10-trioxa-14-azaheptadecanoic acid (SIAIS151003) was prepared according to scheme 3, except that the starting material diacid was 3,3′-((oxybis(ethane-2,1-diyl))bis(oxy))dipropionic acid. The target product SIAIS151003 was obtained as white solid (0.63 g, 59% yield). ¹H NMR (500 MHz, DMSO) δ 8.99 (s, 1H), 8.57 (t, J=6.0 Hz, 1H), 7.92 (d, J=9.4 Hz, 1H), 7.41 (dd, J=18.5, 8.2 Hz, 4H), 4.56 (d, J=9.4 Hz, 1H), 4.47-4.41 (m, 2H), 4.36 (s, 1H), 4.23 (dd, J=15.9, 5.5 Hz, 1H), 3.70-3.57 (m, 8H), 3.51-3.47 (m, 7H), 2.58-2.52 (m, 1H), 2.47-2.42 (m, 2H), 2.45 (s, 3H), 2.39-2.32 (m, 1H), 2.08-2.00 (m, 1H), 1.94-1.88 (m, 1H), 0.94 (s, 9H). HRMS (ESI) m/z: calcd for C₃₂H₄₇N₄O₉S⁺ [M+H]⁺, 663.3058; found, 663.3008.

### Intermediate Example 18

(S)-18-((2S,4R)-4-hydroxy-2-((4-(4-methylthiazol-5-yl)benzyl)carbamoyl)pyrrolidin-1-carbonyl)-19,19-dimethyl-16-oxo-4,7,10,13-tetraoxa-17-azaicosanoic acid (SIAIS151008) was prepared according to scheme 3, except that the starting material diacid was 4,7,10,13-tetraoxahexadecanedioic acid. The target product SIAIS151008 was obtained as white solid (0.53 g, 51% yield). ¹H NMR (500 MHz, DMSO) δ 8.98 (s, 1H), 8.56 (t, J=6.0 Hz, 1H), 7.91 (d, J=9.4 Hz, 1H), 7.40 (dd, J=18.8, 8.3 Hz, 4H), 4.55 (d, J=9.4 Hz, 1H), 4.45-4.40 (m, 2H), 4.35 (s, 1H), 4.22 (dd, J=15.8, 5.5 Hz, 1H), 3.69-3.54 (m, 10H), 3.48 (d, J=2.7 Hz, 9H), 2.56-2.52 (m, 1H), 2.45-2.41 (m, 2H), 2.45 (s, 3H), 2.38-2.32 (m, 1H), 2.06-2.00 (m, 1H), 1.94-1.88 (m, 1H), 0.93 (s, 9H). HRMS (ESI) m/z: calcd for C₃₄H₅₁N₄O₁₀S⁺ [M+H]⁺, 707.3320; found, 707.2945.

### Intermediate Example 19

(S)-21-((2S,4R)-4-hydroxy-2-((4-(4-methylthiazol-5-yl)benzyl)carbamoyl)pyrrolidin-1-carbonyl)-22,22-dimethyl-19-oxo-4,7,10,13,16-pentaoxa-20-azatricosanoic acid (SIAIS151009) was prepared according to scheme 3, except that the starting material diacid was 4,7,10,13,16-pentaoxanonadecanedioic acid. The target product SIAIS151009 was obtained as white solid (0.82 g, 85% yield). ¹H NMR (500 MHz, DMSO) δ 8.98 (s, 1H), 8.56 (d, J=5.7 Hz, 1H), 7.91 (d, J=9.3 Hz, 1H), 7.40 (dd, J=18.6, 7.9 Hz, 4H), 4.55 (d, J=9.3 Hz, 1H), 4.47-4.40 (m, 2H), 4.35 (s, 1H), 4.22 (dd, J=15.7, 5.2 Hz, 1H), 3.68-3.56 (m, 11H), 3.51-3.49 (s, 9H), 2.56-2.53 (m, 1H), 2.45-2.41 (m, 5H), 2.44 (s, 3H), 2.36 (dd, J=13.4, 7.0 Hz, 1H), 2.08-2.00 (m, 1H), 1.94-1.86 (m, 1H), 0.93 (s, 9H). HRMS (ESI) m/z: calcd for C₃₆H₅₅N₄O₁₁S⁺ [M+H]⁺, 751.3583; found, 751.3199.

### Intermediate Example 20

4-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-4-oxobutanoic acid (SIAIS074011) was prepared according to scheme 4, except that the starting material diacid was succinic acid. The target product SIAIS074011 was obtained as white solid (0.82 g, 65% yield). ¹H NMR (500 MHz, CDCl₃) δ 11.88 (s, 1H), 8.85 (s, J=11.2 Hz, 1H), 7.69 (s, 1H), 7.37-7.29 (m, 4H), 6.09 (br, 1H), 4.67-4.54 (m, 3H), 4.49 (s, 1H), 4.29 (dd, J=15.0, 5.0Hz, 1H), 4.05 (d, J=11.3 Hz, 1H), 3.73-3.63 (m, 1H), 2.73-2.58 (m, 1H), 2.57-2.41 (m, 3H), 2.50 (s, 3H), 2.31-2.14 (m, 2H), 0.96 (s, 9H). HRMS (ESI) m/z: calcd for C₂₆H₃₅N₄O₆S⁺ [M+H]⁺, 531.2272; found, 531.2275.

### Intermediate Example 21

5-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-5-oxopentanoic acid (SIAIS074012) was prepared according to scheme 4, except that the starting material diacid was glutaric acid. The target product SIAIS074012 was obtained as white solid (0.85 g, 67% yield). ¹H NMR (500 MHz, CDCl₃) δ 9.08 (s, 1H), 8.65 (br, 1H), 8.10 (s, 1H), 7.38-7.29 (m, 4H), 4.72-4.64 (m, 3H), 4.52 (s, 1H), 4.25 (dd, J=15.4, 5.0 Hz, 1H), 4.09 (d, J=10.5 Hz, 1H), 3.73 (d, J=10.0 Hz, 1H), 2.48 (s, 3H), 2.39-2.13 (m, 6H), 1.92-1.74 (m, 2H), 0.96 (s, 9H). HRMS (ESI) m/z: calcd for C₂₇H₃₇N₄O₆S⁺ [M+H]⁺, 545.2428; found, 545.2428.

### Intermediate Example 22

6-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-6-oxohexanoic acid (SIAIS074013) was prepared according to scheme 4, except that the starting material diacid was adipic acid. The target product SIAIS074013 was obtained as white solid (0.79 g, 55% yield). ¹H NMR (500 MHz, CDCl₃) δ 8.99 (s, 1H), 7.66 (s, 1H), 7.39-7.33 (m, 4H), 7.30 (d, J=7.5 Hz, 1H). 7.14 (br, 1H), 4.67-4.61 (m, 3H), 4.52 (s, 1H). 4.28 (dd, J=15.4, 5.0 Hz, 1H), 4.09 (d, J=11.4 Hz, 1H), 3.74-3.63 (m, 1H), 2.52 (s, 3H), 2.31-2.17 (m, 6H), 1.65-1.53 (m, 4H), 0.96 (s, 9H). HRMS (ESI) m/z: calcd for C₂₈H₄₀N₄O₆S⁺ [M+H]⁺, 559.2585; found, 559.3632.

### Intermediate Example 23

7-(((S)-1-((2S,4R)-4-hydroxy-24(4-(4-methylthiazol-5-yl)benzyl)carbamoyl)pyrrolidin-1-yl)-3,3-dimethyl-1-oxobutan-2-yl)amino)-7-oxoheptanoic acid (SIAIS074014) was prepared according to scheme 4, except that the starting material diacid was pimelic acid. The target product SIAIS074014 was obtained as white solid (0.8 g, 57% yield). ¹H NMR (500 MHz, CDCl₃) δ 8.90 (s, 1H), 7.42-7.38 (m, 1H), 7.41-7.33 (m, 4H), 7.31 (d, J=9.0 Hz, 1H), 6.38 (br, 1H), 4.79-4.46 (m, 3H), 4.55 (s, 1H), 4.28 (dd, J=15.2, 5.1 Hz, 1H), 4.12 (d, J=11.3 Hz, 1H), 3.72-3.63 (m, 1H), 2.51 (s, 3H), 2.38-2.33 (m, 1H), 2.28-2.21 (m, 4H), 2.18-2.12 (m, 1H), 1.62-1.52 (m, 3H), 1.33-1.23 (m, 3H), 0.96 (s, 9H). HRMS (ESI) m/z: calcd for C₂₉H₄₁N₄O₆S⁺ [M+H]⁺, 573.2741; found, 573.3804.

### Intermediate Example 24

8-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-8-oxooctanoic acid (SIAIS074015) was prepared according to scheme 4, except that the starting material diacid was suberic acid. The target product SIAIS074015 was obtained as white solid (0.95 g, 68% yield). ¹H NMR (500 MHz, CDCl₃) δ 8.82 (s, 1H), 7.43 (t, J=6.0 Hz, 1H), 7.34 (s, 4H), 6.98 (d, J=8.5 Hz, 1H), 6.10 (s, 1H), 4.69-4.65 (m, 1H), 4.63-4.51 (m, 2H), 4.55-4.50 (m, 1H), 4.38-4.27 (m, 1H), 4.11 (d, J=16.7 Hz, 1H), 3.72-3.62 (m, 1H), 2.51 (s, 3H), 2.39-2.13 (m, 6H), 1.58-1.54 (m, 4H), 1.33-1.21 (m, 4H), 0.95 (s, 9H). HRMS (ESI) m/z: calcd for C₃₀H₄₃N₄O₆S⁺ [M+H]⁺, 587.2898; found, 587.2917.

### Intermediate Example 25

9-(((S)-1-((2S,4R)-4-hydroxy-24(4-(4-methylthiazol-5-yl)benzyl)carbamoyl)pyrrolidin-1-yl)-3,3-dimethyl-1-oxobutan-2-yl)amino)-9-oxononanoic acid (SIAIS074016) was prepared according to scheme 4, except that the starting material diacid was azelaic acid. The target product SIAIS074016 was obtained as white solid (0.92 g, 64% yield). ¹H NMR (500 MHz, CDCl₃) δ 8.82 (s, 1H), 7.35 (s, 4H), 7.02 (t, J=14.3 Hz, 1H), 5.99 (s, 1H), 4.74-4.49 (m, 4H), 4.30 (dd, J=15.2, 5.1 Hz, 1H), 4.13 (d, J=11.3 Hz, 1H), 3.67 (dd, J=11.5, 3.5 Hz, 1H), 2.51 (s, 3H), 2.42-2.36 (m, 1H), 2.28 (t, J=7.5 Hz, 2H), 2.24-2.12 (m, 3H), 1.67-1.48 (m, 4H), 1.35-1.22 (m, 6H), 0.95 (s, 9H). HRMS (ESI) m/z: calcd for C₃₁H₄₅N₄O₆S⁺ [M+H]⁺, 601.3054; found, 601.3150.

### Intermediate Example 26

10-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-10-oxodecanoic acid (SIAIS074019) was prepared according to scheme 4, except that the starting material diacid was sebacic acid. The target product SIAIS074019 was obtained as white solid (0.96 g, 66% yield). ¹H NMR (500 MHz, CDCl₃) δ 8.79 (s, 1H), 7.39-7.36 (m, 1H), 7.35 (s, 4H), 7.01 (d, J=9.0 Hz, 1H), 5.80 (s, 1H), 4.68-4.52 (m, 4H), 4.29 (dd, J=15.2, 5.0 Hz, 1H), 4.12 (d, J=11.2 Hz, 1H), 3.72-3.62 (m, 1H), 2.51 (s, 3H), 2.41-2.33 (m, 1H), 2.32-2.23 (m, 2H), 2.23-2.11 (m, 3H), 1.65-1.48 (m, 4H), 1.32-1.21 (m, 8H), 0.95 (s, 9H). HRMS (ESI) m/z: calcd for C₃₂H₄₇N₄O₆S⁺ [M+H]⁺ 615.3211; found, 615.4391.

### Intermediate Example 27

11-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-11-oxoundecanoic acid (SIAIS074020) was prepared according to scheme 4, except that the starting material diacid was 1,11-undecanedioic acid. The target product SIAIS074020 was obtained as white solid (1 g, 67% yield). ¹H NMR (500 MHz, CDCl₃) δ 8.77 (s, 1H), 7.39-7.32 (m, 4H), 7.30 (m, 1H), 7.01 (d, J=8.8 Hz, 1H), 5.52 (br, 1H), 4.69-4.59 (m, 3H), 4.53 (s, 1H), 4.29 (dd, J=15.2, 5.0 Hz, 1H), 4.14 (d, J=11.3 Hz, 1H), 3.68-3.64 (m, 1H), 2.51 (s, 3H), 2.44-2.40 (m, 1H), 2.29 (t, J=7.1 Hz, 2H), 2.26-2.12 (m, 3H), 1.68-1.48 (m, 4H), 1.30-1.20 (m, 10H), 0.95 (s, 9H). HRMS (ESI) m/z: calcd for C₃₃H₄₉N₄O₆S⁺ [M+H]⁺, 629.3367; found, 629.4540.

### Intermediate Example 28

14-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-14-oxotetradecanoic acid (SIAIS164185) was prepared according to scheme 4, except that the starting material diacid was 1,12-dodecanedicarboxylic acid. The target product SIAIS164185 was obtained as white solid (523 mg, 70% yield). ¹H NMR (500 MHz, MeOD) δ 8.95 (s, 1H), 7.48 (d, J=8.4 Hz, 2H), 7.44-7.41 (m, 2H), 4.64 (s, 1H), 4.58-4.49 (m, 3H), 4.36 (d, J=15.4 Hz, 1H), 3.91 (d, J=11.0 Hz, 1H), 3.81 (dd, J=10.9, 3.9 Hz, 1H), 2.48 (s, 3H), 2.32-2.22 (m, 11H), 2.12-2.05 (m, 1H), 1.63-1.56 (m, 10H), 1.29-1.28 (m, 8H), 1.04 (s, 9H). HRMS (ESI) m/z: calcd for C₃₆H₅₅N₄O₆S⁺ [M+H]⁺, 671.3837; found, 671.0892.

### Intermediate Example 29

16-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-16-oxohexadecanoic acid (SIAIS164189) was prepared according to scheme 4, except that the starting material diacid was 16-hexadecanedioic acid. The target product SIAIS164189 was obtained as white solid (488 mg, 68% yield). ¹H NMR (500 MHz, MeOD) δ 8.90 (s, 1H), 7.49-7.44 (m, 2H), 7.44-7.40 (m, 2H), 4.64 (s, 1H), 4.59-4.48 (m, 3H), 4.40-4.31 (m, 1H), 3.90 (d, J=11.1 Hz, 1H), 3.80 (dd, J=10.9, 3.9 Hz, 1H), 2.48 (s, 3H), 2.30-2.25 (m, 8H), 2.23-2.19 (m, 1H), 2.11-2.06 (m, 1H), 1.62-1.59 (m, 10H), 1.30-1.29 (m, 6H), 1.04 (s, 9H). HRMS (ESI) m/z: calcd for C₃₈H₅₉N₄O₆S⁺ [M+H]⁺, 699.4150; found, 699.0566.

### Intermediate Example 30

3-((2-(2,6-dioxopiperidin-3-yl)-1-oxoisoindolin-4-yl)amino)-3-oxopropanoic acid (SIAIS171004) was prepared according to scheme 4, except that the starting material diacid was malonic acid. The target product SIAIS171004 was obtained as white solid (0.32 g, 24% yield). ¹H NMR (500 MHz, DMSO) δ 11.02 (s, 1H), 10.03 (s, 1H), 7.86 (d, J=7.1 Hz, 1H), 7.62-7.43 (m, 2H), 5.15 (dd, J=13.4, 4.9 Hz, 1H), 4.36 (dd, J=35.5, 17.5 Hz, 2H), 3.42 (s, 2H), 2.95-2.87 (m, 1H), 2.63-2.59 (m, 1H), 2.38-2.28 (m, 1H), 2.07-2.01 (m, 1H). HRMS (ESI) m/z: calcd for C₁₆H₁₆N₃O₆⁺ [M+H]⁺, 346.1034; found, 346.1015.

### Intermediate Example 31

4-((2-(2,6-dioxopiperidin-3-yl)-1-oxoisoindolin-4-yl)amino)-4-oxobutanoic acid (SIAIS164084) was prepared according to scheme 5, except that the starting material diacid was succinic acid. The target product SIAIS164084 was obtained as white solid (0.11 g, 44% yield). ¹H NMR (500 MHz, DMSO) δ 12.16 (s, 1H), 11.02 (s, 1H), 9.86 (s, 1H), 7.81 (dd, J=7.1, 1.7 Hz, 1H), 7.57-7.40 (m, 2H), 5.15 (dd, J=13.3, 5.1 Hz, 1H), 4.35 (dd, J=35.5, 17.5 Hz, 2H), 2.96-2.87 (m, 1H), 2.65-2.58 (m, 3H), 2.55-2.53 (m, 2H), 2.37-2.29 (m, 1H), 2.06-2.00 (m, 1H). HRMS (ESI) m/z: calcd for C₁₇H₁₈N₃O₆⁺ [M+H]⁺, 360.1190; found, 360.1198.

### Intermediate Example 32

5-((2-(2,6-dioxopiperidin-3-yl)-1-oxoisoindolin-4-yl)amino)-5-oxopentanoic acid (SIAIS171005) was prepared according to scheme 5, except that the starting material diacid was glutaric acid. The target product SIAIS171005 was obtained as white solid (0.52 g, 35% yield). ¹H NMR (500 MHz, DMSO) δ 11.01 (s, 1H), 9.80 (s, 1H), 7.81 (d, J=5.8 Hz, 1H), 7.54-7.46 (m, 2H), 5.15 (dd, J=13.3, 5.1 Hz, 1H), 4.36 (dd, J=35.5, 17.5 Hz, 2H), 2.97-2.85 (m, 1H), 2.77-2.75 (m, 2H), 2.66-2.57 (m, 1H), 2.42-2.39 (m, 1H), 2.35 (dd, J=13.1, 4.4 Hz, 1H), 2.30-2.27 (m, 1H), 2.03-1.97 (m, 1H), 1.85-1.79 (m, 2H). HRMS (ESI) m/z: calcd for C₁₈H₂₀N₃O₆⁺ [M+H]⁺, 374.1347; found, 374.1526.

### Intermediate Example 33

6-((2-(2,6-dioxopiperidin-3-yl)-1-oxoisoindolin-4-yl)amino)-6-oxohexanoic acid (SIAIS164101) was prepared according to scheme 5, except that the starting material diacid was adipic acid. The target product SIAIS164101 was obtained as white solid (0.4 g, 27% yield). ¹H NMR (500 MHz, MeOD) δ 7.70 (d, J=7.9 Hz, 1H), 7.66 (d, J=7.4 Hz, 1H), 7.52 (t, J=7.7 Hz, 1H), 5.16 (dd, J=13.4, 5.2 Hz, 1H), 4.53-4.43 (m, 2H), 2.95-2.87 (m, 1H), 2.81-2.76 (m, 1H), 2.55-2.48 (m, 1H), 2.46 (t, J=7.2 Hz, 2H), 2.36 (t, J=7.0 Hz, 2H), 2.22-2.16 (m, 1H), 1.79-1.66 (m, 4H). HRMS (ESI) m/z: calcd for C₁₉H₂₂N₃O₆⁺ [M+H]⁺, 388.1503; found, 388.1714.

### Intermediate Example 34

7-((2-(2,6-dioxopiperidin-3-yl)-1-oxoisoindolin-4-yl)amino)-7-oxoheptanoic acid (SIAIS164102) was prepared according to scheme 5, except that the starting material diacid was pimelic acid. The target product SIAIS164102 was obtained as white solid (0.45 g, 28% yield). ¹H NMR (500 MHz, MeOD) δ 7.70 (d, J=7.9 Hz, 1H), 7.65 (d, J=7.4 Hz, 1H), 7.52 (t, J=7.7 Hz, 1H), 5.16 (dd, J=13.4, 5.2 Hz, 1H), 4.49 (t, J=10.1 Hz, 2H), 2.94-2.87 (m, 1H), 2.81-2.76 (m, 1H), 2.54-2.48 (m, 1H), 2.45 (t, J=7.5 Hz, 2H), 2.32 (t, J=7.0 Hz, 2H), 2.22-2.16 (m, 1H), 1.77-1.72 (m, 2H), 1.70-1.63 (m, 2H), 1.48-1.42 (m, 2H). HRMS (ESI) m/z: calcd for C₂₀H₂₄N₃O₆⁺ [M+H]⁺, 402.1660; found, 402.1890.

### Intermediate Example 35

(2-(2,6-dioxopiperidin-3-yl)-1-oxoisoindolin-4-yl)aminoacetic acid (SIAIS1204057) was prepared according to scheme 6, except that the starting material tert-butyl bromide was tert-butyl 2-bromoacetate. The target product SIAIS1204057 was obtained as yellow solid (1 g, 48% yield). ¹H NMR (500 MHz, DMSO) δ 11.01 (s, 1H), 7.28 (t, J=7.7 Hz, 1H), 6.98 (d, J=7.3 Hz, 1H), 6.66 (d, J=8.0 Hz, 1H), 5.94 (s, 1H), 5.12 (dd, J=13.3, 5.1 Hz, 1H), 4.26 (d, J=17.0 Hz, 1H), 4.16 (d, J=17.0 Hz, 1H), 3.92 (s, 2H), 2.98-2.85 (m, 1H), 2.62 (d, J=17.3 Hz, 1H), 2.39-2.26 (m, 1H), 2.08-1.99 (m, 1H). HRMS (ESI) m/z: calcd for C₁₅H₁₆N₃O₅⁺ [M+H]⁺, 318.1084; found, 318.1098.

### Intermediate Example 36

4-((2-(2,6-dioxopiperidin-3-yl)-1-oxoisoindolin-4-yl)amino)butanoic acid (SIAIS1204085) was prepared according to scheme 6, except that the starting material tert-butyl bromide was tert-butyl 4-bromobutanoate. The target product SIAIS1204085 was obtained as yellow solid (215 mg, 62% yield). ¹H NMR (500 MHz, DMSO) δ 11.01 (s, 1H), 7.28 (t, J=7.7 Hz, 1H), 6.93 (d, J=7.3 Hz, 1H), 6.77 (d, J=8.0 Hz, 1H), 5.11 (dd, J=13.3, 5.1 Hz, 1H), 4.23 (d, J=17.0 Hz, 1H), 4.13 (d, J=17.0 Hz, 1H), 4.01 (s, 1H), 3.14 (t, J=7.0 Hz, 2H), 2.98-2.86 (m, 1H), 2.66-2.58 (d, J=17.6 Hz, 1H), 2.34 (t, J=7.3 Hz, 2H), 2.32-2.24 (m, 1H), 2.08-1.98 (m, 1H), 1.85-1.75 (m, 2H). HRMS (ESI) m/z: calcd for C₁₇H₂₀N₃O₅⁺ [M+H]⁺, 346.1379; found, 346.1414.

### Intermediate Example 37

5-((2-(2,6-dioxopiperidin-3-yl)-1-oxoisoindolin-4-yl)amino)pentanoic acid (SIAIS1210133) was prepared according to scheme 6, except that the starting material tert-butyl bromide was tert-butyl 5-bromopentanoate. The target product SIAIS1210133 was obtained as yellow solid (215 mg, 60% yield). ¹H NMR (500 MHz, DMSO) δ 11.00 (s, 1H), 7.28 (t, J=7.7 Hz, 1H), 6.92 (t, J=10.9 Hz, 1H), 6.76 (d, J=8.0 Hz, 1H), 5.11 (dd, J=13.3, 5.1 Hz, 1H), 5.07 (s, 1H), 4.23 (d, J=17.2 Hz, 1H), 4.13 (d, J=17.1 Hz, 1H), 3.13 (d, J=6.4 Hz, 2H), 2.97-2.87 (m, 1H), 2.61 (d, J=16.7 Hz, 1H), 2.38-2.21 (m, 3H), 2.06-1.98 (m, 1H), 1.67-1.55 (m, 4H). HRMS (ESI) m/z: calcd for C₁₈H₂₂N₃O₅⁺ [M+H]⁺, 360.1554; found, 360.1551.

### Intermediate Example 38

6-((2-(2,6-dioxopiperidin-3-yl)-1-oxoisoindolin-4-yl)amino)hexanoic acid (SIAIS1204061) was prepared according to scheme 6, except that the starting material tert-butyl bromide was tert-butyl 6-bromohexanoate. The target product SIAIS1204061 was obtained as yellow solid (268 mg, 72% yield). ¹H NMR (500 MHz, DMSO) δ 11.01 (s, 1H), 7.29 (t, J=7.7 Hz, 1H), 6.94 (d, J=7.4 Hz, 1H), 6.76 (d, J=8.0 Hz, 1H), 5.11 (dd, J=13.3, 5.1 Hz, 1H), 4.24 (d, J=17.0 Hz, 1H), 4.14 (d, J=17.0 Hz, 1H), 4.05 (s, 1H), 3.12 (t, J=7.0 Hz, 2H), 2.98-2.87 (m, 1H), 2.66-2.58 (m, 1H), 2.35-2.25 (m, 1H), 2.22 (t, J=7.0 Hz, 2H), 2.07-2.00 (m, 1H), 1.63-1.50 (m, 4H), 1.43-1.37 (m, 2H). HRMS (ESI) m/z: calcd for C₁₉H₂₄N₃O₅⁺ [M+H]⁺, 374.1710; found, 374.1720.

### Intermediate Example 39

7-((2-(2,6-dioxopiperidin-3-yl)-1-oxoisoindolin-4-yl)amino)heptanoic acid (SIAIS1204063) was prepared according to scheme 6, except that the starting material tert-butyl bromide was tert-butyl 7-bromoheptanoate. The target product SIAIS1204063 was obtained as yellow solid (252 mg, 65%). ¹H NMR (500 MHz, DMSO) δ 11.00 (s, 1H), 7.28 (t, J=7.7 Hz, 1H), 6.93 (d, J=7.3 Hz, 1H), 6.75 (d, J=8.0 Hz, 1H), 5.11 (dd, J=13.2, 5.0 Hz, 1H), 4.23 (d, J=17.0 Hz, 1H), 4.13 (d, J=17.0 Hz, 1H), 3.11 (t, J=7.0 Hz, 2H), 2.98-2.84 (m, 1H), 2.67-2.57 (m, 1H), 2.35-2.25 (m, 1H), 2.20 (t, J=7.3 Hz, 2H), 2.07-1.99 (m, 1H), 1.63-1.46 (m, 4H), 1.42-1.27 (m, 4H). HRMS (ESI) m/z: calcd for C₂₀H₂₆N₃O₅⁺ [M+H]⁺, 388.1867; found, 388.1878.

### Intermediate Example 40

### Synthesis of 3-(4-(3-(((S)-1-((2S,4R)-4-hydroxy-2-((4-(4-methylthiazol-5-yl)benzyl)c arb amoyl)pyrrolidin-1-yl)-3,3-dimethyl-1-oxobutan-2-yl)amino)-3-oxopropyl)piperazin-1-yl)propanoic acid (SIAIS1213011)

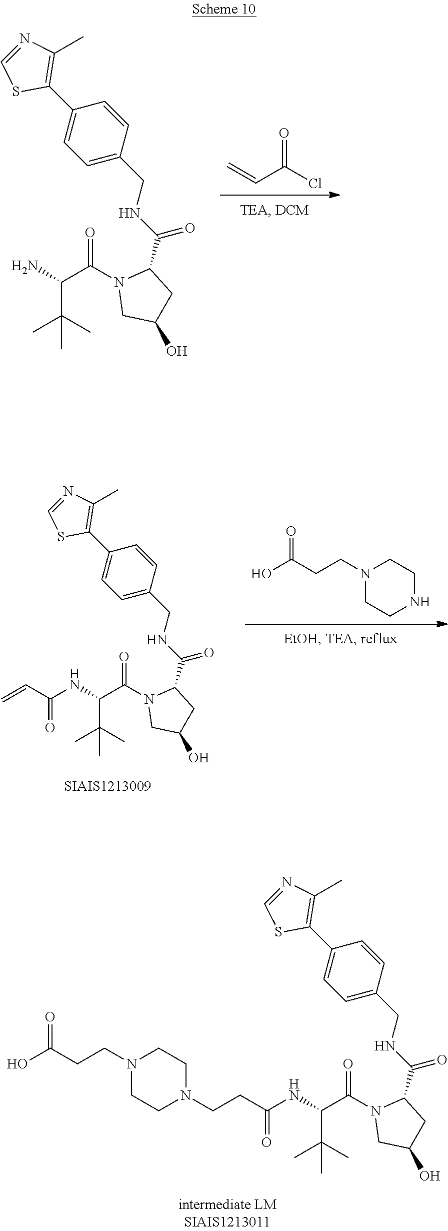

### Synthesis of (2S,4R)-1-((S)-2-acrylamido-3,3-dimethylbutanoyl)-4-hydroxy-N-(4-(4-methylthiazol-5-yl)benzyl)pyrrolidine-2-c arboxamide (SIAIS 1213009)

A single-neck flask was charged with VHL-1 (3.0 mmol, 1.0 equiv), DCM (10 mL) and TEA (9.0 mmol, 3.0 equiv) in turn, followed by addition dropwise of acryloyl chloride (3.6 mmol, 1.2 equiv) at 0° C., and then evacuation and refilling with argon gas for three times. The mixture was stirred at 0° C. under argon gas for 0.5 h. After the reaction was complete as detected by LC-MS, the mixture was rotary evaporated to dryness, and the resulting residue was dissolved in a small amount of acetonitrile and purified by reverse-phase C18 column chromatography (eluent is water (containing 0.05% HCl) and acetonitrile), to yield the desired product as a white solid (1.03 g, 70% yield).

### Synthesis of 3-(4-(3-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-3-oxopropyl)piperazin-1-yl)propanoic acid (SIAIS1213011):

A single-neck flask was charged with 3-(piperazin-1-yl)propanoic acid (1.2 mmol, 1.2 equiv), EtOH (10 mL), SIAIS1213009 (1.0 mmol, 1.0 equiv) and TEA (4.0 mmol, 4.0 equiv) in turn. The mixture was heated to 80° C. for 3 h. After cooling down to room temperature, the mixture was rotary evaporated to dryness, and the resulting residue was dissolved in a small amount of acetonitrile and purified by reverse-phase C18 column chromatography (eluent is water (containing 0.05% HCl) and acetonitrile), to yield the desired product as a pale yellow solid (541 mg, 84% yield). ¹H NMR (500 MHz, MeOD) δ 9.29 (s, 1H), 7.53 (d, J=9.6 Hz, 2H), 7.48 (d, J=8.1 Hz, 2H), 4.64-4.57 (m, 2H), 4.57-4.4.52 (m, 2H), 4.41 (d, J=15.6 Hz, 1H), 3.98 (d, J=11.0 Hz, 1H), 3.81 (dd, J=11.0, 3.7 Hz, 1H), 3.69-3.34 (m, 12H), 2.93-2.75 (m, 4H), 2.57-2.53 (m, 3H), 2.27 (dd, J=13.2, 7.5 Hz, 1H), 2.14-2.07 (m, 1H), 1.08 (s, 9H).HRMS (ESI) m/z: C₃₂H₄₇N₆O₆S⁺ [M+H]⁺, calcd for 643.3272; found,643.3277.

### Intermediate Example 41

### Synthesis of 3-(4-(3-(((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)amino)-3-oxopropyl)phenyl)propanoic acid (SIAIS1213061)

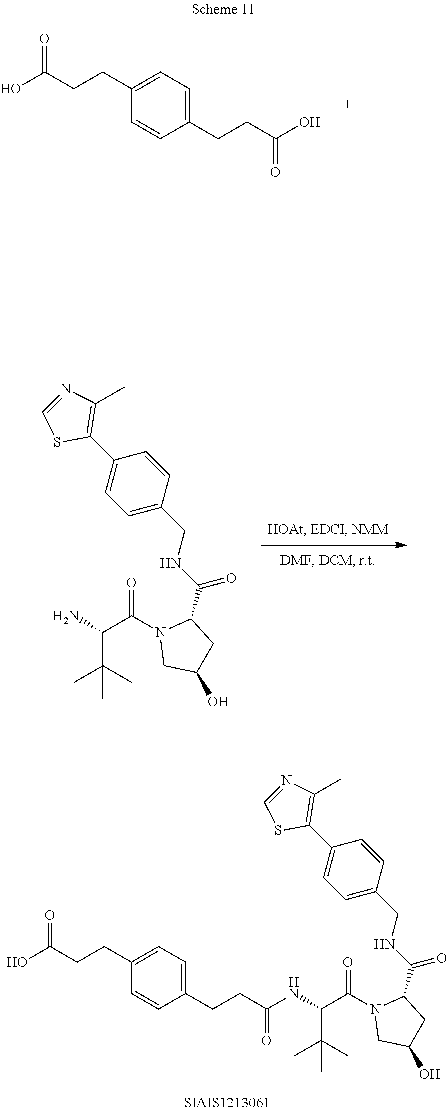 [SMILES: O=C(O)CCc1ccc(CCC(=O)O)cc1 ; Cc1ncsc1-c1ccc(CNC(=O)[C@@H]2C[C@@H](O)CN2C(=O)[C@@H](N)C(C)(C)C)cc1 ; Cc1ncsc1-c1ccc(CNC(=O)[C@@H]2C[C@@H](O)CN2C(=O)[C@@H](NC(=O)CCc2ccc(CCC(=O)O)cc2)C(C)(C)C)cc1]

A single-neck flask was charged with 3,3′-(1,4-phenylene)dipropionic acid (2.0 mmol, 2.0 equiv), DMF (3 mL), DCM (17 mL), HOAt (0.1 mmol, 0.1 equiv), EDCI (2.0 mmol, 2.0 equiv) and NMM (5.0 mmol, 5.0 equiv) in turn, followed by addition in batches of VHL-1 (1.0 mmol, 1.0 equiv) with ice bath-cooling. The resulting reaction mixture was stirred at room temperature overnight, and rotary evaporated to remove DCM. The resulting residue was purified by reverse-phase C₁₈ column chromatography (eluent is water (containing 0.05% HCl) and acetonitrile), to yield the desired product as a white solid (263 mg, 41% yield). ¹H NMR (500 MHz, MeOD) δ 9.66 (s, 1H), 7.54 (d, J=8.3 Hz, 2H), 7.50-7.47 (m, 2H), 7.14-7.08 (m, 4H), 4.61-4.52 (m, 3H), 4.52-4.47 (m, 1H), 4.37 (d, J=15.7 Hz, 1H), 3.89 (d, J=11.1 Hz, 1H), 3.78 (dd, J=11.0, 3.9 Hz, 1H), 2.89-2.83 (m, 4H), 2.63-2.51 (m, 7H), 2.27-2.17 (m, 1H), 2.11-2.02 (m, 1H), 0.93 (s, 9H). HRMS (ESI) m/z: calcd for C₃₄H₄₃N₄O₆S⁺ [M+H]⁺, 635.2898; found,635.2861.

### Intermediate Example 42

### Synthesis of 3-(4-(2-((2-(2,6-dioxopiperidin-3-yl)-1,3-dioxoisoindolin-4-yl)amino)ethyl)piperazin-1-yl) propionic acid (SIAIS 208130)

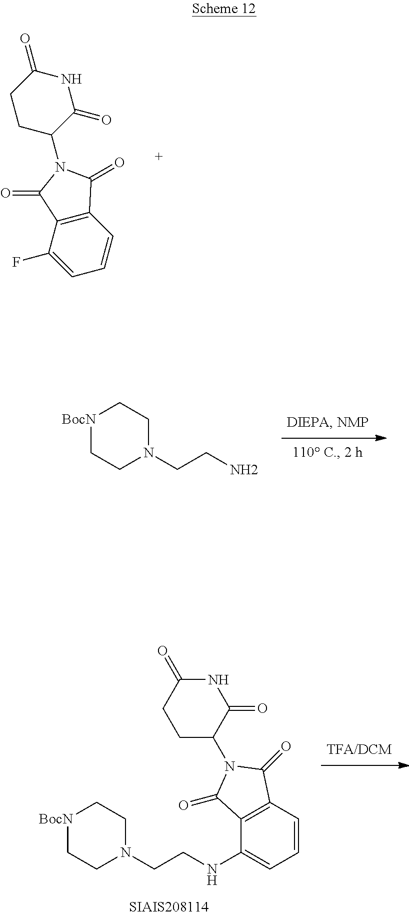 [SMILES: O=C1CCC(N2C(=O)c3cccc(F)c3C2=O)C(=O)N1 ; CC(C)(C)OC(=O)N1CCN(CCN)CC1 ; CC(C)(C)OC(=O)N1CCN(CCNc2cccc3c2C(=O)N(C2CCC(=O)NC2=O)C3=O)CC1]

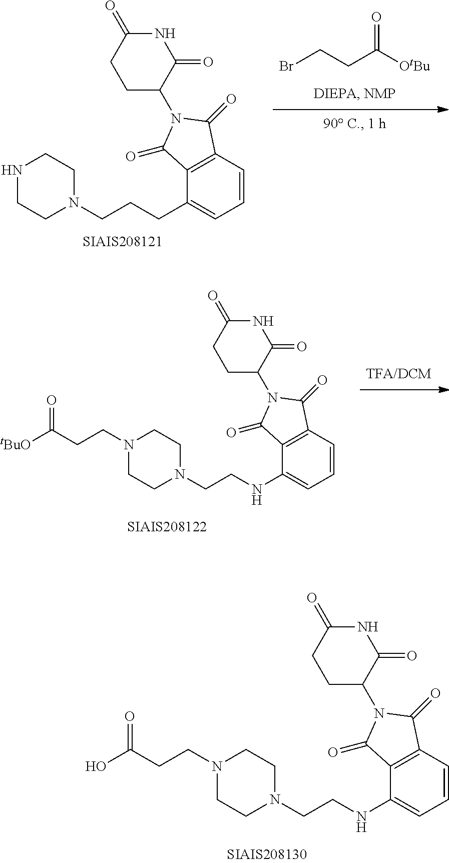 [SMILES: O=C1CCC(N2C(=O)c3cccc(CCCN4CCNCC4)c3C2=O)C(=O)N1 ; CC(C)(C)OC(=O)CCBr ; CC(C)(C)OC(=O)CCN1CCN(CCNc2cccc3c2C(=O)N(C2CCC(=O)NC2=O)C3=O)CC1 ; O=C(O)CCN1CCN(CCNc2cccc3c2C(=O)N(C2CCC(=O)NC2=O)C3=O)CC1]

### Synthesis of tert-butyl 4-(2-((2-(2,6-dioxopiperidin-3-yl)-1,3-dioxoisoindolin-4-yl)amino)ethyl)piperazine-1-carboxylate (SIAIS 208114)

A single-neck flask was charged with 2-(2,6-dioxopiperidin-3-yl)-4-fluoroisoindolin-1,3-dione (1.8 mmol, 1 equiv), NMP (5 mL), tert-butyl 4-(2-aminoethoxy)piperazine-1-carboxylate (3.6 mmol, 2 equiv) and DIPEA (9.0 mmol, 5 equiv) in turn. The mixture was stirred at 110° C. for 2 h. After cooling to room temperature, the reaction solution was purified by reverse-phase C18 column chromatography (eluent is water (containing 0.05% HCl) and acetonitrile), to yield the desired product as a yellow solid (400 mg, 46% yield). HRMS (ESI) m/z: calcd for C₂₄H₃₂N₅O₆⁺ [M+H]⁺, 486.2347; found, 486.2341.

### Synthesis of 2-(2,6-dioxopiperidin-3-yl)-4-((2-(piperazin-1-yl)ethyl)amino)isoindoline-1,3-dione (SIAIS208121)

A single-neck flask was charged with SIAIS208114 (400 mg), DCM (6 mL), and TFA (2 mL) in turn. The mixture was stirred at r.t. for 1 h, and then rotary evaporated to dryness. The resulting residue was treated by addition of water and freeze-drying to afford the yellow solid product which was used directly in the next step. HRMS (ESI) m/z: calcd for C₁₉H₂₄N₅O₄⁺ [M+H]⁺, 386.1823; found, 386.1818.

### Synthesis of tert-butyl 3-(4-(2-((2-(2,6-dioxopiperidin-3-yl)-1,3-dioxoisoindolin-4-yl)amino)ethyl)piperazin-1-yl)prop ano ate (SIAIS 208122)

A single-neck flask was charged with SIAIS208121, NMP (8 mL), tert-butyl 3-bromopropanoate (1.5 eq) and DIEA (3 eq) in turn. The reaction solution was stirred at 90° C. for 1 h. After cooling to r.t., the reaction solution was purified by reverse-phase C₁₈ column chromatography (eluent is water (containing 0.05% HCl) and acetonitrile), to yield the desired product as a yellow solid (180 mg, 43% overall yield over two steps). HRMS (ESI) m/z: calcd for C₂₆H₃₆N₅O₆⁺ [M+H]⁺, 514.2660; found, 514.2667.

### Synthesis of 3-(4-(2-((2-(2,6-dioxopiperidin-3-yl)-1,3-dioxoi soindolin-4-yl)amino)ethyl)piperazin-1-yl)propionic acid (SIAIS 208130)

A single-neck flask was charged with SIAIS208122, DCM (3 mL), and TFA (1 mL) in turn. The mixture was stirred at r.t. for 1 h, and then rotary evaporated to dryness. The resulting residue was purified by reverse-phase C18 column chromatography (eluent is water (containing 0.05% HCl) and acetonitrile), to yield the desired product as a yellow solid (110 mg, 82% yield). ¹H NMR (500 MHz, DMSO) δ 11.10 (s, 1H), 7.62 (dd, J=8.5, 7.2 Hz, 1H), 7.23-7.15 (m, 1H), 7.09 (d, J=7.0 Hz, 1H), 6.78 (s, 1H), 5.06 (dd, J=12.8, 5.4 Hz, 1H), 3.90-3.25 (m, 9H), 2.92-2.84 (m, 1H), 2.76 (s, 2H), 2.63-2.53 (m, 2H), 2.09-1.97 (m, 1H). HRMS (ESI) m/z: calcd for C₂₂H₂₈N₅O₆⁺ [M+H]⁺, 458.2034; found, 458.2039.

### Examples of Compounds of the Present Disclosure

### Example 1

### Synthesis of (Z)—N-(2-(4-(4-chloro-1,2-diphenylbut-1-en-1-yl)phenoxy)ethyl)-3-(2-((2-(2,6-dioxopiperidin-3-yl)-1,3-dioxoisoindolin-4-yl)amino)ethoxy)-N-methylpropanamide (SIAIS180001)

According to scheme 7, Toremifene derivative A ((Z)-2-(4-(4-chloro-1,2-diphenylbut-1-en-1-yl)phenoxy)-N-methylethan-1-amine; 0.035 mmol, 1 equiv), intermediate LM (SIAIS151001) (0.035 mmol, 1 equiv), HOAt (0.07 mmol, 2 equiv), EDCI (0.07 mmol, 2 equiv), anhydrous DMF (2 mL), and NMM (0.175 mmol, 5 equiv) were added in turn to a reaction flask at r.t.. The resulting reaction mixture was stirred at room temperature overnight. After the reaction was complete as detected by LC-MS, the resulting solution was purified by preparative HPLC (eluent (v/v): acetonitrile/(water+0.05% HCl)=10%-100%). The acetonitrile was removed by rotary evaporation, and the resulting residue was lyophilized to yield the target product SIAIS180001 as yellow solid (14.1 mg, 40% yield). ¹H NMR (500 MHz, MeOD) δ 7.52-7.48 (m, 1H), 7.38-7.35 (m, 2H), 7.32-7.24 (m, 3H), 7.21-7.09 (m, 5H), 7.03 (d, J=7.1 Hz, 1H), 7.00 (dd, J=8.6, 3.0 Hz, 1H), 6.76-6.74 (m, 1H), 6.71-6.70 (m, 1H), 6.55-6.51 (m, 2H), 5.00 (dt, J=12.8, 5.1 Hz, 1H), 4.00 (q, J=5.4 Hz, 2H), 3.78-3.72 (m, 3H), 3.64 (dt, J=10.5, 5.4 Hz, 3H), 3.42-3.34 (m, 4H), 3.08 (s, 1.5H, N—CH₃), 2.94 (s, 1.5H, N—CH₃), 2.88 (td, J=7.4, 2.2 Hz, 2H), 2.83-2.73 (m, 1H), 2.72-2.55 (m, 4H), 2.03-1.91 (m, 1H). HRMS (ESI) m/z: calcd for C₄₃H₄₄ClN₄O₇⁺ [M+H]⁺, 763.2893; found, 763.2889.

### Example 2

### Synthesis of (Z)—N-(2-(4-(4-chloro-1,2-diphenylbut-1-en-1-yl)phenoxy)ethyl)-3-(2-(2-((2-(2,6-dioxopiperidin-3-yl)-1,3-dioxoisoindolin-4-yl)amino)ethoxy)ethoxy)-N-methylpropanamide (S IAIS 180002)

Referring to the procedures of Example 1, under appropriate conditions that will be recognized by one skilled in the art, the target compound (SIAIS180002) was prepared by using Toremifene derivative A ((Z)-2-(4-(4-chloro-1,2-diphenylbut-1-en-1-yl)phenoxy)-N-methylethan-1-amine) and intermediate LM (SIAIS151004) as starting materials. The target compound SIAIS180002 was obtained as yellow solid (13.1 mg, 35% yield). ¹H NMR (500 MHz, DMSO) δ 11.09 (s, 1H), 7.60-7.52 (m, 1H), 7.39 (dt, J=7.7, 3.7 Hz, 2H), 7.34-7.25 (m, 3H), 7.23-7.20 (m, 2H), 7.18-7.09 (m, 4H), 7.03 (d, J=7.1 Hz, 1H), 6.75-6.73 (m, 2H), 6.61-6.58 (dt, J=7.7, 3.9 Hz, 3H), 5.04 (dd, J=12.8, 5.4 Hz, 1H), 3.95 (t, J=5.3 Hz, 1H), 3.89 (t, J=5.7 Hz, 1H), 3.65-3.38 (m, 16H), 2.96 (s, 1.5H, N—CH₃), 2.89-2.83 (m, 3H), 2.80 (s,1.5H, N—CH₃), 2.60-2.53 (m, 2H), 2.01-1.98 (m, 1H). HRMS (ESI) m/z: calcd for C₄₅H₄₈ClN₄O₈⁺ [M+H]⁺, 807.3155; found, 807.3153.

### Example 3

### Synthesis of (Z)—N-(2-(4-(4-chloro-1,2-diphenylbut-1-en-1-yl)phenoxy)ethyl)-3-(2-(2-(2-((2-(2,6-dioxopiperidin-3-yl)-1,3-dioxoisoindolin-4-yl)amino)ethoxy)ethoxy)ethoxy)-N-methylpropanamide (SIAIS 180004)

Referring to the procedures of Example 1, under appropriate conditions that will be recognized by one skilled in the art, the target compound (SIAIS180004) was prepared by using Toremifene derivative A ((Z)-2-(4-(4-chloro-1,2-diphenylbut-1-en-1-yl)phenoxy)-N-methylethan-1-amine) and intermediate LM (SIAIS151005) as starting materials. The target compound SIAIS180004 was obtained as yellow solid (11.5 mg, 29% yield). ¹H NMR (500 MHz, DMSO) δ 11.09 (s, 1H), 7.60-7.53 (m, 1H), 7.39 (dd, J=7.4, 6.1 Hz, 2H), 7.33-7.26 (m, 3H), 7.22 (t, J=7.7 Hz, 2H), 7.18-7.11 (m, 4H), 7.03 (d, J=7.0 Hz, 1H), 6.75-6.73 (m, 2H), 6.62-6.59 (m, 3H), 5.05 (dd, J=12.7, 5.4 Hz, 1H), 3.96 (t, J=5.3 Hz, 1H), 3.89 (t, J=5.8 Hz, 1H), 3.66-3.40 (m, 20H), 2.97 (s, 1.5H, N—CH₃), 2.86-2.83 (m, 3H), 2.80 (s, 1.5H, N—CH₃), 2.61-2.55 (m, 2H), 2.05-1.96 (m, 1H). HRMS (ESI) m/z: calcd for C₄₇H₅₂ClN₄O₉⁺ [M+H]⁺, 851.3417; found, 851.3410.

### Example 4

### Synthesis of (Z)—N-(2-(4-(4-chloro-1,2-diphenylbut-1-en-1-yl)phenoxy)ethyl)-1-((2-(2,6-dioxopiperidin-3-yl)-1,3-dioxoisoindolin-4-yl)amino)-N-methyl-3,6,9,12-tetraoxapentadecan-15-amide (SIAIS180006)

Referring to the procedures of Example 1, under appropriate conditions that will be recognized by one skilled in the art, the target compound (SIAIS180006) was prepared by using Toremifene derivative A ((Z)-2-(4-(4-chloro-1,2-diphenylbut-1-en-1-yl)phenoxy)-N-methylethan-1-amine) and intermediate LM (SIAIS151006) as starting materials. The target compound SIAIS180006 was obtained as yellow solid (12.2 mg, 29% yield). ¹H NMR (500 MHz, DMSO) δ 11.09 (s, 1H), 7.57 (t, J=7.7 Hz, 1H), 7.40 (t, J=7.3 Hz, 2H), 7.29 (dd, J=14.1, 7.2 Hz, 3H), 7.24-7.19 (m, 2H), 7.18-7.11 (m, 4H), 7.03 (d, J=7.0 Hz, 1H), 6.74 (dd, J=8.7, 3.3 Hz, 2H), 6.61 (d, J=8.6 Hz, 3H), 5.05 (dd, J=12.7, 5.4 Hz, 1H), 3.96 (t, J=5.3 Hz, 1H), 3.89 (t, J=5.8 Hz, 1H), 3.64-3.39 (m, 24H), 2.97 (s, 1.5H, N—CH₃), 2.87 (dt, J=21.2, 6.5 Hz, 3H), 2.80 (s, 1.5H, N—CH₃), 2.62-2.55 (m, 2H), 2.04-1.97 (m, 1H). HRMS (ESI) m/z: calcd for C₄₉H₅₆ClN₄O₁₀⁺ [M+H]⁺, 895.3679; found, 895.3671.

### Example 5

### Synthesis of (Z)—N-(2-(4-(4-chloro-1,2-diphenylbut-1-en-1-yl)phenoxy)ethyl)-1-((2-(2,6-dioxopiperidin-3-yl)-1,3-dioxoisoindolin-4-yl)amino)-N-methyl-3,6,9,12,15-pentaoxaoctadecan-18-amide (SIAIS180007)

Referring to the procedures of Example 1, under appropriate conditions that will be recognized by one skilled in the art, the target compound (SIAIS180007) was prepared by using Toremifene derivative A ((Z)-2-(4-(4-chloro-1,2-diphenylbut-1-en-1-yl)phenoxy)-N-methylethan-1-amine) and intermediate LM (SIAIS151007) as starting materials. The target compound SIAIS180007 was obtained as yellow solid (12.6 mg, 29% yield). ¹H NMR (500 MHz, DMSO) δ 11.09 (s, 1H), 7.61-7.53 (m, 1H), 7.40 (t, J=7.6 Hz, 2H), 7.30 (dd, J=14.3, 7.2 Hz, 3H), 7.25-7.19 (m, 2H), 7.15 (dd, J=12.8, 8.2 Hz, 4H), 7.03 (d, J=7.0 Hz, 1H), 6.74 (dd, J=8.7, 3.6 Hz, 2H), 6.61-6.60 (m, 3H), 5.05 (dd, J=12.7, 5.4 Hz, 1H), 3.96 (t, J=5.2 Hz, 1H), 3.89 (t, J=5.8 Hz, 1H), 3.65-3.39 (m, 28H), 2.97 (s, 1.5H, N—CH₃), 2.93-2.82 (m, 3H), 2.80 (s, 1.5H, N—CH₃), 2.59-2.56 (m, 2H), 2.06-1.95 (m, 1H). HRMS (ESI) m/z: calcd for C₅₁H₆₀ClN₄O₁₁⁺ [M+H]⁺, 939.3942; found, 939.3952.

### Example 6

### Synthesis of (Z)—N-(2-(4-(4-chloro-1,2-diphenylbut-1-en-1-yl)phenoxy)ethyl)-2-((2-(2,6-dioxopiperidin-3-yl)-1,3-dioxoisoindolin-4-yl)amino)-N-methylac etamide (S IAIS 180008)

Referring to the procedures of Example 1, under appropriate conditions that will be recognized by one skilled in the art, the target compound (SIAIS180008) was prepared by using Toremifene derivative A ((Z)-2-(4-(4-chloro-1,2-diphenylbut-1-en-1-yl)phenoxy)-N-methylethan-1-amine) and intermediate LM (SIAIS151025) as starting materials. The target compound SIAIS180008 was obtained as yellow solid (8.3 mg, 25% yield). ¹H NMR (500 MHz, DMSO) δ 11.10 (s, 1H), 7.60-7.49 (m, 1H), 7.39 (t, J=7.5 Hz, 2H), 7.32-7.28 (m, 3H), 7.22-7.20 (m, 2H), 7.17-7.15 (m, 3H), 7.07-6.96 (m, 3H), 6.76 (dd, J=8.8, 3.3 Hz, 2H), 6.63 (dd, J=8.8, 3.2 Hz, 2H), 5.06 (dd, J=12.7, 5.4 Hz, 1H), 4.24 (d, J=4.7 Hz, 1H), 4.15 (d, J=4.5 Hz, 1H), 4.06 (t, J=5.1 Hz, 1H), 3.97 (t, J=5.6 Hz, 1H), 3.70 (d, J=5.0 Hz, 1H), 3.64 (t, J=5.7 Hz, 1H), 3.52-3.37 (m, 3H), 3.06 (s, 1.5H, N—CH₃), 2.91 (s, 1.5H, N—CH₃), 2.90-2.81 (m, 3H), 2.61-2.57 (m, 1H), 2.06-1.98(m, 1H). HRMS (ESI) m/z: calcd for C₄₀H₃₈ClN₄O₆⁺ [M+H]⁺, 705.2474; found, 705.2482.

### Example 7

### Synthesis of (Z)—N-(2-(4-(4-chloro-1,2-diphenylbut-1-en-1-yl)phenoxy)ethyl)-3-((2-(2,6-dioxopiperidin-3-yl)-1,3-dioxoisoindolin-4-yl)amino)-N-methylprop anamide (SIAIS180009)

Referring to the procedures of Example 1, under appropriate conditions that will be recognized by one skilled in the art, the target compound (SIAIS180009) was prepared by using Toremifene derivative A ((Z)-2-(4-(4-chloro-1,2-diphenylbut-1-en-1-yl)phenoxy)-N-methylethan-1-amine) and intermediate LM (SIAIS151026) as starting materials. The target compound SIAIS180009 was obtained as yellow solid (10.1 mg, 30% yield). ¹H NMR (500 MHz, DMSO) δ 11.09 (s, 1H), 7.60-7.49 (m, 1H), 7.40 (t, J=7.5 Hz, 2H), 7.33-7.25 (m, 3H), 7.24-7.19 (m, 2H), 7.18-7.08 (m, 4H), 7.00 (dd, J=9.0, 7.1 Hz, 1H), 6.78-6.68 (m, 3H), 6.60 (d, J=8.8 Hz, 1H), 6.51 (d, J=8.7 Hz, 1H), 5.03 (dd, J=12.8, 5.2 Hz, 1H), 3.94 (dt, J=11.4, 5.4 Hz, 2H), 3.65-3.39 (m, 7H), 2.95 (s, 1.5H, N—CH₃), 2.89-2.83 (m, 4.5H, N—CH₃), 2.71 (t, J=6.2 Hz, 1H), 2.59-2.56 (m, 2H), 1.99-1.96 (m, 1H). HRMS (ESI) m/z: calcd for C₄₁H₄₀ClN₄O₆⁺ [M+H]⁺, 719.2631; found, 719.2640.

### Example 8

### Synthesis of (Z)—N-(2-(4-(4-chloro-1,2-diphenylbut-1-en-1-yl)phenoxy)ethyl)-4-((2-(2,6-dioxopiperidin-3-yl)-1,3-dioxoisoindolin-4-yl)amino)-N-methylbutanamide (S IAIS 180010)

Referring to the procedures of Example 1, under appropriate conditions that will be recognized by one skilled in the art, the target compound (SIAIS180010) was prepared by using Toremifene derivative A ((Z)-2-(4-(4-chloro-1,2-diphenylbut-1-en-1-yl)phenoxy)-N-methylethan-1-amine) and intermediate LM (SIAIS151019) as starting materials. The target compound SIAIS180010 was obtained as yellow solid (8.3 mg, 24% yield). ¹H NMR (500 MHz, DMSO) δ 11.09 (s, 1H), 7.54 (t, J=7.8 Hz, 1H), 7.40 (t, J=6.8 Hz, 2H), 7.34-7.25 (m, 3H), 7.25-7.19 (m, 2H), 7.18-7.12 (m, 4H), 6.99 (d, J=7.0 Hz, 1H), 6.74 (dd, J=8.8, 2.1 Hz, 2H), 6.66-6.57 (m, 3H), 5.04 (dd, J=12.7, 5.5 Hz, 1H), 3.97 (t, J=5.2 Hz, 1H), 3.92 (t, J=5.7 Hz, 1H), 3.60 (t, J=5.3 Hz, 1H), 3.56 (t, J=5.7 Hz, 1H), 3.50-3.37 (m, 3H), 3.26 (dd, J=18.1, 11.4 Hz, 2H), 2.97 (s, 1.5H, N—CH₃), 2.91-2.81 (m, 4.5H), 2.59-2.55 (m, 1H), 2.43 (t, J=7.0 Hz, 1H), 2.34 (t, J=6.9 Hz, 1H), 2.01-1.97 (m, 1H), 1.81-1.70 (m, 2H). HRMS (ESI) m/z: calcd for C₄₂H₄₂ClN₄O₆⁺ [M+H]⁺, 733.2787; found, 733.2778.

### Example 9

### Synthesis of (Z)—N-(2-(4-(4-chloro-1,2-diphenylbut-1-en-1-yl)phenoxy)ethyl)-5-((2-(2,6-dioxopiperidin-3-yl)-1,3-dioxoisoindolin-4-yl)amino)-N-methylpentanamide (SIAIS180011)

Referring to the procedures of Example 1, under appropriate conditions that will be recognized by one skilled in the art, the target compound (SIAIS180011) was prepared by using Toremifene derivative A ((Z)-2-(4-(4-chloro-1,2-diphenylbut-1-en-1-yl)phenoxy)-N-methylethan-1-amine) and intermediate LM (SIAIS151020) as starting materials. The target compound SIAIS180011 was obtained as yellow solid (11.2 mg, 32% yield). ¹H NMR (500 MHz, DMSO) δ 11.09 (s, 1H), 7.54 (dd, J=15.6, 8.0 Hz, 1H), 7.39 (t, J=6.7 Hz, 2H), 7.33-7.25 (m, 3H), 7.22 (t, J=7.3 Hz, 2H), 7.18-7.11 (m, 3H), 7.07 (t, J=8.3 Hz, 1H), 7.01 (d, J=7.0 Hz, 1H), 6.74 (dd, J=8.8, 2.9 Hz, 2H), 6.60 (dd, J=8.6, 5.6 Hz, 2H), 6.56-6.53 (m, 1H), 5.04 (dd, J=12.7, 5.4 Hz, 1H), 3.97 (t, J=5.2 Hz, 1H), 3.90 (t, J=5.7 Hz, 1H), 3.60 (t, J=5.2 Hz, 1H), 3.54 (t, J=5.7 Hz, 1H), 3.42 (t, J=7.2 Hz, 2H), 3.31-3.22 (m, 2H), 2.97 (s, 1.5H, N—CH₃), 2.90-2.78 (m, 4.5H), 2.65-2.52 (m, 2H), 2.36-2.35 (s, 1H), 2.30 (t, J=6.7 Hz, 1H), 2.02-1.99 (m, 1H), 1.54 (s, 4H). HRMS (ESI) m/z: calcd for C₄₃H₄₄ClN₄O₆⁺ [M+H]⁺, 747.2944; found, 747.2939.

### Example 10

### Synthesis of (Z)—N-(2-(4-(4-chloro-1,2-diphenylbut-1-en-1-yl)phenoxy)ethyl)-6-((2-(2,6-dioxopiperidin-3-yl)-1,3-dioxoisoindolin-4-yl)amino)-N-methylhexanamide (S IAIS 180012)

Referring to the procedures of Example 1, under appropriate conditions that will be recognized by one skilled in the art, the target compound (SIAIS180012) was prepared by using Toremifene derivative A ((Z)-2-(4-(4-chloro-1,2-diphenylbut-1-en-1-yl)phenoxy)-N-methylethan-1-amine) and intermediate LM (SIAIS151027) as starting materials. The target compound SIAIS180012 was obtained as yellow solid (10.9 mg, 31% yield). ¹H NMR (500 MHz, DMSO) δ 11.09 (s, 1H), 7.56 (dd, J=15.6, 7.9 Hz, 1H), 7.39 (t, J=7.5 Hz, 2H), 7.33-7.25 (m, 3H), 7.23-7.20 (m, 2H), 7.17-7.13 (m, 3H), 7.07 (d, J=8.6 Hz, 1H), 7.01 (d, J=7.0 Hz, 1H), 6.74 (dd, J=8.7, 1.6 Hz, 2H), 6.60 (dd, J=8.8, 2.9 Hz, 2H), 6.52 (t, J=5.7 Hz, 1H), 5.04 (dd, J=12.8, 5.4 Hz, 1H), 3.97 (t, J=5.2 Hz, 1H), 3.90 (t, J=5.8 Hz, 1H), 3.60 (t, J=5.2 Hz, 1H), 3.53 (t, J=5.7 Hz, 1H), 3.42 (t, J=7.1 Hz, 2H), 3.26 (dd, J=13.1, 6.5 Hz, 2H), 2.96 (s, 1.6H, N—CH₃), 2.92-2.82 (m, 3H), 2.80 (s, 1.4H, N—CH₃), 2.65-2.52 (m, 2H), 2.32 (t, J=7.4 Hz, 1H), 2.25 (t, J=7.3 Hz, 1H), 2.04-1.95 (m, 1H), 1.62-1.43 (m, 4H), 1.35-1.29 (m, 2H). HRMS (ESI) m/z: calcd for C₄₄H₄₆ClN₄O₆⁺ [M+H]⁺, 761.3100; found, 761.3093.

### Example 11

### Synthesis of (Z)—N-(2-(4-(4-chloro-1,2-diphenylbut-1-en-1-yl)phenoxy)ethyl)-7-((2-(2,6-dioxopiperidin-3-yl)-1,3-dioxoisoindolin-4-yl)amino)-N-methylheptanamide (SIAIS180013)

Referring to the procedures of Example 1, under appropriate conditions that will be recognized by one skilled in the art, the target compound (SIAIS180013) was prepared by using Toremifene derivative A ((Z)-2-(4-(4-chloro-1,2-diphenylbut-1-en-1-yl)phenoxy)-N-methylethan-1-amine) and intermediate LM (SIAIS151086) as starting materials. The target compound SIAIS180013 was obtained as yellow solid (21.9 mg, 61% yield). ¹H NMR (500 MHz, DMSO) δ 11.09 (s, 1H), 7.56 (t, J=7.8 Hz, 1H), 7.39 (t, J=7.4 Hz, 2H), 7.34-7.25 (m, 3H), 7.21 (t, J=6.7 Hz, 2H), 7.18-7.11 (m, 3H), 7.08 (d, J=8.7 Hz, 1H), 7.01 (d, J=7.0 Hz, 1H), 6.74 (dd, J=8.4, 4.9 Hz, 2H), 6.60 (d, J=8.6 Hz, 2H), 6.52 (t, J=5.4 Hz, 1H), 5.05 (dd, J=12.7, 5.4 Hz, 1H), 3.97 (t, J=5.2 Hz, 1H), 3.90 (t, J=5.7 Hz, 1H), 3.59 (t, J=5.0 Hz, 1H), 3.53 (t, J=5.7 Hz, 1H), 3.42 (t, J=7.2 Hz, 2H), 3.27-3.26 (m, 2H), 2.96 (s, 1.6H, N—CH₃), 2.92-2.82 (m, 3H), 2.80 (s, 1.4H, N—CH₃), 2.66-2.52 (m, 2H), 2.29 (t, J=7.3 Hz, 1H), 2.23 (t, J=7.4 Hz, 1H), 2.05-1.96 (m, 1H), 1.56-1.51 (m, 2H), 1.50-1.41 (m, 2H), 1.35-1.24 (m, 4H). HRMS (ESI) m/z: calcd for C₄₅H₄₈ClN₄O₆⁺ [M+H]⁺, 775.3257; found, 775.3249.

### Example 12

### Synthesis of (2S,4R)-1-((S)-2-(tert-butyl)-14-(4-((Z)-4-chloro-1,2-diphenylbut-1-en-1-yl)phenoxy)-12-methyl-4,11-dioxo-6,9-dioxa-3,12-diazatetradec anoyl)-4-hydroxy-N-(4-(4-methylthiazol-5-yl)benzyl)pyrrolidine-2-carboxamide (SIAIS180039)

Referring to the procedures of Example 1, under appropriate conditions that will be recognized by one skilled in the art, the target compound (SIAIS180039) was prepared by using Toremifene derivative A ((Z)-2-(4-(4-chloro-1,2-diphenylbut-1-en-1-yl)phenoxy)-N-methylethan-1-amine) and intermediate LM (SIAIS151010) as starting materials. The target compound SIAIS180039 was obtained as white solid (11.8 mg, 53% yield). ¹H NMR (500 MHz, DMSO) δ 8.97 (d, J=2.5 Hz, 1H), 8.63-8.49 (m, 1H), 7.46-7.35 (m, 7H), 7.33-7.25 (m, 3H), 7.23-7.19 (m, 2H), 7.17-7.13 (m, 3H), 6.74 (dd, J=8.7, 4.0 Hz, 2H), 6.59 (dd, J=8.7, 3.4 Hz, 2H), 4.56 (d, J=9.6 Hz, 1H), 4.44 (t, J=8.2 Hz, 1H), 4.40-4.35 (m, 2H), 4.28-4.09 (m, 4H), 4.00-3.85 (m, 5H), 3.75-3.62 (m, 7H), 3.42 (t, J=7.2 Hz, 2H), 2.92 (s, 1.5H, N—CH₃), 2.83 (t, J=10.0 Hz, 2H), 2.79 (s, 1.5H, N—CH₃), 2.43 (s, 3H), 2.06-2.03 (m, 1H), 1.92-1.87 (m, 1H), 0.92-0.91 (m, 9H). HRMS (ESI) m/z: calcd for C₅₃H₆₃ClN₅O₈S⁺ [M+H]⁺, 964.4080; found, 964.4074.

### Example 13

### Synthesis of (2S,4R)-1-((S)-2-(tert-butyl)-16-(4-((Z)-4-chloro-1,2-diphenylbut-1-en-1-yl)phenoxy)-14-methyl-4,13-dioxo-7,10-dioxa-3,14-diazahexadec anoyl)-4-hydroxy-N-(4-(4-methylthiazol-5-yl)benzyl)pyrrolidine-2-c arboxamide (SIAIS180023)

Referring to the procedures of Example 1, under appropriate conditions that will be recognized by one skilled in the art, the target compound (SIAIS180023) was prepared by using Toremifene derivative A ((Z)-2-(4-(4-chloro-1,2-diphenylbut-1-en-1-yl)phenoxy)-N-methylethan-1-amine) and intermediate LM (SIAIS151002) as starting materials. The target compound SIAIS180023 was obtained as white solid (16.4 mg, 35% yield). ¹H NMR (500 MHz, DMSO) δ 8.98 (s, 1H), 8.56 (t, J=5.9 Hz, 1H), 7.90 (d, J=9.4 Hz, 1H), 7.42-7.37 (m, 6H), 7.29 (dd, J=12.1, 7.2 Hz, 3H), 7.24-7.19 (m, 2H), 7.17-7.13 (m, 3H), 6.77-6.70 (m, 2H), 6.61 (d, J=8.0 Hz, 2H), 4.55 (d, J=9.4 Hz, 1H), 4.43 (dd, J=14.5, 6.8 Hz, 2H), 4.35 (s, 1H), 4.21 (dd, J=15.8, 5.4 Hz, 1H), 3.97 (t, J=5.2 Hz, 1H), 3.89 (t, J=5.8 Hz, 1H), 3.71-3.39 (m, 17H), 2.97 (s, 1.5H, N—CH₃), 2.84 (t, J=6.5 Hz, 2H), 2.80 (s, 1.5H, N—CH₃), 2.58 (t, J=6.8 Hz, 1H), 2.44 (s, 3H), 2.39-2.28 (m, 1H), 2.07-1.98 (m, 1H), 1.93-1.84 (m, 1H), 0.91 (s, 9H). HRMS (ESI) m/z: calcd for C₅₅H₆₇ClN₅O₈S⁺ [M+H]⁺, 992.4393; found, 992.4388.

### Example 14

### Synthesis of (2S,4R)-1-((S)-2-(tert-butyl)-19-(4-((Z)-4-chloro-1,2-diphenylbut-1-en-1-yl)phenoxy)-17-methyl-4,16-dioxo-7,10,13-trioxa-3,17-di azanonadec anoyl)-4-hydroxy-N-(4-(4-methylthi azol-5-yl)benzyl)pyrrolidine-2-c arboxamide (SIAIS180024)

Referring to the procedures of Example 1, under appropriate conditions that will be recognized by one skilled in the art, the target compound (SIAIS180024) was prepared by using Toremifene derivative A ((Z)-2-(4-(4-chloro-1,2-diphenylbut-1-en-1-yl)phenoxy)-N-methylethan-1-amine) and intermediate LM (SIAIS151003) as starting materials. The target compound SIAIS180024 was obtained as white solid (17.3 mg, 36% yield). ¹H NMR (500 MHz, DMSO) δ 8.98 (s, 1H), 8.56 (t, J=6.1 Hz, 1H), 7.91 (d, J=9.4 Hz, 1H), 7.43-7.37 (m, 6H), 7.34-7.26 (m, 3H), 7.25-7.19 (m, 2H), 7.18-7.12 (m, 3H), 6.77-6.71 (m, 2H), 6.61 (d, J=7.7 Hz, 2H), 4.55 (d, J=9.4 Hz, 1H), 4.43 (dd, J=14.6, 7.0 Hz, 2H), 4.35 (s, 1H), 4.21 (dd, J=15.8, 5.5 Hz, 1H), 3.97 (t, J=5.2 Hz, 1H), 3.90 (t, J=5.8 Hz, 1H), 3.68-3.52 (m, 8H), 3.49-3.39 (m, 13H), 2.98 (s, 1.5H, N—CH₃), 2.87-2.82 (m, 2H), 2.80 (s, 1.5H, N—CH₃), 2.58 (t, J=6.7 Hz, 1H), 2.44 (s, 3H), 2.39-2.29 (m, 1H), 2.07-1.98 (m, 1H), 1.92-1.87 (m, 1H), 0.92 (s, 9H). HRMS (ESI) m/z: calcd for C₅₇H₇₁ClN₅O₉S⁺ [M+H]⁺, 1036.4656; found, 1036.4647.

### Example 15

### Synthesis of N1-(2-(4-((Z)-4-chloro-1,2-diphenylbut-1-en-1-yl)phenoxy)ethyl)-N16-((S)-1-((2S,4R)-4-hydroxy-2-((4-(4-methylthiazol-5-yl)benzyl)c arb amoyl)pyrrolidin-1-yl)-3,3-dimethyl-1-oxobutan-2-yl)-N1-methyl-4,7,10,13-tetraoxahexadec anedi amide (S IAIS 180025)

Referring to the procedures of Example 1, under appropriate conditions that will be recognized by one skilled in the art, the target compound (SIAIS180025) was prepared by using Toremifene derivative A ((Z)-2-(4-(4-chloro-1,2-diphenylbut-1-en-1-yl)phenoxy)-N-methylethan-1-amine) and intermediate LM (SIAIS151008) as starting materials. The target compound SIAIS180025 was obtained as white solid (13.5 mg, 54% yield). ¹H NMR (500 MHz, DMSO) δ 9.01 (s, 1H), 8.57 (t, J=6.0 Hz, 1H), 7.91 (d, J=9.4 Hz, 1H), 7.46-7.35 (m, 6H), 7.34-7.26 (m, 3H), 7.24-7.19 (m, 2H), 7.18-7.10 (m, 3H), 6.80-6.70 (m, 2H), 6.61 (dd, J=8.7, 1.4 Hz, 2H), 4.55 (d, J=9.4 Hz, 1H), 4.46-4.39 (m, 2H), 4.35 (s, 1H), 4.22 (dd, J=15.9, 5.5 Hz, 1H), 4.13-3.75 (m, 12H), 3.69-3.52 (m, 8H), 3.45-3.40 (m, 7H), 2.98 (s, 1.5H, N—CH₃), 2.85 (td, J=7.1, 2.6 Hz, 2H), 2.81 (s, 1.5H, N—CH₃), 2.61-2.55 (m, 1H), 2.44 (s, 3H), 2.38-2.30 (m, 1H), 2.07-1.97 (m, 1H), 1.93-1.87 (m, 1H), 0.93 (s, 9H). HRMS (ESI) m/z: calcd for C₅₉H₇₅ClN₅O₁₀S⁺ [M+H]⁺, 1080.4918; found, 1080.4915.

### Example 16

### Synthesis of N1-(2-(4-((Z)-4-chloro-1,2-diphenylbut-1-en-1-yl)phenoxy)ethyl)-N19-((S)-1-((2S,4R)-4-hydroxy-2-((4-(4-methylthiazol-5-yl)benzyl)c arb amoyl)pyrrolidin-1-yl)-3,3-dimethyl-1-oxobutan-2-yl)-N1-methyl-4,7 ,10,13,16-pentaoxanonadec anediamide (SIAIS180022)

Referring to the procedures of Example 1, under appropriate conditions that will be recognized by one skilled in the art, the target compound (SIAIS180022) was prepared by using Toremifene derivative A ((Z)-2-(4-(4-chloro-1,2-diphenylbut-1-en-1-yl)phenoxy)-N-methylethan-1-amine) and intermediate LM (SIAIS151009) as starting materials. The target compound SIAIS180022 was obtained as white solid (18.0 mg, 34% yield). ¹H NMR (500 MHz, DMSO) δ 8.98 (s, 1H), 8.56 (t, J=6.1 Hz, 1H), 7.91 (d, J=9.4 Hz, 1H), 7.43-7.37 (m, 6H), 7.34-7.26 (m, 3H), 7.24-7.19 (m, 2H), 7.19-7.12 (m, 3H), 6.78-6.70 (m, 2H), 6.61 (dd, J=8.7, 1.3 Hz, 2H), 4.55 (d, J=9.4 Hz, 1H), 4.43 (dd, J=14.7, 6.9 Hz, 2H), 4.35 (s, 1H), 4.21 (dd, J=15.9, 5.5 Hz, 1H), 3.97 (t, J=5.2 Hz, 1H), 3.90 (t, J=5.8 Hz, 1H), 3.70-3.51 (m, 9H), 3.50-3.43 (m, 20H), 2.98 (s, 1.5H, N—CH₃), 2.85 (td, J=7.2, 2.7 Hz, 2H), 2.81 (s, 1.5H, N—CH₃), 2.60-2.53 (m, 1H), 2.44 (s, 3H), 2.34 (dt, J=19.8, 5.7 Hz, 1H), 2.07-1.98 (m, 1H), 1.93-1.87 (m, 1H), 0.93 (s, 9H). HRMS (ESI) m/z: calcd for C₆₁H₇₉ClN₅O₁₁S⁺ [M+H]⁺, 1124.5180; found, 1124.5186.

### Example 17

### Synthesis of N1-(2-(4-((Z)-4-chloro-1,2-diphenylbut-1-en-1-yl)phenoxy)ethyl)-N4-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)-N1-methylsuccinamide (S IAIS 180026)

Referring to the procedures of Example 1, under appropriate conditions that will be recognized by one skilled in the art, the target compound (SIAIS180026) was prepared by using Toremifene derivative A ((Z)-2-(4-(4-chloro-1,2-diphenylbut-1-en-1-yl)phenoxy)-N-methylethan-1-amine) and intermediate LM (SIAIS074011) as starting materials. The target compound SIAIS180026 was obtained as white solid (9.1 mg, 43% yield). ¹H NMR (500 MHz, DMSO) δ 8.99 (s, 1H), 8.56-8.54 (m, 1H), 7.88 (d, J=9.2 Hz, 1H), 7.43-7.38 (m, 6H), 7.32-7.28 (m, 3H), 7.24-7.20 (m, 2H), 7.17-7.13 (m, 3H), 6.76-6.73 (m, 2H), 6.65-6.59 (m, 2H), 4.50 (d, J=9.1 Hz, 1H), 4.44-4.40 (m, 2H), 4.33 (s, 1H), 4.22 (dd, J=16.1, 5.5 Hz, 1H), 3.99-3.96 (m, 1H), 3.89 (t, J=5.8 Hz, 1H), 3.66-3.59 (m, 3H), 3.55-3.51 (m, 3H), 2.98 (s, 1.6H, N—CH₃), 2.86-2.84 (m, 2H), 2.80 (s, 1.4H, N—CH₃), 2.46-2.42 (m, 7H), 2.35-2.30 (m, 1H), 2.04-2.00 (m, 1H), 1.92-1.86 (m, 1H), 0.91-0.89 (m, 9H). HRMS (ESI) m/z: calcd for C₅₁H₅₉ClN₅O₆S⁺ [M+H]⁺, 904.3869; found, 904.3873.

### Example 18

### Synthesis of N1-(2-(4-((Z)-4-chloro-1,2-diphenylbut-1-en-1-yl)phenoxy)ethyl)-N5-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)-N1-methylglutaramide (SIAIS180027)

Referring to the procedures of Example 1, under appropriate conditions that will be recognized by one skilled in the art, the target compound (SIAIS180027) was prepared by using Toremifene derivative A ((Z)-2-(4-(4-chloro-1,2-diphenylbut-1-en-1-yl)phenoxy)-N-methylethan-1-amine) and intermediate LM (SIAIS074012) as starting materials. The target compound SIAIS180027 was obtained as white solid (10.8 mg, 50% yield). ¹H NMR (500 MHz, DMSO) δ 8.98 (d, J=1.2 Hz, 1H), 8.55 (t, J=6.0 Hz, 1H), 7.85 (dd, J=9.4, 2.6 Hz, 1H), 7.43-7.37 (m, 6H), 7.33-7.25 (m, 3H), 7.23-7.20 (m, 2H), 7.19-7.11 (m, 3H), 6.79-6.72 (m, 2H), 6.62 (dd, J=8.8, 3.2 Hz, 2H), 4.52 (t, J=9.7 Hz, 1H), 4.45-4.40 (m, 2H), 4.34 (s, 1H), 4.21 (dd, J=15.8, 5.4 Hz, 1H), 3.95 (t, J=5.4 Hz, 1H), 3.90 (t, J=5.9 Hz, 1H), 3.65-3.63 (m, 2H), 3.58 (t, J=5.0 Hz, 1H), 3.55-3.50 (m, 1H), 3.43 (t, J=7.1 Hz, 2H), 2.95 (s, 1.5H, N—CH₃), 2.86-2.83 (m, 2H), 2.80 (s, 1.5H, N—CH₃), 2.43 (s, 3H), 2.38-2.09 (m, 5H), 2.06-1.98 (m, 1H), 1.92-1.87 (m, 1H), 1.70-1.67 (m, 2H), 0.91 (d, J=11.7 Hz, 9H). HRMS (ESI) m/z: calcd for C₅₂H₆₁ClN₅O₆S⁺ [M+H]⁺, 918.4026; found, 918.4022.

### Example 19

### Synthesis of N1-(2-(4-((Z)-4-chloro-1,2-diphenylbut-1-en-1-yl)phenoxy)ethyl)-N6-((S)-1-((2S,4R)-4-hydroxy-2-((4-(4-methylthiazol-5-yl)benzyl)c arb amoyl)pyrrolidin-1-yl)-3,3-dimethyl-1-oxobutan-2-yl)-N1-methyladip amide (SIAIS 180028)

Referring to the procedures of Example 1, under appropriate conditions that will be recognized by one skilled in the art, the target compound (SIAIS180028) was prepared by using Toremifene derivative A ((Z)-2-(4-(4-chloro-1,2-diphenylbut-1-en-1-yl)phenoxy)-N-methylethan-1-amine) and intermediate LM (SIAIS074013) as starting materials. The target compound SIAIS180028 was obtained as white solid (12.4 mg, 57% yield). ¹H NMR (500 MHz, DMSO) δ 9.00 (s, 1H), 8.56 (t, J=6.0 Hz, 1H), 7.84 (dd, J=9.3, 3.5 Hz, 1H), 7.40 (dd, J=18.3, 8.0 Hz, 6H), 7.33-7.26 (m, 3H), 7.25-7.19 (m, 2H), 7.17-7.13 (m, 3H), 6.75 (dd, J=8.7, 7.3 Hz, 2H), 6.62-6.60 (m, 2H), 4.53 (d, J=9.4 Hz, 1H), 4.47-4.39 (m, 2H), 4.34 (s, 1H), 4.21 (dd, J=15.8, 5.3 Hz, 1H), 3.96 (t, J=5.3 Hz, 1H), 3.89 (t, J=5.8 Hz, 2H), 3.63-3.56 (m, 3H), 3.53 (t, J=5.4 Hz, 1H), 3.42 (t, J=6.9 Hz, 2H), 2.97 (s, 1.5H, N—CH₃), 2.84 (td, J=7.1, 3.1 Hz, 2H), 2.79 (s, 1.5H, N—CH₃), 2.44 (s, 3H), 2.31-2.23 (m, 3H), 2.16-1.98 (m, 2H), 1.91-1.90 (m, 1H), 1.46 (dd, J=13.5, 6.5 Hz, 4H), 0.91 (d, J=8.2 Hz, 9H). HRMS (ESI) m/z: calcd for C₅₃H₆₃ClN₅O₆S⁺ [M+H]⁺, 932.4182; found, 932.4178.

### Example 20

### Synthesis of N1-(2-(4-((Z)-4-chloro-1,2-diphenylbut-1-en-1-yl)phenoxy)ethyl)-N7-((S)-1-((2S,4R)-4-hydroxy-2-((4-(4-methylthiazol-5-yl)benzyl)c arb amoyl)pyrrolidin-1-yl)-3,3-dimethyl-1-oxobutan-2-yl)-N1-methylheptanediamide (S IAIS 180029)

Referring to the procedures of Example 1, under appropriate conditions that will be recognized by one skilled in the art, the target compound (SIAIS180029) was prepared by using Toremifene derivative A ((Z)-2-(4-(4-chloro-1,2-diphenylbut-1-en-1-yl)phenoxy)-N-methylethan-1-amine) and intermediate LM (SIAIS074014) as starting materials. The target compound SIAIS180029 was obtained as white solid (11.9 mg, 54% yield). ¹H NMR (500 MHz, DMSO) δ 9.00 (s, 1H), 8.56 (t, J=6.1 Hz, 1H), 7.83 (d, J=8.0 Hz, 1H), 7.40 (dd, J=18.9, 8.1 Hz, 6H), 7.33-7.26 (m, 3H), 7.25-7.19 (m, 2H), 7.19-7.11 (m, 3H), 6.75 (dd, J=8.7, 6.4 Hz, 2H), 6.66-6.52 (m, 2H), 4.53 (d, J=9.4 Hz, 1H), 4.43 (dd, J=16.3, 8.1 Hz, 2H), 4.34 (s, 1H), 4.21 (dd, J=15.9, 5.5 Hz, 1H), 3.96 (t, J=5.2 Hz, 1H), 3.89 (t, J=5.8 Hz, 2H), 3.66-3.64 (m, 1H), 3.59 (t, J=5.0 Hz, 2H), 3.53 (t, J=5.9 Hz, 1H), 3.42 (t, J=7.2 Hz, 2H), 2.96 (s, 1.6H, N—CH₃), 2.88-2.81 (m, 2H), 2.79 (s, 1.4H, N—CH₃), 2.44 (s, 3H), 2.32-2.18 (m, 3H), 2.13-1.99 (m, 2H), 1.92-1.87 (m, 1H), 1.50-1.41 (m, 4H), 1.28-1.18 (m, 2H), 0.92 (d, J=3.5 Hz, 9H). HRMS (ESI) m/z: calcd for C₅₄H₆₅ClN₅O₆S⁺ [M+H]⁺, 946.4339; found, 946.4332.

### Example 21

### Synthesis of N1-(2-(44(Z)-4-chloro-1,2-diphenylbut-1-en-1-yl)phenoxy)ethyl)-N8-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)-N1-methyloctanediamide (S IAIS 180033)

Referring to the procedures of Example 1, under appropriate conditions that will be recognized by one skilled in the art, the target compound (SIAIS180033) was prepared by using Toremifene derivative A ((Z)-2-(4-(4-chloro-1,2-diphenylbut-1-en-1-yl)phenoxy)-N-methylethan-1-amine) and intermediate LM (SIAIS074015) as starting materials. The target compound SIAIS180033 was obtained as white solid (4.3 mg, 19% yield). ¹H NMR (500 MHz, DMSO) δ 8.98 (s, 1H), 8.56 (t, J=5.8 Hz, 1H), 7.83 (d, J=9.3 Hz, 1H), 7.45-7.36 (m, 6H), 7.31-7.27 (m, 3H), 7.22 (t, J=7.7 Hz, 2H), 7.18-7.12 (m, 3H), 6.80-6.70 (m, 2H), 6.60 (d, J=8.7 Hz, 2H), 4.54 (d, J=9.4 Hz, 1H), 4.43 (dd, J=15.6, 7.3 Hz, 2H), 4.34 (s, 1H), 4.21 (dd, J=16.0, 5.4 Hz, 1H), 3.96 (t, J=5.2 Hz, 1H), 3.90 (t, J=5.8 Hz, 1H), 3.65 (d, J=7.5 Hz, 2H), 3.59 (t, J=5.2 Hz, 1H), 3.53 (t, J=5.8 Hz, 2H), 2.96 (s, 1.7H, N—CH₃), 2.84 (t, J=7.1 Hz, 2H), 2.79 (s, 1.3H, N—CH₃), 2.44 (s, 3H), 2.29-2.20 (m, 3H), 2.14-2.07 (m, 1H), 2.06-1.99 (m, 1H), 1.94-1.85 (m, 1H), 1.49-1.41 (m, 6H), 1.23-1.21 (m, 4H), 0.92 (d, J=5.0 Hz, 9H). HRMS (ESI) m/z: calcd for C₅₅H67ClN₅O₆S⁺ [M+H]⁺, 960.4495; found, 960.4490.

### Example 22

### Synthesis of N1-(2-(4-((Z)-4-chloro-1,2-diphenylbut-1-en-1-yl)phenoxy)ethyl)-N9-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)-N1-methylnonanediamide (SIAIS180035)

Referring to the procedures of Example 1, under appropriate conditions that will be recognized by one skilled in the art, the target compound (SIAIS180035) was prepared by using Toremifene derivative A ((Z)-2-(4-(4-chloro-1,2-diphenylbut-1-en-1-yl)phenoxy)-N-methylethan-1-amine) and intermediate LM (SIAIS074016) as starting materials. The target compound SIAIS180035 was obtained as white solid (12.1 mg, 53% yield). ¹H NMR (500 MHz, DMSO) δ 9.0 (s, 1H), 8.56 (t, J=6.0 Hz, 1H), 7.83 (d, J=9.3 Hz, 1H), 7.46-7.35 (m, 6H), 7.29 (dd, J=13.3, 7.2 Hz, 3H), 7.22 (t, J=7.4 Hz, 2H), 7.17-7.13 (m, 3H), 6.75 (t, J=8.0 Hz, 2H), 6.60 (d, J=8.7 Hz, 2H), 4.54 (d, J=9.3 Hz, 1H), 4.43 (dd, J=15.6, 7.2 Hz, 2H), 4.35 (s, 1H), 4.21 (dd, J=15.9, 5.5 Hz, 1H), 3.96 (t, J=5.2 Hz, 1H), 3.90 (t, J=5.7 Hz, 1H), 3.68-3.41 (m, 7H), 2.96 (s, 1.6H, N—CH₃), 2.84 (t, J=7.1 Hz, 2H), 2.79 (s, 1.4H, N—CH₃), 2.44 (s, 3H), 2.29-2.20 (m, 3H), 2.13-2.07 (m, 1H), 2.05-1.97 (m, 1H), 1.94-1.86 (m, 1H), 1.58-1.37 (m, 4H), 1.21 (s, 6H), 0.93 (s, 9H). HRMS (ESI) m/z: calcd for C₅₆H₆₉ClN₅O₆S⁺ [M+H]⁺, 974.4652; found, 974.4647.

### Example 23

### Synthesis of N1-(2-(4-((Z)-4-chloro-1,2-diphenylbut-1-en-1-yl)phenoxy)ethyl)-N10-((S)-1-((2S,4R)-4-hydroxy-2-((4-(4-methylthiazol-5-yl)benzyl)c arb amoyl)pyrrolidin-1-yl)-3,3-dimethyl-1-oxobutan-2-yl)-N1-methyldec anediamide (S IAIS 180036)

Referring to the procedures of Example 1, under appropriate conditions that will be recognized by one skilled in the art, the target compound (SIAIS180036) was prepared by using Toremifene derivative A ((Z)-2-(4-(4-chloro-1,2-diphenylbut-1-en-1-yl)phenoxy)-N-methylethan-1-amine) and intermediate LM (SIAIS074019) as starting materials. The target compound SIAIS180036 was obtained as white solid (12.4 mg, 54% yield). ¹H NMR (500 MHz, DMSO) δ 8.98 (s, 1H), 8.58-8.55 (t, J=10.0 Hz, 1H), 7.83 (d, J=9.3 Hz, 1H), 7.45-7.35 (m, 6H), 7.31-7.27 (m, 3H), 7.25-7.19 (m, 2H), 7.17-7.13 (m, 3H), 6.75 (dd, J=8.7, 6.7 Hz, 2H), 6.60 (d, J=8.6 Hz, 2H), 5.12 (d, J=3.4 Hz, 1H), 4.54 (d, J=9.4 Hz, 1H), 4.43 (dd, J=15.6, 7.3 Hz, 2H), 4.34 (s, 1H), 4.21 (dd, J=15.8, 5.6 Hz, 1H), 3.97 (t, J=5.3 Hz, 1H), 3.90 (t, J=5.8 Hz, 1H), 3.70-3.61 (m, 2H), 3.59 (t, J=5.2 Hz, 1H), 3.52 (t, J=5.7 Hz, 1H), 3.42 (t, J=7.2 Hz, 2H), 2.96 (s, 1.6H, N—CH₃), 2.84 (t, J=7.1 Hz, 2H), 2.79 (s, 1.4H, N—CH₃), 2.44 (s, 3H), 2.30-2.18 (m, 3H), 2.12-2.01 (m, 2H), 1.94-1.85 (m, 1H), 1.52-1.39 (m, 4H), 1.21 (s, 8H), 0.93 (s, 9H). HRMS (ESI) m/z: calcd for C₅₇H₇₁ClN₅O₆S⁺ [M+H]⁺, 988.4808; found, 988.4801.

### Example 24

### Synthesis of (Z)-N1-(2-(4-(4-chloro-1,2-diphenylbut-1-en-1-yl)phenoxy)ethyl)-N3-(2-(2,6-dioxopiperidin-3-yl)-1-oxoisoindolin-4-yl)-N1-methylmalonamide (SIAIS180090)

Referring to the procedures of Example 1, under appropriate conditions that will be recognized by one skilled in the art, the target compound (SIAIS180090) was prepared by using Toremifene derivative A ((Z)-2-(4-(4-chloro-1,2-diphenylbut-1-en-1-yl)phenoxy)-N-methylethan-1-amine) and intermediate LM (SIAIS171004) as starting materials. The target compound SIAIS180090 was obtained as yellow solid (14.8 mg, 44% yield). ¹H NMR (500 MHz, DMSO) δ 11.02 (s, 1H), 10.01 (d, J=12.7 Hz, 1H), 7.84 (dd, J=10.9, 7.6 Hz, 1H), 7.54-7.46 (m, 2H), 7.40 (t, J=7.5 Hz, 2H), 7.32-7.28 (m, 3H), 7.22 (t, J=7.7 Hz, 2H), 7.18-7.13 (m, 3H), 6.77-6.75 (m, 2H), 6.64 (t, J=8.3 Hz, 2H), 5.14 (dd, J=13.2, 5.1 Hz, 1H), 4.39-4.29 (m, 2H), 4.02 (t, J=5.2 Hz, 1H), 3.94 (t, J=5.6 Hz, 1H), 3.70-3.68 (m, 1H), 3.64-3.57 (m, 2H), 3.53 (s, 1H), 3.48-3.39 (m, 2H), 3.05 (s, 1.5H, N—CH₃), 2.96-2.80 (m, 4.5H, N—CH₃), 2.67-2.56 (m, 1H), 2.38-2.26 (m, 1H), 2.05-1.99 (m, 1H). HRMS (ESI) m/z: calcd for C₄₁H₄₀ClN₄O₆⁺ [M+H]⁺, 719.2631; found, 719.2621.

### Example 25

### Synthesis of (Z)—N1-(2-(4-(4-chloro-1,2-diphenylbut-1-en-1-yl)phenoxy)ethyl)-N4-(2-(2,6-dioxopiperidin-3-yl)-1-oxoisoindolin-4-yl)-N1-methylsuccinamide (SIAIS180091)

Referring to the procedures of Example 1, under appropriate conditions that will be recognized by one skilled in the art, the target compound (SIAIS180091) was prepared by using Toremifene derivative A ((Z)-2-(4-(4-chloro-1,2-diphenylbut-1-en-1-yl)phenoxy)-N-methylethan-1-amine) and intermediate LM (SIAIS164084) as starting materials. The target compound SIAIS180091 was obtained as yellow solid (14.5 mg, 42% yield). ¹H NMR (500 MHz, DMSO) δ 11.02 (s, 1H), 9.82 (s, 1H), 7.86-7.78 (m, 1H), 7.51-7.44 (m, 2H), 7.40 (t, J=7.5 Hz, 2H), 7.32-7.28 (m, 3H), 7.22 (t, J=7.4 Hz, 2H), 7.17-7.13 (m, 3H), 6.75 (dd, J=8.7, 3.4 Hz, 2H), 6.63 (dd, J=16.8, 8.8 Hz, 2H), 5.14 (dd, J=13.3, 5.1 Hz, 1H), 4.40-4.29 (m, 2H), 4.00 (t, J=5.1 Hz, 1H), 3.91 (t, J=5.7 Hz, 1H), 3.67-3.65 (m, 1H), 3.56-3.54 (m, 1H), 3.43 (t, J=6.9 Hz, 2H), 3.01 (s, 1.5H, N—CH₃), 2.96-2.79 (m, 4.5H, N—CH₃), 2.73-2.70 (m, 1H), 2.59-2.56 (m, 4H), 2.39-2.19 (m, 1H), 2.02-1.98 (d, J=5.3 Hz, 1H). HRMS (ESI) m/z: calcd for C₄₂H₄₂ClN₄O₆⁺ [M+H]⁺, 733.2787; found, 733.2779.

### Example 26

### Synthesis of (Z)-N1-(2-(4-(4-chloro-1,2-diphenylbut-1-en-1-yl)phenoxy)ethyl)-N5-(2-(2,6-dioxopiperidin-3-yl)-1-oxoisoindolin-4-yl)-N1-methylglutaramide (SIAIS180092)

Referring to the procedures of Example 1, under appropriate conditions that will be recognized by one skilled in the art, the target compound (SIAIS180092) was prepared by using Toremifene derivative A ((Z)-2-(4-(4-chloro-1,2-diphenylbut-1-en-1-yl)phenoxy)-N-methylethan-1-amine) and intermediate LM (SIAIS171005) as starting materials. The target compound SIAIS180092 was obtained as yellow solid (15.1 mg, 43% yield). ¹H NMR (500 MHz, DMSO) δ 11.01 (s, 1H), 9.77 (d, J=6.2 Hz, 1H), 7.82-7.76 (m, 1H), 7.52-7.37 (m, 4H), 7.32-7.26 (m, 3H), 7.25-7.19 (m, 2H), 7.17-7.13 (m, 3H), 6.72 (dd, J=20.2, 8.8 Hz, 2H), 6.60 (dd, J=13.6, 8.8 Hz, 2H), 5.14 (dd, J=13.7, 4.4 Hz, 1H), 4.40-4.30 (m, 2H), 3.96 (t, J=5.2 Hz, 1H), 3.91 (t, J=5.8 Hz, 1H), 3.61 (t, J=5.1 Hz, 1H), 3.55 (t, J=5.8 Hz, 1H), 3.42 (t, J=7.3 Hz, 2H), 3.01-2.75 (m, 6H), 2.63-2.55 (m, 1H), 2.44-2.30 (m, 5H), 2.03-1.94 (m, 1H), 1.85-1.75 (m, 2H). HRMS (ESI) m/z: calcd for C₄₃H₄₄ClN₄O₆⁺ [M+H]⁺, 747.2944; found, 747.2937.

### Example 27

### Synthesis of (Z)—N1-(2-(4-(4-chloro-1,2-diphenylbut-1-en-1-yl)phenoxy)ethyl)-N6-(2-(2,6-dioxopiperidin-3-yl)-1-oxoisoindolin-4-yl)-N1-methyladipamide (SIAIS180093)

Referring to the procedures of Example 1, under appropriate conditions that will be recognized by one skilled in the art, the target compound (SIAIS180093) was prepared by using Toremifene derivative A ((Z)-2-(4-(4-chloro-1,2-diphenylbut-1-en-1-yl)phenoxy)-N-methylethan-1-amine) and intermediate LM (SIAIS164101) as starting materials. The target compound SIAIS180093 was obtained as yellow solid (13.9 mg, 39% yield). ¹H NMR (500 MHz, DMSO) δ 11.01 (s, 1H), 9.78 (s, 1H), 7.79 (dd, J=7.5, 2.1 Hz, 1H), 7.51-7.45 (m, 2H), 7.40 (t, J=7.4 Hz, 2H), 7.33-7.25 (m, 3H), 7.22 (t, J=7.7 Hz, 2H), 7.17-7.13 (m, 3H), 6.74 (d, J=8.3 Hz, 2H), 6.61 (d, J=8.5 Hz, 2H), 5.14 (dd, J=13.3, 5.1 Hz, 1H), 4.36 (q, J=17.5 Hz, 2H), 3.97 (t, J=5.3 Hz, 1H), 3.90 (t, J=5.8 Hz, 1H), 3.61 (t, J=5.2 Hz, 1H), 3.55-3.51 (m, 1H), 3.42 (t, J=7.3 Hz, 2H), 3.01-2.76 (m, 6H), 2.59 (d, J=17.2 Hz, 1H), 2.41-2.32 (m, 4H), 2.29 (t, J=7.2 Hz, 1H), 2.01-1.99 (m, 1H), 1.68-1.45 (m, 4H). HRMS (ESI) m/z: calcd for C₄₄H₄₆ClN₄O₆⁺ [M+H]⁺, 761.3100; found, 761.3095.

### Example 28

### Synthesis of (Z)—N1-(2-(4-(4-chloro-1,2-diphenylbut-1-en-1-yl)phenoxy)ethyl)-N7-(2-(2,6-dioxopiperidin-3-yl)-1-oxoi soindolin-4-yl)-N1-methylheptanedi amide (SIAIS180094)

Referring to the procedures of Example 1, under appropriate conditions that will be recognized by one skilled in the art, the target compound (SIAIS180094) was prepared by using Toremifene derivative A ((Z)-2-(4-(4-chloro-1,2-diphenylbut-1-en-1-yl)phenoxy)-N-methylethan-1-amine) and intermediate LM (SIAIS164102) as starting materials. The target compound SIAIS180094 was obtained as yellow solid (13.3 mg, 37% yield). ¹H NMR (500 MHz, DMSO) δ 11.01 (s, 1H), 9.76 (s, 1H), 7.80 (d, J=7.6 Hz, 1H), 7.52-7.44 (m, 2H), 7.39 (dt, J=7.7, 3.7 Hz, 2H), 7.32-7.26 (m, 3H), 7.23-7.20 (m, 2H), 7.17-7.13 (m, 3H), 6.74 (d, J=8.8 Hz, 2H), 6.60 (d, J=8.5 Hz, 2H), 5.14 (dd, J=13.4, 5.0 Hz, 1H), 4.41-4.31 (m, 2H), 3.97 (t, J=5.2 Hz, 1H), 3.90 (t, J=5.8 Hz, 1H), 3.60 (t, J=5.3 Hz, 1H), 3.53 (t, J=5.7 Hz, 1H), 3.44-3.41 (m, 2H), 2.99-2.76 (m, 6H), 2.59 (d, J=16.3 Hz, 1H), 2.35-2.31 (m, 4H), 2.25 (t, J=7.4 Hz, 1H), 2.02-1.98 (m, 1H), 1.60-1.58 (m, 2H), 1.50-1.49 (m, 2H), 1.34-1.28 (m, 2H). HRMS (ESI) m/z: calcd for C₄₅H₄₈ClN₄O₆⁺ [M+H]⁺, 775.3257; found, 775.3252.

### Example 29

### Synthesis of (2S,4R)-1-((S)-2-(tert-butyl)-14-(4-(4-chloro-1-(4-hydroxyphenyl)-2-phenylbut-1-en-1-yl)phenoxy)-4,11-dioxo-6,9-dioxa-3,12-di azatetradec anoyl)-4-hydroxy-N-(4-(4-methylthi azol-5-yl)benzyl)pyrrolidine-2-c arboxamide (SIAIS208041)

According to scheme 7, Toremifene derivative B (4-(1-(4-(2-aminoethoxy)phenyl)-4-chloro-2-phenylbut-1-en-1-yl)phenol; 0.02539 mmol, 1 equiv), intermediate LM (SIAIS151010) (0.02539 mmol, 1 equiv), HOAt (0.05078 mmol, 2 equiv), EDCI (0.05078 mmol, 2 equiv), anhydrous DMF (2 mL) and NMM (0.127 mmol, 5 equiv) were sequentially added to a reaction flask at room temperature. The resulting reaction mixture was stirred at room temperature overnight. After the reaction was complete as detected by LC-MS, the resulting mixture was purified by preparative HPLC (eluent (v/v): acetonitrile/(water+0.05%HCl)=10%-100%). The acetonitrile was removed by rotary evaporation, and the resulting residue was lyophilized to yield the target product SIAIS208041 as white solid (6.3 mg, 26% yield). ¹H NMR (500 MHz, MeOD) δ 9.29 (d, J=10.0 Hz, 1H), 7.50-7.42 (m, 4H), 7.20-7.14 (m, 3H), 7.14-7.07 (m, 4H), 6.93-6.91 (m, 1H), 6.80-6.74 (m, 2H), 6.68-6.63 (m, 1H), 6.57-6.54 (m, 1H), 6.42-6.36 (m, 1H), 4.70 (d, J=9.5 Hz, 1H), 4.62-4.53 (m, 1H), 4.52-4.46 (m, 2H), 4.41-4.33 (m, 1H), 4.10-3.99 (m, 5H), 3.94-3.85 (m, 2H), 3.81-3.76 (m, 1H), 3.74-3.64 (m, 5H), 3.58-3.47 (m, 1H), 3.39 (t, J=7.4 Hz, 2H), 2.94-2.89 (m, 2H), 2.52-2.50 (m, 3H), 2.26-2.21 (m, 1H), 2.10-2.04 (m, 1H), 1.02-0.99 (m, 9H). HRMS (ESI) m/z: calcd for C₅₂H₆₁ClN₅O₉S⁺ [M+H]⁺, 966.3873; found, 966.3879.

### Example 30

### Synthesis of (2S,4R)-1-((S)-2-(tert-butyl)-16-(4-(4-chloro-1-(4-hydroxyphenyl)-2-phenylbut-1-en-1-yl)phenoxy)-4,13-dioxo-7,10-dioxa-3,14-diazahexadecanoyl)-4-hydroxy-N-(4-(4-methylthiazol-5-yl)benzyl)pyrrolidine-2-carboxamide (SIAIS208017)

Referring to the procedures of Example 29, under appropriate conditions that will be recognized by one skilled in the art, the target compound (SIAIS208017) was prepared by using Toremifene derivative B (4-(1-(4-(2-aminoethoxy)phenyl)-4-chloro-2-phenylbut-1-en-1-yl)phenol) and intermediate LM (SIAIS151002) as starting materials. The target compound SIAIS208017 was obtained as white solid (7.5 mg, 30% yield). ¹H NMR (500 MHz, MeOD) δ 9.31 (s, 1H), 7.57-7.42 (m, 4H), 7.20-7.15 (m, 3H), 7.15-7.07 (m, 4H), 6.97-6.91 (m, 1H), 6.81-6.74 (m, 2H), 6.69-6.64 (m, 1H), 6.56 (d, J=8.8 Hz, 1H), 6.44-6.38 (m, 1H), 4.64 (d, J=4.3 Hz, 1H), 4.60-4.49 (m, 3H), 4.42-4.32 (m, 1H), 4.07 (t, J=5.4 Hz, 1H), 3.90-3.88 (m, 2H), 3.82-3.76 (m, 1H), 3.73 (t, J=6.0 Hz, 1H), 3.70-3.63 (m, 3H), 3.61-3.51 (m, 5H), 3.49 (t, J=5.2 Hz, 1H), 3.39 (t, J=7.4 Hz, 2H), 2.91 (dt, J=13.1, 7.4 Hz, 2H), 2.55-2.40 (m, 7H), 2.27-2.18 (m, 1H), 2.12-2.00 (m, 1H), 1.03-1.01 (m, 9H). HRMS (ESI) m/z: calcd for C₅₄H₆₅ClN₅O₉S⁺ [M+H]⁺, 994.4186; found, 994.4196.

### Example 31

### Synthesis of (2S,4R)-1-((S)-2-(tert-butyl)-19-(4-(4-chloro-1-(4-hydroxyphenyl)-2-phenylbut-1-en-1-yl)phenoxy)-4,16-dioxo-7,10,13-trioxa-3,17-diazanonadecanoyl)-4-hydroxy-N-(4-(4-methylthiazol-5-yl)benzyl)pyrrolidine-2-carboxamide (SIAIS208018)

Referring to the procedures of Example 29, under appropriate conditions that will be recognized by one skilled in the art, the target compound (SIAIS208018) was prepared by using Toremifene derivative B (4-(1-(4-(2-aminoethoxy)phenyl)-4-chloro-2-phenylbut-1-en-1-yl)phenol) and intermediate LM (SIAIS151003) as starting materials. The target compound SIAIS208018 was obtained as white solid (7.2 mg, 27% yield). ¹H NMR (500 MHz, MeOD) δ 9.17 (d, J=1.8 Hz, 1H), 7.49 (d, J=8.0 Hz, 2H), 7.46-7.41 (m, 2H), 7.22-7.15 (m, 3H), 7.15-7.07 (m, 4H), 6.97-6.92 (m, 1H), 6.81-6.74 (m, 2H), 6.69-6.64 (m, 1H), 6.58-6.56 (m, 1H), 6.44-6.38 (m, 1H), 4.64 (s, 1H), 4.58-4.49 (m, 3H), 4.39-4.34 (m, 1H), 4.08 (t, J=5.4 Hz, 1H), 3.91-3.88 (m, 2H), 3.79 (dd, J=11.0, 3.8 Hz, 1H), 3.75-3.66 (m, 4H), 3.60-3.53 (m, 9H), 3.48 (t, J=5.4 Hz, 1H), 3.40 (t, J=7.4 Hz, 2H), 2.92 (dt, J=11.9, 7.4 Hz, 2H), 2.60-2.39 (m, 7H), 2.24-2.20 (m, 1H), 2.10-2.05 (m, 1H), 1.03-1.01 (m, 9H). HRMS (ESI) m/z: calcd for C₅₆H₆₉ClN₅O₁₀S⁺ [M+H]⁺, 1038.4448; found, 1038.4442.

### Example 32

### Synthesis of N1-(2-(4-(4-chloro-1-(4-hydroxyphenyl)-2-phenylbut-1-en-1-yl)phenoxy)ethyl)-N16-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)-4,7,10,13-tetraoxahexadecanediamide (SIAIS208019)

Referring to the procedures of Example 29, under appropriate conditions that will be recognized by one skilled in the art, the target compound (SIAIS208019) was prepared by using Toremifene derivative B (4-(1-(4-(2-aminoethoxy)phenyl)-4-chloro-2-phenylbut-1-en-1-yl)phenol) and intermediate LM (SIAIS151008) as starting materials. The target compound SIAIS208019 was obtained as white solid (7.9 mg, 29% yield). ¹H NMR (500 MHz, MeOD) δ 9.23 (d, J=1.3 Hz, 1H), 7.50 (d, J=8.2 Hz, 2H), 7.44 (d, J=8.1 Hz, 2H), 7.21-7.15 (m, 3H), 7.15-7.07 (m, 4H), 6.95 (d, J=8.7 Hz, 1H), 6.81-6.75 (m, 2H), 6.69-6.64 (m, 1H), 6.57 (d, J=8.8 Hz, 1H), 6.44-6.38 (m, 1H), 4.64 (s, 1H), 4.60-4.47 (m, 3H), 4.39-4.34 (m, 1H), 4.09-4.06 (m, 1H), 3.91-3.88 (m, 2H), 3.79 (dd, J=11.0, 3.8 Hz, 1H), 3.74-3.67 (m Hz, 4H), 3.60-3.51 (m, 11H), 3.54-3.52 (m, 2H), 3.49 (t, J=5.4 Hz, 1H), 3.40 (t, J=7.4 Hz, 2H), 2.95-2.88 (m, 2H), 2.60-2.46 (m, 6H), 2.42 (t, J=6.0 Hz, 1H), 2.24-2.20 (m, 1H), 2.10-2.03 (m, 1H), 1.03-1.01 (m, 9H). HRMS (ESI) m/z: calcd for C₅₈H₇₃ClN₅O₁₁S⁺ [M+H]⁺, 1082.4710; found, 1082.4706.

### Example 33

### Synthesis of N1-(2-(4-(4-chloro-1-(4-hydroxyphenyl)-2-phenylbut-1-en-1-yl)phenoxy)ethyl)-N19-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)-4,7,10,13,16-pentaoxanonadecanediamide (SIAIS208045)

Referring to the procedures of Example 29, under appropriate conditions that will be recognized by one skilled in the art, the target compound (SIAIS208045) was prepared by using Toremifene derivative B (4-(1-(4-(2-aminoethoxy)phenyl)-4-chloro-2-phenylbut-1-en-1-yl)phenol) and intermediate LM (SIAIS151009) as starting materials. The target compound SIAIS208045 was obtained as white solid (8.1 mg, 28% yield). ¹H NMR (500 MHz, MeOD) δ 9.63 (d, J=1.1 Hz, 1H), 7.54 (d, J=8.2 Hz, 2H), 7.48 (d, J=8.2 Hz, 2H), 7.22-7.15 (m, 3H), 7.15-7.07 (m, 4H), 6.97-6.94 (m, 1H), 6.82-6.75 (m, 2H), 6.68-6.64 (m, 1H), 6.60-6.55 (m, 1H), 6.43-6.39 (m, 1H), 4.64 (s, 1H), 4.60-4.47 (m, 3H), 4.41-4.34 (m, 1H), 4.08 (t, J=5.4 Hz, 1H), 3.92-3.88 (m, 2H), 3.83-3.77 (m, 1H), 3.75-3.68 (m, 4H), 3.62-3.56 (m, 13H), 3.55-3.54 (m, 4H), 3.49 (t, J=5.4 Hz, 1H), 3.41-3.38 (m, 2H), 2.95-2.90 (m, 2H), 2.61-2.53 (m, 4H), 2.49-2.42 (m, 3H), 2.25-2.20 (m, 1H), 2.10-2.05 (m, 1H), 1.03-1.01 (m, 9H). HRMS (ESI) m/z: calcd for C₆₀H₇₇ClN₅O₁₂S⁺ [M+H]+, 1126.4972; found, 1126.4981.

### Example 34

### Synthesis of N1-(2-(4-(4-chloro-1-(4-hydroxyphenyl)-2-phenylbut-1-en-1-yl)phenoxy)ethyl)-N4-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)succinamide (SIAIS208020)

Referring to the procedures of Example 29, under appropriate conditions that will be recognized by one skilled in the art, the target compound (SIAIS208020) was prepared by using Toremifene derivative B (4-(1-(4-(2-aminoethoxy)phenyl)-4-chloro-2-phenylbut-1-en-1-yl)phenol) and intermediate LM (SIAIS074011) as starting materials. The target compound SIAIS208020 was obtained as white solid (6.8 mg, 30% yield). ¹H NMR (500 MHz, MeOD) δ 8.98 (s, 1H), 7.47 (d, J=8.3 Hz, 2H), 7.44-7.41 (m, 2H), 7.23-7.06 (m, 7H), 6.95 (d, J=8.7 Hz, 1H), 6.79-6.76 (m, 2H), 6.68-6.64 (m, 1H), 6.57 (d, J=8.8 Hz, 1H), 6.43-6.38 (m, 1H), 4.62-4.44 (m, 4H), 4.35 (d, J=15.5 Hz, 1H), 4.07 (t, J=5.5 Hz, 1H), 3.90-3.85 (m, 2H), 3.79-3.72 (m, 1H), 3.60-3.56 (m, 1H), 3.48-3.45 (m, 1H), 3.42-3.39 (m, 2H), 2.97-2.88 (m, 2H), 2.65-2.42 (m, 7H), 2.22-2.19 (m, 1H), 2.12-2.02 (m, 1H), 1.02-0.99 (m, 9H). HRMS (ESI) m/z: calcd for C₅₀H₅₇ClN₅O₇S⁺ [M+H]⁺, 906.3662; found, 906.3672.

### Example 35

### Synthesis of N1-(2-(4-(4-chloro-1-(4-hydroxyphenyl)-2-phenylbut-1-en-1-yl)phenoxy)ethyl)-N5-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)glutaramide (SIAIS208031)

Referring to the procedures of Example 29, under appropriate conditions that will be recognized by one skilled in the art, the target compound (SIAIS208031) was prepared by using Toremifene derivative B (4-(1-(4-(2-aminoethoxy)phenyl)-4-chloro-2-phenylbut-1-en-1-yl)phenol) and intermediate LM (SIAIS074012) as starting materials. The target compound SIAIS208031 was obtained as white solid (5.2 mg, 22% yield). ¹H NMR (500 MHz, MeOD) δ 9.47 (d, J=5.4 Hz, 1H), 7.52 (d, J=7.3 Hz, 2H), 7.48-7.44 (m, 2H), 7.20-7.06 (m, 7H), 6.94 (d, J=8.7 Hz, 1H), 6.80-6.74 (m, 2H), 6.68-6.63 (m, 1H), 6.58-6.53 (m, 1H), 6.43-6.37 (m, 1H), 4.61-4.47 (m, 4H), 4.35 (dd, J=14.9, 7.1 Hz, 1H), 4.08 (t, J=5.5 Hz, 1H), 3.95-3.88 (m, 2H), 3.82-3.76 (m, 1H), 3.61-3.57 (m, 1H), 3.52-3.43 (m, 1H), 3.42-3.38 (m, 2H), 2.94-2.89 (m, 2H), 2.56-2.49 (m, 3H), 2.34-2.16 (m, 5H), 2.11-2.04 (m, 1H), 1.96-1.82 (m, 2H), 1.03-0.99 (m, 9H). HRMS (ESI) m/z: calcd for C₅₁H₅₉ClN₅O₇S⁺ [M+H]⁺, 920.3818; found, 920.3811.

### Example 36

### Synthesis of N1-(2-(4-(4-chloro-1-(4-hydroxyphenyl)-2-phenylbut-1-en-1-yl)phenoxy)ethyl)-N6-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)adip amide (SIAIS208032)

Referring to the procedures of Example 29, under appropriate conditions that will be recognized by one skilled in the art, the target compound (SIAIS208032) was prepared by using Toremifene derivative B (4-(1-(4-(2-aminoethoxy)phenyl)-4-chloro-2-phenylbut-1-en-1-yl)phenol) and intermediate LM (SIAIS074013) as starting materials. The target compound SIAIS208032 was obtained as white solid (7.5 mg, 32% yield). ¹H NMR (500 MHz, MeOD) δ 9.40 (d, J=5.5 Hz, 1H), 7.54-7.49 (m, 2H), 7.46 (d, J=8.3 Hz, 2H), 7.21-7.05 (m, 7H), 6.95 (d, J=8.7 Hz, 1H), 6.77 (dd, J=8.6, 6.6 Hz, 2H), 6.67-6.65 (m, 1H), 6.57 (d, J=8.8 Hz, 1H), 6.41 (d, J=8.6 Hz, 1H), 4.65-4.45 (m, 4H), 4.37 (d, J=15.6 Hz, 1H), 4.07 (t, J=5.4 Hz, 1H), 3.90 (t, J=5.4 Hz, 2H), 3.82-3.72 (m, 1H), 3.58 (t, J=5.5 Hz, 1H), 3.46 (dd, J=10.8, 5.2 Hz, 1H), 3.40 (t, J=7.5 Hz, 2H), 2.95-2.88 (m, 2H), 2.52 (d, J=5.3 Hz, 3H), 2.37-2.14 (m, 5H), 2.12-2.02 (m, 1H), 1.61 (d, J=20.4 Hz, 4H), 1.02-1.00 (m, 9H). HRMS (ESI) m/z: calcd for C₅₂H₆₁ClN₅O₇S⁺ [M+H]⁺, 934.3975; found, 934.3975.

### Example 37

### Synthesis of N1-(2-(4-(4-chloro-1-(4-hydroxyphenyl)-2-phenylbut-1-en-1-yl)phenoxy)ethyl)-N7-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)heptanedi amide (SIAIS208033)

Referring to the procedures of Example 29, under appropriate conditions that will be recognized by one skilled in the art, the target compound (SIAIS208033) was prepared by using Toremifene derivative B (4-(1-(4-(2-aminoethoxy)phenyl)-4-chloro-2-phenylbut-1-en-1-yl)phenol) and intermediate LM (SIAIS074014) as starting materials. The target compound SIAIS208033 was obtained as white solid (8.1 mg, 34% yield). ¹H NMR (500 MHz, MeOD) δ 8.95 (s, 1H), 7.47 (d, J=8.3 Hz, 2H), 7.43-7.39 (m, 2H), 7.21-7.06 (m, 7H), 6.95 (d, J=8.7 Hz, 1H), 6.80-6.75 (m, 2H), 6.68-6.64 (m, 1H), 6.59-6.55 (m, 1H), 6.43-6.39 (m, 1H), 4.62 (d, J=2.5 Hz, 1H), 4.58-4.49 (m, 3H), 4.35 (d, J=15.5 Hz, 1H), 4.07 (t, J=5.5 Hz, 1H), 3.93-3.87 (m, 2H), 3.81-3.76 (m, 1H), 3.60-3.55 (m, 1H), 3.46 (t, J=5.6 Hz, 1H), 3.41-3.38 (m, 2H), 2.95-2.86 (m, 2H), 2.47 (s, 3H), 2.29-2.15 (m Hz, 5H), 2.11-2.03 (m, 1H), 1.68-1.54 (m, 4H), 1.38-1.32 (m, 2H), 1.02-1.00 (m, 9H). HRMS (ESI) m/z: calcd for C₅₃H₆₃ClN₅O₇S⁺ [M+H]⁺, 948.4131; found, 948.4137.

### Example 38

### Synthesis of N1-(2-(4-(4-chloro-1-(4-hydroxyphenyl)-2-phenylbut-1-en-1-yl)phenoxy)ethyl)-N8-((S)-1-((2S,4R)-4-hydroxy-24(4-(4-methylthiazol-5-yl)benzyl)carbamoyl)pyrrolidin-1-yl)-3,3-dimethyl-1-oxobutan-2-yl)octanediamide (SIAIS208034)

Referring to the procedures of Example 29, under appropriate conditions that will be recognized by one skilled in the art, the target compound (SIAIS208034) was prepared by using Toremifene derivative B (4-(1-(4-(2-aminoethoxy)phenyl)-4-chloro-2-phenylbut-1-en-1-yl)phenol) and intermediate LM (SIAIS074015) as starting materials. The target compound SIAIS208034 was obtained as white solid (8.4 mg, 34% yield). ¹H NMR (500 MHz, MeOD) δ 9.20 (s, 1H), 7.49 (d, J=8.1 Hz, 2H), 7.46-7.43 (m, 2H), 7.21-7.05 (m, 7H), 6.95-6.91 (m, 1H), 6.79-6.76 (m, 2H), 6.68-6.62 (m, 1H), 6.57-6.53 (m, 1H), 6.43-6.37 (m, 1H), 4.63 (s, 1H), 4.60-4.47 (m, 3H), 4.39-4.32 (m, 1H), 4.08-4.05 (m, 1H), 3.95-3.85 (m, 2H), 3.79 (dd, J=11.0, 3.8 Hz, 1H), 3.58-3.54 (m, 1H), 3.46 (t, J=5.4 Hz, 1H), 3.40 (t, J=7.4 Hz, 2H), 2.95-2.88 (m, 2H), 2.50 (s, 3H), 2.30-2.13 (m, 5H), 2.10-2.03 (m, 1H), 1.61-1.57 (m, 4H), 1.36-1.27 (m, 4H), 1.03-1.00 (m, 9H). HRMS (ESI) m/z: calcd for C₅₄H₆₅ClN₅O₇S⁺ [M+H]⁺, 962.4288; found, 962.4280.

### Example 39

### Synthesis of N1-(2-(4-(4-chloro-1-(4-hydroxyphenyl)-2-phenylbut-1-en-1-yl)phenoxy)ethyl)-N9-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)nonanediamide (SIAIS208035)

Referring to the procedures of Example 29, under appropriate conditions that will be recognized by one skilled in the art, the target compound (SIAIS208035) was prepared by using Toremifene derivative B (4-(1-(4-(2-aminoethoxy)phenyl)-4-chloro-2-phenylbut-1-en-1-yl)phenol) and intermediate LM (SIAIS074016) as starting materials. The target compound SIAIS208035 was obtained as white solid (9.7 mg, 40% yield). ¹H NMR (500 MHz, MeOD) δ 9.31 (s, 1H), 7.51 (d, J=8.2 Hz, 2H), 7.48-7.43 (m, 2H), 7.21-7.06 (m, 7H), 6.96-6.91 (m, 1H), 6.80-6.74 (m, 2H), 6.69-6.64 (m, 1H), 6.58-6.53 (m, 1H), 6.43-6.38 (m, 1H), 4.63 (s, 1H), 4.61-4.46 (m, 3H), 4.37 (d, J=15.6 Hz, 1H), 4.06 (t, J=5.4 Hz, 1H), 3.92-3.88 (m, 2H), 3.80 (dd, J=10.9, 3.8 Hz, 1H), 3.56 (t, J=5.4 Hz, 1H), 3.46 (t, J=5.4 Hz, 1H), 3.40 (t, J=7.4 Hz, 2H), 2.95-2.89 (m, 2H), 2.52 (s, 3H), 2.32-2.12 (m, 5H), 2.10-2.02 (m, 1H), 1.67-1.51 (m, 4H), 1.37-1.23 (m, 6H), 1.03-1.00 (m, 9H). HRMS (ESI) m/z: calcd for C₅₅H₆₇ClN₅O₇S⁺ [M+H]⁺, 976.4444; found, 976.4441.

### Example 40

### Synthesis of N1-(2-(4-((Z)-4-chloro-1-(4-hydroxyphenyl)-2-phenylbut-1-en-1-yl)phenoxy)ethyl)-N10-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)dec anediamide (SIAIS 208036)

Referring to the procedures of Example 29, under appropriate conditions that will be recognized by one skilled in the art, the target compound (SIAIS208036) was prepared by using Toremifene derivative B (4-(1-(4-(2-aminoethoxy)phenyl)-4-chloro-2-phenylbut-1-en-1-yl)phenol) and intermediate LM (SIAIS074019) as starting materials. The target compound SIAIS208036 was obtained as white solid (9.0 mg, 36% yield). ¹H NMR (500 MHz, MeOD) δ 9.23 (s, 1H), 7.50 (d, J=8.3 Hz, 2H), 7.44 (d, J=8.3 Hz, 2H), 7.22-7.06 (m, 7H), 6.96-6.91 (m, 1H), 6.82-6.74 (m, 2H), 6.68-6.63 (m, 1H), 6.58-6.53 (m, 1H), 6.44-6.37 (m, 1H), 4.63 (s, 1H), 4.60-4.47 (m, 3H), 4.36 (d, J=15.6 Hz, 1H), 4.07 (t, J=5.4 Hz, 1H), 3.89 (t, J=9.4 Hz, 2H), 3.80 (dd, J=11.0, 3.8 Hz, 1H), 3.57 (t, J=5.4 Hz, 1H), 3.46 (t, J=5.4 Hz, 1H), 3.40 (t, J=7.4 Hz, 2H), 2.95-2.89 (m, 2H), 2.51 (s, 3H), 2.31-2.12 (m, 5H), 2.10-2.04 (m, 1H), 1.65-1.52 (m, 4H), 1.29-1.27 (m, 8H), 1.03-1.01 (m, 9H). HRMS (ESI) m/z: calcd for C₅₆H₆₉ClN₅O₇S⁺ [M+H]⁺, 990.4601; found, 990.4611.

### Example 41

### Synthesis of N1-(2-(4-(4-chloro-1-(4-hydroxyphenyl)-2-phenylbut-1-en-1-yl)phenoxy)ethyl)-N11-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)undecanediamide (SIAIS208037)

Referring to the procedures of Example 29, under appropriate conditions that will be recognized by one skilled in the art, the target compound (SIAIS208037) was prepared by using Toremifene derivative B (4-(1-(4-(2-aminoethoxy)phenyl)-4-chloro-2-phenylbut-1-en-1-yl)phenol) and intermediate LM (SIAIS074020) as starting materials. The target compound SIAIS208037 was obtained as white solid (9.6 mg, 38% yield). ¹H NMR (500 MHz, MeOD) δ 9.15 (s, 1H), 7.49 (d, J=8.3 Hz, 2H), 7.46-7.43 (m, 2H), 7.22-7.05 (m, 7H), 6.94 (d, J=8.7 Hz, 1H), 6.78 (dd, J=9.6, 8.8 Hz, 2H), 6.66 (d, J=8.7 Hz, 1H), 6.60-6.52 (m, 1H), 6.45-6.38 (m, 1H), 4.63 (s, 1H), 4.60-4.47 (m, 3H), 4.36 (d, J=15.6 Hz, 1H), 4.07 (t, J=5.4 Hz, 1H), 3.92-3.88 (m, 2H), 3.80 (dd, J=10.5, 3.2 Hz, 1H), 3.58-3.55(m, 1H), 3.46 (t, J=5.4 Hz, 1H), 3.40 (t, J=7.4 Hz, 2H), 2.92 (dt, J=11.9, 7.5 Hz, 2H), 2.50 (s, 3H), 2.32-2.12 (m, 5H), 2.10-2.05 (m, 1H), 1.59-1.57 (m, 4H), 1.28 (d, J=9.2 Hz, 10H), 1.03-1.01 (m, 9H). HRMS (ESI) m/z: calcd for C₅₇H₇₁ClN₅O₇S⁺ [M+H]⁺, 1004.4757; found, 1004.4761.

### Example 42

### Synthesis of N1-(2-(4-(4-chloro-1-(4-hydroxyphenyl)-2-phenylbut-1-en-1-yl)phenoxy)ethyl)-N14-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)tetradecanediamide (SIAIS208038)

Referring to the procedures of Example 29, under appropriate conditions that will be recognized by one skilled in the art, the target compound (SIAIS208038) was prepared by using Toremifene derivative B (4-(1-(4-(2-aminoethoxy)phenyl)-4-chloro-2-phenylbut-1-en-1-yl)phenol) and intermediate LM (SIAIS164185) as starting materials. The target compound SIAIS208038 was obtained as white solid (10.2 mg, 38% yield). ¹H NMR (500 MHz, MeOD) δ 9.23 (s, 1H), 7.50 (d, J=8.2 Hz, 2H), 7.47-7.44 (m, 2H), 7.22-7.15 (m, 3H), 7.15-7.08 (m, 4H), 6.97-6.91 (m, 1H), 6.81-6.74 (m, 2H), 6.66 (d, J=8.7 Hz, 1H), 6.58-6.55 (m, 1H), 6.41 (d, J=8.6 Hz, 1H), 4.63 (s, 1H), 4.61-4.46 (m, 3H), 4.36 (d, J=15.6 Hz, 1H), 4.07 (t, J=5.4 Hz, 1H), 3.92-3.89 (m, 2H), 3.80 (dd, J=11.0, 3.8 Hz, 1H), 3.58-3.55 (m, 1H), 3.47 (t, J=5.4 Hz, 1H), 3.40 (t, J=7.4 Hz, 2H), 2.92 (dt, J=11.9, 7.5 Hz, 2H), 2.51 (s, 3H), 2.31-2.22 (m, 5H), 2.21-2.03 (m, 1H), 1.63-1.54 (m, 4H), 1.28 (d, J=13.1 Hz, 16H), 1.11-0.85 (m, 9H). HRMS (ESI) m/z: calcd for C₆₀H₇₇ClN₅O₇S⁺ [M+H]⁺, 1046.5227; found, 1046.5224.

### Example 43

### Synthesis of N1-(2-(4-(4-chloro-1-(4-hydroxyphenyl)-2-phenylbut-1-en-1-yl)phenoxy)ethyl)-N16-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)hexadecanediamide (SIAIS208039)

Referring to the procedures of Example 29, under appropriate conditions that will be recognized by one skilled in the art, the target compound (SIAIS208039) was prepared by using Toremifene derivative B (4-(1-(4-(2-aminoethoxy)phenyl)-4-chloro-2-phenylbut-1-en-1-yl)phenol) and intermediate LM (SIAIS164189) as starting materials. The target compound SIAIS208039 was obtained as white solid (12.1 mg, 44% yield). ¹H NMR (500 MHz, MeOD) δ 9.01 (s, 1H), 7.48 (d, J=8.2 Hz, 2H), 7.45-7.42 (m, 2H), 7.23-7.07 (m, 7H), 6.94 (d, J=8.7 Hz, 1H), 6.82-6.74 (m, 2H), 6.66 (d, J=8.7 Hz, 1H), 6.56 (d, J=8.8 Hz, 1H), 6.41 (d, J=8.7 Hz, 1H), 4.63 (s, 1H), 4.58-4.50 (m, 3H), 4.36 (d, J=15.5 Hz, 1H), 4.08 (t, J=5.3 Hz, 1H), 3.92-3.89 (m, 2H), 3.80 (dd, J=10.9, 3.9 Hz, 1H), 3.57 (t, J=5.3 Hz, 1H), 3.48-3.45 (m, 1H), 3.40 (t, J=7.4 Hz, 2H), 2.93 (dt, J=11.9, 7.5 Hz, 2H), 2.49 (s, 3H), 2.32-2.13 (m, 5H), 2.11-2.03 (m, 1H), 1.60-1.56 (m, 4H), 1.27 (d, J=14.9 Hz, 20H), 1.03-1.01 (m, 9H). HRMS (ESI) m/z: calcd for C₆₂H₈₁ClN₅O₇S⁺ [M+H]⁺, 1074.5540; found, 1074.5539.

### Example 44

### Synthesis of (N-(2-(4-(4-chloro-1-(4-hydroxyphenyl)-2-phenylbut-1-en-1-yl)phenoxy)ethyl)-3-(2-((2-(2,6-dioxopiperidin-3-yl)-1,3-dioxoisoindolin-4-yl)amino)ethoxy)propanamide (SIAIS208138)

Referring to the procedures of Example 29, under appropriate conditions that will be recognized by one skilled in the art, the target compound (SIAIS208138) was prepared by using Toremifene derivative B (4-(1-(4-(2-aminoethoxy)phenyl)-4-chloro-2-phenylbut-1-en-1-yl)phenol) and intermediate LM (SIAIS151001) as starting materials. The target compound SIAIS208138 was obtained as yellow solid (7.8 mg, 40% yield). ¹H NMR (500 MHz, DMSO) δ 11.09 (s, 1H), 9.35 (d, J=126.6 Hz, 1H), 8.08 (dt, J=41.3, 5.4 Hz, 1H), 7.58-7.54 (m, 1H), 7.24-7.08 (m, 7H), 7.07-7.00 (m, 2H), 6.93 (d, J=8.7 Hz, 1H), 6.76 (d, J=8.5 Hz, 1H), 6.71 (d, J=8.8 Hz, 1H), 6.62-6.52 (m, 3H), 6.40 (d, J=8.7 Hz, 1H), 5.15-4.92 (m, 1H), 3.99 (t, J=5.6 Hz, 1H), 3.82 (t, J=5.6 Hz, 1H), 3.66 (t, J=6.4 Hz, 1H), 3.62 (t, J=6.4 Hz, 1H), 3.56 (dt, J=14.7, 5.4 Hz, 2H), 3.46-3.38 (m, 5H), 3.32-3.26 (m, 1H), 2.93-2.80 (m, 3H), 2.61-2.51 (m, 2H), 2.37 (t, J=6.4 Hz, 1H), 2.32 (t, J=6.4 Hz, 1H), 2.02-1.98 (m, 1H). HRMS (ESI) m/z: calcd for C₄₂H₄₂ClN₄O₈⁺ [M+H]⁺, 765.2686; found, 765.2682.

### Example 45

### Synthesis of N-(2-(4-(4-chloro-1-(4-hydroxyphenyl)-2-phenylbut-1-en-1-yl)phenoxy)ethyl)-3-(2-(2-((2-(2,6-dioxopiperidin-3-yl)-1,3-dioxoisoindolin-4-yl)amino)ethoxy)ethoxy)propanamide (SIAIS208139)

Referring to the procedures of Example 29, under appropriate conditions that will be recognized by one skilled in the art, the target compound (SIAIS208139) was prepared by using Toremifene derivative B (4-(1-(4-(2-aminoethoxy)phenyl)-4-chloro-2-phenylbut-1-en-1-yl)phenol) and intermediate LM (SIAIS151004) as starting materials. The target compound SIAIS208139 was obtained as yellow solid (8.5 mg, 41% yield). ¹H NMR (500 MHz, DMSO) δ 11.09 (s, 1H), 9.37 (d, J=125.3 Hz, 1H), 8.06 (dt, J=41.9, 5.4 Hz, 1H), 7.60-7.51 (m, 1H), 7.22-7.17 (m, 3H), 7.16-7.11 (m, 4H), 7.04 (t, J=8.5 Hz, 2H), 6.94 (d, J=8.7 Hz, 1H), 6.77 (d, J=8.5 Hz, 1H), 6.71 (d, J=8.8 Hz, 1H), 6.59 (dd, J=8.6, 6.2 Hz, 3H), 6.41 (d, J=8.6 Hz, 1H), 5.12-4.97 (m, 1H), 3.99 (t, J=5.5 Hz, 1H), 3.81 (t, J=5.5 Hz, 1H), 3.64-3.55 (m, 4H), 3.54-3.47 (m, 3H), 3.47-3.41 (m, 6H), 3.32-3.31 (m, 1H), 2.90-2.82 (m, 3H), 2.59-2.53 (m, 2H), 2.34 (t, J=6.4 Hz, 1H), 2.29 (t, J=6.4 Hz, 1H), 2.05-1.97 (m, 1H). HRMS (ESI) m/z: calcd for C₄₄H₄₆ClN₄O₉⁺ [M+H]⁺, 809.2948; found, 809.2951.

### Example 46

### Synthesis of N-(2-(4-(4-chloro-1-(4-hydroxyphenyl)-2-phenylbut-1-en-1-yl)phenoxy)ethyl)-3-(2-(2-(2-((2-(2,6-dioxopiperidin-3-yl)-1,3-dioxoisoindolin-4-yl)amino)ethoxy)ethoxy)ethoxy)propanamide (SIAIS208140)

Referring to the procedures of Example 29, under appropriate conditions that will be recognized by one skilled in the art, the target compound (SIAIS208140) was prepared by using Toremifene derivative B (4-(1-(4-(2-aminoethoxy)phenyl)-4-chloro-2-phenylbut-1-en-1-yl)phenol) and intermediate LM (SIAIS151005) as starting materials. The target compound SIAIS208140 was obtained as yellow solid (9.3 mg, 43% yield). ¹H NMR (500 MHz, DMSO) δ 11.08 (s, 1H), 9.37 (d, J=110.9 Hz, 1H), 8.06 (dt, J=41.9, 5.5 Hz, 1H), 7.62-7.53 (m, 1H), 7.22-7.16 (m, 3H), 7.14-7.12 (m, 4H), 7.08-7.01 (m, 2H), 6.94 (d, J=8.8 Hz, 1H), 6.79-6.74 (m, 1H), 6.74-6.69 (m, 1H), 6.61-6.58 (m, 3H), 6.43-6.38 (m, 1H), 5.11-4.99 (m, 1H), 3.99 (t, J=5.6 Hz, 1H), 3.82 (t, J=5.6 Hz, 1H), 3.62-3.56 (m, 4H), 3.55-3.40 (m, 13H), 3.31-3.26 (m, 1H), 2.94-2.81 (m, 3H), 2.62-2.52 (m, 2H), 2.34 (t, J=6.4 Hz, 1H), 2.29 (t, J=6.4 Hz, 1H), 2.07-1.96 (m, 1H). HRMS (ESI) m/z: calcd for C₄₆H₅₀ClN₄O₁₀⁺ [M+H]⁺, 853.3210; found, 853.3206.

### Example 47

### Synthesis of N-(2-(4-(4-chloro-1-(4-hydroxyphenyl)-2-phenylbut-1-en-1-yl)phenoxy)ethyl)-1-((2-(2,6-dioxopiperidin-3-yl)-1,3-dioxoisoindolin-4-yl)amino)-3,6,9,12-tetraoxapentadecan-15-amide (SIAIS208141)

Referring to the procedures of Example 29, under appropriate conditions that will be recognized by one skilled in the art, the target compound (SIAIS208141) was prepared by using Toremifene derivative B (4-(1-(4-(2-aminoethoxy)phenyl)-4-chloro-2-phenylbut-1-en-1-yl)phenol) and intermediate LM (SIAIS151006) as starting materials. The target compound SIAIS208141 was obtained as yellow solid (10.1 mg, 44% yield). ¹H NMR (500 MHz, DMSO) δ 11.10 (s, 1H), 9.37 (d, J=126.2 Hz, 1H), 8.07 (dt, J=41.8, 5.5 Hz, 1H), 7.57 (t, J=7.8 Hz, 1H), 7.23-7.16 (m, 3H), 7.14-7.12 (m, 4H), 7.04 (dd, J=9.4, 7.9 Hz, 2H), 6.94 (d, J=8.7 Hz, 1H), 6.77 (d, J=8.5 Hz, 1H), 6.72 (d, J=8.8 Hz, 1H), 6.61-6.58 (m, 3H), 6.41 (d, J=8.6 Hz, 1H), 5.05 (dd, J=12.7, 5.4 Hz, 1H), 3.99 (t, J=5.6 Hz, 1H), 3.82 (t, J=5.6 Hz, 1H), 3.63-3.56 (m, 4H), 3.56-3.53 (m, 3H), 3.52-3.50 (m, 2H), 3.48-3.42 (m, 12H), 3.32-3.31 (m, 1H), 2.91-2.84 (m, 3H), 2.62-2.52 (m, 2H), 2.39-2.31 (m, 1H), 2.29 (t, J=6.4 Hz, 1H), 2.06-1.97 (m, 1H). HRMS (ESI) m/z: calcd for C₄₈H₅₄ClN₄O₁₁⁺ [M+H]⁺, 897.3472; found, 897.3470.

### Example 48

### Synthesis of N-(2-(4-(4-chloro-1-(4-hydroxyphenyl)-2-phenylbut-1-en-1-yl)phenoxy)ethyl)-2-((2-(2,6-dioxopiperidin-3-yl)-1,3-dioxoisoindolin-4-yl)amino)acetamide (SIAIS208142)

Referring to the procedures of Example 29, under appropriate conditions that will be recognized by one skilled in the art, the target compound (SIAIS208142) was prepared by using Toremifene derivative B (4-(1-(4-(2-aminoethoxy)phenyl)-4-chloro-2-phenylbut-1-en-1-yl)phenol) and intermediate LM (SIAIS151025) as starting materials. The target compound SIAIS208142 was obtained as yellow solid (5.1 mg, 28% yield). ¹H NMR (500 MHz, DMSO) δ 11.10 (s, 1H), 9.40 (d, J=125.9 Hz, 1H), 8.36 (dt, J=41.5, 5.5 Hz, 1H), 7.54-7.44 (m, 1H), 7.23-7.16 (m, 3H), 7.16-7.11 (m, 3H), 7.07-7.02 (m, 2H), 6.99-6.90 (m, 2H), 6.87-6.80 (m, 1H), 6.77 (d, J=8.5 Hz, 1H), 6.72 (d, J=8.8 Hz, 1H), 6.60 (dd, J=10.6, 8.7 Hz, 2H), 6.42 (d, J=8.6 Hz, 1H), 5.15-4.95 (m, 1H), 4.03 (t, J=5.4 Hz, 1H), 3.98 (d, J=5.7 Hz, 1H), 3.92 (d, J=5.6 Hz, 1H), 3.85 (t, J=5.5 Hz, 1H), 3.50 (dd, J=11.0, 5.5 Hz, 1H), 3.46-3.41 (m, 3H), 2.96-2.82 (m, 3H), 2.64-2.53 (m, 2H), 2.03-2.01 (m, 1H). HRMS (ESI) m/z: calcd for C₃₉H₃₆ClN₄O₇⁺ [M+H]⁺, 707.2267; found, 707.2262.

### Example 49

### Synthesis of N-(2-(4-(4-chloro-1-(4-hydroxyphenyl)-2-phenylbut-1-en-1-yl)phenoxy)ethyl)-3-((2-(2,6-dioxopiperidin-3-yl)-1,3-dioxoisoindolin-4-yl)amino)propanamide (SIAIS208143)

Referring to the procedures of Example 29, under appropriate conditions that will be recognized by one skilled in the art, the target compound (SIAIS208143) was prepared by using Toremifene derivative B (4-(1-(4-(2-aminoethoxy)phenyl)-4-chloro-2-phenylbut-1-en-1-yl)phenol) and intermediate LM (SIAIS151026) as starting materials. The target compound SIAIS208143 was obtained as yellow solid (7.6 mg, 42% yield). ¹H NMR (500 MHz, DMSO) δ 11.09 (s, 1H), 9.44 (s, 1H), 8.04 (dt, J=41.7, 5.5 Hz, 1H), 7.55 (dd, J=15.8, 7.4 Hz, 1H), 7.26-7.10 (m, 6H), 7.10-7.03 (m, 2H), 7.00 (dd, J=7.0, 2.8 Hz, 1H), 6.94 (d, J=8.7 Hz, 1H), 6.79-6.74 (m, 1H), 6.73-6.69 (m, 1H), 6.64-6.52 (m, 3H), 6.44-6.37 (m, 1H), 5.04 (dd, J=12.7, 5.5 Hz, 1H), 4.00 (t, J=5.6 Hz, 1H), 3.82 (t, J=5.6 Hz, 1H), 3.48-3.40 (m, 3H), 3.30-3.26 (m, 3H), 2.93-2.81 (m, 3H), 2.60-2.52 (m, 2H), 2.15 (t, J=6.8 Hz, 1H), 2.10 (t, J=6.8 Hz, 1H), 2.03-2.00 (m, 1H). HRMS (ESI) m/z: calcd for C₄₀H₃₈ClN₄O₇⁺ [M+H]⁺, 721.2424; found, 721.2415.

### Example 50

### Synthesis of N-(2-(4-(4-chloro-1-(4-hydroxyphenyl)-2-phenylbut-1-en-1-yl)phenoxy)ethyl)-5-((2-(2,6-dioxopiperidin-3-yl)-1,3-dioxoisoindolin-4-yl)amino)pentanamide (SIAIS208144)

Referring to the procedures of Example 29, under appropriate conditions that will be recognized by one skilled in the art, the target compound (SIAIS208144) was prepared by using Toremifene derivative B (4-(1-(4-(2-aminoethoxy)phenyl)-4-chloro-2-phenylbut-1-en-1-yl)phenol) and intermediate LM (SIAIS151020) as starting materials. The target compound SIAIS208144 was obtained as yellow solid (7.6 mg, 40% yield). ¹H NMR (500 MHz, DMSO) δ 11.09 (s, 1H), 9.35 (d, J=126.3 Hz, 1H), 7.99 (dt, J=42.1, 5.5 Hz, 1H), 7.63-7.47 (m, 1H), 7.23-7.10 (m, 6H), 7.09-7.03 (m, 2H), 7.01 (d, J=7.1 Hz, 1H), 6.94 (d, J=8.7 Hz, 1H), 6.78-6.74 (m, 1H), 6.71 (d, J=8.8 Hz, 1H), 6.62-6.57 (m, 2H), 6.52-6.49 (m, 1H), 6.42-6.38 (m, 1H), 5.04 (dd, J=12.7, 5.4 Hz, 1H), 3.99 (t, J=5.6 Hz, 1H), 3.82 (t, J=5.6 Hz, 1H), 3.44-3.40 (m, 3H), 3.31-3.20 (m, 3H), 2.94-2.81 (m, 3H), 2.63-2.52 (m, 2H), 2.13-1.97 (m, 3H), 1.56-1.44 (m, 4H). HRMS (ESI) m/z: calcd for C₄₂H₄₂ClN₄O₇⁺ [M+H]⁺, 749.2737; found, 749.2743.

### Example 51

### Synthesis of N-(2-(4-(4-chloro-1-(4-hydroxyphenyl)-2-phenylbut-1-en-1-yl)phenoxy)ethyl)-7-((2-(2,6-dioxopiperidin-3-yl)-1,3-dioxoisoindolin-4-yl)amino)heptanamide (SIAIS208145)

Referring to the procedures of Example 29, under appropriate conditions that will be recognized by one skilled in the art, the target compound (SIAIS208145) was prepared by using Toremifene derivative B (4-(1-(4-(2-aminoethoxy)phenyl)-4-chloro-2-phenylbut-1-en-1-yl)phenol) and intermediate LM (SIAIS151086) as starting materials. The target compound SIAIS208145 was obtained as yellow solid (8.2 mg, 42% yield). ¹H NMR (500 MHz, DMSO) δ 11.09 (s, 1H), 9.34 (d, J=119.0 Hz, 1H), 7.99 (dt, J=42.2, 5.6 Hz, 1H), 7.63-7.47 (m, 1H), 7.23-7.10 (m, 6H), 7.09-7.04 (m, 2H), 7.01 (d, J=7.1 Hz, 1H), 6.94 (d, J=8.8 Hz, 1H), 6.76 (d, J=8.6 Hz, 1H), 6.71 (d, J=8.8 Hz, 1H), 6.63-6.57 (m, 2H), 6.53-6.49 (m, 1H), 6.40 (d, J=8.7 Hz, 1H), 5.04 (dd, J=12.8, 5.4 Hz, 1H), 3.99 (t, J=5.6 Hz, 1H), 3.82 (t, J=5.6 Hz, 1H), 3.44-3.40 (m, 4H), 3.28-3.23 (m, 2H), 2.87 (dt, J=14.1, 8.1 Hz, 3H), 2.65-2.55 (m, 2H), 2.09 (t, J=7.4 Hz, 1H), 2.06-2.00 (m, 2H), 1.58-1.43 (m, 4H), 1.35-1.23 (m, 4H). HRMS (ESI) m/z: calcd for C₄₄H₄₆ClN₄O₇⁺ [M+H]⁺, 777.3050; found, 777.3053.

### Example 52

### Synthesis of N-(2-(4-(4-chloro-1-(4-hydroxyphenyl)-2-phenylbut-1-en-1-yl)phenoxy)ethyl)-2-((2-(2,6-dioxopiperidin-3-yl)-1-oxoi soindolin-4-yl)amino)acetamide (SIAIS251029)

Referring to the procedures of Example 29, under appropriate conditions that will be recognized by one skilled in the art, the target compound (SIAIS251029) was prepared by using Toremifene derivative B (4-(1-(4-(2-aminoethoxy)phenyl)-4-chloro-2-phenylbut-1-en-1-yl)phenol) and intermediate LM (SIAIS1204057) as starting materials. The target compound SIAIS251029 was obtained as yellow solid (5.9 mg, 34% yield). ¹H NMR (500 MHz, DMSO) δ 11.01 (s, 1H), 9.35 (d, J=127.5 Hz, 1H), 8.18 (dt, J=39.7, 5.7 Hz, 1H), 7.23-7.11 (m, 8H), 7.07 (d, J=8.5 Hz, 1H), 6.97-6.90 (m, 2H), 6.77 (d, J=8.5 Hz, 1H), 6.72 (d, J=8.8 Hz, 1H), 6.61 (d, J=8.6 Hz, 1H), 6.58-6.49 (m, 2H), 6.41 (d, J=8.6 Hz, 1H), 5.16-5.08 (m, 1H), 4.30-4.24 (m, 1H), 4.21-4.15 (m, 1H), 4.00 (t, J=5.6 Hz, 1H), 3.83 (t, J=5.6 Hz, 1H), 3.78 (s, 1H), 3.73 (s, 1H), 3.49-3.42 (m, 4H), 2.96-2.82 (m, 3H), 2.64-2.60 (m, 1H), 2.36-2.28 (m, 1H), 2.03-1.97 (m, 1H). HRMS (ESI) m/z: calcd for C₃₉H₃₈ClN₄O₆⁺ [M+H]⁺, 693.2474; found, 693.2469.

### Example 53

### Synthesis of N-(2-(4-(4-chloro-1-(4-hydroxyphenyl)-2-phenylbut-1-en-1-yl)phenoxy)ethyl)-4-((2-(2,6-dioxopiperidin-3-yl)-1-oxoi soindolin-4-yl)amino)butanamide (SIAIS251030)

Referring to the procedures of Example 29, under appropriate conditions that will be recognized by one skilled in the art, the target compound (SIAIS251030) was prepared by using Toremifene derivative B (4-(1-(4-(2-aminoethoxy)phenyl)-4-chloro-2-phenylbut-1-en-1-yl)phenol) and intermediate LM (SIAIS1204085) as starting materials. The target compound SIAIS251030 was obtained as yellow solid (9.3 mg, 51% yield). ¹H NMR (500 MHz, DMSO) δ 11.00 (s, 1H), 9.76-8.95 (m, 1H), 8.07 (d, J=42.2 Hz, 1H), 7.28-7.23 (m, 1H), 7.22-7.10 (m, 7H), 7.06 (d, J=8.5 Hz, 1H), 6.95-6.92 (m, 2H), 6.79-6.69 (m, 3H), 6.60 (t, J=8.0 Hz, 2H), 6.41 (d, J=8.6 Hz, 1H), 5.11 (dd, J=13.2, 5.1 Hz, 1H), 4.25-4.20 (m, 1H), 4.17-4.10 (m, 1H), 4.00 (t, J=5.5 Hz, 1H), 3.84-3.81 (m, 1H), 3.65-3.41 (m, 5H), 3.37-3.30 (m, 1H), 3.12-3.08 (m, 2H), 2.96-2.81 (m, 3H), 2.63-2.59 (m, 1H), 2.32-2.27 (m, 1H), 2.06-1.97 (m, 1H), 1.86-1.73 (m, 2H). HRMS (ESI) m/z: calcd for C₄₁H₄₂ClN₄O₆⁺ [M+H]⁺, 721.2787; found, 721.2781.

### Example 54

### Synthesis of N-(2-(4-(4-chloro-1-(4-hydroxyphenyl)-2-phenylbut-1-en-1-yl)phenoxy)ethyl)-5-((2-(2,6-dioxopiperidin-3-yl)-1-oxoisoindolin-4-yl)amino)pentanamide (SIAIS251031)

Referring to the procedures of Example 29, under appropriate conditions that will be recognized by one skilled in the art, the target compound (SIAIS251031) was prepared by using Toremifene derivative B (4-(1-(4-(2-aminoethoxy)phenyl)-4-chloro-2-phenylbut-1-en-1-yl)phenol) and intermediate LM (SIAIS1210133) as starting materials. The target compound SIAIS251031 was obtained as yellow solid (8.8 mg, 47% yield). ¹H NMR (500 MHz, DMSO) δ 11.00 (s, 1H), 8.10-7.92 (m, 1H), 7.28 (t, J=7.6 Hz, 1H), 7.24-7.09 (m, 7H), 7.05 (d, J=8.4 Hz, 1H), 6.94 (d, J=7.7 Hz, 2H), 6.81-6.69 (m, 3H), 6.61-6.58 (m, 2H), 6.40 (d, J=8.5 Hz, 1H), 5.11 (dd, J=13.2, 5.0 Hz, 1H), 4.23 (dd, J=17.1, 3.3 Hz, 1H), 4.13 (dd, J=17.1, 3.5 Hz, 1H), 4.00 (t, J=5.4 Hz, 1H), 3.84-3.80 (m, 1H), 3.79-3.56 (m, 2H), 3.42 (d, J=6.3 Hz, 3H), 3.31 (d, J=5.5 Hz, 1H), 3.11-3.06 (m, 2H), 2.99-2.81 (m, 3H), 2.63-2.59 (m, 1H), 2.36-2.25 (m, 1H), 2.03-2.01 (m, 1H), 1.63-1.46 (m, 4H). HRMS (ESI) m/z: calcd for C₄₂H₄₄ClN₄O₆⁺ [M+H]⁺, 735.2944; found, 735.2938.

### Example 55

### Synthesis of N-(2-(4-(4-chloro-1-(4-hydroxyphenyl)-2-phenylbut-1-en-1-yl)phenoxy)ethyl)-6-((2-(2,6-dioxopiperidin-3-yl)-1-oxoisoindolin-4-yl)amino)hexanamide (SIAIS251032)

Referring to the procedures of Example 29, under appropriate conditions that will be recognized by one skilled in the art, the target compound (SIAIS251032) was prepared by using Toremifene derivative B (4-(1-(4-(2-aminoethoxy)phenyl)-4-chloro-2-phenylbut-1-en-1-yl)phenol) and intermediate LM (SIAIS1204061) as starting materials. The target compound SIAIS251032 was obtained as yellow solid (6.6 mg, 35% yield). ¹H NMR (500 MHz, DMSO) δ 11.00 (d, J=5.4 Hz, 1H), 8.14-7.92 (m, 1H), 7.31-7.10 (m, 8H), 7.07-7.04 (m, 1H), 6.96-6.93 (m, 2H), 6.78-6.70 (m, 3H), 6.62-6.58 (m, 2H), 6.42-6.39 (m, 1H), 5.15-5.07 (m, 1H), 4.25-4.22 (m, 1H), 4.17-4.09 (m, 1H), 4.00 (d, J=5.5 Hz, 1H), 3.82 (d, J=5.8 Hz, 1H), 3.73-3.51 (m, 2H), 3.46-3.42 (m, 3H), 3.33-3.31 (m, 1H), 3.12-3.08 (m, 2H), 2.97-2.81 (m, 3H), 2.63-2.59 (m, 1H), 2.36-2.28 (m, 1H), 2.16-2.09 (m, 2H), 2.03-2.02 (m, 1H), 1.62-1.50 (m, 4H). HRMS (ESI) m/z: calcd for C₄₃H₄₆ClN₄O₆⁺ [M+H]⁺, 749.3100; found, 749.3096.

### Example 56

### Synthesis of N-(2-(4-(4-chloro-1-(4-hydroxyphenyl)-2-phenylbut-1-en-1-yl)phenoxy)ethyl)-7-((2-(2,6-dioxopiperidin-3-yl)-1-oxoisoindolin-4-yl)amino)heptanamide (SIAIS251033)

Referring to the procedures of Example 29, under appropriate conditions that will be recognized by one skilled in the art, the target compound (SIAIS251033) was prepared by using Toremifene derivative B (4-(1-(4-(2-aminoethoxy)phenyl)-4-chloro-2-phenylbut-1-en-1-yl)phenol) and intermediate LM (SIAIS1204063) as starting materials. The target compound SIAIS251033 was obtained as yellow solid (6.8 mg, 35% yield). ¹H NMR (500 MHz, DMSO) δ 11.00 (s, 1H), 8.00 (dt, J=41.7, 5.4 Hz, 1H), 7.28 (td, J=7.8, 1.7 Hz, 1H), 7.23-7.08 (m, 7H), 7.05 (d, J=8.5 Hz, 1H), 6.94 (d, J=8.4 Hz, 2H), 6.79-6.69 (m, 3H), 6.62-6.56 (m, 2H), 6.42-6.38 (m, 1H), 5.11 (dd, J=13.3, 5.1 Hz, 1H), 4.24 (d, J=17.2 Hz, 1H), 4.14 (dd, J=17.2, 1.7 Hz, 1H), 3.99 (t, J=5.6 Hz, 1H), 3.82 (t, J=5.6 Hz, 1H), 3.74-3.54 (m, 2H), 3.44-3.39 (m, 3H), 3.31 (dd, J=11.1, 5.5 Hz, 1H), 3.09 (dd, J=13.9, 6.9 Hz, 2H), 2.96-2.84 (m, 3H), 2.63-2.59 (m, 1H), 2.34-2.25 (m, 1H), 2.02-2.01 (m, 1H), 1.57-1.44 (m, 4H), 1.39-1.23 (m, 4H). HRMS (ESI) m/z: calcd for C₄₄H₄₈ClN₄O₆⁺[M+H]⁺, 763.3257; found, 763.3252.

### Example 57

### Synthesis of (2S,4R)-14(S)-2-(3-(4-(3-((2-(44(Z)-4-chloro-1,2-diphenylbut-1-en-1-yl)phenoxy)ethyl)(methyl)amino)-3-oxopropyl)piperazin-1-yl)prop anamido)-3,3-dimethylbutanoyl)-4-hydroxy-N-(4-(4-methylthiazol-5-yl)benzyl)pyrrolidine-2-carboxamide (SIAIS208105)

Referring to the procedures of Example 1, under appropriate conditions that will be recognized by one skilled in the art, the target compound (SIAIS208105) was prepared by using Toremifene derivative A ((Z)-2-(4-(4-chloro-1,2-diphenylbut-1-en-1-yl)phenoxy)-N-methylethan-1-amine) and intermediate LM (SIAIS1213011) as starting materials. The target compound SIAIS208105 was obtained as white solid (5.5 mg, 23% yield). ¹H NMR (500 MHz, MeOD) δ 9.80 (s, 1H), 7.59-7.53 (m, 2H), 7.52-7.48 (m, 2H), 7.20-7.13 (m, 8H), 6.88-6.84 (m, 1H), 6.83-6.78 (m, 2H), 6.70 (dd, J=6.9, 4.8 Hz, 1H), 6.64 (d, J=8.8 Hz, 1H), 6.58-6.56 (m, 1H), 4.59-4.51 (m, 4H), 4.40 (d, J=15.8 Hz, 1H), 4.24-4.18 (m, 1H), 4.05 (t, J=5.0 Hz, 1H), 4.02-3.95 (m, 2H), 3.81-3.66 (m, 5H), 3.58-3.50 (m, 5H), 3.41-3.38 (m, 3H), 3.12-3.11 (m, 2H), 3.01-2.96 (m, 2H), 2.94-2.86 (m, 8H), 2.58 (s, 3H), 2.27-2.23 (m, 1H), 2.12-2.02 (m, 1H), 1.07-1.03 (m, 9H). HRMS (ESI) m/z: calcd for C₅₇H₇₁ClN₇O₆S⁺ [M+H]⁺, 1016.4870; found, 1016.4875.

### Example 58

### Synthesis of (2S,4R)-14(S)-2-(3-(4-(3-((2-(4-(4-chloro-1-(4-hydroxyphenyl)-2-phenylbut-1-en-1-yl)phenoxy)ethyl)amino)-3-oxopropyl)piperazin-1-yl)prop anamido)-3,3-dimethylbutanoyl)-4-hydroxy-N-(4-(4-methylthiazol-5-yl)benzyl)pyrrolidine-2-carboxamide (SIAIS208107)

Referring to the procedures of Example 29, under appropriate conditions that will be recognized by one skilled in the art, the target compound (SIAIS208107) was prepared by using Toremifene derivative B (4-(1-(4-(2-aminoethoxy)phenyl)-4-chloro-2-phenylbut-1-en-1-yl)phenol) and intermediate LM (SIAIS1213011) as starting materials. The target compound SIAIS208107 was obtained as white solid (5.7 mg, 22% yield). ¹H NMR (500 MHz, MeOD) δ 9.67 (s, 1H), 7.58-7.52 (m, 2H), 7.49 (d, J=7.9 Hz, 2H), 7.22-7.07 (m, 7H), 6.95 (d, J=8.6 Hz, 1H), 6.79 (dd, J=8.6, 1.3 Hz, 2H), 6.69-6.65 (m, 1H), 6.57 (d, J=8.8 Hz, 1H), 6.41 (d, J=8.7 Hz, 1H), 4.59-4.51 (m, 4H), 4.45-4.36 (m, 1H), 4.10 (t, J=5.2 Hz, 1H), 3.97 (d, J=11.0 Hz, 1H), 3.92 (t, J=5.3 Hz, 1H), 3.80-3.45 (m, 16H), 3.40 (t, J=7.4 Hz, 2H), 2.96-2.88 (m, 2H), 2.87-2.82 (m, 2H), 2.78 (t, J=6.5 Hz, 1H), 2.56 (s, 3H), 2.31-2.19 (m, 1H), 2.12-2.03 (m, 1H), 1.07-1.03 (m, 9H). HRMS (ESI) m/z: calcd for C₅₆H₆₉ClN₇O₇S⁺ [M+H]⁺, 1018.4662; found, 1018.4654.

### Example 59

### Synthesis of (2S,4R)-1-((S)-2-(3-(4-(34(2-(4-((Z)-4-chloro-1,2-diphenylbut-1-en-1-yl)phenoxy)ethyl)(methyl)amino)-3-oxopropyl)phenyl)prop anamido)-3,3-dimethylbutanoyl)-4-hydroxy-N-(4-(4-methylthiazol-5-yl)benzyl)pyrrolidine-2-c arboxamide (SIAIS208125)

Referring to the procedures of Example 1, under appropriate conditions that will be recognized by one skilled in the art, the target compound (SIAIS208125) was prepared by using Toremifene derivative A ((Z)-2-(4-(4-chloro-1,2-diphenylbut-1-en-1-yl)phenoxy)-N-methylethan-1-amine) and intermediate LM (SIAIS1213061) as starting materials. The target compound SIAIS208125 was obtained as white solid (6.3 mg, 27% yield). ¹H NMR (500 MHz, MeOD) δ 9.14 (s, 1H), 7.48 (d, J=8.4 Hz, 2H), 7.45-7.41 (m, 2H), 7.39-7.35 (m, 2H), 7.30-7.27 (m, 3H), 7.19-7.13 (m, 5H), 7.09 (d, J=8.1 Hz, 3H), 7.04 (d, J=8.1 Hz, 1H), 6.81-6.76 (m, 2H), 6.56-6.53 (m, 1H), 6.49-6.47 (m, 1H), 4.61-4.45 (m, 4H), 4.35 (d, J=15.6 Hz, 1H), 3.95 (t, J=5.4 Hz, 1H), 3.92-3.88 (m, 2H), 3.79-3.72 (m, 1H), 3.64-3.60 (m, 2H), 3.41-3.37 (m, 2H), 2.95-2.88 (m, 5H), 2.86-2.80 (m, 4H), 2.71-2.67 (m, 1H), 2.62-2.45 (m, 6H), 2.27-2.18 (m, 1H), 2.12-2.01 (m, 1H), 0.94-0.91 (m, 9H). HRMS (ESI) m/z: calcd for C₅₉H₆₇ClN₅O₆S⁺ [M+H]⁺, 1008.4495; found, 1008.4490.

### Example 60

### Synthesis of (2S,4R)-1-((S)-2-(3-(4-(3-((2-(4-(4-chloro-1-(4-hydroxyphenyl)-2-phenylbut-1-en-1-yl)phenoxy)ethyl)amino)-3-oxopropyl)phenyl)propanamido)-3,3-dimethylbutanoyl)-4-hydroxy-N-(4-(4-methylthiazol-5-yl)benzyl)pyrrolidine-2-carboxamide (SIAIS208127)

Referring to the procedures of Example 29, under appropriate conditions that will be recognized by one skilled in the art, the target compound (SIAIS208127) was prepared by using Toremifene derivative B (4-(1-(4-(2-aminoethoxy)phenyl)-4-chloro-2-phenylbut-1-en-1-yl)phenol) and intermediate LM (SIAIS1213061) as starting materials. The target compound SIAIS208127 was obtained as white solid (6.7 mg, 28% yield). ¹H NMR (500 MHz, MeOD) δ 9.15 (s, 1H), 7.49-7.46 (m, 2H), 7.43-7.41 (m, 2H), 7.22-7.16 (m, 3H), 7.15-7.06 (m, 8H), 6.91 (d, J=8.7 Hz, 1H), 6.80-6.76 (m, 2H), 6.68-6.65 (m, 1H), 6.54 (d, J=8.8 Hz, 1H), 6.43-6.40 (m, 1H), 4.62-4.47 (m, 4H), 4.34 (d, J=15.5 Hz, 1H), 3.98 (t, J=5.4 Hz, 1H), 3.89 (d, J=10.6 Hz, 1H), 3.81-3.76 (m, 2H), 3.52 (t, J=5.3 Hz, 1H), 3.42-3.38 (m, 2H), 2.96-2.77 (m, 7H), 2.59-2.40 (m, 7H), 2.27-2.19 (m, 1H), 2.11-2.01 (m, 1H), 0.94-0.92 (m, 9H). HRMS (ESI) m/z: calcd for C₅₈H₆₅ClN₅O₇S⁺ [M+H]⁺, 1010.4288; found, 1010.4283.

### Example 61

### Synthesis of (Z)—N-(2-(4-(4-chloro-1,2-diphenylbut-1-en-1-yl)phenoxy)ethyl)-3-(4-(2-((2-(2,6-dioxopiperidin-3-yl)-1,3-dioxoisoindolin-4-yl)amino)ethyl)piperazin-1-yl)-N-methylpropanamide (SIAIS208135)

Referring to the procedures of Example 1, under appropriate conditions that will be recognized by one skilled in the art, the target compound (SIAIS208135) was prepared by using Toremifene derivative A ((Z)-2-(4-(4-chloro-1,2-diphenylbut-1-en-1-yl)phenoxy)-N-methylethan-1-amine) and intermediate LM (SIAIS208130) as starting materials. The target compound SIAIS208135 was obtained as yellow solid (10.5 mg, 36% yield). ¹H NMR (500 MHz, DMSO) δ 11.10 (s, 1H), 7.66-7.58 (m, 1H), 7.40 (t, J=7.6 Hz, 2H), 7.33-7.27 (m, 3H), 7.25-7.12 (m, 6H), 7.09 (d, J=7.1 Hz, 1H), 6.82-6.74 (m, 2H), 6.68 (d, J=8.8 Hz, 1H), 6.62 (d, J=8.9 Hz, 1H), 5.06 (dd, J=12.7, 5.4 Hz, 1H), 4.00 (t, J=5.2 Hz, 1H), 3.93 (t, J=5.7 Hz, 1H), 3.81-3.40 (m, 16H), 3.29-3.12 (m, 3H), 3.04-2.82 (m, 8H), 2.63-2.52 (m, 2H), 2.07-1.98 (m, 1H). HRMS (ESI) m/z: calcd for C₄₇H₅₂ClN₆O₆⁺ [M+H]⁺, 831.3631; found, 831.3638.

### Example 62

### Synthesis of N-(2-(4-(4-chloro-1-(4-hydroxyphenyl)-2-phenylbut-1-en-1-yl)phenoxy)ethyl)-3-(4-(2-((2-(2,6-dioxopiperidin-3-yl)-1,3-dioxoisoindolin-4-yl)amino)ethyl)piperazin-1-yl)prop anamide (S IAIS 208137)

Referring to the procedures of Example 29, under appropriate conditions that will be recognized by one skilled in the art, the target compound (SIAIS208137) was prepared by using Toremifene derivative B (4-(1-(4-(2-aminoethoxy)phenyl)-4-chloro-2-phenylbut-1-en-1-yl)phenol) and intermediate LM (SIAIS208130) as starting materials. The target compound SIAIS208137 was obtained as yellow solid (9.8 mg, 31% yield). ¹H NMR (500 MHz, DMSO) δ 11.09 (s, 1H), 9.34 (d, J=126.4 Hz, 1H), 8.27 (d, J=52.4 Hz, 1H), 7.63-7.54 (m, 1H), 7.23-7.16 (m, 3H), 7.14-7.12 (m, 3H), 7.10-7.01 (m, 3H), 6.95 (d, J=8.7 Hz, 1H), 6.76 (d, J=8.6 Hz, 1H), 6.72 (d, J=8.8 Hz, 2H), 6.60 (d, J=7.9 Hz, 2H), 6.40 (d, J=8.7 Hz, 1H), 5.06 (dd, J=12.8, 5.4 Hz, 1H), 4.01 (t, J=5.3 Hz, 1H), 3.83 (t, J=5.4 Hz, 1H), 3.44-3.41 (m, 3H), 2.92-2.82 (m, 3H), 2.64-2.22 (m, 17H), 2.02-1.91 (m, 3H). HRMS (ESI) m/z: calcd for C₄₆H₅₀ClN₆O₇⁺ [M+H]⁺, 833.3424; found, 833.3421.

### Example 63

### Synthesis of (2S,4R)-1-((S)-2-(4-(4-(2-(4-(4-chloro-1-(4-hydroxyphenyl)-2-phenylbut-1-en-1-yl)phenoxy)ethyl)piperazin-1-yl)-4-oxobutanamido)-3,3-dimethylbutanoyl)-4-hydroxy-N-(4-(4-methylthi azol-5-yl)benzyl)pyrrolidine-2-c arboxamide (SIAIS 251041)

According to scheme 7, Toremifene derivative C (4-(4-chloro-2-phenyl-1-(4-(2-(piperazin-1-yflethoxy)phenyl)but-1-en-1-yOphenol; 0.0216 mmol, 1 equiv) and intermediate LM (SIAIS074011; 0.0216 mmol, 1 equiv), HOAt (0.0432 mmol, 2 equiv), EDCI (0.0432 mmol, 2 equiv), anhydrous DMF (2 mL), and NMM (0.108 mmol, 5 equiv) were added sequentially to a reaction flask at RT. The resulting reaction mixture was stirred at room temperature overnight. After the reaction was complete as detected by LC-MS, the resulting mixture was purified by preparative HPLC (eluent (v/v): acetonitrile/(water+0.05% HCl)=10%-100%). The acetonitrile was removed by rotary evaporation, and the resulting residue was lyophilized to yield the target product SIAIS251041 as white solid (11.4 mg, 54% yield). ¹H NMR (500 MHz, MeOD) δ 8.99 (s, 1H), 7.49 (d, J=8.3 Hz, 2H), 7.45 (d, J=8.3 Hz, 2H), 7.29 (d, J=8.7 Hz, 1H), 7.23-7.19 (m, 2H), 7.17-7.15 (m, 3H), 7.12-7.11 (m, 1H), 7.08 (d, J=8.8 Hz, 1H), 6.87 (d, J=8.8 Hz, 1H), 6.81 (d, J=8.6 Hz, 1H), 6.72-6.67 (m, 2H), 6.46-6.40 (m, 1H), 4.63-4.49 (m, 4H), 4.47-4.44 (m, 1H), 4.39 (d, J=15.6 Hz, 1H), 4.28 (s, 1H), 3.90 (t, J=9.0 Hz, 1H), 3.83-3.48 (m, 11H), 3.42 (t, J=7.4 Hz, 2H), 2.96 (t, J=7.4 Hz, 1H), 2.91 (t, J=7.5 Hz, 1H), 2.76-2.55 (m, 4H), 2.50 (s, 3H), 2.24-2.22 (m, 1H), 2.14-2.07 (m, 1H), 1.09-0.96 (m, 9H). HRMS (ESI) m/z: calcd for C₅₄H₆₄ClN₆O₇S⁺ [M+H]⁺, 975.4240; found, 975.4233.

### Example 64

### Synthesis of (2S,4R)-1-((S)-2-(5-(4-(2-(4-(4-chloro-1-(4-hydroxyphenyl)-2-phenylbut-1-en-1-yl)phenoxy)ethyl)piperazin-1-yl)-5-oxopentanamido)-3,3-dimethylbutanoyl)-4-hydroxy-N-(4-(4-methylthi azol-5-yl)benzyl)pyrrolidine-2-c arboxamide (SIAIS 251042)

Referring to the procedures of Example 63, under appropriate conditions that will be recognized by one skilled in the art, the target compound (SIAIS251042) was prepared by using Toremifene derivative C (4-(4-chloro-2-phenyl-1-(4-(2-(piperazin-1-yl)ethoxy)phenyl)but-1-en-1-yl)phenol) and intermediate LM (SIAIS074012) as starting materials. The target compound SIAIS251042 was obtained as white solid (10.8 mg, 51% yield). ¹H NMR (500 MHz, MeOD) δ 9.04 (d, J=18.7 Hz, 1H), 7.49 (d, J=7.6 Hz, 2H), 7.44 (d, J=7.8 Hz, 2H), 7.29-7.26 (m, 1H), 7.23-7.19 (m, 2H), 7.18-7.14 (m, 3H), 7.13-7.10 (m, 1H), 7.08 (d, J=8.7 Hz, 1H), 6.87-6.84 (m, 1H), 6.81 (d, J=8.5 Hz, 1H), 6.71-6.67 (m, 2H), 6.47-6.40 (m, 1H), 4.65-4.49 (m, 4H), 4.48-4.44 (m, 1H), 4.40 (dd, J=15.5, 4.5 Hz, 1H), 4.28 (d, J=4.1 Hz, 1H), 3.98-3.92 (m, 1H), 3.90-3.48 (m, 11H), 3.42 (t, J=7.4 Hz, 2H), 2.96 (t, J=7.4 Hz, 1H), 2.93-2.90 (m, 1H), 2.54-2.43 (m, 5H), 2.40-2.35 (m, 2H), 2.28-2.19 (m, 1H), 2.12-2.07 (m, 1H), 1.98-1.86 (m, 2H), 1.06-1.03 (m, 9H). HRMS (ESI) m/z: calcd for C₅₅H₆₆ClN₆O₇S⁺ [M+H]⁺, 989.4397; found, 989.4394.

### Example 65

### Synthesis of (2S,4R)-1-((S)-2-(6-(4-(2-(4-(4-chloro-1-(4-hydroxyphenyl)-2-phenylbut-1-en-1-yl)phenoxy)ethyl)piperazin-1-yl)-6-oxohexanamido)-3,3-dimethylbutanoyl)-4-hydroxy-N-(4-(4-methylthi azol-5-yl)benzyl)pyrrolidine-2-c arboxamide (S IAIS 251043)

Referring to the procedures of Example 63, under appropriate conditions that will be recognized by one skilled in the art, the target compound (SIAIS251043) was prepared by using Toremifene derivative C (4-(4-chloro-2-phenyl-1-(4-(2-(piperazin-1-yl)ethoxy)phenyl)but-1-en-1-yl)phenol) and intermediate LM (SIAIS074013) as starting materials. The target compound SIAIS251043 was obtained as white solid (11.9 mg, 55% yield). ¹H NMR (500 MHz, MeOD) δ 9.17-9.11 (m, 1H), 7.51-7.48 (m, 2H), 7.46 (d, J=8.2 Hz, 2H), 7.29-7.27 (m, 1H), 7.23-7.19 (m, 2H), 7.18-7.13 (m, 3H), 7.13-7.10 (m, 1H), 7.08 (d, J=8.8 Hz, 1H), 6.87 (d, J=8.8 Hz, 1H), 6.83-6.79 (m, 1H), 6.72-6.66 (m, 2H), 6.46-6.42 (m, 1H), 4.66-4.49 (m, 4H), 4.48-4.44 (m, 1H), 4.42-4.39 (m, 1H), 4.31-4.27 (m, 1H), 3.93 (d, J=11.1 Hz, 1H), 3.87-3.51 (m, 11H), 3.42 (t, J=7.5 Hz, 2H), 2.96 (t, J=7.3 Hz, 1H), 2.91 (t, J=7.4 Hz, 1H), 2.55-2.43 (m, 5H), 2.38-2.29 (m, 2H), 2.26-2.22 (m, 1H), 2.14-2.05 (m, 1H), 1.72-1.62 (m, 4H), 1.06-1.03 (m, 9H). HRMS (ESI) m/z: calcd for C₅₆H₆₈ClN₆O₇S⁺ [M+H]⁺, 1003.4553; found, 1003.4553.

### Example 66

### Synthesis of (2S,4R)-14(S)-2-(8-(4-(2-(4-(4-chloro-1-(4-hydroxyphenyl)-2-phenylbut-1-en-1-yl)phenoxy)ethyl)piperazin-1-yl)-8-oxooctanamido)-3,3-dimethylbutanoyl)-4-hydroxy-N-(4-(4-methylthiazol-5-yl)benzyl)pyrrolidine-2-c arboxamide (SIAIS 251045)

Referring to the procedures of Example 63, under appropriate conditions that will be recognized by one skilled in the art, the target compound (SIAIS251045) was prepared by using Toremifene derivative C (4-(4-chloro-2-phenyl-1-(4-(2-(piperazin-1-yl)ethoxy)phenyl)but-1-en-1-yl)phenol) and intermediate LM (SIAIS074015) as starting materials. The target compound SIAIS251045 was obtained as white solid (12.7 mg, 57% yield). ¹H NMR (500 MHz, MeOD) 9.50-9.47 (m, 1H), 7.54 (d, J=8.1 Hz, 2H), 7.51-7.48 (m, 2H), 7.29 (d, J=8.6 Hz, 1H), 7.25-7.18 (m, 2H), 7.18-7.14 (m, 3H), 7.11 (d, J=8.4 Hz, 1H), 7.08 (d, J=8.6 Hz, 1H), 6.87 (d, J=8.7 Hz, 1H), 6.81 (d, J=8.5 Hz, 1H), 6.72-6.67 (m, 2H), 6.44 (d, J=8.6 Hz, 1H), 4.67-4.50 (m, 4H), 4.49-4.45 (m, 1H), 4.41 (d, J=15.6 Hz, 1H), 4.30-4.28 (m, 1H), 3.93 (d, J=11.0 Hz, 1H), 3.86-3.50 (m, 11H), 3.42 (t, J=7.4 Hz, 2H), 2.96 (t, J=7.4 Hz, 1H), 2.91 (t, J=7.4 Hz, 1H), 2.57-2.56 (m, 3H), 2.50-2.41 (m, 2H), 2.38-2.19 (m, 3H), 2.14-2.03 (m, 1H), 1.69-1.55 (m, 4H), 1.46-1.33 (m, 4H), 1.08-0.89 (m, 9H). HRMS (ESI) m/z: calcd for C₅₈H₇₂ClN₆O₇S⁺ [M+H]⁺, 1031.4866; found, 1031.4858.

### Example 67

### Synthesis of (2S,4R)-1-((S)-2-(9-(4-(2-(4-(4-chloro-1-(4-hydroxyphenyl)-2-phenylbut-1-en-1-yl)phenoxy)ethyl)piperazin-1-yl)-9-oxononanamido)-3,3-dimethylbutanoyl)-4-hydroxy-N-(4-(4-methylthiazol-5-yl)benzyl)pyrrolidine-2-carboxamide (SIAIS251046)

Referring to the procedures of Example 63, under appropriate conditions that will be recognized by one skilled in the art, the target compound (SIAIS251046) was prepared by using Toremifene derivative C (4-(4-chloro-2-phenyl-1-(4-(2-(piperazin-1-yl)ethoxy)phenyl)but-1-en-1-yl)phenol) and intermediate LM (SIAIS074016) as starting materials. The target compound SIAIS251046 was obtained as white solid (12.1 mg, 54% yield). ¹H NMR (500 MHz, MeOD) δ 9.55-9.34 (m, 1H), 7.54 (d, J=8.2 Hz, 2H), 7.49 (d, J=8.1 Hz, 2H), 7.30-7.27 (m, 1H), 7.25-7.19 (m, 2H), 7.18-7.14 (m, 3H), 7.12-7.11 (m, 1H), 7.09-7.07 (m, 1H), 6.87 (d, J=8.8 Hz, 1H), 6.83-6.78 (m, 1H), 6.73-6.66 (m, 2H), 6.45-6.42 (m, 1H), 4.68-4.50 (m, 4H), 4.49-4.45 (m, 1H), 4.41 (d, J=15.6 Hz, 1H), 4.32-4.27 (m, 1H), 3.93 (d, J=11.1 Hz, 1H), 3.86-3.49 (m, 11H), 3.42 (t, J=7.4 Hz, 2H), 2.96 (t, J=7.4 Hz, 1H), 2.92 (t, J=7.4 Hz, 1H), 2.56 (s, 3H), 2.51-2.42 (m, 2H), 2.36-2.20 (m, 3H), 2.12-2.07 (m, 1H), 1.67-1.58 (m, 4H), 1.42-1.33 (m, 6H), 1.05-1.03 (m, 9H). HRMS (ESI) m/z: calcd for C₅₉H₇₄ClN₆O₇S⁺ [M+H]⁺, 1045.5023; found, 1045.5021.

### Example 68

### Synthesis of (2S,4R)-14(S)-2-(10-(4-(2-(4-(4-chloro-1-(4-hydroxyphenyl)-2-phenylbut-1-en-1-yl)phenoxy)ethyl)piperazin-1-yl)-10-oxodecanamido)-3,3-dimethylbutanoyl)-4-hydroxy-N-(4-(4-methylthiazol-5-yl)benzyl)pyrrolidine-2-carboxamide (SIAIS251047)

Referring to the procedures of Example 63, under appropriate conditions that will be recognized by one skilled in the art, the target compound (SIAIS251047) was prepared by using Toremifene derivative C (4-(4-chloro-2-phenyl-1-(4-(2-(piperazin-1-yl)ethoxy)phenyl)but-1-en-1-yl)phenol) and intermediate LM (SIAIS074019) as starting materials. The target compound SIAIS251047 was obtained as white solid (11.7 mg, 51% yield). ¹H NMR (500 MHz, MeOD) δ 9.16-9.04 (m, 1H), 7.50 (d, J=8.3 Hz, 2H), 7.48-7.45 (m, 2H), 7.29 (d, J=8.7 Hz, 1H), 7.24-7.19 (m, 2H), 7.18-7.13 (m, 3H), 7.12-7.11 (m, 1H), 7.10-7.05 (m, 1H), 6.87 (d, J=8.8 Hz, 1H), 6.84-6.78 (m, 1H), 6.73-6.67 (m, 2H), 6.45-6.42 (m, 1H), 4.67-4.50 (m, 4H), 4.49-4.45 (m, 1H), 4.39 (d, J=15.5 Hz, 1H), 4.31-4.26 (m, 1H), 3.93 (d, J=11.1 Hz, 1H), 3.86-3.51 (m, 11H), 3.42 (t, J=7.4 Hz, 2H), 2.96 (t, J=7.4 Hz, 1H), 2.91 (t, J=7.4 Hz, 1H), 2.52 (s, 3H), 2.49-2.42 (m, 2H), 2.38-2.20 (m, 3H), 2.12-2.08 (m, 1H), 1.67-1.57 (m, 4H), 1.40-1.31 (m, 8H), 1.05-1.03 (m, 9H). HRMS (ESI) m/z: calcd for C₆₀H₇₆ClN₆O₇S⁺ [M+H]⁺, 1059.5179; found, 1059.5175.

### Example 69

### Synthesis of (2S,4R)-1-((S)-2-(2-(2-(4-(2-(4-(4-chloro-1-(4-hydroxyphenyl)-2-phenylbut-1-en-1-yl)phenoxy)ethyl)piperazin-1-yl)-2-oxoethoxy)ac etamido)-3,3-dimethylbutanoyl)-4-hydroxy-N-(4-(4-methylthi azol-5-yl)benzyl)pyrrolidine-2-c arboxamide (SIAIS251048)

Referring to the procedures of Example 63, under appropriate conditions that will be recognized by one skilled in the art, the target compound (SIAIS251048) was prepared by using Toremifene derivative C (4-(4-chloro-2-phenyl-1-(4-(2-(piperazin-1-yl)ethoxy)phenyl)but-1-en-1-yl)phenol) and intermediate LM (SIAIS164112) as starting materials. The target compound SIAIS251048 was obtained as white solid (9.8 mg, 46% yield). ¹H NMR (500 MHz, MeOD) δ 9.22-9.17 (m, 1H), 7.54-7.43 (m, 4H), 7.29-7.26 (m, 1H), 7.24-7.19 (m, 2H), 7.18-7.14 (m, 3H), 7.13 -7.09 (m, 1H), 7.07 (d, J=8.7 Hz, 1H), 6.86 (t, J=7.0 Hz, 1H), 6.83-6.78 (m, 1H), 6.71-6.66 (m, 2H), 6.45-6.42 (m, 1H), 4.70-4.69 (m, 1H), 4.61-4.55 (m, 1H), 4.55-4.38 (m, 7H), 4.27 (s, 1H), 4.23-4.11 (m, 2H), 3.97-3.49 (m, 11H), 3.42 (t, J=7.4 Hz, 2H), 2.96 (t, J=7.3 Hz, 1H), 2.91 (t, J=7.4 Hz, 1H), 2.52 (s, 3H), 2.29-2.22 (m, 1H), 2.13-2.08 (m, 1H), 1.11-1.05 (m, 9H). HRMS (ESI) m/z: calcd for C₅₄H₆₄ClN₆O₈S⁺ [M+H]⁺, 991.4189; found, 991.4180.

### Example 70

### Synthesis of (2S,4R)-1-((S)-2-(3-(2-(3-(4-(2-(4-(4-chloro-1-(4-hydroxyphenyl)-2-phenylbut-1-en-1-yl)phenoxy)ethyl)piperazin-1-yl)-3-oxopropoxy)ethoxy)prop anamido)-3,3-dimethylbutanoyl)-4-hydroxy-N-(4-(4-methylthiazol-5-yl)benzyl)pyrrolidine-2-c arboxamide (SIAIS251049)

Referring to the procedures of Example 63, under appropriate conditions that will be recognized by one skilled in the art, the target compound (SIAIS251049) was prepared by using Toremifene derivative C (4-(4-chloro-2-phenyl-1-(4-(2-(piperazin-1-yl)ethoxy)phenyl)but-1-en-1-yl)phenol) and intermediate LM (SIAIS151002) as starting materials. The target compound SIAIS251049 was obtained as white solid (13.3 mg, 58% yield). ¹H NMR (500 MHz, MeOD) δ 9.06 (s, 1H), 7.49 (d, J=8.1 Hz, 2H), 7.46-7.44 (m, 2H), 7.28 (d, J=8.7 Hz, 1H), 7.23-7.19 (m, 2H), 7.17-7.15 (m, 3H), 7.11 (d, J=8.5 Hz, 1H), 7.08 (d, J=8.7 Hz, 1H), 6.87 (d, J=8.8 Hz, 1H), 6.81 (d, J=8.6 Hz, 1H), 6.72-6.67 (m, 2H), 6.43 (d, J=8.7 Hz, 1H), 4.67-4.65 (m, 1H), 4.61-4.56 (m, 2H), 4.53-4.45 (m, 3H), 4.42-4.38 (m, 1H), 4.30-4.28 (m, 1H), 3.90 (t, J=9.8 Hz, 1H), 3.83-3.69 (m, 6H), 3.67-3.46 (m, 11H), 3.42 (t, J=7.3 Hz, 2H), 2.96 (t, J=7.3 Hz, 1H), 2.91 (t, J=7.4 Hz, 1H), 2.81-2.44 (m, 7H), 2.27-2.19 (m, 1H), 2.14-2.03 (m, 2H), 1.09-0.89 (m, 9H). HRMS (ESI) m/z: calcd for C₅₈H₇₂ClN₆O₉S [M+H]⁺, 1063.4765; found, 1063.4762.

### Example 71

### Synthesis of (2S,4R)-14(S)-2-(tert-butyl)-16-(4-(2-(4-(4-chloro-1-(4-hydroxyphenyl)-2-phenylbut-1-en-1-yl)phenoxy)ethyl)piperazin-1-yl)-4,16-dioxo-7,10,13-trioxa-3-azahexadecanoyl)-4-hydroxy-N-(4-(4-methylthiazol-5-yl)benzyl)pyrrolidine-2-carboxamide (SIAIS251050)

Referring to the procedures of Example 63, under appropriate conditions that will be recognized by one skilled in the art, the target compound (SIAIS251050) was prepared by using Toremifene derivative C (4-(4-chloro-2-phenyl-1-(4-(2-(piperazin-1-yl)ethoxy)phenyl)but-1-en-1-yl)phenol) and intermediate LM (SIAIS151003) as starting materials. The target compound SIAIS251050 was obtained as white solid (13.0 mg, 54% yield). ¹H NMR (500 MHz, MeOD) δ 9.55-9.39 (m, 1H), 7.55-7.52 (m, 2H), 7.51-7.46 (m, 2H), 7.29 (d, J=8.6 Hz, 1H), 7.24-7.18 (m, 2H), 7.17-7.15 (m, 3H), 7.12-7.10 (m, 1H), 7.08 (d, J=8.7 Hz, 1H), 6.87 (d, J=8.7 Hz, 1H), 6.83-6.78 (m, 1H), 6.73-6.66 (m, 2H), 6.46-6.42 (m, 1H), 4.66-4.65 (m, 1H), 4.61-4.55 (m, 1H), 4.54-4.45 (m, 3H), 4.42 (d, J=15.7 Hz, 1H), 4.32-4.28 (m, 1H), 3.90 (t, J=8.8 Hz, 1H), 3.82-3.70 (m, 7H), 3.65-3.53 (m, 16H), 3.42 (t, J=7.4 Hz, 2H), 2.97-2.90 (m, 2H), 2.85-2.44 (m, 7H), 2.28-2.21 (m, 1H), 2.12-2.06 (m, 1H), 1.09-0.88 (m, 9H). HRMS (ESI) m/z: calcd for C₆₀H₇₆ClN₆O₁₀S⁺ [M+H]⁺, 1107.5027; found, 1107.5024.

### Example 72

### Synthesis of (2S,4R)-14(S)-2-(tert-butyl)-19-(4-(2-(4-(4-chloro-1-(4-hydroxyphenyl)-2-phenylbut-1-en-1-yl)phenoxy)ethyl)piperazin-1-yl)-4,19-dioxo-7,10,13,16-tetraoxa-3-azanonadecanoyl)-4-hydroxy-N-(4-(4-methylthiazol-5-yl)benzyl)pyrrolidine-2-carboxamide (SIAIS251051)

Referring to the procedures of Example 63, under appropriate conditions that will be recognized by one skilled in the art, the target compound (SIAIS251051) was prepared by using Toremifene derivative C (4-(4-chloro-2-phenyl-1-(4-(2-(piperazin-1-yl)ethoxy)phenyl)but-1-en-1-yl)phenol) and intermediate LM (SIAIS151008) as starting materials. The target compound SIAIS251051 was obtained as white solid (14.6 mg, 59% yield). ¹H NMR (500 MHz, MeOD) δ 9.55-9.49 (m, 1H), 7.54 (d, J=7.0 Hz, 2H), 7.51-7.47 (m, 2H), 7.28 (d, J=8.6 Hz, 1H), 7.23-7.19 (m, 2H), 7.18-7.13 (m, 3H), 7.12-7.06 (m, 2H), 6.87 (d, J=8.8 Hz, 1H), 6.83-6.78 (m, 1H), 6.73-6.65 (m, 2H), 6.47-6.41 (m, 1H), 4.66-4.62 (m, 1H), 4.60-4.57 (m, 1H), 4.55-4.45 (m, 3H), 4.41 (d, J=15.7 Hz, 1H), 4.32-4.27 (m, 1H), 3.92-3.89 (m, 1H), 3.79-3.69 (m, 8H), 3.68-3.51 (m, 19H), 3.42 (t, J=7.4 Hz, 2H), 3.02-2.41 (m, 9H), 2.29-2.22 (m, 1H), 2.13-2.05 (m, 1H), 1.08-1.01 (m, 9H). HRMS (ESI) m/z: calcd for C₆₂H₈₀ClN₆O₁₁S [M+H]⁺, 1151.5289; found, 1151.5287.

### Example 73

### Synthesis of N1-(2-(4-(1,2-bi s(4-hydroxyphenyl)but-1-en-1-yl)phenoxy)ethyl)-N9-((S)-1-((2S,4R)-4-hydroxy-24(4-(4-methylthiazol-5-yl)benzypc arb amoyl)pyrrolidin-1-yl)-3,3-dimethyl-1-oxobutan-2-yl)nonanediamide (S IAIS 208167)

According to scheme 7, Tamoxifene derivative A (4,4′-(1-(4-(2-aminoethoxy)phenyl)but-1-ene-1,2-diyl)diphenol; 0.03995 mmol, 1 equiv), intermediate LM (SIAIS074016; 0.03995 mmol, 1 equiv), HOAt (0.0799 mmol, 2 equiv), EDCI (0.0799 mmol, 2 equiv), anhydrous DMF (2 mL), and NMM (0.1998 mmol, 5 equiv) were added sequentially to a reaction flask at RT. The resulting reaction mixture was stirred at room temperature overnight. After the reaction was complete as detected by LC-MS, the resulting mixture was purified by preparative HPLC (eluent (v/v): acetonitrile/(water+0.05%HCl)=10%-100%). The acetonitrile was removed by rotary evaporation, and the residue was lyophilized to yield the target product SIAIS208167 as white solid (21.8 mg, 57% yield). ¹H NMR (500 MHz, MeOD) δ 9.75 (s, 1H), 7.57 (d, J=8.2 Hz, 2H), 7.53-7.50 (m, 2H), 7.11 (d, J=8.6 Hz, 1H), 7.00 (d, J=8.5 Hz, 1H), 6.95-6.89 (m, 3H), 6.80-6.74 (m, 2H), 6.69-6.65 (m, 1H), 6.60-6.58 (m, 3H), 6.44 (d, J=8.6 Hz, 1H), 4.65 (s, 1H), 4.62-4.56 (m, 2H), 4.51 (s, 1H), 4.40 (d, J=15.7 Hz, 1H), 4.07 (t, J=5.4 Hz, 1H), 3.94-3.91 (m, 2H), 3.82 (dd, J=10.9, 2.4 Hz, 1H), 3.58 (t, J=5.4 Hz, 1H), 3.49 (t, J=5.4 Hz, 1H), 2.58 (s, 3H), 2.49-2.38 (m, 2H), 2.30-2.16 (m, 5H), 2.12-2.06 (m, 1H), 1.65-1.55 (m, 4H), 1.33-1.31 (m, 6H), 1.05-1.03 (m, 9H), 0.92 (t, J=7.4 Hz, 3H). HRMS (ESI) m/z: calcd for C₅₅H₆₈N₅O₈S [M+H]⁺, 958.4783; found, 958.4777.

### Example 74

### Synthesis of N1-(2-(4-(1,2-bis(4-hydroxyphenyl)but-1-en-1-yl)phenoxy)ethyl)-N10-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)decanediamide (S IAIS 208168)

Referring to the procedures of Example 73, under appropriate conditions that will be recognized by one skilled in the art, the target compound (SIAIS208168) was prepared by using Tamoxifene derivative A (4,4′-(1-(4-(2-aminoethoxy)phenyl)but-1-ene-1,2-diyl)diphenol) and intermediate LM (SIAIS074019) as starting materials. The target compound SIAIS208168 was obtained as white solid (20.6 mg, 53% yield). ¹H NMR (500 MHz, MeOD) δ 9.67 (s, 1H), 7.56 (d, J=8.2 Hz, 2H), 7.52-7.49 (m, 2H), 7.11-7.09 (m, 1H), 7.03-6.98 (m, 1H), 6.95-6.88 (m, 3H), 6.80-6.74 (m, 2H), 6.69-6.64 (m, 1H), 6.62-6.56 (m, 3H), 6.46-6.41 (m, 1H), 4.65 (s, 1H), 4.61-4.57 (m, 2H), 4.51 (s, 1H), 4.39 (d, J=15.7 Hz, 1H), 4.07 (t, J=5.4 Hz, 1H), 3.94-3.91 (m, 2H), 3.82 (dd, J=11.0, 2.8 Hz, 1H), 3.58 (t, J=5.4 Hz, 1H), 3.49 (t, J=5.4 Hz, 1H), 2.58 (s, 3H), 2.47-2.41 (m, 2H), 2.32-2.17 (m, 5H), 2.12-2.07 (m, 1H), 1.61-1.60 (m, 4H), 1.31-1.30 (m, 8H), 1.05-1.03 (m, 9H), 0.92 (t, J=7.4 Hz, 3H). HRMS (ESI) m/z: calcd for C₅₆H₇₀N₅O₈S [M+H]⁺, 972.4940; found, 972.4932.

### Example 75

### Synthesis of N1-(2-(4-(1,2-bi s(4-hydroxyphenyl)but-1-en-1-yl)phenoxy)ethyl)-N11-((S)-1-((2S,4R)-4-hydroxy-2-((4-(4-methylthiazol-5-yl)benzyl)c arb amoyl)pyrrolidin-1-yl)-3,3-dimethyl-1-oxobutan-2-yl)undec anedi amide (SIAIS208169)

Referring to the procedures of Example 73, under appropriate conditions that will be recognized by one skilled in the art, the target compound (SIAIS208169) was prepared by using Tamoxifene derivative A (4,4′-(1-(4-(2-aminoethoxy)phenyl)but-1-ene-1,2-diyl)diphenol) and intermediate LM (SIAIS074020) as starting materials. The target compound SIAIS208169 was obtained as white solid (22.3 mg, 57% yield). ¹H NMR (500 MHz, MeOD) δ 9.65 (s, 1H), 7.54 (d, J=8.3 Hz, 2H), 7.50-7.47 (m, 2H), 7.11-7.06 (m, 1H), 7.00-6.96 (m, 1H), 6.93-6.87 (m, 3H), 6.77-6.71 (m, 2H), 6.66-6.62 (m, 1H), 6.59-6.54 (m, 3H), 6.44-6.39 (m, 1H), 4.63 (d, J=1.7 Hz, 1H), 4.58-4.55 (m, 2H), 4.49 (s, 1H), 4.37 (d, J=15.7 Hz, 1H), 4.05 (t, J=5.4 Hz, 1H), 3.93-3.88 (m, 2H), 3.84-3.74 (m, 1H), 3.56 (t, J=5.4 Hz, 1H), 3.49-3.43 (m, 1H), 2.55 (s, 3H), 2.45-2.38 (m, 2H), 2.30-2.14 (m, 5H), 2.09-2.04 (m, 1H), 1.65-1.52 (m, 4H), 1.28-1.27 (m, 10H), 1.03-1.01 (m, 9H), 0.90 (t, J=7.4 Hz, 3H). HRMS (ESI) m/z: calcd for C₅₇H₇₂N₅O₈S [M+H]⁺, 986.5096; found, 986.5095.

### Example 76

### Synthesis of N-(2-(4-(1,2-bi s(4-hydroxyphenyl)but-1-en-1-yl)phenoxy)ethyl)-3-(4-(2-((2-(2,6-dioxopiperidin-3-yl)-1,3-dioxoisoindolin-4-yl)amino)ethyl)piperazin-1-yl)propanamide (SIAIS208172)

Referring to the procedures of Example 73, under appropriate conditions that will be recognized by one skilled in the art, the target compound (SIAIS208172) was prepared by using Tamoxifene derivative A (4,4′-(1-(4-(2-aminoethoxy)phenyl)but-1-ene-1,2-diyl)diphenol) and intermediate LM (SIAIS208130) as starting materials. The target compound SIAIS208172 was obtained as yellow solid (17.9 mg, 55% yield). ¹H NMR (500 MHz, DMSO) δ 11.10 (s, 1H), 9.43-9.04 (m, 2H), 8.41 (d, J=35.1 Hz, 1H), 7.67-7.59 (m, 1H), 7.23 (d, J=7.8 Hz, 1H), 7.10 (d, J=6.9 Hz, 1H), 7.06 (d, J=8.6 Hz, 1H), 6.92 (t, J=8.8 Hz, 2H), 6.87 (d, J=8.5 Hz, 2H), 6.82 (s, 1H), 6.74-6.70 (m, 2H), 6.62-6.54 (m, 4H), 6.42-6.41 (m, 1H), 5.06 (dd, J=12.8, 5.4 Hz, 1H), 4.01 (t, J=5.5 Hz, 1H), 3.87 (t, J=5.4 Hz, 1H), 3.62-3.38 (m, 15H), 2.93-2.82 (m, 1H), 2.69-2.66 (m, 2H), 2.61 (s, 1H), 2.57-2.53 (m, 2H), 2.35-2.30 (m, 2H), 2.07-1.98 (m, 1H), 0.84 (t, J=7.4 Hz, 3H). HRMS (ESI) m/z: calcd for C₄₆H₅₁N₆O₈⁺ [M+H]⁺, 815.3763; found, 815.3760.

### Example 77

### Synthesis of (2S,4R)-1-((S)-16-(4-(1,2-bis(4-hydroxyphenyl)but-1-en-1-yl)phenoxy)-2-(tert-butyl)-4,13-dioxo-7,10-dioxa-3,14-diazahexadecanoyl)-4-hydroxy-N-(4-(4-methylthiazol-5-yl)benzyl)pyrrolidine-2-c arboxamide (SIAIS208173)

Referring to the procedures of Example 73, under appropriate conditions that will be recognized by one skilled in the art, the target compound (SIAIS208173) was prepared by using Tamoxifene derivative A (4,4′-(1-(4-(2-aminoethoxy)phenyl)but-1-ene-1,2-diyl)diphenol) and intermediate LM (SIAIS151002) as starting materials. The target compound SIAIS208173 was obtained as white solid (18.3 mg, 47% yield). ¹H NMR (500 MHz, MeOD) δ 9.77-9.76 (m, 1H), 7.54 (d, J=6.7 Hz, 2H), 7.52-7.46 (m, 2H), 7.09 (d, J=8.6 Hz, 1H), 6.98 (d, J=8.5 Hz, 1H), 6.91-6.89 (m, 3H), 6.75 (t, J=8.9 Hz, 2H), 6.67-6.62 (m, 1H), 6.60-6.54 (m, 3H), 6.42 (d, J=8.6 Hz, 1H), 4.65 (d, J=2.5 Hz, 1H), 4.57 (dd, J=11.1, 9.0 Hz, 2H), 4.49 (s, 1H), 4.37 (dd, J=15.6, 5.9 Hz, 1H), 4.05 (t, J=5.4 Hz, 1H), 3.90 (t, J=5.4 Hz, 2H), 3.79 (dd, J=11.0, 3.8 Hz, 1H), 3.74-3.62 (m, 4H), 3.60-3.46 (m, 6H), 2.56 (s, 3H), 2.53-2.36 (m, 6H), 2.25-2.18 (m, 1H), 2.09-2.04 (m, 1H), 1.03-1.01 (m, 9H), 0.90 (t, J=7.4 Hz, 3H). HRMS (ESI) m/z: calcd for C₅₄H₆₆N₅O₁₀S [M+H]⁺, 976.4525; found, 976.4518.

### Example 78

### Synthesis of (2S,4R)-1-((S)-19-(4-(1,2-bis(4-hydroxyphenyl)but-1-en-1-yl)phenoxy)-2-(tert-butyl)-4,16-dioxo-7,10,13-trioxa-3,17-diazanonadec anoyl)-4-hydroxy-N-(4-(4-methylthiazol-5-yl)benzyl)pyrrolidine-2-carboxamide (SIAIS208174)

Referring to the procedures of Example 73, under appropriate conditions that will be recognized by one skilled in the art, the target compound (SIAIS208174) was prepared by using Tamoxifene derivative A (4,4′-(1-(4-(2-aminoethoxy)phenyl)but-1-ene-1,2-diyl)diphenol) and intermediate LM (SIAIS151003) as starting materials. The target compound SIAIS208174 was obtained as white solid (18.7 mg, 46% yield). ¹H NMR (500 MHz, MeOD) δ 9.75 (s, 1H), 7.55 (d, J=8.2 Hz, 2H), 7.49 (d, J=7.9 Hz, 2H), 7.10-7.07 (m, 1H), 7.01-6.95 (m, 1H), 6.93-6.87 (m, 3H), 6.78-6.71 (m, 2H), 6.67-6.62 (m, 1H), 6.60-6.54 (m, 3H), 6.45-6.40 (m, 1H), 4.65 (s, 1H), 4.59-4.55 (m, 2H), 4.50-4.48 (m, 1H), 4.38 (dd, J=15.7, 2.4 Hz, 1H), 4.06 (t, J=5.4 Hz, 1H), 3.94-3.87 (m, 2H), 3.79 (dd, J=11.0, 3.8 Hz, 1H), 3.74-3.68 (m, 4H), 3.61-3.53 (m, 9H), 3.50 (t, J=5.4 Hz, 1H), 2.59-2.52 (m, 4H), 2.51-2.38 (m, 5H), 2.25-2.21 (m, 1H), 2.09-2.04 (m, 1H), 1.03-1.01 (m, 9H), 0.90 (t, J=7.4 Hz, 3H). HRMS (ESI) m/z: calcd for C₅₆H₇₀N₅O₁₁S [M+H]⁺, 1020.4787; found, 1020.4786.

### Example 79

### Synthesis of (2S,4R)-1-((S)-2-(tert-butyl)-19-(4-(4-chloro-1,2-bis(4-hydroxyphenyl)but-1-en-1-yl)phenoxy)-4,16-dioxo-7,10,13-trioxa-3,17-diazanonadecanoyl)-4-hydroxy-N-(4-(4-methylthiazol-5-yl)benzyl)pyrrolidine-2-carboxamide (SIAIS307146)

According to scheme 7, Toremifene derivative D (4,4′-(1-(4-(2-aminoethoxy)phenyl)-4-chlorobut-1-ene-1,2-diyOdiphenol; 0.0244 mmol, 1 equiv), intermediate LM (SIAIS151003; 0.0244 mmol, 1 equiv), HOAt (0.0488 mmol, 2 equiv), EDCI (0.0488 mmol, 2 equiv), anhydrous DMF (2 mL), and NMM (0.122 mmol, 5 equiv) were added sequentially to a reaction flask at room temperature. The resulting reaction mixture was stirred at room temperature overnight. After the reaction was complete as detected by LC-MS, the resulting mixture was purified by preparative HPLC (eluent (v/v): acetonitrile/(water+0.05%HCl)=10% -100%). The acetonitrile was removed by rotary evaporation, and the resulting residue was lyophilized to yield the target product SIAIS307146 as white solid (12.9 mg, 50% yield). ¹H NMR (500 MHz, MeOD) δ 9.67 (s, 1H), 7.54 (d, J=7.7 Hz, 2H), 7.48 (d, J=7.9 Hz, 2H), 7.17 (d, J=8.0 Hz, 1H), 7.06 (d, J=8.2 Hz, 1H), 6.97-6.91 (m, 3H), 6.77 (t, J=6.9 Hz, 2H), 6.67 (d, J=8.2 Hz, 1H), 6.60 (t, J=6.9 Hz, 3H), 6.43 (d, J=8.3 Hz, 1H), 4.64 (s, 1H), 4.61-4.53 (m, 2H), 4.49 (s, 1H), 4.37 (d, J=15.9 Hz, 1H), 4.07 (t, J=5.2 Hz, 1H), 3.94-3.86 (m, 2H), 3.79 (d, J=7.7 Hz, 1H), 3.70 (dt, J=17.6, 5.6 Hz, 4H), 3.59-3.54 (m, 9H), 3.51-3.47 (m, 1H), 3.41 (t, J=7.3 Hz, 2H), 2.89-2.84 (m, 2H), 2.59-2.41 (m, 4H), 2.45 (dt, J=12.2, 5.8 Hz, 3H), 2.25-2.18 (m, 1H), 2.11-2.02 (m, 1H), 1.03-1.01 (m, J=9.8 Hz, 9H). HRMS (ESI) m/z: calcd for C₅₆H₆₉ClN₅O₁₁S⁺ [M+H]⁺, 1054.4397; found, 1054.4391.

### Example 80

### Synthesis of N1-(2-(4-(4-chloro-1,2-bis(4-hydroxyphenyl)but-1-en-1-yflphenoxy)ethyl)-N16-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)-4,7,10,13-tetraoxahexadecanediamide (SIAIS 307147)

Referring to the procedures of Example 79, under appropriate conditions that will be recognized by one skilled in the art, the target compound (SIAIS307147) was prepared by using Toremifene derivative D (4,4′-(1-(4-(2-aminoethoxy)phenyl)-4-chlorobut-1-ene-1,2-diyl)diphenol) and intermediate LM (SIAIS151008) as starting materials. The target compound SIAIS307147 was obtained as white solid (12.6 mg, 47% yield). ¹H NMR (500 MHz, MeOD) δ 9.69-9.53 (m, 1H), 7.56-7.51 (m, 2H), 7.51-7.45 (m, 2H), 7.17 (d, J=8.1 Hz, 1H), 7.06 (d, J=8.0 Hz, 1H), 6.97-6.91 (m, 3H), 6.78 (t, J=7.1 Hz, 2H), 6.67 (d, J=7.9 Hz, 1H), 6.60 (t, J=7.1 Hz, 3H), 6.43 (d, J=8.1 Hz, 1H), 4.64 (s, 1H), 4.61-4.53 (m, 2H), 4.52-4.47 (m, 1H), 4.37 (d, J=15.8 Hz, 1H), 4.08 (t, J=5.2 Hz, 1H), 3.95-3.86 (m, 2H), 3.82-3.68 (m, 5H), 3.64-3.53 (m, 13H), 3.51-3.48 (m, 1H), 3.41 (t, J=7.3 Hz, 2H), 2.91-2.83 (m, 2H), 2.59-2.40 (m, 7H), 2.26-2.19 (m, 1H), 2.11-2.01 (m, 1H), 1.06-0.95 (m, 9H). HRMS (ESI) m/z: calcd for C₅₈H₇₃ClN₅O₁₂S [M+H]⁺, 1098.4659; found, 1098.4650.

### Example 81

### Synthesis of N1-(2-(4-(4-chloro-1,2-bis(4-hydroxyphenyl)but-1-en-1-yl)phenoxy)ethyl)-N19-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)-4,7,10,13,16-pentaoxanonadecanediamide (SIAIS 307148)

Referring to the procedures of Example 79, under appropriate conditions that will be recognized by one skilled in the art, the target compound (SIAIS307148) was prepared by using Toremifene derivative D (4,4′-(1-(4-(2-aminoethoxy)phenyl)-4-chlorobut-1-ene-1,2-diyl)diphenol) and intermediate LM (SIAIS151009) as starting materials. The target compound SIAIS307148 was obtained as white solid (11.4 mg, 41% yield). ¹H NMR (500 MHz, MeOD) δ 9.70 (s, 1H), 7.55 (d, J=7.5 Hz, 2H), 7.49 (d, J=7.7 Hz, 2H), 7.17 (d, J=7.8 Hz, 1H), 7.06 (d, J=7.4 Hz, 1H), 6.95 (s, 3H), 6.77 (t, J=6.4 Hz, 2H), 6.67 (d, J=7.9 Hz, 1H), 6.63-6.57 (m, 3H), 6.43 (d, J=7.9 Hz, 1H), 4.64 (s, 1H), 4.61-4.53 (m, 2H), 4.49 (s, 1H), 4.37 (d, J=15.6 Hz, 1H), 4.10-4.05 (m, 1H), 3.96-3.86 (m, 2H), 3.83-3.77 (m, 1H), 3.75-3.68 (m, 4H), 3.64-3.48 (18H), 3.44-3.38 (m, 2H), 2.92-2.82 (m, 2H), 2.61-2.52 (m, 4H), 2.50-2.40 (m, 3H), 2.25-2.18 (m, 1H), 2.12-2.01 (m, 1H), 1.08-0.97 (m, 9H). HRMS (ESI) m/z: calcd for C₆₀H₇₇ClN₅O₁₃S [M+H]⁺, 1142.4922; found, 1142.4918.

### Example 82

### Synthesis of N1-(2-(4-(4-chloro-1,2-bis(4-hydroxyphenyl)but-1-en-1-yl)phenoxy)ethyl)-N5-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)glutaramide (SIAIS307149)

Referring to the procedures of Example 79, under appropriate conditions that will be recognized by one skilled in the art, the target compound (SIAIS307149) was prepared by using Toremifene derivative D (4,4′-(1-(4-(2-aminoethoxy)phenyl)-4-chlorobut-1-ene-1,2-diyl)diphenol) and intermediate LM (SIAIS074012) as starting materials. The target compound SIAIS307149 was obtained as white solid (12.8 mg, 56% yield). ¹H NMR (500 MHz, MeOD) δ 9.62 (s, 1H), 7.53 (d, J=7.6 Hz, 2H), 7.47 (d, J=8.1 Hz, 2H), 7.15 (d, J=7.5 Hz, 1H), 7.05 (d, J=7.4 Hz, 1H), 6.97-6.90 (m, 3H), 6.76 (d, J=7.1 Hz, 2H), 6.63-6.56 (m, 1H), 6.61 (d, J=8.5 Hz, 3H), 6.42 (d, J=7.8 Hz, 1H), 4.61-4.53 (m, 3H), 4.49 (s, 1H), 4.36 (d, J=16.2 Hz, 1H), 4.10-4.04 (m, 1H), 3.95-3.89 (m, 2H), 3.79 (d, J=11.1 Hz, 1H), 3.64-3.56 (m, 1H), 3.52-3.45 (m, 1H), 3.41 (t, J=7.1 Hz, 2H), 2.94-2.80 (m, 2H), 2.55 (s, 3H), 2.37-2.18 (m, 5H), 2.12-2.01 (m, 1H), 1.95-1.84 (m, 2H), 1.06-0.97 (m, 9H). HRMS (ESI) m/z: calcd for C₅₁H₅₉ClN₅O₈S [M+H]⁺, 936.3767; found, 936.3763.

### Example 83

### Synthesis of N1-(2-(4-(4-chloro-1,2-bis(4-hydroxyphenyl)but-1-en-1-yl)phenoxy)ethyl)-N6-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)adip amide (SIAIS 307150)

Referring to the procedures of Example 79, under appropriate conditions that will be recognized by one skilled in the art, the target compound (SIAIS307150) was prepared by using Toremifene derivative D (4,4′-(1-(4-(2-aminoethoxy)phenyl)-4-chlorobut-1-ene-1,2-diyl)diphenol) and intermediate LM (SIAIS074013) as starting materials. The target compound SIAIS307150 was obtained as white solid (10.0 mg, 43% yield). ¹H NMR (500 MHz, MeOD) δ 9.68 (d, J=54.8 Hz, 1H), 7.57-7.52 (m, 2H), 7.51-7.46 (m, 2H), 7.16 (d, J=7.8 Hz, 1H), 7.06 (d, J=7.7 Hz, 1H), 6.94 (t, J=6.9 Hz, 3H), 6.77 (dd, J=7.2, 4.1 Hz, 2H), 6.67 (d, J=7.7 Hz, 1H), 6.60 (t, J=7.7 Hz, 3H), 6.43 (d, J=7.7 Hz, 1H), 4.63-4.53 (m, 3H), 4.49 (s, 1H), 4.37 (d, J=15.8 Hz, 1H), 4.07 (t, J=5.1 Hz, 1H), 3.91 (d, J=9.7 Hz, 2H), 3.79 (d, J=7.6 Hz, 1H), 3.57 (t, J=5.2 Hz, 1H), 3.48 (t, J=5.0 Hz, 1H), 3.41 (t, J=7.3 Hz, 2H), 2.90-2.84 (m, 2H), 2.56 (d, J=3.7 Hz, 3H), 2.30-2.19 (m, 5H), 2.12-2.02 (m, 1H), 1.63-1.60 (m, 4H), 1.02-1.00 (m, 9H). HRMS (ESI) m/z: calcd for C₅₂H₆₁ClN₅O₈S⁺ [M+H]⁺, 950.3924; found, 950.3913.

### Example 84

### Synthesis of N1-(2-(4-(4-chloro-1,2-bis(4-hydroxyphenyl)but-1-en-1-yl)phenoxy)ethyl)-N7-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)heptanediamide (SIAIS307151)

Referring to the procedures of Example 79, under appropriate conditions that will be recognized by one skilled in the art, the target compound (SIAIS307151) was prepared by using Toremifene derivative D (4,4′-(1-(4-(2-aminoethoxy)phenyl)-4-chlorobut-1-ene-1,2-diyl)diphenol) and intermediate LM (SIAIS074014) as starting materials. The target compound SIAIS307151 was obtained as white solid (11.3 mg, 48% yield). ¹H NMR (500 MHz, MeOD) δ 9.57 (s, 1H), 7.53 (d, J=7.7 Hz, 2H), 7.47 (d, J=7.8 Hz, 2H), 7.16 (d, J=7.7 Hz, 1H), 7.06 (d, J=7.3 Hz, 1H), 6.93 (d, J=6.8 Hz, 3H), 6.77 (t, J=6.3 Hz, 2H), 6.67 (d, J=7.8 Hz, 1H), 6.60 (t, J=7.0 Hz, 3H), 6.43 (d, J=7.7 Hz, 1H), 4.62 (s, 1H), 4.59-4.53 (m, 2H), 4.49 (s, 1H), 4.37 (d, J=15.5 Hz, 1H), 4.10-4.03 (m, 1H), 3.90 (d, J=10.0 Hz, 2H), 3.79 (d, J=8.9 Hz, 1H), 3.59-3.55 (m, 1H), 3.49-3.45 (m, 1H), 3.41 (t, J=7.1 Hz, 2H), 2.91-2.83 (m, 2H), 2.55 (s, 3H), 2.31-2.14 (m, 5H), 2.11-2.00 (m, 1H), 1.67-1.55 (m, 4H), 1.34-1.29 (m, 2H), 1.07-0.97 (m, 9H). HRMS (ESI) m/z: calcd for C₅₃H₆₃ClN₅O₈S +[M+H]⁺, 964.4080; found, 964.4070.

### Example 85

### Synthesis of N1-(2-(4-(4-chloro-1,2-bis(4-hydroxyphenyl)but-1-en-1-yl)phenoxy)ethyl)-N8-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)octanediamide (SIAIS307152)

Referring to the procedures of Example 79, under appropriate conditions that will be recognized by one skilled in the art, the target compound (SIAIS307152) was prepared by using Toremifene derivative D (4,4′-(1-(4-(2-aminoethoxy)phenyl)-4-chlorobut-1-ene-1,2-diyl)diphenol) and intermediate LM (SIAIS074015) as starting materials. The target compound SIAIS307152 was obtained as white solid (10.7 mg, 45% yield). ¹H NMR (500 MHz, MeOD) δ 9.64 (d, J=22.6 Hz, 1H), 7.54 (d, J=7.8 Hz, 2H), 7.48 (d, J=8.0 Hz, 2H), 7.16 (d, J=7.7 Hz, 1H), 7.06 (d, J=7.5 Hz, 1H), 6.97-6.91 (m, 3H), 6.77 (dd, J=7.8, 3.5 Hz, 2H), 6.63-6.57 (m, 1H), 6.60 (dd, J=12.3, 5.8 Hz, 3H), 6.43 (d, J=7.7 Hz, 1H), 4.63 (s, 1H), 4.60-4.53 (m, 2H), 4.49 (s, 1H), 4.37 (d, J=15.7 Hz, 1H), 4.07 (t, J=5.1 Hz, 1H), 3.95-3.87 (m, 2H), 3.79 (d, J=7.8 Hz, 1H), 3.57 (t, J=5.1 Hz, 1H), 3.47 (t, J=5.4 Hz, 1H), 3.41 (t, J=7.4 Hz, 2H), 2.90-2.84 (m, 2H), 2.56 (s, 3H), 2.30-2.13 (m, 5H), 2.11-2.02 (m, 1H), 1.65-1.54 (m, 4H), 1.33-1.30 (m, 4H), 1.03-1.01 (m, 9H). HRMS (ESI) m/z: calcd for C₅₄H₆₅ClN₅O₈S⁺ [M+H]⁺, 978.4237; found, 978.4232.

### Example 86

### Synthesis of N1-(2-(4-(4-chloro-1,2-bis(4-hydroxyphenyl)but-1-en-1-yl)phenoxy)ethyl)-N9-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)nonanediamide (SIAIS307153)

Referring to the procedures of Example 79, under appropriate conditions that will be recognized by one skilled in the art, the target compound (SIAIS307153) was prepared by using Toremifene derivative D (4,4′-(1-(4-(2-aminoethoxy)phenyl)-4-chlorobut-1-ene-1,2-diyl)diphenol) and intermediate LM (SIAIS074016) as starting materials. The target compound SIAIS307153 was obtained as white solid (12.4 mg, 51% yield). ¹H NMR (500 MHz, MeOD) δ 9.57 (s, 1H), 7.53 (d, J=7.8 Hz, 2H), 7.47 (d, J=7.3 Hz, 2H), 7.16 (d, J=7.9 Hz, 1H), 7.06 (d, J=8.0 Hz, 1H), 6.97-6.90 (m, 3H), 6.77 (d, J=7.4 Hz, 2H), 6.67 (d, J=8.3 Hz, 1H), 6.63-6.56 (m, 3H), 6.43 (d, J=8.0 Hz, 1H), 4.63 (s, 1H), 4.56 (d, J=13.8 Hz, 2H), 4.49 (s, 1H), 4.37 (d, J=15.3 Hz, 1H), 4.06 (s, 1H), 3.91 (d, J=8.3 Hz, 2H), 3.80 (d, J=9.8 Hz, 1H), 3.56 (s, 1H), 3.47 (s, 1H), 3.41 (t, J=7.0 Hz, 2H), 2.91-2.83 (m, 2H), 2.55 (s, 3H), 2.29-2.13 (m, 5H), 2.10-2.01 (m, 1H), 1.65-1.53 (m, 4H), 1.31-1.29 (m, 6H), 1.03-1.01 (m, 9H). HRMS (ESI) m/z: calcd for C₅₅H₆₇ClN₅O₈S⁺ [M+H]⁺, 992.4393; found, 992.4389.

### Example 87

### Synthesis of N1-(2-(4-(4-chloro-1,2-bis(4-hydroxyphenyl)but-1-en-1-yl)phenoxy)ethyl)-N10-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)decanediamide (S IAIS 307154)

Referring to the procedures of Example 79, under appropriate conditions that will be recognized by one skilled in the art, the target compound (SIAIS307154) was prepared by using Toremifene derivative D (4,4′-(1-(4-(2-aminoethoxy)phenyl)-4-chlorobut-1-ene-1,2-diyl)diphenol) and intermediate LM (SIAIS074019) as starting materials. The target compound SIAIS307154 was obtained as white solid (10.8 mg, 44% yield). ¹H NMR (500 MHz, MeOD) δ 9.44 (s, 1H), 7.52 (d, J=7.7 Hz, 2H), 7.46 (d, J=7.5 Hz, 2H), 7.16 (d, J=7.3 Hz, 1H), 7.06 (d, J=7.1 Hz, 1H), 6.98-6.90 (m, 3H), 6.77 (d, J=8.5 Hz, 2H), 6.67 (d, J=7.1 Hz, 1H), 6.63-6.57 (m, 3H), 6.43 (d, J=7.0 Hz, 1H), 4.63 (s, 1H), 4.60-4.53 (m, 2H), 4.49 (s, 1H), 4.37 (d, J=15.9 Hz, 1H), 4.09-4.04 (m, 1H), 3.91 (d, J=10.2 Hz, 2H), 3.80 (d, J=10.2 Hz, 1H), 3.59-3.54 (m, 1H), 3.48-3.44 (m, 1H), 3.41 (t, J=7.4 Hz, 2H), 2.87 (dd, J=17.2, 8.5 Hz, 2H), 2.53 (s, 3H), 2.31-2.13 (m, 5H), 2.10-2.03 (m, 1H), 1.64-1.52 (m, 4H), 1.33-1.25 (m, 8H), 1.03-1.01 (m, 9H). HRMS (ESI) m/z: calcd for C₅₆H₆₉ClN₅O₈S⁺ [M+H]⁺, 1006.4550; found, 1006.4545.

### Example 88

### Synthesis of N1-(2-(4-(4-chloro-1,2-bis(4-hydroxyphenyl)but-1-en-1-yl)phenoxy)ethyl)-N11-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)undecanediamide (SIAIS307155)

Referring to the procedures of Example 79, under appropriate conditions that will be recognized by one skilled in the art, the target compound (SIAIS307155) was prepared by using Toremifene derivative D (4,4′-(1-(4-(2-aminoethoxy)phenyl)-4-chlorobut-1-ene-1,2-diyl)diphenol) and intermediate LM (SIAIS074020) as starting materials. The target compound SIAIS307155 was obtained as white solid (10.2 mg, 41% yield). ¹H NMR (500 MHz, MeOD) δ 9.52 (d, J=27.3 Hz, 1H), 7.53 (d, J=8.0 Hz, 2H), 7.47 (d, J=8.0 Hz, 2H), 7.16 (d, J=7.6 Hz, 1H), 7.06 (d, J=7.8 Hz, 1H), 6.94 (dd, J=11.9, 7.2 Hz, 3H), 6.77 (d, J=8.1 Hz, 2H), 6.67 (d, J=7.4 Hz, 1H), 6.62-6.58 (m, 3H), 6.43 (d, J=7.8 Hz, 1H), 4.60-4.55 (m, 1H), 4.56 (d, J=14.7 Hz, 2H), 4.49 (s, 1H), 4.37 (d, J=15.6 Hz, 1H), 4.07 (t, J=5.0 Hz, 1H), 3.94-3.87 (m, 2H), 3.80 (d, J=11.2 Hz, 1H), 3.59-3.54 (m, 1H), 3.47 (t, J=5.0 Hz, 1H), 3.41 (t, J=7.4 Hz, 2H), 2.87 (dd, J=17.6, 7.5 Hz, 2H), 2.54 (s, 3H), 2.33-2.17 (m, 5H), 2.10-2.04 (m, 1H), 1.63-1.54 (m, 4H), 1.34-1.23 (m, 10H), 1.03-1.01 (m, 9H). HRMS (ESI) m/z: calcd for C₅₇H₇₁ClN₅O₈S⁺ [M+H]⁺, 1020.4706; found, 1020.4700.

Biological Activity Assay

Evaluation of protein degradation of the compounds of the present disclosure

Experimental Materials

| Reagents and biological materials | Manufacturers |
|---|---|
| Human breast cancer cell line: T47D | Cell Bank of Type Culture |
| | Collection of Chinese Academy |
| | of Sciences (Shanghai, China) |
| Human breast cancer cell line: MCF-7 | ATCC |
| RPMI1640 | Gibco |
| Eagle's Minimum Essential Medium | Gibco |
| Fetal bovine serum | Gibco |
| Penicillin Steptomycin (PS) | Gibco |
| DMSO | Sigma-Aldrich |
| ERα (#8644S) | Cell Signaling Technology |
| β-Actin (13E5) (#5125S) | Cell Signaling Technology |
| GAPDH (#8884S) | Cell Signaling Technology |
| Anti-rabbit IgG HRP-linked (#7074S) | Cell Signaling Technology |
| Recombinant human insulin | Meilun biotech Co., Ltd. |
| PAGE Gel | Bio-Rad Laboratories, Inc. |
| PageRuler ™ Prestained Protein: 26616 | Thermofisher Scientific |
| Pierce Detergent Compatible Bradford | Thermofisher Scientific |
| Assay Kit (#23246) | |
| Western Blot Blocking Buffer (Fish | Takara Bio Inc. |
| Gelatin) | |
| Immobilon Western Chemiluminescent | Millipore |
| HRP Substrate | |
| Cell Counting Kit - 8 | Sigma-Aldrich |

Cell Culture

T47D cells were cultured in an incubator with 5% CO₂ at 37° C. Complete medium was RPMI1640 supplemented with 10% Foetal bovine serum (FBS), 100 U/ml Penicillin and Streptomycin, and 0.77 μg/mL recombinant human insulin.

MCF-7 were cultured in ncubator with 5% CO₂ at 37° C. Complete medium was EMEM supplemented with 10% FBS, 100 U/mL Penicillin and Streptomycin, and 0.77 μg/mL recombinant human insulin.

Western blotting analysis of T47D cell line:
Cells were plated in a 24-well plate at a cell seeding desity of 1.5×10⁵ cells/mL, with 1 ml cell suspensions per well.
After 24 h the cells were adherent, and the cells in each well were treated with 1 μL a certain concentration of ER protein regulators of the present disclosure. 1 μL DMSO served as a negative control, and Toremifene derivative B as a positive control.
After drug treatment for 16 hours, the culture medium was removed and the cells were washed with PBS twice.
Cells lysate and denaturation: the cells were lyzed by adding 40 μL cell lysates, grinded, and treated for 2 cycles each consisting of denaturation at 95° C. for 8 mM, and then cooling on ice for 5 min.
The protein concentration was determined by using the Brandford kit.
Protein loading: 15 μg proteins were loaded on gel for electrophoresis; Electrophoresis: the starting voltage was 80V, and as the dye enters the separation gel, the voltage was adjusted to 120V; Transfering film: the proteins were transferred to nitrocellulose membranes (NC membranes) at constant current 400 mA for lh. Afterwards, the membranes were block by using the Blocking Buffer, and the antibody incubation and development were performed. (The above procedures were all based on the antibody product manual of the manufacturer)

DC₅₀ value (the drug concentration required for degrading proteins by 50%, abbreviated as DC₅₀) reads method: comparing the gray values of the Western blotting bands for the drug treatment with the gray values of the Western blotting band for the DMSO control, and reading the drug concentration range corresponding to the gray value of the Western blotting bands for the drug treatment which is equal to half of the gray value of the Western blotting band for the DMSO control.

DC₅₀ value could also be calculated as follows: using software ImageJ to quantify the gray values of the Western blotting bands for the drug treatment, fitting the relationship curve between drug concentrations and gray values, and from the fitted curve, calculating the drug concentration corresponding to half of the gray value of the Western blotting band for the DMSO control.

Western blotting anaylsis of MCF-7 cell line:

The procedures were the same as those in T47D cells line, except that Toremifene was used as positive control.

Assay for determination of inhibition of MCF-7 cell proliferation:
Cells were seeded in a 96-well plate at a cell seeding desity of 4000 cells/well.
After 24 h the cells were adherent, and Tamoxifen and Toremifene (as the positive controls), and ER protein regulators were added at 10 different concentrations, respectively, which were prepared through serial dilution at a dilution-fold starting from an initial concentration of the above drugs. 10 μL CCK-8 reagent was added to the test well without adding the drugs, and after 4 h the O.D.value of the test well was recorded as the initial cell level.
After 4 days, 10 μL CCK-8 reagent was added, and the O.D.values was measured, and inhibition level was calculated.

Results

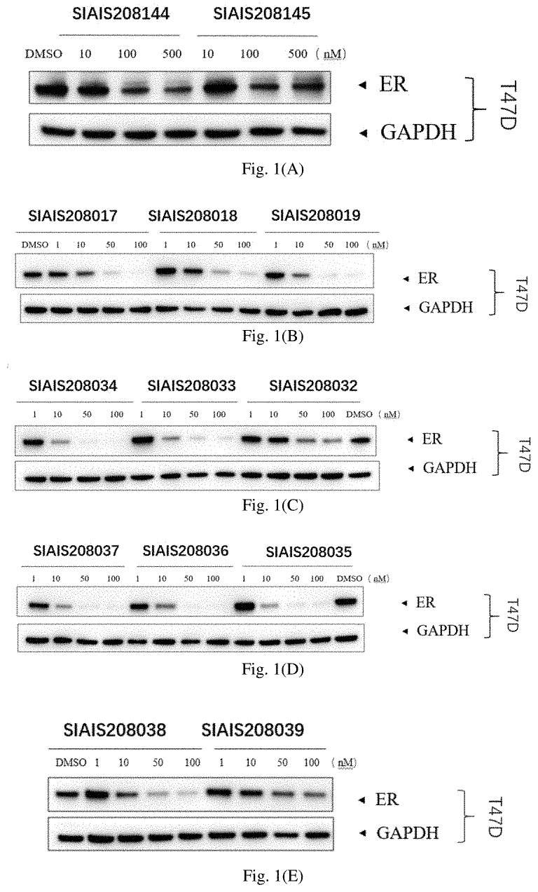

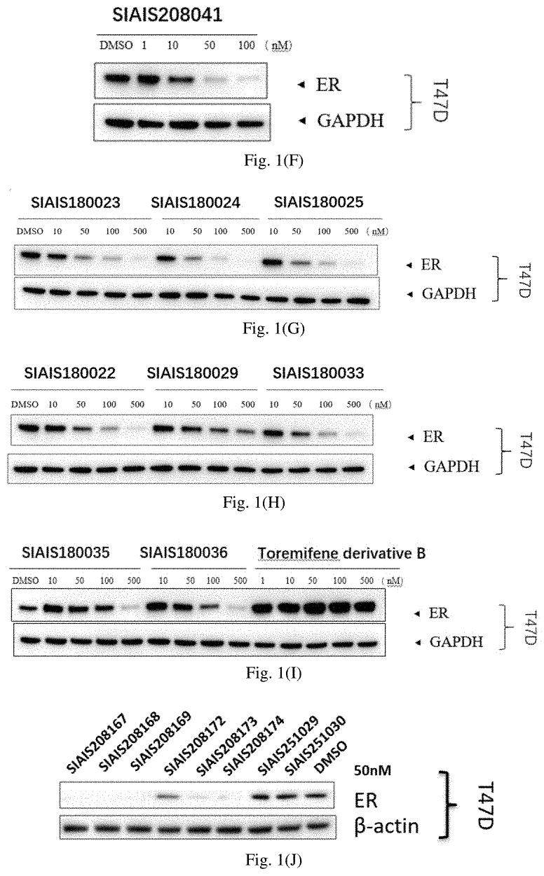

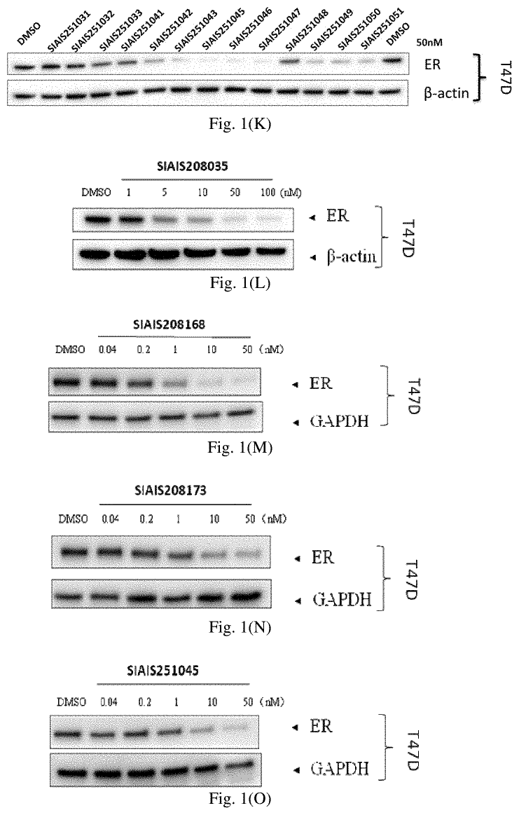

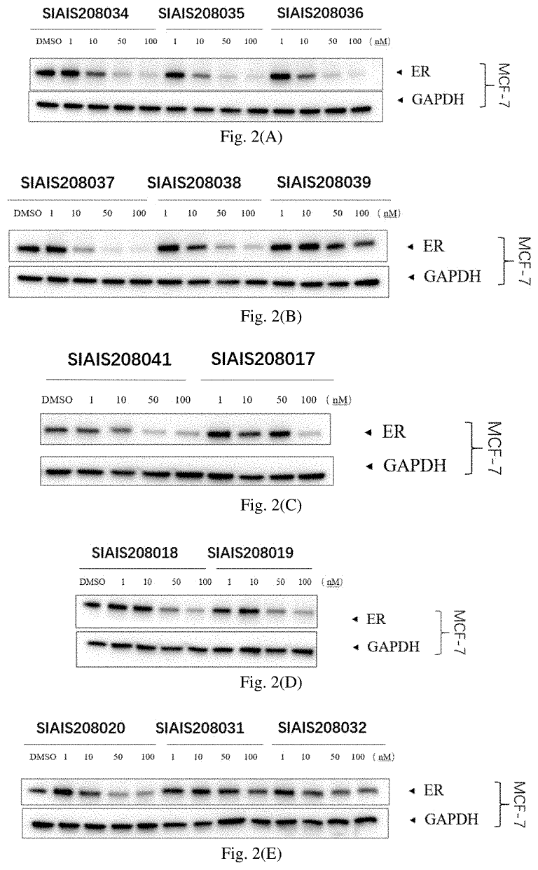

The results of Western blotting successfully confirmed that ER protein regulators of the present disclosure degraded ER protein. In breast cancer cell line T47D, the results of the effects of regulating the ER protein were shown in FIGS. 1(A)-(O) and Table 2, wherein it can be seen that Toremifene derivative B (as the positive control) did not degrade the ER protein in the T47D cell line. In breast cancer cell line MCF-7, the results of the effects of regulating the ER protein were shown in FIGS. 2(A)-(O) and Table 3, wherein it can be seen that Toremifene (as the positive control) did not degrade the ER protein in the MCF-7 cell line.

TABLE 2
Degradation of ER protein induced by the ER protein
regulators of the present disclosure in T47D cells

| Compounds | DC₅₀ (nM)/T47Dcell line |
|---|---|
| SIAIS208144 | <100 |
| SIAIS208145 | <100 |
| SIAIS208135 | <200 |
| SIAIS208107 | <200 |
| SIAIS208125 | <200 |
| SIAIS208127 | <100 |
| SIAIS208017 | <50 |
| SIAIS208018 | <50 |
| SIAIS208019 | <10 |
| SIAIS208020 | <10 |
| SIAIS208031 | <100 |
| SIAIS208034 | <10 |
| SIAIS208033 | <10 |
| SIAIS208032 | <50 |
| SIAIS208037 | <10 |
| SIAIS208036 | <10 |
| SIAIS208035 | <10 |
| SIAIS208038 | <50 |
| SIAIS208039 | <50 |
| SIAIS208041 | <10 |
| SIAIS180023 | <50 |
| SIAIS180024 | <50 |
| SIAIS180025 | <50 |
| SIAIS180022 | <50 |
| SIAIS180029 | 100 |
| SIAIS180033 | <50 |
| SIAIS180035 | <200 |
| SIAIS180036 | <200 |
| SIAIS208167 | <50 |
| SIAIS208168 | <1 |
| SIAIS208169 | <50 |
| SIAIS208172 | <50 |
| SIAIS208173 | <10 |
| SIAIS208174 | <50 |
| SIAIS251033 | 50 |
| SIAIS251042 | <50 |
| SIAIS251043 | <50 |
| SIAIS251045 | <10 |
| SIAIS251046 | <50 |
| SIAIS251047 | <50 |
| SIAIS251049 | <50 |
| SIAIS251050 | <50 |
| SIAIS251051 | <50 |

TABLE 3
Degradation of ER protein induced by the ER protein
regulators of the present disclosure in MCF-7 cells

| Compounds | DC₅₀ (nM)/MCF-7 cell line |
|---|---|
| SIAIS208034 | <10 |
| SIAIS208035 | <10 |
| SIAIS208036 | <10 |
| SIAIS208037 | <10 |
| SIAIS208038 | <10 |
| SIAIS208039 | <100 |
| SIAIS208041 | 10 |
| SIAIS208017 | <100 |
| SIAIS208018 | <50 |
| SIAIS208019 | <50 |
| SIAIS208020 | <50 |
| SIAIS208031 | <100 |
| SIAIS208032 | <100 |
| SIAIS208033 | <10 |

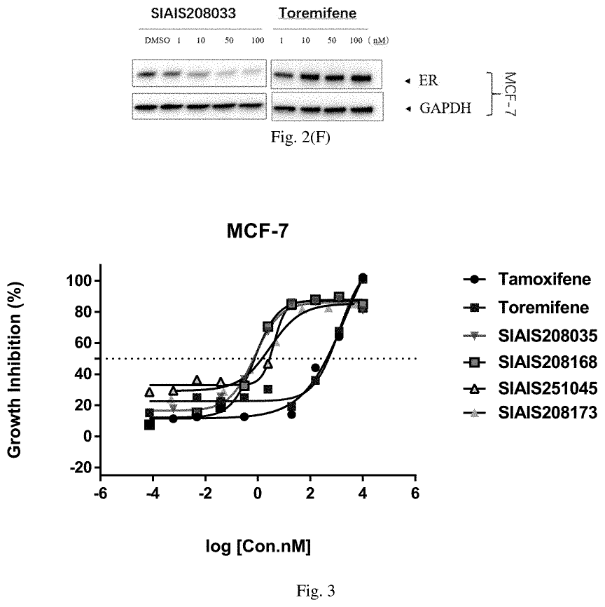

We further selected four compounds of the present disclosure for proliferation inhibition assay in MCF-7 cell line. The result showed that the abilities of inhibiting cell growth of these compounds were suprerior than Tamoxifen and Toremifene. A shown in FIG. 3, the IC₅₀ values of SIAIS208035, SIAIS208168, SIAIS208173, and SIAIS251045 were 0.75 nM, 0.75 nM, 3.6 nM, and 4.2 nM, respectively, while the IC₅₀ values of Tamoxifene and Toremifene were 602 nM and 869 nM, respectively. Compared to the positive controls, the cell growth inhibitory effects of the four PROTAD molecules of the present disclosure have been increased by hundreds or even thousands of times.

1. A compound of formula (I) 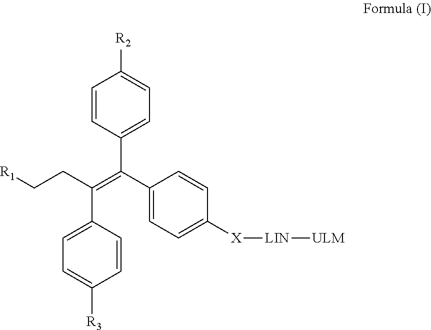
or salts, enantiomers, stereoisomers, solvates, or polymorphs thereof, wherein X is covalently bonded to ULM through a linking group LIN;
wherein R₁ represents halogen, R₂ represents H, halogen, or OH, and R₃ represents H, halogen, or OH; or R₁ represents H, and R₂ and R₃ are both halogen or OH;
X represents CH₂, O, or NH;
LIN is a linking group and represents -alkylene-,
wherein the alkylene group is a linear or branched alkylene group optionally interrupted one or more times by one or more groups selected from the group consisting of O, CO, CON(R₄), N(R₅)CO, N(R₆), alkynylene, alkenylene, cycloalkylene, arylene, heterocyclylene, heteroarylene, or any combination thereof, and
wherein the linear or branched alkylene group is optionally substituted with one or more substituents; and R₄, R₅, and R₆ are each independently selected from the group consisting of H and C₁₋₃ alkyl; and
ULM is a small-molecule ligand of VHL or CRBN protease having a ubiquitylation function.

2. The compound of formula (I) or a salt, enantiomer, stereoisomer, solvate, polymorph thereof of claim 1, wherein:
R₁ represents halogen, R₂ and R₃ both represent H, and X represents O; or
R₁ represents halogen, R₂ represents OH, R₃ represents H, and X represents O; or
R₁ represents halogen, R₂ represents H, R₃ represents OH, and X represents O; or
R₁ represents halogen, R₂ and R₃ both represent OH, and X represents O; or
R₁ represents H, R₂ and R₃ both represent OH, and X represents O; or
R₁ represents H, R₂ and R₃ both represent halogen, and X represents O.

3.-7. (canceled)

8. The compound of formula (I) or a salt, enantiomer, stereoisomer, solvate, or polymorph thereof of claim 1, wherein the ULM represents the following structure of formula (II): 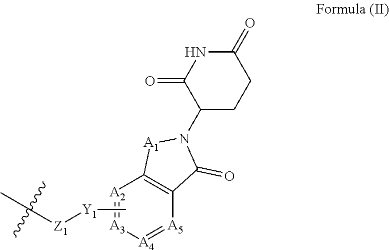
wherein A₁ represents CH₂ or CO; A₂, A₃, A₄, and A₅ are the same or different and each independently represent CH or N, wherein A₂, A₃, A₄, and A₅ are not N at the same time; Y₁ represents CH₂, NH, or O; and Z₁ represents CO or Z₁ is absent.

9. The compound of formula (I) or a salt, enantiomer, stereoisomer, solvate, or polymorph thereof of claim 1, wherein the ULM represents the following structure of formula (III): 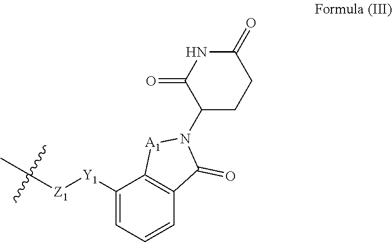
wherein A₁ represents CH₂ or CO; Y₁ represents CH₂, NH, or O; and Z₁ represents CO or Z₁ is absent.

10. The compound of formula (I) or a salt, enantiomer, stereoisomer, solvate, or polymorph thereof of claim 1, wherein the ULM represents the following structure of formula (IV): 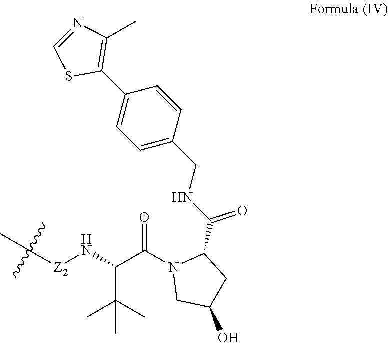
wherein Z₂ represents CO or Z₂ is absent.

11. The compound of formula (I) or a salt, enantiomer, stereoisomer, solvate, or polymorph thereof of claim 1, wherein the LIN represents:
a linear or branched C₁-C₃₀ alkylene chain; —(CH₂)ₙ₁—(O(CH₂)ₙ₂)ₘ₁—; —(CH₂)ₙ₁—(O(CH₂)ₙ₂)ₘ₁—O—(CH₂)ₙ₃—; —(CR₇R₈)ₙ₁—(O(CR₉R₁₀)ₙ₂)ₘ₁—; —(CR₁₁R₁₂)ₙ₁—(O(CR₁₃R₁₄)ₙ₂)ₘ₁—O—(CR₁₅R₁₆)ₙ₃—; —(CH₂)ₙ₁—N(R₆)—(CH₂)ₙ₂—; —(CH₂)ₙ₁—N(R₅)CO—(CH₂)ₙ₂—; —(CH₂)ₙ₁—(N(R₅)CO—(CH₂)ₙ₂)ₘ₁—; —(CH₂)ₙ₁—N(R₅)CO—(CH₂)ₙ₂—(O(CH₂)ₙ₃)ₘ₁—; —(CH₂)ₙ₁—(N(R₅)CO—(CH₂)ₙ₂)ₘ₁—O—(CH₂)ₙ₃—; —(CH₂)ₙ₁—N(R₅)CO—(CH₂)ₙ₂—(O(CH₂)ₙ₃)ₘ₁—O(CH₂)ₙ₄—; —(CH₂)ₙ₁-piperazinylene-(CH₂)ₙ₂—; —(CH₂)ₙ₁—N(R₅)CO—(CH₂)ₙ₂-piperazinylene-(CH₂)ₙ₃—; —(CH₂)ₙ₁—(N(R₅)CO—(CH₂)ₙ₂)ₘ₁—piperazinylene-(CH₂)ₙ₃—; —(CH₂)ₙ₁-piperazinylene-CO—(CH₂)ₙ₂—(O(CH₂)ₙ₃)ₘ₁—; —(CH₂)ₙ₁-piperazinylene-CO—(CH₂)ₙ₂—(O(CH₂)ₙ₃)ₘ₁—O(CH₂)ₙ₄—; —(CH₂)ₙ₁—piperazinylene-CO—(CH₂)ₙ₂—; —(CH₂)ₙ₁-phenylene-(CH₂)ₙ₂—; —(CH₂)ₙ₁—N(R₅)CO—(CH₂)ₙ₂-phenylene-(CH₂)ₙ₃—; —(CH₂)ₙ₁—(N(R₅)CO—(CH₂)ₙ₂)ₘ₁-phenylene-(CH₂)ₙ₃—; a linear or branched alkylene chain interrupted one or more times by one or more selected from the group consisting of CO, alkynylene, alkenylene, cycloalkylene, arylene, heterocyclylene, or heteroarylene, or any combination thereof; or —(CH₂)ₙ₁—(O(CH₂)ₙ₂)ₘ₁— in which backbone carbon chain is interrupted one or more times by one or more selected from the group consisting of CO, arylene, heterocyclylene, heteroarylene, or any combination thereof;
wherein,
R₅ and R₆ are each independently selected from the group consisting of H and C₁₋₃ alkyl;
R₇, R₈, R₉, R₁₀, R₁₁, R₁₂, R₁₃, R₁₄, R₁₅, and R₁₆ each independently represent H, linear or branched C₁₋₁₀ alkyl or C₃-C₁₀cycloalkyl, wherein in the same group LIN, R₇, R₈, R₉, and R₁₀ are not H at the same time; or R₁₁, R₁₂, R₁₃, R₁₄, R₁₅, and R₁₆ are not H at the same time; and
n1, n2, n3, n4, and ml each independently represent an integer of 1, 2, 3, 4, 5, 6, 7, 8, 9, 10, 11, 12, 13, 14, 15, 16, 17, 18, 19, or 20.

12. The compound of formula (I) or a salt, enantiomer, stereoisomer, solvate, or polymorph thereof of claim 1, wherein the LIN represents:
—(CH₂)₂O(CH₂)₂O(CH₂)₂—;
—CH₂O(CH₂)₂OCH₂—;
—CH₂O(CH₂)₂O(CH₂)₂—;
—(CH₂)₃O(CH₂)₂—;
—(CH₂)₃O(CH₂)₂O(CH₂)₂—;
—(CH₂)₃O(CH₂)₃—;
—(CH₂)₂O(CH₂)₂—;
—(CH₂)₂O(CH₂)₂OCH₂—;
—(CH₂)₂O(CH₂)₂O(CH₂)₃—;
—(CH₂)₂O(CH₂)₂O(CH₂)₂O(CH₂)₂—;
—(CH₂)₂O(CH₂)₂O(CH₂)₂O(CH₂)₃—;
—(CH₂)₅O(CH₂)₂O(CH₂)₂O(CH₂)₅—;
—(CH₂)₅O(CH₂)₂O(CH₂)₂O(CH₂)₆—;
—(CH₂)₂O(CH₂)₂O(CH₂)₂O(CH₂)₂O(CH₂)₂—;
—(CH₂)₂O(CH₂)₂O(CH₂)₂O(CH₂)₂O(CH₂)₃—;
—(CH₂)₂O(CH₂)₂O(CH₂)₂O(CH₂)₂O(CH₂)₂O(CH₂)₂—;
—(CH₂)₃O(CH₂)₂O(CH₂)₂O(CH₂)₂O(CH₂)₂O(CH₂)₂—; or
—(CH₂)₃O(CH₂)₂O(CH₂)₂O(CH₂)₂O(CH₂)₂O(CH₂)₃—.

13. The compound of formula (I) or a salt, enantiomer, stereoisomer, solvate, or polymorph thereof of claim 11, wherein the LIN represents:
—CH₂—; —(CH₂)₂—; —(CH₂)₃—; —(CH₂)₄—; —(CH₂)₅—; —(CH₂)₆—; —(CH₂)₇—; —(CH₂)₈—; —(CH₂)₉—; —(CH₂)₁₀—; —(CH₂)₁₁—; —(CH₂)₁₂—; —(CH₂)₁₃—; —(CH₂)₁₄—; —(CH₂)₁₅—; —(CH₂)₁₆—; —(CH₂)₁₇—; —(CH₂)₁₈—; —(CH₂)₁₉—; or —(CH₂)₂₀—.

14. The compound of formula (I) or a salt, enantiomer, stereoisomer, solvate, or polymorph thereof of claim 1, wherein the substituent is selected from the group consisting of hydroxyl, amino, mercapto, and halogen.

15. The compound of formula (I) or a salt, enantiomer, stereoisomer, solvate, or polymorph thereof of claim 14, wherein the LIN is a linear or branched C₁-C₃₀ alkylene group substituted by one or more substituents selected from the group consisting of hydroxyl, amino, mercapto, halogen, or combination thereof.

16. The compound of formula (I) or a salt, enantiomer, stereoisomer, solvate, or polymorph thereof of claim 11, wherein the LIN represents:
—(CH₂)₁—NH—(CH₂)₁—; —(CH₂)₂—NH—(CH₂)₁—; —(CH₂)₂—NH—(CH₂)₂—; —(CH₂)₂—NH—(CH₂)₃—; —(CH₂)₂—NH—(CH₂)₄—; —(CH₂)₂—NH—(CH₂)₅—; —(CH₂)₂—NH—(CH₂)₆—; —(CH₂)₂—NH—(CH₂)₇—; —(CH₂)₂—NH—(CH₂)₈—; —(CH₂)₂—NH—(CH₂)₉—; —(CH₂)₂—NH—(CH₂)₁₀—; —(CH₂)₂—NH—(CH₂)₁₁—; —(CH₂)₂—NH—(CH₂)₁₂—; —(CH₂)₁—N(CH₃)—(CH₂)₈—; —(CH₂)₂—N(CH₃)—(CH₂)₁—; —(CH₂)₂—N(CH₃)—(CH₂)₂—; —(CH₂)₂—N(CH₃)—(CH₂)₃—; —(CH₂)₂—N(CH₃)—(CH₂)₄—; —(CH₂)₂—N(CH₃)—(CH₂)₅—; —(CH₂)₂—N(CH₃)—(CH₂)₆—; —(CH₂)₂—N(CH₃)—(CH₂)₇—; —(CH₂)₂—N(CH₃)—(CH₂)₈—; —(CH₂)₂—N(CH₃)—(CH₂)₉—; —(CH₂)₂—N(CH₃)—(CH₂)₁₀—; —(CH₂)₂—N(CH₃)—(CH₂)₁₁—; or —(CH₂)₂—N(CH₃)—(CH₂)₁₂—; or
—(CH₂)₂—NHCO—CH₂—; —(CH₂)₂—NHCO—(CH₂)₂—; —(CH₂)₂—NHCO—(CH₂)₃—; —(CH₂)₂—NHCO—(CH₂)₄—; —(CH₂)₂—NHCO—(CH₂)₅—; —(CH₂)₂—NHCO—(CH₂)₆—; —(CH₂)₂—NHCO—(CH₂)₇—; —(CH₂)₂—NHCO—(CH₂)₈—; —(CH₂)₂—NHCO—(CH₂)₉—; —(CH₂)₂—NHCO—(CH₂)₁₀—; —(CH₂)₂—NHCO—(CH₂)₁₁—; —(CH₂)₂—NHCO—(CH₂)₁₂—; —(CH₂)₂—NHCO—(CH₂)₁₃—; —(CH₂)₂—NHCO—(CH₂)₁₄—; —(CH₂)₂—NHCO—(CH₂)₁₅—; —(CH₂)₂—N(CH₃)CO—CH₂—; —(CH₂)₂—N(CH₃)CO—(CH₂)₂—; —(CH₂)₂—N(CH₃)CO—(CH₂)₃—; —(CH₂)₂—N(CH₃)CO—(CH₂)₄—; —(CH₂)₂—N(CH₃)CO—(CH₂)₅—; —(CH₂)₂—N(CH₃)CO—(CH₂)₆—; —(CH₂)₂—N(CH₃)CO—(CH₂)₇—; —(CH₂)₂—N(CH₃)CO—(CH₂)₈—; —(CH₂)₂—N(CH₃)CO—(CH₂)₉—; —(CH₂)₂—N(CH₃)CO—(CH₂)₁₀—; —(CH₂)₂—N(CH₃)CO—(CH₂)₁₁—; —(CH₂)₂—N(CH₃)CO—(CH₂)₁₂—; —(CH₂)₂—N(CH₃)CO—(CH₂)₁₃—; —(CH₂)₂—N(CH₃)CO—(CH₂)₁₄—; or —(CH₂)₂—N(CH₃)CO—(CH₂)₁₅—; or
—(CH₂)₂—NHCO—(CH₂)₂—O(CH₂)₂—; —(CH₂)₂—NHCO—(CH₂)₂—(O(CH₂)₂)₂—; —(CH₂)₂—NHCO—(CH₂)₂—(O(CH₂)₂)₃—; —(CH₂)₂—NHCO—(CH₂)₂—(O(CH₂)₂)₄—; —(CH₂)₂—NHCO—(CH₂)₂—(O(CH₂)₂)₅—; —(CH₂)₂—NHCO—(CH₂)₂—(O(CH₂)₂)₆—; —(CH₂)₂—NHCO—(CH₂)₂—(O(CH₂)₂)₇—; —(CH₂)₂—NHCO—(CH₂)₂—(O(CH₂)₂)₈—; —(CH₂)₂—NHCO—(CH₂)₂—(O(CH₂)₂)₉—; —(CH₂)₂—NHCO—(CH₂)₂—(O(CH₂)₂)₁₀—; —(CH₂)₂—N(CH₃)CO—(CH₂)₂—O(CH₂)₂—; —(CH₂)₂—N(CH₃)CO—(CH₂)₂—(O(CH₂)₂)₂—; —(CH₂)₂—N(CH₃)CO—(CH₂)₂—(O(CH₂)₂)₃—; —(CH₂)₂—N(CH₃)CO—(CH₂)₂—(O(CH₂)₂)₄—; —(CH₂)₂—N(CH₃)CO—(CH₂)₂—(O(CH₂)₂)₅—; —(CH₂)₂—N(CH₃)CO—(CH₂)₂—(O(CH₂)₂)₆; —(CH₂)₂—N(CH₃)CO—(CH₂)₂—(O(CH₂)₂)₇—; —(CH₂)₂—N(CH₃)CO—(CH₂)₂—(O(CH₂)₂)₈—; —(CH₂)₂—N(CH₃)CO—(CH₂)₂—(O(CH₂)₂)₉—; or —(CH₂)₂—N(CH₃)CO—(CH₂)₂—(O(CH₂)₂)₁₀—; or
—(CH₂)₂—NHCO—CH₂—O(CH₂)₂—OCH₂—; —(CH₂)₂—NHCO—(CH₂)₂—O(CH₂)₂—OCH₂—; —(CH₂)₂—NHCO—(CH₂)₂—(O(CH₂)₂)₂—OCH₂—; —(CH₂)₂—NHCO—(CH₂)₂—(O(CH₂)₂)₂—O(CH₂)₃—; —(CH₂)₂—N(CH₃)CO—CH₂—O(CH₂)₂—OCH₂—; —(CH₂)₂—N(CH₃)CO—CH₂—(O(CH₂)₂)₂—OCH₂—; —(CH₂)₂—N(CH₃)CO—CH₂—(O(CH₂)₂)₃—OCH₂—; or —(CH₂)₂—N(CH₃)CO—CH₂—(O(CH₂)₂)₂—O(CH₂)₃—; or
—(CH₂)₂—NHCO—(CH₂)₂-piperazinylene-(CH₂)₂—; —(CH₂)₂—NHCO—(CH₂)₂-piperazinylene-(CH₂)₃—; —(CH₂)₂—NHCO—(CH₂)₃-piperazinylene-(CH₂)₂—; —(CH₂)₂—NHCO—(CH₂)₃-piperazinylene-(CH₂)₃—; —(CH₂)₂—NHCO—CH₂-piperazinylene-(CH₂)₂—; —(CH₂)₂—N(CH₃)CO—(CH₂)₂-piperazinylene-(CH₂)₂—; —(CH₂)₂—N(CH₃)CO—CH₂-piperazinylene-(CH₂)₂—; —(CH₂)₂—N(CH₃)CO—(CH₂)₂-piperazinylene-(CH₂)₃—; —(CH₂)₂—N(CH₃)CO—(CH₂)₃-piperazinylene-(CH₂)₂—; —(CH₂)₂—N(CH₃)CO—(CH₂)₃-piperazinylene-CH₂)₃—; —(CH₂ NHCO—(CH₂)₂)₂)₂-piperazinylene-CH₂—; —(CH₂)₂—(NHCO—(CH₂)₂)₂-piperazinylene-(CH₂)₂—; —(CH₂)₂—(NHCO—(CH₂)₂)₂-piperazinylene-(CH₂)₃—; —(CH₂)₂—(NHCO—(CH₂)₂)₂-piperazinylene-(CH₂)₄—; —(CH₂)₂—(NHCO—(CH₂)₂)₃-piperazinylene-(CH₂)₂—; —(CH₂)₂—(N(CH₃)CO—(CH₂)₂)₂)₂-piperazinylene-CH₂—; —(CH₂)₂—(N(CH₃)CO—(CH₂)₂)₂)₂-piperazinylene-(CH₂)₂—; —(CH₂)₂—(N(CH₃)CO—(CH₂)₂)₂)₂-piperazinylene-(CH₂)₃—; —(CH₂)₂—(N(CH₃)CO—(CH₂)₂)₂-piperazinylene-(CH₂)₄—; or —(CH₂)₂—(N(CH₃)CO—(CH₂)₂)₃—piperazinylene-(CH₂)₂—; or
—CH₂-piperazinylene-CH₂—; —CH₂-piperazinylene-(CH₂)₂—; —CH₂-piperazinylene-(CH₂)₃—; —CH₂-piperazinylene-(CH₂)₄—; —CH₂-piperazinylene-(CH₂)₅—; —(CH₂)₂-piperazinylene-CH₂—; —(CH₂)₂-piperazinylene-(CH₂)₂—; —(CH₂)₂-piperazinylene-(CH₂)₃—; —(CH₂)₂-piperazinyline-(CH₂)₄—; —(CH₂)₂-piperazinylene-(CH₂)₅—; —(CH₂)₂-piperazinylene-(CH₂)₆—; —(CH₂)₂-piperazinylene-(CH₂)₇—; —(CH₂)₂-pjperazinylene-(CH₂)₈—; —(CH₂)₂-piperazinylene-(CH₂)₉—; or —(CH₂)₂-piperazinylene-(CH₂)₁₀—; or
—(CH₂)₂-piperazinylene-CO—CH₂—O(CH₂)₂—; —(CH₂)₂-piperazinylene-CO—CH₂-OCH₂—; —(CH₂)₂-piperazinylene-CO—CH₂—O(CH₂)₂—OCH₂—; —(CH₂)₂-piperazinylene-CO—(CH₂)₂—O(CH₂)₂—; —(CH₂)₂-piperazinylene-CO—(CH₂)₂—(O(CH₂)₂)₂—; —(CH₂)₂-piperazinylene-CO—(CH₂)₂—(O(CH₂)₂)₃—; —(CH₂)₂-piperazinylene-CO—(CH₂)₂—(O(CH₂)₂)₄—; —(CH₂)₂-piperazinylene-CO—(CH₂)₂—(O(CH₂)₂)₅—; —(CH₂)₂-piperazinylene-CO—(CH₂)₂—(O(CH₂)₂)₆—; —(CH₂)₂-piperazinylene-CO—(CH₂)₂—(O(CH₂)₂)₇—; —(CH₂)₂-piperazinylene-CO—(CH₂)₂-(O(CH₂)₂)₈—; —(CH₂)₂-piperazinylene-CO—(CH₂)₂—(O(CH₂)₂)₉—; or —(CH₂)₂-piperazinylene-CO—(CH₂)₂—(O(CH₂)₂)₁₀—; or
—(CH₂)₂-piperazinylene-CO—CH₂—; —(CH₂)₂-piperazinylene-CO—(CH₂)₂—; —(CH₂)₂-piperazinylene-CO—(CH₂)₃—; —(CH₂)₂-piperazinylene-CO—(CH₂)₄—; —(CH₂)₂-piperazinylene-CO—(CH₂)₅—; —(CH₂)₂-piperazinylene-CO—(CH₂)₆—; —(CH₂)₂-piperazinylene-CO—(CH₂)₇—; —(CH₂)₂-piperazinylene-CO—(CH₂)₈—; —(CH₂)₂-piperazinylene-CO—(CH₂)₉—; or —(CH₂)₂-piperazinylene-CO—(CH₂)₁₀—; or
CH₂-phenylene-CH₂—; —(CH₂)₂-phenylene-(CH₂)₂—; —(CH₂)₂-phenylene-(CH₂)₃—; —(CH₂)₂-phenylene-(CH₂)₄—; —(CH₂)₂-phenylene-(CH₂)₅—; —(CH₂)₃-phenylene-(CH₂)₂—; —(CH₂)₄-phenylene-(CH₂)₂—; or —(CH₂)₄-phenylene-(CH₂)₃—; or
—(CH₂)₂—NHCO—(CH₂)₂-phenylene-(CH₂)₂—; —(CH₂)₂—NHCO—CH₂-phenylene-(CH₂)₂—; —(CH₂)₂—NHCO—(CH₂)₃-phenylene-(CH₂)₂—; —(CH₂)₂—NHCO—(CH₂)₂-phenylene-(CH₂)₃—; —(CH₂)₂—N(CH₃)CO—(CH₂)₂-phenylene-(CH₂)₂—; —(CH₂)₂—N(CH₃)CO—(CH₂)₃-phenylene-(CH₂)₂—; —(CH₂)₂—N(CH₃)CO—(CH₂)₂-phenylene-(CH₂)₃—; —(CH₂)₂—(NHCO—(CH₂)₂)₂-phenylene-(CH₂)₂—; —(CH₂)₂—(NHCO—(CH₂)₂)₂-phenylene-(CH₂)₃—; —(CH₂)₂—(N(CH₃)CO—(CH₂)₂)₂-phenylene-(CH₂)₂—; or —(CH₂)₂—(N(CH₃)CO—(CH₂)₂)₃-phenylene —(CH₂)₂—.

17.-25. (canceled)

26. The compound of formula (I) or a salt, enantiomer, stereoisomer, solvate, or polymorph thereof of claim 1, wherein
R₁ represents halogen, R₂ and R₃ represent H, and X represents O;
ULM represents the following structure of formula (IV): 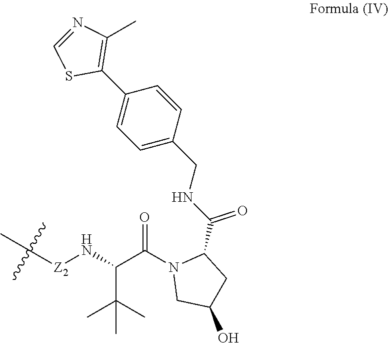
wherein Z₂ represents CO or Z₂ is absent; and
LIN represents alkylene,
wherein the alkylene group is a linear or branched alkylene group interrupted one or more times by one or more groups selected from the group consisting of O, CON(R₄), N(R₅)CO, or any combination thereof, wherein the linear or branched alkylene group is optionally substituted with one or more substituents selected from the group consisting of hydroxyl, amino, mercapto, and halogen; and R₄ and R₅ are each independently selected from the group consisting of H and C₁₋₃ alkyl.

27. The compound of formula (I) or a salt, enantiomer, stereoisomer, solvate, or polymorph thereof of claim 26, wherein the LIN represents —(CH₂)ₙ₁—N(R₅)CO—(CH₂)ₙ₂—, or —(CH₂)ₙ₁—N(R₅)CO—(CH₂)ₙ₂—(O(CH₂)ₙ₃)ₘ₁—, wherein the hydrogen atom(s) on the carbon atom(s) of backbone carbon chain of the LIN group is/are optionally replaced by one or more substituents selected from the group consisting of hydroxyl, amino, mercapto, and halogen; R₅ is selected from the group consisting of H and C₁₋₃ alkyl; and n1, n2, n3, and m1 each independently represent an integer of 1, 2, 3, 4, 5, 6, 7, 8, 9, 10, 11, 12, 13, 14, 15, 16, 17, 18, 19, or 20.

28. The compound of formula (I) or a salt, enantiomer, stereoisomer, solvate, or polymorph thereof of cclaim 26, wherein the LIN represents:
—(CH₂)₂—N(CH₃)CO—(CH₂)₂—O(CH₂)₂—; —(CH₂)₂—N(CH₃)CO—(CH₂)₂—(O(CH₂)₂)₂—; —(CH₂)₂—N(CH₃)CO—(CH₂)₂—(O(CH₂)₂)₃—; —(CH₂)₂—N(CH₃)CO—(CH₂)₂—(O(CH₂)₂)₄—; —(CH₂)₂—N(CH₃)CO—(CH₂)₂—(O(CH₂)₂)₅—; —(CH₂)₂—N(CH₃)CO—(CH₂)₂—(O(CH₂)₂)₆—; —(CH₂)₂—N(CH₃)CO—(CH₂)₂—(O(CH₂)₂)₇—; —(CH₂)₂—N(CH₃)CO—(CH₂)₂—(O(CH₂)₂)₈—; —(CH₂)₂—N(CH₃)CO—(CH₂)₂—(O(CH₂)₂)₉—; or —(CH₂)₂—N(CH₃)CO—(CH₂)₂—(O(CH₂)₂)₁₀—; or
—(CH₂)₂—N(CH₃)CO—CH₂—; —(CH₂)₂—N(CH₃)CO—(CH₂)₂—; —(CH₂)₂—N(CH₃)CO—(CH₂)₃—; —(CH₂)₂—N(CH₃)CO—(CH₂)₄—; —(CH₂)₂—N(CH₃)CO—(CH₂)₅—; —(CH₂)₂—N(CH₃)CO—(CH₂)₆—; —(CH₂)₂—N(CH₃)CO—(CH₂)₇—; —(CH₂)₂—N(CH₃)CO—(CH₂)₈—; —(CH₂)₂—N(CH₃)CO—(CH₂)₉—; —(CH₂)₂—N(CH₃)CO—(CH₂)₁₀—; —(CH₂)₂—N(CH₃)CO—(CH₂)₁₁—; —(CH₂)₂—N(CH₃)CO—(CH₂)₁₂—; —(CH₂)₂—N(CH₃)CO—(CH₂)₁₃—; —(CH₂)₂—N(CH₃)CO—(CH₂)₁₄—; or —(CH₂)₂—N(CH₃)CO—(CH₂)₁₅—.

29. (canceled)

30. The compound of formula (I) or a salt, enantiomer, stereoisomer, solvate, or polymorph thereof of claim 1, wherein
R₁ represents halogen, R₂ and R₃ represent H, and X represents O;
ULM represents the following structure of formula (II): 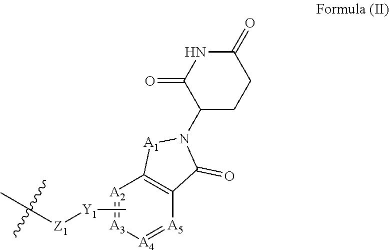
wherein Y₁ represents CH₂, NH, or O; Z₁ represents CO or Z₁ is absent; A₁ represents CH₂ or CO; A₂, A₃, A₄, and A₅ are the same or different and each independently represent CH or N, provided that A₂, A₃, A₄, and A₅ are not N at the same time; and
LIN represents alkylene,
wherein the alkylene group is a linear or branched alkylene group interrupted one or more times by one or more groups selected from the group consisting of CON(R₄), N(R₅)CO, heterocyclylene, heteroarylene, or any combination thereof, wherein the linear or branched alkylene group is optionally substituted with one or more substituents selected from the group consisting of hydroxyl, amino, mercapto, and halogen; R₄ and R₅ are each independently selected from the group consisting of H and C₁₋₃ alkyl; and the heterocyclylene and heteroarylene group are optionally substituted with the substituent(s) selected from the group consisting of C₁₋₃ alkyl, C₁₋₃ alkoxy, cyano, trifluoromethyl, heterocyclyl, halogen, amino, or hydroxyl.

31. The compound of formula (I) or a salt, enantiomer, stereoisomer, solvate, or polymorph thereof of claim 30, wherein the LIN is —(CH₂)ₙ₁—N(R₅)CO—(CH₂)ₙ₂-piperazinylene-(CH₂)ₙ₃—, wherein the hydrogen atom(s) on the carbon atom(s) of backbone carbon chain of the LIN group is/are optionally replaced by one or more substituents selected from the group consisting of hydroxyl, amino, mercapto, and halogen; R₅ is selected from the group consisting of H and C₁₋₃ alkyl; and n1, n2, and n3 each independently represent an integer of 1, 2, 3, 4, 5, 6, 7, 8, 9, 10, 11, 12, 13, 14, 15, 16 , 17, 18, 19, or 20, and wherein said piperazinylene group is optionally substituted with a substituent selected from the group consisting of C₁₋₃ alkyl, C₁₋₃ alkoxy, cyano, trifluoromethyl, heterocyclyl, halogen, amino, or hydroxyl.

32. The compound of formula (I) or a salt, enantiomer, stereoisomer, solvate, or polymorph thereof of claim 30, wherein the ULM represents the following structure of formula (III): 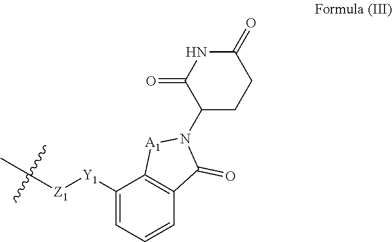
wherein A₁ represents CH₂ or CO; Y₁ represents NH; and Z₁ represents CO or Z₁ is absent.

33. The compound of formula (I) or a salt, enantiomer, stereoisomer, solvate, or polymorph thereof of cclaim 30, wherein the LIN represents:
—(CH₂)₂—N(CH₃)CO—(CH₂)₂-piperazinylene-(CH₂)₂—; —(CH₂)₂—N(CH₃)CO—CH₂-piperazinylene-(CH₂)₂—; —(CH₂)₂—N(CH₃)CO—(CH₂)₂-piperazinylene-(CH₂)₃—; —(CH₂)₂—N(CH₃)CO—(CH₂)₃-piperazinylene-(CH₂)₂—; or —(CH₂)₂—N(CH₃)CO—(CH₂)₃-piperazinylene-(CH₂)₃—.

34. The compound of formula (I) or a salt, enantiomer, stereoisomer, solvate, or polymorph thereof of claim 1, wherein
R₁ represents halogen, R₂ represents OH, R₃ represents H, and X represents O;
ULM represents the following structure of formula (II): 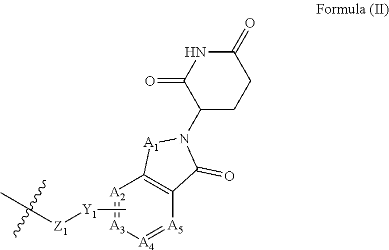
wherein Y₁ represents CH₂, NH, or O; Z₁ represents CO or Z₁ is absent; A₁ represents CH₂ or CO; A₂, A₃, A₄, and A₅ are the same or different and each independently represent CH or N, provided that A₂, A₃, A₄, and A₅ are not N at the same time; and
LIN represents alkylene,
wherein the alkylene group is a linear or branched alkylene group interrupted one or more times by the group selected from the group consisting of CON(R₄), N(R₅)CO, or any combination thereof, wherein the linear or branched alkylene group is optionally substituted with one or more substituents selected from the group consisting of hydroxyl, amino, mercapto, and halogen; R₄ and R₅ are each independently selected from the group consisting of H and C₁-3 alkyl.

35. The compound of formula (I) or a salt, enantiomer, stereoisomer, solvate, or polymorph thereof of claim 34, wherein the LIN represents —(CH₂)ₙ₁—N(R₅)CO—(CH₂)ₙ₂—, wherein the hydrogen atom(s) on the carbon atom(s) of backbone carbon chain of the LIN group is/are optionally replaced by one or more substituents selected from the group consisting of hydroxyl, amino, mercapto, and halogen; R₅ is selected from the group consisting of H and C₁₋₃ alkyl; and n1 and n2 each independently represent an integer of 1, 2, 3, 4, 5, 6, 7, 8, 9, 10, 11, 12, 13, 14, 15, 16 , 17, 18, 19, or 20.

36. The compound of formula (I) or a salt, enantiomer, stereoisomer, solvate, or polymorph thereof of claim 34, wherein the ULM represents the following structure of formula (III): 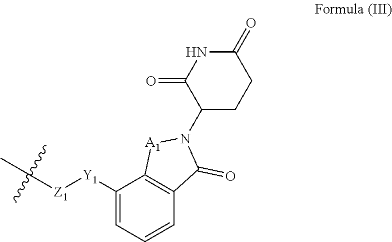
wherein A₁ represents CH₂ or CO; Y₁ represents NH; and Z₁ represents CO or Z₁ is absent.

37. The compound of formula (I) or a salt, enantiomer, stereoisomer, solvate, or polymorph thereof of claim 34, wherein the LIN represents:
—(CH₂)₂—NHCO—CH₂—; —(CH₂)₂—NHCO—(CH₂)₂—; —(CH₂)₂—NHCO—(CH₂)₃—; —(CH₂)₂—NHCO—(CH₂)₄—; —(CH₂)₂—NHCO—(CH₂)₅—; —(CH₂)₂—NHCO—(CH₂)₆—; —(CH₂)₂—NHCO—(CH₂)₇—; —(CH₂)₂—NHCO—(CH₂)₈—; —(CH₂)₂—NHCO—(CH₂)₉—; —(CH₂)₂—NHCO—(CH₂)₁₀—; —(CH₂)₂—NHCO—(CH₂)₁₁—; —(CH₂)₂—NHCO—(CH₂)₁₂—; —(CH₂)₂—NHCO—(CH₂)₁₃—; —(CH₂)₂—NHCO—(CH₂)₁₄—; or —(CH₂)₂—NHCO—(CH₂)₁₅—.

38. The compound of formula (I) or a salt, enantiomer, stereoisomer, solvate, or polymorph thereof of claim 1, wherein
R₁ represents halogen, R₂ represents OH, R₃ represents H, and X represents O;
ULM represents the following structure of formula (IV): 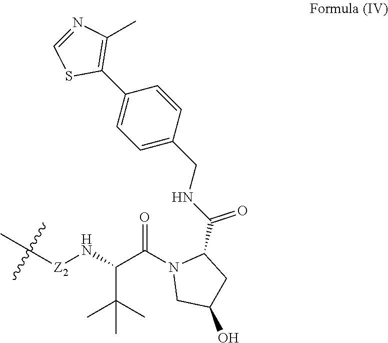
wherein Z₂ represents CO or Z₂ is absent; and
LIN represents alkylene,
wherein the alkylene group is a linear or branched alkylene group interrupted one or more times by the group selected from the group consisting of O, CO, CON(R₄), N(R₅)CO, heterocyclylene, heteroarylene, or any combination thereof, wherein the linear or branched alkylene group is optionally substituted with one or more substituents selected from the group consisting of hydroxyl, amino, mercapto, and halogen; R₄ and R₅ are each independently selected from the group consisting of H and C₁₋₃ alkyl; and the heterocyclylene and heteroarylene group are optionally substituted with the substituent selected from the group consisting of C₁₋₃ alkyl, C₁₋₃ alkoxy, cyano, trifluoromethyl, heterocyclyl, halogen, amino, or hydroxyl.

39. The compound of formula (I) or a salt, enantiomer, stereoisomer, solvate, or polymorph thereof of claim 38, wherein the LIN represents:
—(CH₂)ₙ₁—N(R₅)CO—(CH₂)ₙ₂—(OCH₂)ₙ₃)ₘ₁—; —CH₂)ₙ₁—N(R₅)CO—(CH₂)ₙ₂—(O(CH₂)ₙ₃)ₘ₁—O(CH₂)ₙ₄—; —(CH₂)ₙ₁—N(R₅)CO—(CH₂)ₙ₂—; —(CH₂)ₙ₁—N(R₅)CO—(CH₂)ₙ₂-piperazinylene-(CH₂)ₙ₃—; —(CH₂)ₙ₁-piperazinylene-CO—(CH₂)ₙ₂—; or —(CH₂)ₙ₁-piperazinylene-CO—(CH₂)ₙ₂—(O(CH₂)ₙ₃)ₘ₁—, wherein the hydrogen atom(s) on the carbon atom(s) of backbone carbon chain of the LIN group is/are optionally replaced by one or more substituents selected from the group consisting of hydroxyl, amino, mercapto, and halogen; R₅ is selected from the group consisting of H and C₁₋₃ alkyl; and n1, n2, n3, n4, and ml each independently represent an integer of 1, 2, 3, 4, 5, 6, 7, 8, 9, 10, 11, 12, 13, 14, 15, 16 , 17, 18, 19, or 20, and wherein said piperazinylene group is optionally substituted with the substituent selected from the group consisting of C₁₋₃ alkyl, C₁₋₃ alkoxy, cyano, trifluoromethyl, heterocyclyl, halogen, amino, or hydroxyl.

40. The compound of formula (I) or a salt, enantiomer, stereoisomer, solvate, or polymorph thereof of claim 38, wherein the LIN represents:
—(CH₂)₂—NHCO—CH₂—O(CH₂)₂—OCH₂—; —(CH₂)₂—NHCO—(CH₂)₂—O(CH₂)₂—OCH₂—; —(CH₂)₂—NHCO—(CH₂)₂—(O(CH₂)₂)₂—OCH₂—; —(CH₂)₂—NHCO—(CH₂)₂—(O(CH₂)₂)₂—O(CH₂)₃—; —(CH₂)₂—N(CH₃)CO—CH₂—O(CH₂)₂—OCH₂—; —(CH₂)₂—N(CH₃)CO—CH₂—(O(CH₂)₂)₂—OCH₂—; —(CH₂)₂—N(CH₃)CO—CH₂—(O(CH₂)₂)₃—OCH₂—; —(CH₂)₂—N(CH₃)CO—CH₂—(O(CH₂)₂)₂—O(CH₂)₃—; —(CH₂)₂—NHCO—(CH₂)₂—O(CH₂)₂—; —(CH₂)₂—NHCO—(CH₂)₂—(O(CH₂)₂)₂—; —(CH₂)₂—NHCO—(CH₂)₂—(O(CH₂)₂)₃—; —(CH₂)₂—NHCO—(CH₂)₂—(O(CH₂)₂)₄—; —(CH₂)₂—NHCO—(CH₂)₂—(O(CH₂)₂)₅—; —(CH₂)₂—NHCO—(CH₂)₂—(O(CH₂)₂)₆—; —(CH₂)₂—NHCO—(CH₂)₂—(O(CH₂)₂)₇—; —(CH₂)₂—NHCO—(CH₂)₂—(O(CH₂)₂)₈—; —(CH₂)₂—NHCO—(CH₂)₂—(O(CH₂)₂)₉—; or —(CH₂)₂—NHCO—(CH₂)₂—(O(CH₂)₂)₁₀—
—(CH₂)₂—NHCO—CH₂—; —(CH₂)₂—NHCO—(CH₂)₂—; —(CH₂)₂—NHCO—(CH₂)₃—; —(CH₂)₂—NHCO—(CH₂₁₄—; —(CH₂)₂—NHCO—(CH₂)₅—; —(CH₂)₂—NHCO—(CH₂)₆—; —(CH₂)₂—NHCO—(CH₂₁₇—; —(CH₂)₂—NHCO—(CH₂)₈—; —(CH₂)₂—NHCO—(CH₂)₉—; —(CH₂)₂—NHCO—(CH₂)₁₀—; —(CH₂)₂—NHCO—(CH₂)₁₁—; —(CH₂)₂—NHCO—(CH₂)₁₂—; —(CH₂)₂—NHCO—(CH₂)₁₃—; —(CH₂)₂—NHCO—(CH₂)₁₄—; —(CH₂)₂—NHCO—(CH₂)₁₅—; —(CH₂)₂—NHCO—(CH₂)₁₆—; —(CH₂)₂—NHCO—(CH₂)₁₇—; —(CH₂)₂—NHCO—(CH₂)₁₈—; —(CH₂)₂—NHCO—(CH₂)₁₉—; or —(CH₂)₂—NHCO—(CH₂)₂₀—; or
—(CH₂)₂—NHCO—(CH₂)₂-piperazinylene-(CH₂)₂—; —(CH₂)₂—NHCO—(CH₂)₂-piperazinylene-(CH₂)₃—; —(CH₂)₂—NHCO—(CH₂)₃-piperazinylene-(CH₂)₂—; —(CH₂)₂—NHCO—(CH₂)₃-piperazinylene-(CH₂)₃—; or —(CH₂)₂—NHCO—CH₂-piperazinylene-(CH₂)₁—; or
—(CH₂)₂-piperazinylene-CO—CH₂—O(CH₂)₂—; —(CH₂)₂-piperazinylene-CO—CH₂—OCH₂—; —(CH₂)₂-piperazinylene-CO—CH₂—O(CH₂)₂—OCH₂—; —(CH₂)₂-piperazinylene-CO—(CH₂)₂—O(CH₂)₂—; —(CH₂)₂-piperazinylene-CO—(CH₂)₂—(O(CH₂)₂)₂—; —(CH₂)₂-piperazinylene-CO—(CH₂)₂—(O(CH₂)₂)₃—; —(CH₂)₂-piperazinylene-CO—(CH₂)₂—(O(CH₂)₂)₄—; —(CH₂)₂-piperazinylene-CO—(CH₂)₂O(CH₂)₂)₅—; —(CH₂)₂-piperazinylene-CO—(CH₂)₂—(O(CH₂)₂)₆—; —(CH₂)₂-piperazinylene-CO—(CH₂)₂—(O(CH₂)₂)₇—; —(CH₂)₂-piperazinylene-CO—(CH₂)₂—O(CH₂)₂)₈—; —(CH₂)₂-piperazinylene-CO—(CH₂)₂—(O(CH₂)₂)₉—; or —(CH₂)₂-piperazinylene-CO—(CH₂)₂—(O(CH₂)₂)₁₀—; or
(CH₂)₂-piperazinylene-CO—CH₂—; —(CH₂)₂-piperazinylene-CO—(CH₂)₂—; —(CH₂)₂-piperazinylene-CO—(CH₂)₃—; —(CH₂)₂-piperazinylene-CO—(CH₂)₄—; —(CH₂)₂-piperazinylene-CO—(CH₂)₅—; —(CH₂)₂-piperazinylene-CO—(CH₂)₆—; —(CH₂)₂-piperazinylene-CO—(CH₂)₇—; —(CH₂)₂-piperazinylene-CO—(CH₂)₈—; —(CH₂)₂-piperazinylene-CO—(CH₂)₉—; —(CH₂)₂-piperazinylene-CO—(CH₂)₁₀—; —(CH₂)₂-piperazinylene-CO—(CH₂)₁₁—; —(CH₂)₂-piperazinylene-CO—(CH₂(CH₂—; —(CH₂)₂—piperazinylene-CO—(CH₂)₁₃—; —(CH₂)₂-piperazinylene-CO—(CH₂)₁₄—; or —(CH₂)₂—piperazinylene-CO—(CH₂)₁₅—.

41.-44. (canceled)

45. The compound of formula (I) or a salt, enantiomer, stereoisomer, solvate, or polymorph thereof of claim 1, wherein
R₁ represents H, R₂ and R₃ each independently represent OH, and X represents O;
ULM represents the following structure of formula (IV): 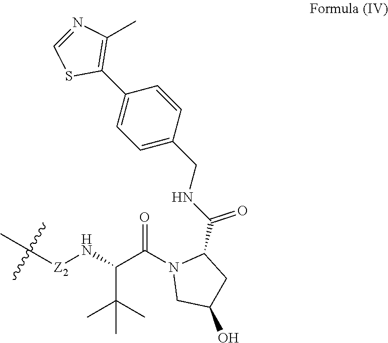
wherein Z₂ represents CO or Z₂ is absent; and
LIN represents alkylene,
wherein the alkylene group is a linear or branched alkylene group interrupted one or more times by the group selected from the group consisting of O, CON(R₄), N(R₅)CO, or any combination thereof, wherein the linear or branched alkylene group is optionally substituted with one or more substituents selected from the group consisting of hydroxyl, amino, mercapto, and halogen; and R₄ and R₅ are each independently selected from the group consisting of H and C₁₋₃ alkyl.

46. The compound of formula (I) or a salt, enantiomer, stereoisomer, solvate, or polymorph thereof of claim 45, wherein the LIN represents —(CH₂)ₙ₁—N(R₅)CO—(CH₂)ₙ₂—(O(CH₂)ₙ₃)ₘ₁—, or —(CH₂)ₙ₁—N(R₅)CO—(CH₂)ₙ₂—, wherein the hydrogen atom(s) on the carbon atom(s) of backbone carbon chain of the LIN group is/are optionally replaced by one or more substituents selected from the group consisting of hydroxyl, amino, mercapto, and halogen; R₅ is selected from the group consisting of H and C₁₋₃ alkyl; and n1, n2, n3, and m1 each independently represent an integer of 1, 2, 3, 4, 5, 6, 7, 8, 9, 10, 11, 12, 13, 14, 15, 16, 17, 18, 19, or 20.

47. The compound of formula (I) or a salt, enantiomer, stereoisomer, solvate, or polymorph thereof of claim 45, wherein the LIN represents:
—(CH₂)₂—NHCO—(CH₂)₂—O(CH₂)₂—; —(CH₂)₂—NHCO—(CH₂)₂—(O(CH₂)₂)₂—; —(CH₂)₂—NHCO—(CH₂)₂—(O(CH₂)₂)₃—; —(CH₂)₂—NHCO—(CH₂)₂—(O(CH₂)₂)₄—; —(CH₂)₂—NHCO—(CH₂)₂—(O(CH₂)₂)₅—; —(CH₂)₂—NHCO—(CH₂)₂—(O(CH₂)₂)₆—; —(CH₂)₂—NHCO—(CH₂)₂—(O(CH₂)₂)₇—; —(CH₂)₂—NHCO—(CH₂)₂—(O(CH₂)₂)₈—; —(CH₂)₂—NHCO—(CH₂)₂—(O(CH₂)₂)₉—; or —(CH₂)₂—NHCO—(CH₂)₂—(O(CH₂)₂)₁₀—; or
—(CH₂)₂—NHCO—CH₂—; —(CH₂)₂-NHCO—(CH₂)₂—; —(CH₂)₂-NHCO—(CH₂)₃—; —(CH₂)₂—NHCO—(CH₂)₄—; —(CH₂)₂—NHCO—(CH₂)₅—; —(CH₂)₂—NHCO—(CH₂)₆—; —(CH₂)₂—NHCO—(CH₂)₇—; —(CH₂)₂—NHCO—(CH₂)₈—; —(CH₂)₂—NHCO—(CH₂)₉—; —(CH₂)₂—NHCO—(CH₂)₁₀—; —(CH₂)₂—NHCO—(CH₂)₁₁—; —(CH₂)₂—NHCO—(CH₂)₁₂—; —(CH₂)₂—NHCO—(CH₂)₁₃—; —(CH₂)₂—NHCO—(CH₂)₁₄—; or —(CH₂)₂—NHCO—(CH₂)₁₅—.

48. (canceled)

49. The compound of formula (I) or a salt, enantiomer, stereoisomer, solvate, or polymorph thereof of claim 1, wherein
R₁ represents H, R₂ and R₃ each independently represent OH, and X represents O;
ULM represents the following structure of formula (II): 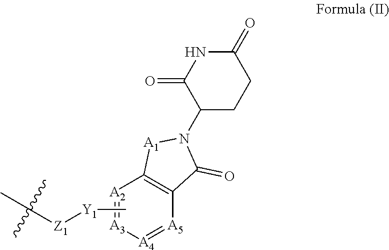
wherein Y₁ represents CH₂, NH, or O; Z₁ represents CO or Z₁ is absent; A₁ represents CH₂ or CO; A₂, A₃, A₄, and A₅ are the same or different and each independently represent CH or N, provided that A₂, A₃, A₄, and A₅ are not N at the same time; and
LIN represents alkylene,
wherein the alkylene group is a linear or branched alkylene group interrupted one or more times by one or more groups selected from the group consisting of CON(R₄), N(R₅)CO, heterocyclylene, heteroarylene, or any combination thereof, wherein the linear or branched alkylene group is optionally substituted with one or more substituents selected from the group consisting of hydroxyl, amino, mercapto, and halogen; R₄ and R₅ are each independently selected from the group consisting of H and C₁₋₃ alkyl; and the heterocyclylene and heteroarylene group are optionally substituted with the substituent selected from the group consisting of C₁₋₃ alkyl, C₁₋₃ alkoxy, cyano, trifluoromethyl, heterocyclyl, halogen, amino, or hydroxyl.

50. The compound of formula (I) or a salt, enantiomer, stereoisomer, solvate, or polymorph thereof of claim 49, wherein the LIN is —(CH₂)ₙ₁—N(R₅)CO—(CH₂)ₙ₂-piperazinylene-(CH₂)ₙ₃—, wherein the hydrogen atom(s) on the carbon atom(s) of backbone carbon chain of the LIN group is/are optionally replaced by one or more substituents selected from the group consisting of hydroxyl, amino, mercapto, and halogen; R₅ is selected from the group consisting of H and C₁₋₃ alkyl; and n1, n2, and n3 each independently represent an integer of 1, 2, 3, 4, 5, 6, 7, 8, 9, 10, 11, 12, 13, 14, 15, 16 , 17, 18, 19, or 20, and wherein said piperazinylene group is optionally substituted with the substituent selected from the group consisting of C₁₋₃ alkyl, C₁₋₃ alkoxy, cyano, trifluoromethyl, heterocyclyl, halogen, amino, or hydroxyl.

51. The compound of formula (I) or a salt, enantiomer, stereoisomer, solvate, or polymorph thereof of claim 49, wherein the ULM represents the following structure of formula (III): 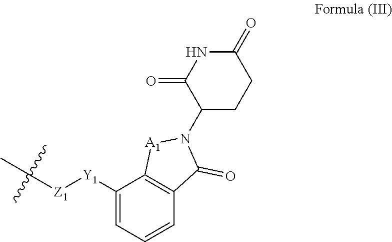
wherein A₁ represents CH₂ or CO; Y₁ represents NH; and Z₁ is absent.

52. The compound of formula (I) or a salt, enantiomer, stereoismer, solvate, or polymorph thereof of claim 49, wherein the LIN represents:
—(CH₂)₂—NHCO—(CH₂)₂-piperazinylene-(CH₂)₂—; —(CH₂)₂—NHCO—(CH₂)₂-piperazinylene-(CH₂)₃—; —(CH₂)₂—NHCO—(CH₂)₃-piperazinylene-(CH₂)₂—; —(CH₂)₂—NHCO—(CH₂)₃-piperazinylene-(CH₂)₃—; or —(CH₂)₂—NHCO—CH₂-piperazinylene-(CH₂)₂—.

53. The compound of formula (I), or a salt, enantiomer, stereoisomer, solvate, or polymorph thereof according to claim 1, which is selected from:
(Z)—N-(2-(4-(4-chloro-1,2-diphenylbut-1-en-1-yl)phenoxy)ethyl)-3-(2-((2-(2,6-dioxopiperidin-3-yl)-1,3-dioxoisoindolin-4-yl)amino)ethoxy)-N-methylpropanamide;
(Z)—N-(2-(4-(4-chloro-1,2-diphenylbut-1-en-1-yl)phenoxy)ethyl)-3-(2-(2-((2-(2,6-dioxopiperidin-3-yl)-1,3-dioxoisoindolin-4-yl)amino)ethoxy)ethoxy)-N-methylpropanamide;
(Z)—N-(2-(4-(4-chloro-1,2-diphenylbut-1-en-1-yl)phenoxy)ethyl)-3-(2-(2-(2-((2-(2,6-dioxopiperidin-3-yl)-1,3-dioxoisoindolin-4-yl)amino)ethoxy)ethoxy)ethoxy)-N-methylpropanamide;
(Z)—N-(2-(4-(4-chloro-1,2-diphenylbut-1-en-1-yl)phenoxy)ethyl)-1-((2-(2,6-dioxopiperidin-3-yl)-1,3-dioxoisoindolin-4-yl)amino)-N-methyl-3,6,9,12-tetraoxapentadecan-15-amide;
(Z)—N-(2-(4-(4-chloro-1,2-diphenylbut-1-en-1-yl)phenoxy)ethyl)-1-((2-(2,6-dioxopiperidin-3-yl)-1,3-dioxoisoindolin-4-yl)amino)-N-methyl-3,6,9,12,15-pentaoxaoctadecan-18-amide;
(Z)—N-(2-(4-(4-chloro-1,2-diphenylbut-1-en-1-yl)phenoxy)ethyl)-2-((2-(2,6-dioxopiperidin-3-yl)-1,3-dioxoisoindolin-4-yl)amino)-N-methylacetamide;
(Z)—N-(2-(4-(4-chloro-1,2-diphenylbut-1-en-1-yl)phenoxy)ethyl)-3-((2-(2,6-dioxopiperidin-3-yl)-1,3-dioxoisoindolin-4-yl)amino)-N-methylpropanamide;
(Z)—N-(2-(4-(4-chloro-1,2-diphenylbut-1-en-1-yl)phenoxy)ethyl)-4-((2-(2,6-dioxopiperidin-3-yl)-1,3-dioxoisoindolin-4-yl)amino)-N-methylbutanamide;
(Z)—N-(2-(4-(4-chloro-1,2-diphenylbut-1-en-1-yl)phenoxy)ethyl)-5-((2-(2,6-dioxopiperidin-3-yl)-1,3-dioxoisoindolin-4-yl)amino)-N-methylpentanamide;
(Z)—N-(2-(4-(4-chloro-1,2-diphenylbut-1-en-1-yl)phenoxy)ethyl)-6-((2-(2,6-dioxopiperidin-3-yl)-1,3-dioxoisoindolin-4-yl)amino)-N-methylhexanamide;
(Z)—N-(2-(4-(4-chloro-1,2-diphenylbut-1-en-1-yl)phenoxy)ethyl)-7-((2-(2,6-dioxopiperidin-3-yl)-1,3-dioxoisoindolin-4-yl)amino)-N-methylheptanamide;
(Z)—N-(2-(4-(4-chloro-1,2-diphenylbut-1-en-1-yl)phenoxy)ethyl)-3-(3-((2-(2,6-dioxopiperidin-3-yl)-1-oxoisoindolin-4-yl)amino)-3-oxopropoxy)-N-methylpropanamide;
(Z)—N-(2-(4-(4-chloro-1,2-diphenylbut-1-en-1-yl)phenoxy)ethyl)-3-(2-(3-((2-(2,6-dioxopiperidin-3-yl)-1-oxoisoindolin-4-yl)amino)-3-oxopropoxy)ethoxy)-N-methylpropanamide;
(Z)—N-(2-(4-(4-chloro-1,2-diphenylbut-1-en-1-yl)phenoxy)ethyl)-3-(2-(2-(3-((2-(2,6-dioxopiperidin-3-yl)-1-oxoisoindolin-4-yl)amino)-3-oxopropoxy)ethoxy)ethoxy)-N-methylpropanamide;
(Z)-N1-(2-(4-(4-chloro-1,2-diphenylbut-1-en-1-yl)phenoxy)ethyl)-N16-(2-(2,6-dioxopiperidin-3-yl)-1-oxoisoindolin-4-yl)-N1-methyl-4,7,10,13-tetraoxahexadecanediamide;
(Z)-N1-(2-(4-(4-chloro-1,2-diphenylbut-1-en-1-yl)phenoxy)ethyl)-N19-(2-(2,6-dioxopiperidin-3-yl)-1-oxoisoindolin-4-yl)-N1-methyl-4,7,10,13,16-pentaoxanonadecanediamide;
(Z)—N-(2-(4-(4-chloro-1,2-diphenylbut-1-en-1-yl)phenoxy)ethyl)-3-(2-((2-(2,6-dioxopiperidin-3-yl)-1-oxoisoindolin-4-yl)amino)ethoxy)-N-methylpropanamide;
(Z)—N-(2-(4-(4-chloro-1,2-diphenylbut-1-en-1-yl)phenoxy)ethyl)-3-(2-(2-((2-(2,6-dioxopiperidin-3-yl)-1-oxoisoindolin-4-yl)amino)ethoxy)ethoxy)-N-methylpropanamide;
(Z)—N-(2-(4-(4-chloro-1,2-diphenylbut-1-en-1-yl)phenoxy)ethyl)-3-(2-(2-(2-((2-(2,6-dioxopiperidin-3-yl)-1-oxoisoindolin-4-yl)amino)ethoxy)ethoxy)ethoxy)-N-methylpropanamide;
(Z)—N-(2-(4-(4-chloro-1,2-diphenylbut-1-en-1-yl)phenoxy)ethyl)-1-((2-(2,6-dioxopiperidin-3-yl)-1-oxoisoindolin-4-yl)amino)-N-methyl-3,6,9,12-tetraoxapentadecan-15-amide;
(Z)—N-(2-(4-(4-chloro-1,2-diphenylbut-1-en-1-yl)phenoxy)ethyl)-1-((2-(2,6-dioxopiperidin-3-yl)-1-oxoisoindolin-4-yl)amino)-N-methyl-3,6,9,12,15-pentaoxaoctadecan-18-amide;
(Z)-N1-(2-(4-(4-chloro-1,2-diphenylbut-1-en-1-yl)phenoxy)ethyl)-N3-(2-(2,6-dioxopiperidin-3-yl)-1-oxoisoindolin-4-yl)-N1-methylmalonamide;
(Z)-N1-(2-(4-(4-chloro-1,2-diphenylbut-1-en-1-yl)phenoxy)ethyl)-N4-(2-(2,6-dioxopiperidin-3-yl)-1-oxoisoindolin-4-yl)-N1-methylsuccinamide;
(Z)-N1-(2-(4-(4-chloro-1,2-diphenylbut-1-en-1-yl)phenoxy)ethyl)-N5-(2-(2,6-dioxopiperidin-3-yl)-1-oxoisoindolin-4-yl)-N1-methylglutaramide;
(Z)-N1-(2-(4-(4-chloro-1,2-diphenylbut-1-en-1-yl)phenoxy)ethyl)-N6-(2-(2,6-dioxopiperidin-3-yl)-1-oxoisoindolin-4-yl)-N1-methyladipamide;
(Z)-N1-(2-(4-(4-chloro-1,2-diphenylbut-1-en-1-yl)phenoxy)ethyl)-N7-(2-(2,6-dioxopiperidin-3-yl)-1-oxoisoindolin-4-yl)-N1-methylheptanediamide;
(Z)-N1-(2-(4-(4-chloro-1,2-diphenylbut-1-en-1-yl)phenoxy)ethyl)-N8-(2-(2,6-dioxopiperidin-3-yl)-1-oxoisoindolin-4-yl)-N1-methyloctanediamide;
(Z)—N-(2-(4-(4-chloro-1,2-diphenylbut-1-en-1-yl)phenoxy)ethyl)-3-((2-(2,6-dioxopiperidin-3-yl)-1-oxoisoindolin-4-yl)amino)-N-methylpropanamide;
(Z)—N-(2-(4-(4-chloro-1,2-diphenylbut-1-en-1-yl)phenoxy)ethyl)-4-((2-(2,6-dioxopiperidin-3-yl)-1-oxoisoindolin-4-yl)amino)-N-methylbutanamide;
(Z)—N-(2-(4-(4-chloro-1,2-diphenylbut-1-en-1-yl)phenoxy)ethyl)-5-((2-(2,6-dioxopiperidin-3-yl)-1-oxoisoindolin-4-yl)amino)-N-methylpentanamide;
(Z)—N-(2-(4-(4-chloro-1,2-diphenylbut-1-en-1-yl)phenoxy)ethyl)-6-((2-(2,6-dioxopiperidin-3-yl)-1-oxoisoindolin-4-yl)amino)-N-methylhexanamide;
(Z)—N-(2-(4-(4-chloro-1,2-diphenylbut-1-en-1-yl)phenoxy)ethyl)-7-((2-(2,6-dioxopiperidin-3-yl)-1-oxoisoindolin-4-yl)amino)-N-methylheptanamide;
(Z)—N-(2-(4-(4-chloro-1,2-diphenylbut-1-en-1-yl)phenoxy)ethyl)-8-((2-(2,6-dioxopiperidin-3-yl)-1-oxoisoindolin-4-yl)amino)-N-methyloctanamide;
(2S,4R)-1-((S)-2-(tert-butyl)-14-(4-((Z)-4-chloro-1,2-diphenylbut-1-en-1-yl)phenoxy)-12-methyl-4,11-dioxo-6,9-dioxa-3,12-diazatetradecanoyl)-4-hydroxy-N-(4-(4-methylthiazol-5-yl)benzyl)pyrrolidine-2-carboxamide;
(2S,4R)-1-((S)-2-(tert-butyl)-16-(4-((Z)-4-chloro-1,2-diphenylbut-1-en-1-yl)phenoxy)-14-methyl-4,13-dioxo-7,10-dioxa-3,14-diazahexadecanoyl)-4-hydroxy-N-(4-(4-methylthiazol-5-yl)benzyl)pyrrolidine-2-carboxamide;
(2S,4R)-1-((S)-2-(tert-butyl)-19-(4-((Z)-4-chloro-1,2-diphenylbut-1-en-1-yl)phenoxy)-17-methyl-4,16-dioxo-7,10,13-trioxa-3,17-diazanonadecanoyl)-4-hydroxy-N-(4-(4-methylthiazol-5-yl)benzyl)pyrrolidine-2-carboxamide;
N1-(2-(4-((Z)-4-chloro-1,2-diphenylbut-1-en-1-yl)phenoxy)ethyl)-N16-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)-N1-methyl-4,7,10,13-tetraoxahexadecanediamide;
N1-(2-(4-((Z)-4-chloro-1,2-diphenylbut-1-en-1-yl)phenoxy)ethyl)-N19-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)-N1-methyl-4,7,10,13,16-pentaoxanonadecanediamide;
N1-(2-(4-((Z)-4-chloro-1,2-diphenylbut-1-en-1-yl)phenoxy)ethyl)-N4-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)-N1-methylsuccinamide;
N1-(2-(4-((Z)-4-chloro-1,2-diphenylbut-1-en-1-yl)phenoxy)ethyl)-N5-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)-N1-methylglutaramide;
N1-(2-(4-((Z)-4-chloro-1,2-diphenylbut-1-en-1-yl)phenoxy)ethyl)-N6-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)-N1-methyladipamide;
N1-(2-(4-((Z)-4-chloro-1,2-diphenylbut-1-en-1-yl)phenoxy)ethyl)-N7-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)-N1-methylheptanediamide;
N1-(2-(4-((Z)-4-chloro-1,2-diphenylbut-1-en-1-yl)phenoxy)ethyl)-N8-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)-N1-methyloctanediamide;
N1-(2-(4-((Z)-4-chloro-1,2-diphenylbut-1-en-1-yl)phenoxy)ethyl)-N9-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)-N1-methylnonanediamide;
N1-(2-(4-((Z)-4-chloro-1,2-diphenylbut-1-en-1-yl)phenoxy)ethyl)-N10-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)-N1-methyldecanediamide;
(2S,4R)-1-((S)-2-(3-(4-(3-((2-(4-((Z)-4-chloro-1,2-diphenylbut-1-en-1-yl)phenoxy)ethyl)(methyl)amino)-3-oxopropyl)piperazin-1-yl)propanamido)-3,3-dimethylbutanoyl)-4-hydroxy-N-(4-(4-methylthiazol-5-yl)benzyl)pyrrolidine-2-carboxamide;
(2S,4R)-1-((S)-2-(3-(4-(3-((2-(4-((Z)-4-chloro-1,2-diphenylbut-1-en-1-yl)phenoxy)ethyl)(methyl)amino)-3-oxopropyl)phenyl)propanamido)-3,3-dimethylbutanoyl)-4-hydroxy-N-(4-(4-methylthiazol-5-yl)benzyl)pyrrolidine-2-carboxamide;
(Z)—N-(2-(4-(4-chloro-1,2-diphenylbut-1-en-1-yl)phenoxy)ethyl)-3-(4-(2-((2-(2,6-dioxopiperidin-3-yl)-1,3-dioxoisoindolin-4-yl)amino)ethyl)piperazin-1-yl)-N-methylpropanamide;
(Z)—N-(2-(4-(4-chloro-1,2-diphenylbut-1-en-1-yl)phenoxy)ethyl)-3-(4-(2-((2-(2,6-dioxopiperidin-3-yl)-1,3-dioxoisoindolin-4-yl)amino)ethyl)phenyl)-N-methylpropanamide;
(2S,4R)-1-((S)-2-(7-((2-(4-((Z)-4-chloro-1,2-diphenylbut-1-en-1-yl)phenoxy)ethyl)(methyl)amino)heptanamido)-3,3-dimethylbutanoyl)-4-hydroxy-N-(4-(4-methylthiazol-5-yl)benzyl)pyrrolidine-2-carboxamide;
(2S,4R)-1-((S)-2-((7-((2-(4-((Z)-4-chloro-1,2-diphenylbut-1-en-1-yl)phenoxy)ethyl)(methyl)amino)-7-oxoheptyl)amino)-3,3-dimethylbutanoyl)-4-hydroxy-N-(4-(4-methylthiazol-5-yl)benzyl)pyrrolidine-2-carboxamide;
(2S,4R)-1-((S)-2-((7-((2-(4-((Z)-4-chloro-1,2-diphenylbut-1-en-1-yl)phenoxy)ethyl)(methyl)amino)heptyl)amino)-3,3-dimethylbutanoyl)-4-hydroxy-N-(4-(4-methylthiazol-5-yl)benzyl)pyrrolidine-2-carboxamide;
(Z)-4-((2-(3-(4-(2-(4-(4-chloro-1,2-diphenylbut-1-en-1-yl)phenoxy)ethyl)piperazin-1-yl)-3-oxopropoxy)ethyl)amino)-2-(2,6-dioxopiperidin-3-yl)isoindoline-1,3-dione;
(Z)-4-((2-(2-(3-(4-(2-(4-(4-chloro-1,2-diphenylbut-1-en-1-yl)phenoxy)ethyl)piperazin-1-yl)-3-oxopropoxy)ethoxy)ethyl)amino)-2-(2,6-dioxopiperidin-3-yl)isoindoline-1,3-dione;
(Z)-4-((2-(2-(2-(3-(4-(2-(4-(4-chloro-1,2-diphenylbut-1-en-1-yl)phenoxy)ethyl)piperazin-1-yl)-3-oxopropoxy)ethoxy)ethoxy)ethyl)amino)-2-(2,6-dioxopiperidin-3-yl)isoindoline-1,3-dione;
(Z)-4-((15-(4-(2-(4-(4-chloro-1,2-diphenylbut-1-en-1-yl)phenoxy)ethyl)piperazin-1-yl)-15-oxo-3,6,9,12-tetraoxapentadecyl)amino)-2-(2,6-dioxopiperidin-3-yl)isoindoline-1,3-dione;
(Z)-4-((18-(4-(2-(4-(4-chloro-1,2-diphenylbut-1-en-1-yl)phenoxy)ethyl)piperazin-1-yl)-18-oxo-3,6,9,12,15-pentaoxaoctadecyl)amino)-2-(2,6-dioxopiperidin-3-yl)isoindoline-1,3-dione;
(Z)-4-((2-(4-(2-(4-(4-chloro-1,2-diphenylbut-1-en-1-yl)phenoxy)ethyl)piperazin-1-yl)-2-oxoethyl)amino)-2-(2,6-dioxopiperidin-3-yl)isoindoline-1,3-dione;
(Z)-4-((3-(4-(2-(4-(4-chloro-1,2-diphenylbut-1-en-1-yl)phenoxy)ethyl)piperazin-1-yl)-3-oxopropyl)amino)-2-(2,6-dioxopiperidin-3-yl)isoindoline-1,3-dione;
(Z)-4-((4-(4-(2-(4-(4-chloro-1,2-diphenylbut-1-en-1-yl)phenoxy)ethyl)piperazin-1-yl)-4-oxobutyl)amino)-2-(2,6-dioxopiperidin-3-yl)isoindoline-1,3-dione;
(Z)-4-((5-(4-(2-(4-(4-chloro-1,2-diphenylbut-1-en-1-yl)phenoxy)ethyl)piperazin-1-yl)-5-oxopentyl)amino)-2-(2,6-dioxopiperidin-3-yl)isoindoline-1,3-dione;
(Z)-4-((6-(4-(2-(4-(4-chloro-1,2-diphenylbut-1-en-1-yl)phenoxy)ethyl)piperazin-1-yl)-6-oxohexyl)amino)-2-(2,6-dioxopiperidin-3-yl)isoindoline-1,3-dione;
(Z)-4-((7-(4-(2-(4-(4-chloro-1,2-diphenylbut-1-en-1-yl)phenoxy)ethyl)piperazin-1-yl)-7-oxoheptyl)amino)-2-(2,6-dioxopiperidin-3-yl)isoindoline-1,3-dione;
(Z)-3-(3-(4-(2-(4-(4-chloro-1,2-diphenylbut-1-en-1-yl)phenoxy)ethyl)piperazin-1-yl)-3-oxopropoxy)-N-(2-(2,6-dioxopiperidin-3-yl)-1-oxoisoindolin-4-yl)propanamide;
(Z)-3-(2-(3-(4-(2-(4-(4-chloro-1,2-diphenylbut-1-en-1-yl)phenoxy)ethyl)piperazin-1-yl)-3-oxopropoxy)ethoxy)-N-(2-(2,6-dioxopiperidin-3-yl)-1-oxoisoindolin-4-yl)propanamide;
(Z)-3-(2-(2-(3-(4-(2-(4-(4-chloro-1,2-diphenylbut-1-en-1-yl)phenoxy)ethyl)piperazin-1-yl)-3-oxopropoxy)ethoxy)ethoxy)-N-(2-(2,6-dioxopiperidin-3-yl)-1-oxoisoindolin-4-yl)propanamide;
(Z)-16-(4-(2-(4-(4-chloro-1,2-diphenylbut-1-en-1-yl)phenoxy)ethyl)piperazin-1-yl)-N-(2-(2,6-dioxopiperidin-3-yl)-1-oxoisoindolin-4-yl)-16-oxo-4,7,10,13-tetraoxahexadecanamide;
(Z)-19-(4-(2-(4-(4-chloro-1,2-diphenylbut-1-en-1-yl)phenoxy)ethyl)piperazin-1-yl)-N-(2-(2,6-dioxopiperidin-3-yl)-1-oxoisoindolin-4-yl)-19-oxo-4,7,10,13,16-pentaoxanonadecanamide;
(Z)-3-(4-((2-(3-(4-(2-(4-(4-chloro-1,2-diphenylbut-1-en-1-yl)phenoxy)ethyl)piperazin-1-yl)-3-oxopropoxy)ethyl)amino)-1-oxoisoindolin-2-yl)piperidine-2,6-dione;
(Z)-3-(4-((2-(2-(3-(4-(2-(4-(4-chloro-1,2-diphenylbut-1-en-1-yl)phenoxy)ethyl)piperazin-1-yl)-3-oxopropoxy)ethoxy)ethyl)amino)-1-oxoisoindolin-2-yl)piperidine-2,6-dione;
(Z)-3-(4-((2-(2-(2-(3-(4-(2-(4-(4-chloro-1,2-diphenylbut-1-en-1-yl)phenoxy)ethyl)piperazin-1-yl)-3-oxopropoxy)ethoxy)ethoxy)ethyl)amino)-1-oxoisoindolin-2-yl)piperidine-2,6-dione;
(Z)-3-(4-((15-(4-(2-(4-(4-chloro-1,2-diphenylbut-1-en-1-yl)phenoxy)ethyl)piperazin-1-yl)-15-oxo-3,6,9,12-tetraoxapentadecyl)amino)-1-oxoisoindolin-2-yl)piperidine-2,6-dione;
(Z)-3-(4-((18-(4-(2-(4-(4-chloro-1,2-diphenylbut-1-en-1-yl)phenoxy)ethyl)piperazin-1-yl)-18-oxo-3,6,9,12,15-pentaoxaoctadecyl)amino)-1-oxoisoindolin-2-yl)piperidine-2,6-dione;
(Z)-3-(4-(2-(4-(4-chloro-1,2-diphenylbut-1-en-1-yl)phenoxy)ethyl)piperazin-1-yl)-N-(2-(2,6-dioxopiperidin-3-yl)-1-oxoisoindolin-4-yl)-3-oxopropanamide;
(Z)-4-(4-(2-(4-(4-chloro-1,2-diphenylbut-1-en-1-yl)phenoxy)ethyl)piperazin-1-yl)-N-(2-(2,6-dioxopiperidin-3-yl)-1-oxoisoindolin-4-yl)-4-oxobutanamide;
(Z)-5-(4-(2-(4-(4-chloro-1,2-diphenylbut-1-en-1-yl)phenoxy)ethyl)piperazin-1-yl)-N-(2-(2,6-dioxopiperidin-3-yl)-1-oxoisoindolin-4-yl)-5-oxopentanamide;
(Z)-6-(4-(2-(4-(4-chloro-1,2-diphenylbut-1-en-1-yl)phenoxy)ethyl)piperazin-1-yl)-N-(2-(2,6-dioxopiperidin-3-yl)-1-oxoisoindolin-4-yl)-6-oxohexanamide;
(Z)-7-(4-(2-(4-(4-chloro-1,2-diphenylbut-1-en-1-yl)phenoxy)ethyl)piperazin-1-yl)-N-(2-(2,6-dioxopiperidin-3-yl)-1-oxoisoindolin-4-yl)-7-oxoheptanamide;
(Z)-3-(4-((2-(4-(2-(4-(4-chloro-1,2-diphenylbut-1-en-1-yl)phenoxy)ethyl)piperazin-1-yl)-2-oxoethyl)amino)-1-oxoisoindolin-2-yl)piperidine-2,6-dione;
(Z)-3-(4-((3-(4-(2-(4-(4-chloro-1,2-diphenylbut-1-en-1-yl)phenoxy)ethyl)piperazin-1-yl)-3-oxopropyl)amino)-1-oxoisoindolin-2-yl)piperidine-2,6-dione;
(Z)-3-(4-((4-(4-(2-(4-(4-chloro-1,2-diphenylbut-1-en-1-yl)phenoxy)ethyl)piperazin-1-yl)-4-oxobutyl)amino)-1-oxoisoindolin-2-yl)piperidine-2,6-dione;
(Z)-3-(4-((5-(4-(2-(4-(4-chloro-1,2-diphenylbut-1-en-1-yl)phenoxy)ethyl)piperazin-1-yl)-5-oxopentyl)amino)-1-oxoisoindolin-2-yl)piperidine-2,6-dione;
(Z)-3-(4-((6-(4-(2-(4-(4-chloro-1,2-diphenylbut-1-en-1-yl)phenoxy)ethyl)piperazin-1-yl)-6-oxohexyl)amino)-1-oxoisoindolin-2-yl)piperidine-2,6-dione;
(Z)-3-(4-((7-(4-(2-(4-(4-chloro-1,2-diphenylbut-1-en-1-yl)phenoxy)ethyl)piperazin-1-yl)-7-oxoheptyl)amino)-1-oxoisoindolin-2-yl)piperidine-2,6-dione;
(2S,4R)-1-((S)-2-(2-(2-(2-(4-(2-(4-((Z)-4-chloro-1,2-diphenylbut-1-en-1-yl)phenoxy)ethyl)piperazin-1-yl)-2-oxoethoxy)ethoxy)acetamido)-3,3-dimethylbutanoyl)-4-hydroxy-N-(4-(4-methylthiazol-5-yl)benzyl)pyrrolidine-2-carboxamide;
(2S,4R)-1-((S)-2-(3-(2-(3-(4-(2-(4-((Z)-4-chloro-1,2-diphenylbut-1-en-1-yl)phenoxy)ethyl)piperazin-1-yl)-3-oxopropoxy)ethoxy)propanamido)-3,3-dimethylbutanoyl)-4-hydroxy-N-(4-(4-methylthiazol-5-yl)benzyl)pyrrolidine-2-carboxamide;
(2S,4R)-1-((S)-2-(tert-butyl)-16-(4-(2-(4-((Z)-4-chloro-1,2-diphenylbut-1-en-1-yl)phenoxy)ethyl)piperazin-1-yl)-4,16-dioxo-7,10,13-trioxa-3-azahexadecanoyl)-4-hydroxy-N-(4-(4-methylthiazol-5-yl)benzyl)pyrrolidine-2-carboxamide;
(2S,4R)-1-((S)-2-(tert-butyl)-19-(4-(2-(4-((Z)-4-chloro-1,2-diphenylbut-1-en-1-yl)phenoxy)ethyl)piperazin-1-yl)-4,19-dioxo-7,10,13,16-tetraoxa-3-azanonadecanoyl)-4-hydroxy-N-(4-(4-methylthiazol-5-yl)benzyl)pyrrolidine-2-carboxamide;
(2S,4R)-1-((S)-2-(tert-butyl)-22-(4-(2-(4-((Z)-4-chloro-1,2-diphenylbut-1-en-1-yl)phenoxy)ethyl)piperazin-1-yl)-4,22-dioxo-7,10,13,16,19-pentaoxa-3-azadocosanoyl)-4-hydroxy-N-(4-(4-methylthiazol-5-yl)benzyl)pyrrolidine-2-carboxamide;
(2S,4R)-1-((S)-2-(4-(4-(2-(4-((Z)-4-chloro-1,2-diphenylbut-1-en-1-yl)phenoxy)ethyl)piperazin-1-yl)-4-oxobutanamido)-3,3-dimethylbutanoyl)-4-hydroxy-N-(4-(4-methylthiazol-5-yl)benzyl)pyrrolidine-2-carboxamide;
(2S,4R)-1-((S)-2-(5-(4-(2-(4-((Z)-4-chloro-1,2-diphenylbut-1-en-1-yl)phenoxy)ethyl)piperazin-1-yl)-5-oxopentanamido)-3,3-dimethylbutanoyl)-4-hydroxy-N-(4-(4-methylthiazol-5-yl)benzyl)pyrrolidine-2-carboxamide;
(2S,4R)-1-((S)-2-(6-(4-(2-(4-((Z)-4-chloro-1,2-diphenylbut-1-en-1-yl)phenoxy)ethyl)piperazin-1-yl)-6-oxohexanamido)-3,3-dimethylbutanoyl)-4-hydroxy-N-(4-(4-methylthiazol-5-yl)benzyl)pyrrolidine-2-carboxamide;
(2S,4R)-1-((S)-2-(7-(4-(2-(4-((Z)-4-chloro-1,2-diphenylbut-1-en-1-yl)phenoxy)ethyl)piperazin-1-yl)-7-oxoheptanamido)-3,3-dimethylbutanoyl)-4-hydroxy-N-(4-(4-methylthiazol-5-yl)benzyl)pyrrolidine-2-carboxamide;
(2S,4R)-1-((S)-2-(8-(4-(2-(4-((Z)-4-chloro-1,2-diphenylbut-1-en-1-yl)phenoxy)ethyl)piperazin-1-yl)-8-oxooctanamido)-3,3-dimethylbutanoyl)-4-hydroxy-N-(4-(4-methylthiazol-5-yl)benzyl)pyrrolidine-2-carboxamide;
(2S,4R)-1-((S)-2-(9-(4-(2-(4-((Z)-4-chloro-1,2-diphenylbut-1-en-1-yl)phenoxy)ethyl)piperazin-1-yl)-9-oxononanamido)-3,3-dimethylbutanoyl)-4-hydroxy-N-(4-(4-methylthiazol-5-yl)benzyl)pyrrolidine-2-carboxamide;
(2S,4R)-1-((S)-2-(10-(4-(2-(4-((Z)-4-chloro-1,2-diphenylbut-1-en-1-yl)phenoxy)ethyl)piperazin-1-yl)-10-oxodecanamido)-3,3-dimethylbutanoyl)-4-hydroxy-N-(4-(4-methylthiazol-5-yl)benzyl)pyrrolidine-2-carboxamide;
(2S,4R)-1-((S)-2-(7-(4-(2-(4-((Z)-4-chloro-1,2-diphenylbut-1-en-1-yl)phenoxy)ethyl)piperazin-1-yl)heptanamido)-3,3-dimethylbutanoyl)-4-hydroxy-N-(4-(4-methylthiazol-5-yl)benzyl)pyrrolidine-2-carboxamide;
(2S,4R)-1-((S)-2-((7-(4-(2-(4-((Z)-4-chloro-1,2-diphenylbut-1-en-1-yl)phenoxy)ethyl)piperazin-1-yl)-7-oxoheptyl)amino)-3,3-dimethylbutanoyl)-4-hydroxy-N-(4-(4-methylthiazol-5-yl)benzyl)pyrrolidine-2-carboxamide;
(2S,4R)-1-((S)-2-((7-(4-(2-(4-((Z)-4-chloro-1,2-diphenylbut-1-en-1-yl)phenoxy)ethyl)piperazin-1-yl)heptyl)amino)-3,3-dimethylbutanoyl)-4-hydroxy-N-(4-(4-methylthiazol-5-yl)benzyl)pyrrolidine-2-carboxamide;
(N-(2-(4-(4-chloro-1-(4-hydroxyphenyl)-2-phenylbut-1-en-1-yl)phenoxy)ethyl)-3-(2-((2-(2,6-dioxopiperidin-3-yl)-1,3-dioxoisoindolin-4-yl)amino)ethoxy)propanamide;
N-(2-(4-(4-chloro-1-(4-hydroxyphenyl)-2-phenylbut-1-en-1-yl)phenoxy)ethyl)-3-(2-(2-((2-(2,6-dioxopiperidin-3-yl)-1,3-dioxoisoindolin-4-yl)amino)ethoxy)ethoxy)propanamide;
N-(2-(4-(4-chloro-1-(4-hydroxyphenyl)-2-phenylbut-1-en-1-yl)phenoxy)ethyl)-3-(2-(2-(2-((2-(2,6-dioxopiperidin-3-yl)-1,3-dioxoisoindolin-4-yl)amino)ethoxy)ethoxy)ethoxy)propanamide;
N-(2-(4-(4-chloro-1-(4-hydroxyphenyl)-2-phenylbut-1-en-1-yl)phenoxy)ethyl)-1-((2-(2,6-dioxopiperidin-3-yl)-1,3-dioxoisoindolin-4-yl)amino)-3 ,6,9,12-tetraoxapentadecan-15-amide;
N-(2-(4-(4-chloro-1-(4-hydroxyphenyl)-2-phenylbut-1-en-1-yl)phenoxy)ethyl)-1-((2-(2,6-dioxopiperidin-3-yl)-1,3-dioxoisoindolin-4-yl)amino)-3 ,6,9,12,15-pentaoxaoctadecan-18-amide;
N-(2-(4-(4-chloro-1-(4-hydroxyphenyl)-2-phenylbut-1-en-1-yl)phenoxy)ethyl)-2-((2-(2,6-dioxopiperidin-3-yl)-1,3-dioxoisoindolin-4-yl)amino)acetamide;
N-(2-(4-(4-chloro-1-(4-hydroxyphenyl)-2-phenylbut-1-en-1-yl)phenoxy)ethyl)-3-((2-(2,6-dioxopiperidin-3-yl)-1,3-dioxoisoindolin-4-yl)amino)propanamide;
N-(2-(4-(4-chloro-1-(4-hydroxyphenyl)-2-phenylbut-1-en-1-yl)phenoxy)ethyl)-4-((2-(2,6-dioxopiperidin-3-yl)-1,3-dioxoisoindolin-4-yl)amino)butanamide;
N-(2-(4-(4-chloro-1-(4-hydroxyphenyl)-2-phenylbut-1-en-1-yl)phenoxy)ethyl)-5-((2-(2,6-dioxopiperidin-3-yl)-1,3-dioxoisoindolin-4-yl)amino)pentanamide;
N-(2-(4-(4-chloro-1-(4-hydroxyphenyl)-2-phenylbut-1-en-1-yl)phenoxy)ethyl)-6-((2-(2,6-dioxopiperidin-3-yl)-1,3-dioxoisoindolin-4-yl)amino)hexanamide;
N-(2-(4-(4-chloro-1-(4-hydroxyphenyl)-2-phenylbut-1-en-1-yl)phenoxy)ethyl)-7-((2-(2,6-dioxopiperidin-3-yl)-1,3-dioxoisoindolin-4-yl)amino)heptanamide;
N-(2-(4-(4-chloro-1-(4-hydroxyphenyl)-2-phenylbut-1-en-1-yl)phenoxy)ethyl)-3-(3-((2-(2,6-dioxopiperidin-3-yl)-1-oxoisoindolin-4-yl)amino)-3-oxopropoxy)propanamide;
N-(2-(4-(4-chloro-1-(4-hydroxyphenyl)-2-phenylbut-1-en-1-yl)phenoxy)ethyl)-3-(2-(3-((2-(2,6-dioxopiperidin-3-yl)-1-oxoisoindolin-4-yl)amino)-3-oxopropoxy)ethoxy)propanamide;
N-(2-(4-(4-chloro-1-(4-hydroxyphenyl)-2-phenylbut-1-en-1-yl)phenoxy)ethyl)-3-(2-(2-(3-((2-(2,6-dioxopiperidin-3-yl)-1-oxoisoindolin-4-yl)amino)-3-oxopropoxy)ethoxy)ethoxy)propanamide;
N1-(2-(4-(4-chloro-1-(4-hydroxyphenyl)-2-phenylbut-1-en-1-yl)phenoxy)ethyl)-N16-(2-(2,6-dioxopiperidin-3-yl)-1-oxoisoindolin-4-yl)-4,7,10,13-tetraoxahexadecanediamide;
N1-(2-(4-(4-chloro-1-(4-hydroxyphenyl)-2-phenylbut-1-en-1-yl)phenoxy)ethyl)-N19-(2-(2,6-dioxopiperidin-3-yl)-1-oxoisoindolin-4-yl)-4,7,10,13,16-pentaoxanonadecanediamide;
N-(2-(4-(4-chloro-1-(4-hydroxyphenyl)-2-phenylbut-1-en-1-yl)phenoxy)ethyl)-3-(2-((2-(2,6-dioxopiperidin-3-yl)-1-oxoisoindolin-4-yl)amino)ethoxy)propanamide;
N-(2-(4-(4-chloro-1-(4-hydroxyphenyl)-2-phenylbut-1-en-1-yl)phenoxy)ethyl)-3-(2-(2-((2-(2,6-dioxopiperidin-3-yl)-1-oxoisoindolin-4-yl)amino)ethoxy)ethoxy)propanamide;
N-(2-(4-(4-chloro-1-(4-hydroxyphenyl)-2-phenylbut-1-en-1-yl)phenoxy)ethyl)-3-(2-(2-(2-((2-(2,6-dioxopiperidin-3-yl)-1-oxoisoindolin-4-yl)amino)ethoxy)ethoxy)ethoxy)propanamide;
N-(2-(4-(4-chloro-1-(4-hydroxyphenyl)-2-phenylbut-1-en-1-yl)phenoxy)ethyl)-1-((2-(2,6-dioxopiperidin-3-yl)-1-oxoisoindolin-4-yl)amino)-3 ,6,9,12-tetraoxapentadecan-15-amide;
N-(2-(4-(4-chloro-1-(4-hydroxyphenyl)-2-phenylbut-1-en-1-yl)phenoxy)ethyl)-1-((2-(2,6-dioxopiperidin-3-yl)-1-oxoisoindolin-4-yl)amino)-3,6,9,12,15-pentaoxaoctadecan-18-amide;
N1-(2-(4-(4-chloro-1-(4-hydroxyphenyl)-2-phenylbut-1-en-1-yl)phenoxy)ethyl)-N3-(2-(2,6-dioxopiperidin-3-yl)-1-oxoisoindolin-4-yl)malonamide;
N1-(2-(4-(4-chloro-1-(4-hydroxyphenyl)-2-phenylbut-1-en-1-yl)phenoxy)ethyl)-N4-(2-(2,6-dioxopiperidin-3-yl)-1-oxoisoindolin-4-yl)succinamide;
N1-(2-(4-(4-chloro-1-(4-hydroxyphenyl)-2-phenylbut-1-en-1-yl)phenoxy)ethyl)-N5-(2-(2,6-dioxopiperidin-3-yl)-1-oxoisoindolin-4-yl)glutaramide;
N1-(2-(4-(4-chloro-1-(4-hydroxyphenyl)-2-phenylbut-1-en-1-yl)phenoxy)ethyl)-N6-(2-(2,6-dioxopiperidin-3-yl)-1-oxoisoindolin-4-yl)adipamide;
N1-(2-(4-(4-chloro-1-(4-hydroxyphenyl)-2-phenylbut-1-en-1-yl)phenoxy)ethyl)-N7-(2-(2,6-dioxopiperidin-3-yl)-1-oxoisoindolin-4-yl)heptanediamide;
N1-(2-(4-(4-chloro-1-(4-hydroxyphenyl)-2-phenylbut-1-en-1-yl)phenoxy)ethyl)-N8-(2-(2,6-dioxopiperidin-3-yl)-1-oxoisoindolin-4-yl)octanediamide;
N-(2-(4-(4-chloro-1-(4-hydroxyphenyl)-2-phenylbut-1-en-1-yl)phenoxy)ethyl)-2-((2-(2,6-dioxopiperidin-3-yl)-1-oxoisoindolin-4-yl)amino)acetamide;
N-(2-(4-(4-chloro-1-(4-hydroxyphenyl)-2-phenylbut-1-en-1-yl)phenoxy)ethyl)-3-((2-(2,6-dioxopiperidin-3-yl)-1-oxoisoindolin-4-yl)amino)propanamide;
N-(2-(4-(4-chloro-1-(4-hydroxyphenyl)-2-phenylbut-1-en-1-yl)phenoxy)ethyl)-4-((2-(2,6-dioxopiperidin-3-yl)-1-oxoisoindolin-4-yl)amino)butanamide;
N-(2-(4-(4-chloro-1-(4-hydroxyphenyl)-2-phenylbut-1-en-1-yl)phenoxy)ethyl)-5-((2-(2,6-dioxopiperidin-3-yl)-1-oxoisoindolin-4-yl)amino)pentanamide;
N-(2-(4-(4-chloro-1-(4-hydroxyphenyl)-2-phenylbut-1-en-1-yl)phenoxy)ethyl)-6-((2-(2,6-dioxopiperidin-3-yl)-1-oxoisoindolin-4-yl)amino)hexanamide;
N-(2-(4-(4-chloro-1-(4-hydroxyphenyl)-2-phenylbut-1-en-1-yl)phenoxy)ethyl)-7-((2-(2,6-dioxopiperidin-3-yl)-1-oxoisoindolin-4-yl)amino)heptanamide;
N-(2-(4-(4-chloro-1-(4-hydroxyphenyl)-2-phenylbut-1-en-1-yl)phenoxy)ethyl)-8-((2-(2,6-dioxopiperidin-3-yl)-1-oxoisoindolin-4-yl)amino)octanamide;
(2S,4R)-1-((S)-2-(tert-butyl)-14-(4-(4-chloro-1-(4-hydroxyphenyl)-2-phenylbut-1-en-1-yl)phenoxy)-4,11-dioxo-6,9-dioxa-3,12-diazatetradecanoyl)-4-hydroxy-N-(4-(4-methylthiazol-5-yl)benzyl)pyrrolidine-2-carboxamide;
(2S,4R)-1-((S)-2-(tert-butyl)-16-(4-(4-chloro-1-(4-hydroxyphenyl)-2-phenylbut-1-en-1-yl)phenoxy)-4,13-dioxo-7,10-dioxa-3,14-diazahexadecanoyl)-4-hydroxy-N-(4-(4-methylthiazol-5-yl)benzyl)pyrrolidine-2-carboxamide;
(2S,4R)-1-((S)-2-(tert-butyl)-19-(4-(4-chloro-1-(4-hydroxyphenyl)-2-phenylbut-1-en-1-yl)phenoxy)-4,16-dioxo-7,10,13-trioxa-3,17-diazanonadecanoyl)-4-hydroxy-N-(4-(4-methylthiazol-5-yl)benzyl)pyrrolidine-2-carboxamide;
N1-(2-(4-(4-chloro-1-(4-hydroxyphenyl)-2-phenylbut-1-en-1-yl)phenoxy)ethyl)-N16-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)-4,7,10,13-tetraoxahexadecanediamide;
N1-(2-(4-(4-chloro-1-(4-hydroxyphenyl)-2-phenylbut-1-en-1-yl)phenoxy)ethyl)-N19-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)-4,7,10,13,16-pentaoxanonadecanediamide;
N1-(2-(4-(4-chloro-1-(4-hydroxyphenyl)-2-phenylbut-1-en-1-yl)phenoxy)ethyl)-N4-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)succinamide;
N1-(2-(4-(4-chloro-1-(4-hydroxyphenyl)-2-phenylbut-1-en-1-yl)phenoxy)ethyl)-N5-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)glutaramide;
N1-(2-(4-(4-chloro-1-(4-hydroxyphenyl)-2-phenylbut-1-en-1-yl)phenoxy)ethyl)-N6-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)adipamide;
N1-(2-(4-(4-chloro-1-(4-hydroxyphenyl)-2-phenylbut-1-en-1-yl)phenoxy)ethyl)-N7-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)heptanediamide;
N1-(2-(4-(4-chloro-1-(4-hydroxyphenyl)-2-phenylbut-1-en-1-yl)phenoxy)ethyl)-N8-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)octanediamide;
N1-(2-(4-(4-chloro-1-(4-hydroxyphenyl)-2-phenylbut-1-en-1-yl)phenoxy)ethyl)-N9-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)nonanediamide;
N1-(2-(4-((Z)-4-chloro-1-(4-hydroxyphenyl)-2-phenylbut-1-en-1-yl)phenoxy)ethyl)-N10-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)decanediamide;
N1-(2-(4-(4-chloro-1-(4-hydroxyphenyl)-2-phenylbut-1-en-1-yl)phenoxy)ethyl)-N11-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)undecanediamide;
N1-(2-(4-(4-chloro-1-(4-hydroxyphenyl)-2-phenylbut-1-en-1-yl)phenoxy)ethyl)-N14-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)tetradecanediamide;
N1-(2-(4-(4-chloro-1-(4-hydroxyphenyl)-2-phenylbut-1-en-1-yl)phenoxy)ethyl)-N16-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)hexadecanediamide;
(2S,4R)-1-((S)-2-(3-(4-(3-((2-(4-(4-chloro-1-(4-hydroxyphenyl)-2-phenylbut-1-en-1-yl)phenoxy)ethyl)amino)-3-oxopropyl)piperazin-1-yl)propanamido)-3,3-dimethylbutanoyl)-4-hydroxy-N-(4-(4-methylthiazol-5-yl)benzyl)pyrrolidine-2-carboxamide;
(2S,4R)-1-((S)-2-(3-(4-(3-((2-(4-(4-chloro-1-(4-hydroxyphenyl)-2-phenylbut-1-en-1-yl)phenoxy)ethyl)amino)-3-oxopropyl)phenyl)propanamido)-3,3-dimethylbutanoyl)-4-hydroxy-N-(4-(4-methylthiazol-5-yl)benzyl)pyrrolidine-2-carboxamide;
N-(2-(4-(4-chloro-1-(4-hydroxyphenyl)-2-phenylbut-1-en-1-yl)phenoxy)ethyl)-3-(4-(2-((2-(2,6-dioxopiperidin-3-yl)-1,3-dioxoisoindolin-4-yl)amino)ethyl)piperazin-1-yl)propanamide;
N-(2-(4-(4-chloro-1-(4-hydroxyphenyl)-2-phenylbut-1-en-1-yl)phenoxy)ethyl)-3-(4-(2-((2-(2,6-dioxopiperidin-3-yl)-1,3-dioxoisoindolin-4-yl)amino)ethyl)phenyl)propanamide;
(2S,4R)-1-((S)-2-(7-((2-(4-(4-chloro-1-(4-hydroxyphenyl)-2-phenylbut-1-en-1-yl)phenoxy)ethyl)amino)heptanamido)-3,3-dimethylbutanoyl)-4-hydroxy-N-(4-(4-methylthiazol-5-yl)benzyl)pyrrolidine-2-carboxamide;
(2S,4R)-1-((S)-2-((7-((2-(4-(4-chloro-1-(4-hydroxyphenyl)-2-phenylbut-1-en-1-yl)phenoxy)ethyl)amino)-7-oxoheptyl)amino)-3,3-dimethylbutanoyl)-4-hydroxy-N-(4-(4-methylthiazol-5-yl)benzyl)pyrrolidine-2-carboxamide;
(2S,4R)-1-((S)-2-((7-((2-(4-(4-chloro-1-(4-hydroxyphenyl)-2-phenylbut-1-en-1-yl)phenoxy)ethyl)amino)heptyl)amino)-3,3-dimethylbutanoyl)-4-hydroxy-N-(4-(4-methylthiazol-5-yl)benzyl)pyrrolidine-2-carboxamide;
4-((2-(3-(4-(2-(4-(4-chloro-1-(4-hydroxyphenyl)-2-phenylbut-1-en-1-yl)phenoxy)ethyl)piperazin-1-yl)-3-oxopropoxy)ethyl)amino)-2-(2,6-dioxopiperidin-3-yl)isoindoline-1,3-dione;
4-((2-(2-(3-(4-(2-(4-(4-chloro-1-(4-hydroxyphenyl)-2-phenylbut-1-en-1-yl)phenoxy)ethyl)piperazin-1-yl)-3-oxopropoxy)ethoxy)ethyl)amino)-2-(2,6-dioxopiperidin-3-yl)isoindoline-1,3-dione;
4-((2-(2-(2-(3-(4-(2-(4-(4-chloro-1-(4-hydroxyphenyl)-2-phenylbut-1-en-1-yl)phenoxy)ethyl)piperazin-1-yl)-3-oxopropoxy)ethoxy)ethoxy)ethyl)amino)-2-(2,6-dioxopiperidin-3-yl)isoindoline-1,3-dione;
4-((15-(4-(2-(4-(4-chloro-1-(4-hydroxyphenyl)-2-phenylbut-1-en-1-yl)phenoxy)ethyl)piperazin-1-yl)-15-oxo-3,6,9,12-tetraoxapentadecyl)amino)-2-(2,6-dioxopiperidin-3-yl)isoindoline-1,3-dione;
4-((18-(4-(2-(4-(4-chloro-1-(4-hydroxyphenyl)-2-phenylbut-1-en-1-yl)phenoxy)ethyl)piperazin-1-yl)-18-oxo-3,6,9,12,15-pentaoxaoctadecyl)amino)-2-(2,6-dioxopiperidin-3-yl)isoindoline-1,3-dione;
4-((2-(4-(2-(4-(4-chloro-1-(4-hydroxyphenyl)-2-phenylbut-1-en-1-yl)phenoxy)ethyl)piperazin-1-yl)-2-oxoethyl)amino)-2-(2,6-dioxopiperidin-3-yl)isoindoline-1,3-dione;
4-((3-(4-(2-(4-(4-chloro-1-(4-hydroxyphenyl)-2-phenylbut-1-en-1-yl)phenoxy)ethyl)piperazin-1-yl)-3-oxopropyl)amino)-2-(2,6-dioxopiperidin-3-yl)isoindoline-1,3-dione;
4-((4-(4-(2-(4-(4-chloro-1-(4-hydroxyphenyl)-2-phenylbut-1-en-1-yl)phenoxy)ethyl)piperazin-1-yl)-4-oxobutyl)amino)-2-(2,6-dioxopiperidin-3-yl)isoindoline-1,3-dione;
4-((5-(4-(2-(4-(4-chloro-1-(4-hydroxyphenyl)-2-phenylbut-1-en-1-yl)phenoxy)ethyl)piperazin-1-yl)-5-oxopentyl)amino)-2-(2,6-dioxopiperidin-3-yl)isoindoline-1,3-dione;
4-((6-(4-(2-(4-(4-chloro-1-(4-hydroxyphenyl)-2-phenylbut-1-en-1-yl)phenoxy)ethyl)piperazin-1-yl)-6-oxohexyl)amino)-2-(2,6-dioxopiperidin-3-yl)isoindoline-1,3-dione;
4-((7-(4-(2-(4-(4-chloro-1-(4-hydroxyphenyl)-2-phenylbut-1-en-1-yl)phenoxy)ethyl)piperazin-1-yl)-7-oxoheptyl)amino)-2-(2.6-dioxopiperidin-3-yl)isoindoline-1,3-dione;
3-(3-(4-(2-(4-(4-chloro-1-(4-hydroxyphenyl)-2-phenylbut-1-en-1-yl)phenoxy)ethyl)piperazin-1-yl)-3-oxopropoxy)-N-(2-(2,6-dioxopiperidin-3-yl)-1-oxoisoindolin-4-yl)propanamide;
3-(2-(3-(4-(2-(4-(4-chloro-1-(4-hydroxyphenyl)-2-phenylbut-1-en-1-yl)phenoxy)ethyl)piperazin-1-yl)-3-oxopropoxy)ethoxy)-N-(2-(2,6-dioxopiperidin-3-yl)-1-oxoisoindolin-4-yl)propanamide;
3-(2-(2-(3-(4-(2-(4-(4-chloro-1-(4-hydroxyphenyl)-2-phenylbut-1-en-1-yl)phenoxy)ethyl)piperazin-1-yl)-3-oxopropoxy)ethoxy )ethoxy)-N-(2-(2,6-dioxopiperidin-3-yl)-1-oxoisoindolin-4-yl)propanamide;
16-(4-(2-(4-(4-chloro-1-(4-hydroxyphenyl)-2-phenylbut-1-en-1-yl)phenoxy)ethyl)piperazin-1-yl)-N-(2-(2,6-dioxopiperidin-3-yl)-1-oxoisoindolin 4-yl)-16-oxo-4,7,10,13-tetraoxahexadecanamide;
19-(4-(2-(4-(4-chloro-1-(4-hydroxyphenyl)-2-phenylbut-1-en-1-yl)phenoxy)ethyl)piperazin-1-yl)-N-(2-(2,6-dioxopiperidin-3-yl)-1-oxoisoindolin 4-yl)-19-oxo-4,7,10,13,16-pentaoxanonadecanamide;
3-(4-((2-(3-(4-(2-(4-(4-chloro-1-(4-hydroxyphenyl)-2-phenylbut-1-en-1-yl)phenoxy)ethyl)piperazin-1-yl)-3-oxopropoxy)ethyl)amino)-1-oxoisoindolin-2-yl)piperidine-2,6-dione;
3-(4-((2-(2-(3-(4-(2-(4-(4-chloro-1-(4-hydroxyphenyl)-2-phenylbut-1-en-1-yl)phenoxy)ethyl)piperazin-1-yl)-3-oxopropoxy)ethoxy)ethyl)amino)-1-oxoisoindolin-2-yl)piperidinc-2,6-dione;
3-(4-((2-(2-(2-(3-(4-(2-(4-(4-chloro-1-(4-hydroxyphenyl)-2-phenylbut-1-en-1-yl)phenoxy)ethyl)piperazin-1-yl)-3-oxopropoxy)ethoxy)ethoxy)elhyl)amino)-1-oxoisoindolin-2-yl)piperidine-2,6-dione;
3-(4-((15-(4-(2-(4-(4-chloro-1-(4-hydroxyphenyl)-2-phenylbut-1-en-1-yl)phenoxy)ethyl)piperazin-1-yl)-15-oxo-3,6,9,12-tetraoxapentadecyl)amino)-1-oxoisoindolin-2-yl)piperidine-2,6-dione;
3-(4-((18-(4-(2-(4-(4-chloro-1-(4-hydroxyphenyl)-2-phenylbut-1-en-1-yl)phenoxy)ethyl)piperazin-1-yl)-18-oxo-3,6,9,12,15-pentaoxaoctadecyl)amino)-1-oxoisoindolin-2-yl)piperidine-2,6-dione;
3-(4-(2-(4-(4-chloro-1-(4-hydroxyphenyl)-2-phenylbut-1-en-1-yl)phenoxy)ethyl)piperazin-1-yl)-N-(2-(2,6-dioxopiperidin-3-yl)-1-oxoisoindolin 4-yl)-3-oxopropanamide;
4-(4-(2-(4-(4-chloro-1-(4-hydroxyphenyl)-2-phenylbut-1-en-1-yl)phenoxy)ethyl)piperazin-1-yl)-N-(2-(2,6-dioxopiperidin-3-yl)-1-oxoisoindolin 4-yl)-4-oxobutanamide:
5-(4-(2-(4-(4-chloro- l-(4-hydroxyphenyl)-2-phenylbut-1-en-1-yl)phenoxy)ethyl)piperazin-1-yl)-N-(2-(2,6-dioxopiperidin-3-yl)-1-oxoisoindolin 4-yl)-5-oxopentanamide;
6-(4-(2-(4-(4-chloro-1-(4-hydroxyphenyl)-2-phenylbut-1-en-1-yl)phenoxy)ethyl)piperazin-1-yl)-N-(2-(2,6-dioxopiperidin-3-yl)-1-oxoisoindolin 4-yl)-6-oxohexanamide;
7-(4-(2-(4-(4-chloro-1-(4-hydroxyphenyl)-2-phenylbut-1-en-1-yl)phenoxy)ethyl)piperazin-1-yl)-N-(2-(2,6-dioxopiperidin-3-yl)-1-oxoisoindolin 4-yl)-7-oxoheptanamide;
3-(4-((2-(4-(2-(4-(4-chloro-1-(4-hydroxyphenyl)-2-phenylbut-1-en-1-yl)phenoxy)ethyl)piperazin-1-yl)-2-oxoethyl)amino)-1-oxoisoindolin-2-yl)piperidine-2,6-dione;
3-(4-((3-(4-(2-(4-(4-chloro-1-(4-hydroxyphenyl)-2-phenylbut-1-en-1-yl)phenoxy)ethyl)piperazin-1-yl)-3-oxopropyl)amino)-1-oxoisoindolin-2-yl)piperidine-2,6-dione;
3-(4-((4-(4-(2-(4-(4-chloro-1-(4-hydroxyphenyl)-2-phenylbut-1-en-1-yl)phenoxy)ethyl)piperazin-1-yl)-4-oxobutyl)amino)-1-oxoisoindolin-2-yl)piperidine-2,6-dione;
3-(4-((5-(4-(2-(4-(4-chloro-1-(4-hydroxyphenyl)-2-phenylbut-1-en-1-yl)phenoxy)ethyl)piperazin-1-yl)-5-oxopentyl)amino)-1-oxoisoindolin-2-yl)piperidine-2,6-dione;
3-(4-((6-(4-(2-(4-(4-chloro-1-(4-hydroxyphenyl)-2-phenylbut-1-en-1-yl)phenoxy)ethyl)piperazin-1-yl)-6-oxohexyl)amino)-1-oxoisoindolin-2-yl)piperidine-2,6-dione;
3-(4-((7-(4-(2-(4-(4-chloro-1-(4-hydroxyphenyl)-2-phenylbut-1-en-1-yl)phenoxy)ethyl)piperazin-1-yl)-7-oxoheptyl)amino)-1-oxoisoindolin-2-yl)piperidine-2,6-dione;
(2S,4R)-1-((S)-2-(2-(2-(4-(2-(4-(4-chloro-1-(4-hydroxyphenyl)-2-phenylbut-1-en-1-yl)phenoxy)ethyl)piperazin-1-yl)-2-oxoethoxy)acetamido)-3,3-dimethylbutanoyl)-4-hydroxy-N-(4-(4-methylthiazol-5-yl)benzyl)pyrrolidine-2-carboxamide;
(2S,4R)-1-((S)-2-(2-(2-(2-(4-(2-(4-(4-chloro-1-(4-hydroxyphenyl)-2-phenylbut-1-en-1-yl)phenoxy)ethyl)piperazin-1-yl)-2-oxoethoxy)ethoxy)acetamido)-3,3-dimethylbutanoyl)-4-hydroxy-N-(4-(4-methylthiazol-5-yl)benzyl)pyrrolidine-2-carboxamide;
(2S,4R)-1-((S)-2-(3-(2-(3-(4-(2-(4-(4-chloro-1-(4-hydroxyphenyl)-2-phenylbut-1-en-1-yl)phenoxy)ethyl)piperazin-1-yl)-3-oxopropoxy)ethoxy)propanamido)-3,3-dimethylbutanoyl)-4-hydroxy-N-(4-(4-methylthiazol-5-yl)benzyl)pyrrolidine-2-carboxamide;
(2S,4R)-1-((S)-2-(tert-butyl)-16-(4-(2-(4-(4-chloro-1-(4-hydroxyphenyl)-2-phenylbut-1-en-1-yl)phenoxy)ethyl)piperazin-1-yl)-4,16-dioxo-7,10,13-trioxa-3-azahexadecanoyl)-4-hydroxy-N-(4-(4-methylthiazol-5-yl)benzyl)pyrrolidine-2-carboxamide;
(2S,4R)-1-((S)-2-(tert-butyl)-19-(4-(2-(4-(4-chloro-1-(4-hydroxyphenyl)-2-phenylbut-1-en-1-yl)phenoxy)ethyl)piperazin-1-yl)-4,19-dioxo-7,10,13,16-tetraoxa-3-azanonadecanoyl)-4-hydroxy-N-(4-(4-methylthiazol-5-yl)benzyl)pyrrolidine-2-carboxamide;
(2S,4R)-1-((S)-2-(tert-butyl)-22-(4-(2-(4-(4-chloro-1-(4-hydroxyphenyl)-2-phenylbut-1-en-1-yl)phenoxy)ethyl)piperazin-1-yl)-4,22-dioxo-7,10,13,16,19-pentaoxa-3-azadocosanoyl)-4-hydroxy-N-(4-(4-methylthiazol-5-yl)benzyl)pyrrolidine-2-carboxamide;
(2S,4R)-1-((S)-2-(4-(4-(2-(4-(4-chloro-1-(4-hydroxyphenyl)-2-phenylbut-1-en-1-yl)phenoxy)ethyl)piperazin-1-yl)-4-oxobutanamido)-3,3-dimethylbutanoyl)-4-hydroxy-N-(4-(4-methylthiazol-5-yl)benzyl)pyrrolidine-2-carboxamide;
(2S,4R)-1-((S)-2-(5-(4-(2-(4-(4-chloro-1-(4-hydroxyphenyl)-2-phenylbut-1-en-1-yl)phenoxy)ethyl)piperazin-1-yl)-5-oxopentanamido)-3,3-dimethylbutanoyl)-4-hydroxy-N-(4-(4-methylthiazol-5-yl)benzyl)pyrrolidine-2-carboxamide;
(2S,4R)-1-((S)-2-(6-(4-(2-(4-(4-chloro-1-(4-hydroxyphenyl)-2-phenylbut-1-en-1-yl)phenoxy)ethyl)piperazin-1-yl)-6-oxohexanamido)-3,3-dimethylbutanoyl)-4-hydroxy-N-(4-(4-methylthiazol-5-yl)benzyl)pyrrolidine-2-carboxamide;
(2S,4R)-1-((S)-2-(7-(4-(2-(4-(4-chloro-1-(4-hydroxyphenyl)-2-phenylbut-1-en-1-yl)phenoxy)ethyl)piperazin-1-yl)-7-oxoheptanamido)-3,3-dimethylbutanoyl)-4-hydroxy-N-(4-(4-methylthiazol-5-yl)benzyl)pyrrolidine-2-carboxamide;
(2S,4R)-1-((S)-2-(8-(4-(2-(4-(4-chloro-1-(4-hydroxyphenyl)-2-phenylbut-1-en-1-yl)phenoxy)ethyl)piperazin-1-yl)-8-oxooctanamido)-3,3-dimethylbutanoyl)-4-hydroxy-N-(4-(4-methylthiazol-5-yl)benzyl)pyrrolidine-2-carboxamide;
(2S,4R)-1-((S)-2-(9-(4-(2-(4-(4-chloro-1-(4-hydroxyphenyl)-2-phenylbut-1-en-1-yl)phenoxy)ethyl)piperazin-1-yl)-9-oxononanamido)-3,3-dimethylbutanoyl)-4-hydroxy-N-(4-(4-methylthiazol-5-yl)benzyl)pyrrolidine-2-carboxamide;
(2S,4R)-1-((S)-2-(10-(4-(2-(4-(4-chloro-1-(4-hydroxyphenyl)-2-phenylbut-1-en-1-yl)phenoxy)ethyl)piperazin-1-yl)-10-oxodecanamido)-3,3-dimethylbutanoyl)-4-hydroxy-N-(4-(4-methylthiazol-5-yl)benzyl)pyrrolidine-2-carboxamide;
(2S,4R)-1-((S)-2-(7-(4-(2-(4-(4-chloro-1-(4-hydroxyphenyl)-2-phenylbut-1-en-1-yl)phenoxy)ethyl)piperazin-1-yl)heptanamido)-3,3-dimethylbutanoyl)-4-hydroxy-N-(4-(4-methylthiazol-5-yl)benzyl)pyrrolidine-2-carboxamide;
(2S,4R)-1-((S)-2-((7-(4-(2-(4-(4-chloro-1-(4-hydroxyphenyl)-2-phenylbut-1-en-1-yl)phenoxy)ethyl)piperazin-1-yl)-7-oxoheptyl)amino)-3,3-dimethylbutanoyl)-4-hydroxy-N-(4-(4-methylthiazol-5-yl)benzyl)pyrrolidine-2-carboxamide;
(2S,4R)-1-((S)-2-((7-(4-(2-(4-(4-chloro-1-(4-hydroxyphenyl)-2-phenylbut-1-en-1-yl)phenoxy)ethyl)piperazin-1-yl)heptyl)amino)-3,3-dimethylbutanoyl)-4-hydroxy-N-(4-(4-methylthiazol-5-yl)benzyl)pyrrolidine-2-carboxamide;
N-(2-(4-(4-chloro-1,2-bis (4-hydroxyphenyl)but-1-en-1-yl)phenoxy)ethyl)-3-(2-((2-(2,6-dioxopiperidin-3-yl)-1,3-dioxoisoindolin-4-yl)amino)ethoxy)prop anamide;
N-(2-(4-(4-chloro-1,2-bis (4-hydroxyphenyl)but-1-en-1-yl)phenoxy)ethyl)-3-(2-(2-((2-(2,6-dioxopiperidin-3-yl)-1,3-dioxoisoindolin-4-yl)amino)ethoxy)ethoxy)propanamide;
N-(2-(4-(4-chloro-1,2-bis (4-hydroxyphenyl)but-1-en-1-yl)phenoxy)ethyl)-3-(2-(2-(2-((2-(2,6-dioxopiperidin-3-yl)-1,3-dioxoisoindolin-4-yl)amino)ethoxy)ethoxy)ethoxy)propanamide;
N-(2-(4-(4-chloro-1,2-bis (4-hydroxyphenyl)but-1-en-1-yl)phenoxy)ethyl)-1-((2-(2,6-dioxopiperidin-3-yl)-1,3-dioxoisoindolin-4-yl)amino)-3,6,9,12-tetraoxapentadecan-15-amide;
N-(2-(4-(4-chloro-1,2-bis (4-hydroxyphenyl)but-1-en-1-yl)phenoxy)ethyl)-1-((2-(2,6-dioxopiperidin-3-yl)-1,3-dioxoisoindolin-4-yl)amino)-3,6,9,12,15-pentaoxaoctadecan-18-amide;
N-(2-(4-(4-chloro-1,2-bis (4-hydroxyphenyl)but-1-en-1-yl)phenoxy)ethyl)-2-((2-(2,6-dioxopiperidin-3-yl)-1,3-dioxoisoindolin-4-yl)amino)acetamide;
N-(2-(4-(4-chloro-1,2-bis (4-hydroxyphenyl)but-1-en-1-yl)phenoxy)ethyl)-3-((2-(2,6-dioxopiperidin-3-yl)-1,3-dioxoisoindolin-4-yl)amino)propanamide;
N-(2-(4-(4-chloro-1,2-bis (4-hydroxyphenyl)but-1-en-1-yl)phenoxy)ethyl)-4-((2-(2,6-dioxopiperidin-3-yl)-1,3-dioxoisoindolin-4-yl)amino)butanamide;
N-(2-(4-(4-chloro-1,2-bis (4-hydroxyphenyl)but-1-en-1-yl)phenoxy)ethyl)-5-((2-(2,6-dioxopiperidin-3-yl)-1,3-dioxoisoindolin-4-yl)amino)pentanamide;
N-(2-(4-(4-chloro-1,2-bis (4-hydroxyphenyl)but-1-en-1-yl)phenoxy)ethyl)-6-((2-(2,6-dioxopiperidin-3-yl)-1,3-dioxoisoindolin-4-yl)amino)hexanamide;
N-(2-(4-(4-chloro-1,2-bis (4-hydroxyphenyl)but-1-en-1-yl)phenoxy)ethyl)-7-((2-(2,6-dioxopiperidin-3-yl)-1,3-dioxoisoindolin-4-yl)amino)heptanamide;
N-(2-(4-(4-chloro-1,2-bis (4-hydroxyphenyl)but-1-en-1-yl)phenoxy)ethyl)-3-(3-((2-(2,6-dioxopiperidin-3-yl)-1-oxoisoindolin-4-yl)amino)-3-oxopropoxy)propanamide;
N-(2-(4-(4-chloro-1,2-bis(4-hydroxyphenyl)but-1-en-1-yl)phenoxy)ethyl)-3-(2-(3-((2-(2,6-dioxopiperidin-3-yl)-1-oxoisoindolin-4-yl)amino)-3-oxopropoxy)ethoxy)propanamide;
N-(2-(4-(4-chloro-1,2-bis(4-hydroxyphenyl)but-1-en-1-yl)phenoxy)ethyl)-3-(2-(2-(3-((2-(2,6-dioxopiperidin-3-yl)-1-oxoisoindolin-4-yl)amino)-3-oxopropoxy)ethoxy)ethoxy)propanamide;
N1-(2-(4-(4-chloro-1,2-bis(4-hydroxyphenyl)but-1-en-1-yl)phenoxy)ethyl)-N16-(2-(2,6-dioxopiperidin-3-yl)-1-oxoisoindolin-4-yl)-4,7,10,13-tetraoxahexadecanediamide;
N1-(2-(4-(4-chloro-1,2-bis(4-hydroxyphenyl)but-1-en-1-yl)phenoxy)ethyl)-N19-(2-(2,6-dioxopiperidin-3-yl)-1-oxoisoindolin-4-yl)-4,7,10,13,16-pentaoxanonadecanediamide;
N-(2-(4-(4-chloro-1,2-bis(4-hydroxyphenyl)but-1-en-1-yl)phenoxy)ethyl)-3-(2-((2-(2,6-dioxopiperidin-3-yl)-1-oxoisoindolin-4-yl)amino)ethoxy)propanamide;
N-(2-(4-(4-chloro-1,2-bis(4-hydroxyphenyl)but-1-en-1-yl)phenoxy)ethyl)-3-(2-(2-((2-(2,6-dioxopiperidin-3-yl)-1-oxoisoindolin-4-yl)amino)ethoxy)ethoxy)propanamide;
N-(2-(4-(4-chloro-1,2-bis(4-hydroxyphenyl)but-1-en-1-yl)phenoxy)ethyl)-3-(2-(2-(2-((2-(2,6-dioxopiperidin-3-yl)-1-oxoisoindolin-4-yl)amino)ethoxy)ethoxy)ethoxy)propanamide;
N-(2-(4-(4-chloro-1,2-bis(4-hydroxyphenyl)but-1-en-1-yl)phenoxy)ethyl)-1-((2-(2,6-dioxopiperidin-3-yl)-1-oxoisoindolin-4-yl)amino)-3 ,6,9,12-tetraoxapentadecan-15-amide;
N-(2-(4-(4-chloro-1,2-bis(4-hydroxyphenyl)but-1-en-1-yl)phenoxy)ethyl)-1-((2-(2,6-dioxopiperidin-3-yl)-1-oxoisoindolin-4-yl)amino)-3 ,6,9,12,15-pentaoxaoctadecan-18-amide;
N1-(2-(4-(4-chloro-1,2-bis(4-hydroxyphenyl)but-1-en-1-yl)phenoxy)ethyl)-N3-(2-(2,6-dioxopiperidin-3-yl)-1-oxoisoindolin-4-yl)malonamide;
N1-(2-(4-(4-chloro-1,2-bis(4-hydroxyphenyl)but-1-en-1-yl)phenoxy)ethyl)-N4-(2-(2,6-dioxopiperidin-3-yl)-1-oxoisoindolin-4-yl)succinamide;
N1-(2-(4-(4-chloro-1,2-bis(4-hydroxyphenyl)but-1-en-1-yl)phenoxy)ethyl)-N5-(2-(2,6-dioxopiperidin-3-yl)-1-oxoisoindolin-4-yl)glutaramide;
N1-(2-(4-(4-chloro-1,2-bis(4-hydroxyphenyl)but-1-en-1-yl)phenoxy)ethyl)-N6-(2-(2,6-dioxopiperidin-3-yl)-1-oxoisoindolin-4-yl)adipamide;
N1-(2-(4-(4-chloro-1,2-bis(4-hydroxyphenyl)but-1-en-1-yl)phenoxy)ethyl)-N7-(2-(2,6-dioxopiperidin-3-yl)-1-oxoisoindolin-4-yl)heptanediamide;
N1-(2-(4-(4-chloro-1,2-bis(4-hydroxyphenyl)but-1-en-1-yl)phenoxy)ethyl)-N8-(2-(2,6-dioxopiperidin-3-yl)-1-oxoisoindolin-4-yl)octanediamide;
N-(2-(4-(4-chloro-1,2-bis(4-hydroxyphenyl)but-1-en-1-yl)phenoxy)ethyl)-3-((2-(2,6-dioxopiperidin-3-yl)-1-oxoisoindolin-4-yl)amino)propanamide;
N-(2-(4-(4-chloro-1,2-bis(4-hydroxyphenyl)but-1-en-1-yl)phenoxy)ethyl)-4-((2-(2,6-dioxopiperidin-3-yl)-1-oxoisoindolin-4-yl)amino)butanamide;
N-(2-(4-(4-chloro-1,2-bis(4-hydroxyphenyl)but-1-en-1-yl)phenoxy)ethyl)-5-((2-(2,6-dioxopiperidin-3-yl)-1-oxoisoindolin-4-yl)amino)pentanamide;
N-(2-(4-(4-chloro-1,2-bis(4-hydroxyphenyl)but-1-en-1-yl)phenoxy)ethyl)-6-((2-(2,6-dioxopiperidin-3-yl)-1-oxoisoindolin-4-yl)amino)hexanamide;
N-(2-(4-(4-chloro-1,2-bis(4-hydroxyphenyl)but-1-en-1-yl)phenoxy)ethyl)-7-((2-(2,6-dioxopiperidin-3-yl)-1-oxoisoindolin-4-yl)amino)heptanamide;
N-(2-(4-(4-chloro-1,2-bis(4-hydroxyphenyl)but-1-en-1-yl)phenoxy)ethyl)-8-((2-(2,6-dioxopiperidin-3-yl)-1-oxoisoindolin-4-yl)amino)octanamide;
(2S,4R)-1-((S)-2-(tert-butyl)-14-(4-(4-chloro-1,2-bis(4-hydroxyphenyl)but-1-en-1-yl)phenoxy)-4,11-dioxo-6,9-dioxa-3,12-diazatetradecanoyl)-4-hydroxy-N-(4-(4-methylthiazol-5-yl)benzyl)pyrrolidine-2-carboxamide;
(2S,4R)-1-((S)-2-(tert-butyl)-16-(4-(4-chloro-1,2-bis(4-hydroxyphenyl)but-1-en-1-yl)phenoxy)-4,13-dioxo-7,10-dioxa-3,14-diazahexadec anoyl)-4-hydroxy-N-(4-(4-methylthiazol-5-yl)benzyl)pyrrolidine-2-carboxamide;
(2S,4R)-1-((S)-2-(tert-butyl)-19-(4-(4-chloro-1,2-bis(4-hydroxyphenyl)but-1-en-1-yl)phenoxy)-4,16-dioxo-7,10,13-trioxa-3,17-diazanonadecanoyl)-4-hydroxy-N-(4-(4-methylthiazol-5-yl)benzyl)pyrrolidine-2-c arboxamide;
N1-(2-(4-(4-chloro-1,2-bis(4-hydroxyphenyl)but-1-en-1-yl)phenoxy)ethyl)-N16-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)-4,7,10,13-tetraoxahexadecanediamide;
N1-(2-(4-(4-chloro-1,2-bis(4-hydroxyphenyl)but-1-en-1-yl)phenoxy)ethyl)-N19-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)-4,7,10,13,16-pentaoxanonadec anediamide;
N1-(2-(4-(4-chloro-1,2-bis(4-hydroxyphenyl)but-1-en-1-yl)phenoxy)ethyl)-N4-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)succinamide;
N1-(2-(4-(4-chloro-1,2-bis(4-hydroxyphenyl)but-1-en-1-yl)phenoxy)ethyl)-N5-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)glutaramide;
N1-(2-(4-(4-chloro-1,2-bis(4-hydroxyphenyl)but-1-en-1-yl)phenoxy)ethyl)-N6-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)adipamide;
N1-(2-(4-(4-chloro-1,2-bis(4-hydroxyphenyl)but-1-en-1-yl)phenoxy)ethyl)-N7-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)heptanediamide;
N1-(2-(4-(4-chloro-1,2-bis(4-hydroxyphenyl)but-1-en-1-yl)phenoxy)ethyl)-N8-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)octanediamide;
N1-(2-(4-(4-chloro-1,2-bis(4-hydroxyphenyl)but-1-en-1-yl)phenoxy)ethyl)-N9-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)nonanediamide ;
N1-(2-(4-(4-chloro-1,2-bis(4-hydroxyphenyl)but-1-en-1-yl)phenoxy)ethyl)-N10-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)decanediamide;
N1-(2-(4-(4-chloro-1,2-bis(4-hydroxyphenyl)but-1-en-1-yl)phenoxy)ethyl)-N11-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)undecanediamide;
N1-(2-(4-(4-chloro-1,2-bis(4-hydroxyphenyl)but-1-en-1-yl)phenoxy)ethyl)-N14-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)tetradecanediamide;
N1-(2-(4-(4-chloro-1,2-bis(4-hydroxyphenyl)but-1-en-1-yl)phenoxy)ethyl)-N16-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)hexadec anediamide;
(2S,4R)-1-((S)-2-(3-(4-(3-((2-(4-(4-chloro-1,2-bis(4-hydroxyphenyl)but-1-en-1-yl)phenoxy)ethyl)amino)-3-oxopropyl)piperazin-1-yl)propanamido)-3,3-dimethylbutanoyl)-4-hydroxy-N-(4-(4-methylthiazol-5-yl)benzyl)pyrrolidine-2-carboxamide;
(2S,4R)-1-((S)-2-(3-(4-(3-((2-(4-(4-chloro-1,2-bis(4-hydroxyphenyl)but-1-en-1-yl)phenoxy)ethyl)amino)-3-oxopropyl)phenyl)propanamido)-3,3-dimethylbutanoyl)-4-hydroxy-N-(4-(4-methylthiazol-5-yl)benzyl)pyrrolidine-2-carboxamide;
N-(2-(4-(4-chloro-1,2-bis (4-hydroxyphenyl)but-1-en-1-yl)phenoxy)ethyl)-3-(4-(2-((2-(2,6-dioxopiperidin-3-yl)-1,3-dioxoisoindolin-4-yl)amino)ethyl)piperazin-1-yl)propanamide;
N-(2-(4-(4-chloro-1,2-bis (4-hydroxyphenyl)but-1-en-1-yl)phenoxy)ethyl)-3-(4-(2-((2-(2,6-dioxopiperidin-3-yl)-1,3-dioxoisoindolin-4-yl)amino)ethyl)phenyl)propanamide;
(2S,4R)-1-((S)-2-(7-((2-(4-(4-chloro-1,2-bis(4-hydroxyphenyl)but-1-en-1-yl)phenoxy)ethyl)amino)heptanamido)-3,3-dimethylbutanoyl)-4-hydroxy-N-(4-(4-methylthiazol-5-yl)benzyl)pyrrolidine-2-carboxamide;
(2S,4R)-1-((S)-2-((7-((2-(4-(4-chloro-1,2-bis(4-hydroxyphenyl)but-1-en-1-yl)phenoxy)ethyl)amino)-7-oxoheptyl)amino)-3,3-dimethylbutanoyl)-4-hydroxy-N-(4-(4-methylthiazol-5-yl)benzyl)pyrrolidine-2-carboxamide;
(2S,4R)-1-((S)-2-((7-((2-(4-(4-chloro-1,2-bis(4-hydroxyphenyl)but-1-en-1-yl)phenoxy)ethyl)amino)heptyl)amino)-3,3-dimethylbutanoyl)-4-hydroxy-N-(4-(4-methylthiazol-5-yl)benzyl)pyrrolidine-2-carboxamide;
4-((2-(3-(4-(2-(4-(4-chloro-1,2-bis (4-hydroxyphenyl)but-1-en-1-yl)phenoxy)ethyl)piperazin-1-yl)-3-oxopropoxy)ethyl)amino)-2-(2,6-dioxopiperidin-3-yl)isoindoline-1,3-dione;
4-((2-(2-(3-(4-(2-(4-(4-chloro-1,2-bis(4-hydroxyphenyl)but-1-en-1-yl)phenoxy)ethyl)piperazin-1-yl)-3-oxopropoxy)ethoxy)ethyl)amino)-2-(2,6-dioxopiperidin-3-yl)isoindoline-1,3-dione;
4-((2-(2-(2-(3-(4-(2-(4-(4-chloro-1,2-bis(4-hydroxyphenyl)but-1-en-1-yl)phenoxy)ethyl)piperazin-1-yl)-3-oxopropoxy)ethoxy)ethoxy)ethyl)amino)-2-(2,6-dioxopiperidin-3-yl)isoindoline-1,3-dione;
4-((15-(4-(2-(4-(4-chloro-1,2-bis(4-hydroxyphenyl)but-1-en-1-yl)phenoxy)ethyl)piperazin-1-yl)-15-oxo-3,6,9,12-tetraoxapcntadecyl)amino)-2-(2,6-dioxopiperidin-3-yl)isoindoline-1,3-dione;
4-(( 18-(4-(2-(4-(4-chloro-1,2-bis(4-hydroxyphenyl)but-1-en-1-yl)phenoxy)ethyl)piperazin-1-yl)-18-oxo-3,6,9,12,15-pcntaoxaoctadecyl)amino)-2-(2.6-dioxopiperidin-3-yl)isoindoline-1,3-dione;
4-((2-(4-(2-(4-(4-chloro-1,2-bis(4-hydroxyphenyl)but-1-en-1-yl)phenoxy)ethyl)piperazin-1-yl)-2-oxoethyl)amino)-2-(2,6-dioxopiperidin-3-yl)isoindoline-1,3-dione;
4-((3-(4-(2-(4-(4-chloro-1.2-bis(4-hydroxyphenyl)but-1-en-1-yl)phenoxy)ethyl)piperazin-1-yl)-3-oxopropyl)amino)-2-(2,6-dioxopiperidin-3-yl)isoindoline-1,3-dione;
4-((4-(4-(2-(4-(4-chloro-1,2-bis(4-hydroxyphenyl)but-1-en-1-yl)phenoxy)ethyl)piperazin-1-yl)-4-oxobutyl)amino)-2-(2,6-dioxopiperidin-3-yl)isoindoline-1,3-dione;
4-((5-(4-(2-(4-(4-chloro-1.2-bis(4-hydroxyphenyl)but-1-en-1-yl)phenoxy)ethyl)piperazin-1-yl)-5-oxopentyl)amino)-2-(2,6-dioxopiperidin-3-yl)isoindoline-1,3-dione;
4-((6-(4-(2-(4-(4-chloro-1,2-bis(4-hydroxyphenyl)but-1-en-1-yl)phenoxy)ethyl)piperazin-1-yl)-6-oxohexyl)amino)-2-(2,6-dioxopiperidin-3-yl)isoindoline-1,3-dione;
4-((7-(4-(2-(4-(4-chloro-1.2-bis(4-hydroxyphenyl)but-1-en-1-yl)phenoxy)ethyl)piperazin-1-yl)-7-oxoheplyl)amino)-2-(2,6-dioxopiperidin-3-yl)isoindoline-1,3-dione;
3-(4-((2-(3-(4-(2-(4-(4-chloro-1,2-bis(4-hydroxyphenyl)but-1-en-1-yl)phenoxy)ethyl)piperazin-1-yl)-3-oxopropoxy)ethyl)amino)-1-oxoisoindolin-2-yl)piperidine-2,6-dione;
3-(4-((2-(2-(3-(4-(2-(4-(4-chloro-1,2-bis(4-hydroxyphenyl)but-1-en-1-yl)phenoxy)ethyl)piperazin-1-yl)-3-oxopropoxy)ethoxy)ethyl)amino)-1-oxoisoindolin-2-yl)piperidine-2,6-dione;
3-(4-((2-(2-(2-(3-(4-(2-(4-(4-chloro-1,2-bis(4-hydroxyphenyl)but-1-en-1-yl)phenoxy)ethyl)piperazin-1-yl)-3-oxopropoxy)ethoxy)ethoxy)ethyl)amino)-1-oxoisoindolin-2-yl)piperidinc-2,6-dione;
3-(4-((15-(4-(2-(4-(4-chloro-1,2-bis(4-hydroxy phenyl)but-1-en-1-yl)phenoxy)ethyl)piperazin-1-yl)-15-oxo-3,6,9,12-tetraoxapentadecyl)amino)-1-oxoisoindolin-2-yl)piperidine-2,6-dione;
3-(4-(( 18-(4-(2-(4-(4-chloro-1,2-bis(4-hydroxyphenyl)but-1-en-1-yl)phenoxy)ethyl)piperazin-1-yl)-18-oxo-3,6,9,12,15-pentaoxaoctadecyl)amino)-1-oxoisoindolin-2-yl)piperidine-2,6-dione;
3-(4-((2-(4-(2-(4-(4-chloro-1,2-bis(4-hydroxyphenyl)but-1-en-1-yl)phenoxy)ethyl)piperazin-1-yl)-2-oxoethyl)amino)-1-oxoisoindolin-2-yl)piperidine-2,6-dione;
3-(4-((3-(4-(2-(4-(4-chloro-1,2-bis(4-hydroxyphenyl)but-1-en-1-yl)phenoxy)ethyl)piperazin-1-yl)-3-oxopropyl)amino)-1-oxoisoindolin-2-yl)piperidine-2,6-dione;
3-(4-((4-(4-(2-(4-(4-chloro-1,2-bis(4-hydroxyphenyl)but-1-en-1-yl)phenoxy)ethyl)piperdzin-1-yl)-4-oxobulyl)amino)-1-oxoisoindolin-2-yl)piperidine-2,6-dione;
3-(4-((5-(4-(2-(4-(4-chloro-1,2-bis(4-hydroxyphenyl)but-1-en-1-yl)phenoxy)ethyl)piperazin-1-yl)-5-oxopentyl)amino)-1-oxoisoindolin-2-yl)piperidine-2,6-dione;
3-(4-((6-(4-(2-(4-(4-chloro-1,2-bis(4-hydroxyphenyl)but-1-en-1-yl)phenoxy)ethyl)piperazin-1-yl)-6-oxohexyl)amino)-1-oxoisoindolin-2-yl)piperidine-2,6-dione;
3-(4-((7-(4-(2-(4-(4-chloro-1,2-bis(4-hydroxyphenyl)but-1-en-1-yl)phenoxy)ethyl)piperazin-1-yl)-7-oxoheptyl)amino)-1-oxoisoindolin-2-yl)piperidine-2,6-dione;
(2S,4R)-1-((S)-2-(2-(2-(2-(4-(2-(4-(4-chloro-1,2-bis(4-hydroxyphenyl)but-1-en-1-yl)phenoxy)ethyl)piperazin-1-yl)-2-oxoethoxy)ethoxy)acetamido)-3,3-dimethylbutanoyl)-4-hydroxy-N-(4-(4-methylthiazol-5-yl)benzyl)pyrrolidine-2-carboxamide;
(2S,4R)-1-((S)-2-(3-(2-(3-(4-(2-(4-(4-chloro-1,2-bis(4-hydroxyphenyl)but-1-en-1-yl)phenoxy)ethyl)piperazin-1-yl)-3-oxopropoxy)ethoxy)propanamido)-3,3-dimethylbutanoyl)-4-hydroxy-N-(4-(4-methylthiazol-5-yl)benzyl)pyrrolidine-2-carboxamide;
(2S,4R)-1-((S)-2-(tert-butyl)-16-(4-(2-(4-(4-chloro-1,2-bis (4-hydroxyphenyl)but-1-en-1-yl)phenoxy)ethyl)piperazin-1-yl)-4,16-dioxo-7,10,13-trioxa-3-azahexadecanoyl)-4-hydroxy-N-(4-(4-methylthiazol-5-yl)benzyl)pyrrolidine-2-carboxamide;
(2S,4R)-1-((S)-2-(tert-butyl)-19-(4-(2-(4-(4-chloro-1,2-bis (4-hydroxyphenyl)but-1-en-1-yl)phenoxy)ethyl)piperazin-1-yl)-4,19-dioxo-7,10,13,16-tetraoxa-3-azanonadecanoyl)-4-hydroxy-N-(4-(4-methylthiazol-5-yl)benzyl)pyrrolidine-2-carboxamide;
(2S,4R)-1-((S)-2-(tert-butyl)-22-(4-(2-(4-(4-chloro-1,2-bis (4-hydroxyphenyl)but-1-en-1-yl)phenoxy)ethyl)piperazin-1-yl)-4,22-dioxo-7,10,13,16,19-pentaoxa-3-azadocosanoyl)-4-hydroxy-N-(4-(4-methylthiazol-5-yl)benzyl)pyrrolidine-2-carboxamide;
(2S,4R)-1-((S)-2-(4-(4-(2-(4-(4-chloro-1,2-bis(4-hydroxyphenyl)but-1-en-1-yl)phenoxy)ethyl)piperazin-1-yl)-4-oxobutanamido)-3,3-dimethylbutanoyl)-4-hydroxy-N-(4-(4-methylthiazol-5-yl)benzyl)pyrrolidine-2-carboxamide;
(2S,4R)-1-((S)-2-(5-(4-(2-(4-(4-chloro-1,2-bis(4-hydroxyphenyl)but-1-en-1-yl)phenoxy)ethyl)piperazin-1-yl)-5-oxopentanamido)-3,3-dimethylbutanoyl)-4-hydroxy-N-(4-(4-methylthiazol-5-yl)benzyl)pyrrolidine-2-carboxamide;
(2S,4R)-1-((S)-2-(6-(4-(2-(4-(4-chloro-1,2-bis(4-hydroxyphenyl)but-1-en-1-yl)phenoxy)ethyl)piperazin-1-yl)-6-oxohexanamido)-3,3-dimethylbutanoyl)-4-hydroxy-N-(4-(4-methylthiazol-5-yl)benzyl)pyrrolidine-2-carboxamide;
(2S,4R)-1-((S)-2-(7-(4-(2-(4-(4-chloro-1,2-bis(4-hydroxyphenyl)but-1-en-1-yl)phenoxy)ethyl)piperazin-1-yl)-7-oxoheptanamido)-3,3-dimethylbutanoyl)-4-hydroxy-N-(4-(4-methylthiazol-5-yl)benzyl)pyrrolidine-2-carboxamide;
(2S,4R)-1-((S)-2-(8-(4-(2-(4-(4-chloro-1,2-bis(4-hydroxyphenyl)but-1-en-1-yl)phenoxy)ethyl)piperazin-1-yl)-8-oxooctanamido)-3,3-dimethylbutanoyl)-4-hydroxy-N-(4-(4-methylthiazol-5-yl)benzyl)pyrrolidine-2-carboxamide;
(2S,4R)-1-((S)-2-(9-(4-(2-(4-(4-chloro-1,2-bis(4-hydroxyphenyl)but-1-en-1-yl)phenoxy)ethyl)piperazin-1-yl)-9-oxononanamido)-3,3-dimethylbutanoyl)-4-hydroxy-N-(4-(4-methylthiazol-5-yl)benzyl)pyrrolidine-2-carboxamide;
(2S,4R)-1-((S)-2-(10-(4-(2-(4-(4-chloro-1,2-bis(4-hydroxyphenyl)but-1-en-1-yl)phenoxy)ethyl)piperazin-1-yl)-10-oxodecanamido)-3,3-dimethylbutanoyl)-4-hydroxy-N-(4-(4-methylthiazol-5-yl)benzyl)pyrrolidine-2-carboxamide;
(2S,4R)-1-((S)-2-(7-(4-(2-(4-(4-chloro-1,2-bis(4-hydroxyphenyl)but-1-en-1-yl)phenoxy)ethyl)piperazin-1-yl)heptanamido)-3,3-dimethylbutanoyl)-4-hydroxy-N-(4-(4-methylthiazol-5-yl)benzyl)pyrrolidine-2-carboxamide;
(2S,4R)-1-((S)-2-((7-(4-(2-(4-(4-chloro-1,2-bis(4-hydroxyphenyl)but-1-en-1-yl)phenoxy)ethyl)piperazin-1-yl)-7-oxoheptyl)amino)-3,3-dimethylbutanoyl)-4-hydroxy-N-(4-(4-methylthiazol-5-yl)benzyl)pyrrolidine-2-carboxamide;
(2S,4R)-1-((S)-2-((7-(4-(2-(4-(4-chloro-1,2-bis(4-hydroxyphenyl)but-1-en-1-yl)phenoxy)ethyl)piperazin-1-yl)heptyl)amino)-3,3-dimethylbutanoyl)-4-hydroxy-N-(4-(4-methylthiazol-5-yl)benzyl)pyrrolidine-2-carboxamide;
N-(2-(4-(1,2-bis(4-hydroxyphenyl)but-1-en-1-yl)phenoxy)ethyl)-3-(2-((2-(2,6-dioxopiperidin-3-yl)-1,3-dioxoisoindolin-4-yl)amino)ethoxy)prop anamide;
N-(2-(4-(1,2-bis(4-hydroxyphenyl)but-1-en-1-yl)phenoxy)ethyl)-3-(2-(2-((2-(2,6-dioxopiperidin-3-yl)-1,3-dioxoisoindolin-4-yl)amino)ethoxy)ethoxy)propanamide;
N-(2-(4-(1,2-bis(4-hydroxyphenyl)but-1-en-1-yl)phenoxy)ethyl)-3-(2-(2-(2-((2-(2,6-dioxopiperidin-3-yl)-1,3-dioxoisoindolin-4-yl)amino)ethoxy)ethoxy)ethoxy)propanamide;
N-(2-(4-(1,2-bis(4-hydroxyphenyl)but-1-en-1-yl)phenoxy)ethyl)-1-((2-(2,6-dioxopiperidin-3-yl)-1,3-dioxoisoindolin-4-yl)amino)-3 ,6,9,12-tetraoxapentadecan-15-amide;
N-(2-(4-(1,2-bis(4-hydroxyphenyl)but-1-en-1-yl)phenoxy)ethyl)-1-((2-(2,6-dioxopiperidin-3-yl)-1,3-dioxoisoindolin-4-yl)amino)-3,6,9,12,15-pentaoxaoctadecan-18-amide;
N-(2-(4-(1,2-bis(4-hydroxyphenyl)but-1-en-1-yl)phenoxy)ethyl)-2-((2-(2,6-dioxopiperidin-3-yl)-1,3-dioxoisoindolin-4-yl)amino)acetamide;
N-(2-(4-(1,2-bis(4-hydroxyphenyl)but-1-en-1-yl)phenoxy)ethyl)-3-((2-(2,6-dioxopiperidin-3-yl)-1,3-dioxoisoindolin-4-yl)amino)propanamide;
N-(2-(4-(1,2-bis(4-hydroxyphenyl)but-1-en-1-yl)phenoxy)ethyl)-4-((2-(2,6-dioxopiperidin-3-yl)-1,3-dioxoisoindolin-4-yl)amino)butanamide;
N-(2-(4-(1,2-bis(4-hydroxyphenyl)but-1-en-1-yl)phenoxy)ethyl)-5-((2-(2,6-dioxopiperidin-3-yl)-1,3-dioxoisoindolin-4-yl)amino)pentanamide;
N-(2-(4-(1,2-bis(4-hydroxyphenyl)but-1-en-1-yl)phenoxy)ethyl)-6-((2-(2,6-dioxopiperidin-3-yl)-1,3-dioxoisoindolin-4-yl)amino)hexanamide;
N-(2-(4-(1,2-bis(4-hydroxyphenyl)but-1-en-1-yl)phenoxy)ethyl)-7-((2-(2,6-dioxopiperidin-3-yl)-1,3-dioxoisoindolin-4-yl)amino)heptanamide;
N-(2-(4-(1,2-bis(4-hydroxyphenyl)but-1-en-1-yl)phenoxy)ethyl)-3-(3-((2-(2,6-dioxopiperidin-3-yl)-1-oxoisoindolin-4-yl)amino)-3-oxopropoxy)propanamide;
N-(2-(4-(1,2-bis(4-hydroxyphenyl)but-1-en-1-yl)phenoxy)ethyl)-3-(2-(3-((2-(2,6-dioxopiperidin-3-yl)-1-oxoisoindolin-4-yl)amino)-3-oxopropoxy)ethoxy)propanamide;
N-(2-(4-(1,2-bis(4-hydroxyphenyl)but-1-en-1-yl)phenoxy)ethyl)-3-(2-(2-(3-((2-(2,6-dioxopiperidin-3-yl)-1-oxoisoindolin-4-yl)amino)-3-oxopropoxy)ethoxy)ethoxy)propanamide;
N1-(2-(4-(1,2-bis(4-hydroxyphenyl)but-1-en-1-yl)phenoxy)ethyl)-N16-(2-(2,6-dioxopiperidin-3-yl)-1-oxoisoindolin-4-yl)-4,7,10,13-tetraoxahexadecanediamide;
N1-(2-(4-(1,2-bis(4-hydroxyphenyl)but-1-en-1-yl)phenoxy)ethyl)-N19-(2-(2,6-dioxopiperidin-3-yl)-1-oxoisoindolin-4-yl)-4,7,10,13,16-pentaoxanonadecanediamide;
N-(2-(4-(1,2-bis(4-hydroxyphenyl)but-1-en-1-yl)phenoxy)ethyl)-3-(2-((2-(2,6-dioxopiperidin-3-yl)-1-oxoisoindolin-4-yl)amino)ethoxy)propanamide;
N-(2-(4-(1,2-bis(4-hydroxyphenyl)but-1-en-1-yl)phenoxy)ethyl)-3-(2-(2-((2-(2,6-dioxopiperidin-3-yl)-1-oxoisoindolin-4-yl)amino)ethoxy)ethoxy)propanamide;
N-(2-(4-(1,2-bis(4-hydroxyphenyl)but-1-en-1-yl)phenoxy)ethyl)-3-(2-(2-(2-((2-(2,6-dioxopiperidin-3-yl)-1-oxoisoindolin-4-yl)amino)ethoxy)ethoxy)ethoxy)propanamide;
N-(2-(4-(1,2-bis(4-hydroxyphenyl)but-1-en-1-yl)phenoxy)ethyl)-1-((2-(2,6-dioxopiperidin-3-yl)-1-oxoisoindolin-4-yl)amino)-3 ,6,9,12-tetraoxapentadecan-15-amide;
N-(2-(4-(1,2-bis(4-hydroxyphenyl)but-1-en-1-yl)phenoxy)ethyl)-1-((2-(2,6-dioxopiperidin-3-yl)-1-oxoisoindolin-4-yl)amino)-3,6,9,12,15-pentaoxaoctadecan-18-amide;
N1-(2-(4-(1,2-bis(4-hydroxyphenyl)but-1-en-1-yl)phenoxy)ethyl)-N3-(2-(2,6-dioxopiperidin-3-yl)-1-oxoisoindolin-4-yl)malonamide;
N1-(2-(4-(1,2-bis(4-hydroxyphenyl)but-1-en-1-yl)phenoxy)ethyl)-N4-(2-(2,6-dioxopiperidin-3-yl)-1-oxoisoindolin-4-yl)succinamide;
N1-(2-(4-(1,2-bis(4-hydroxyphenyl)but-1-en-1-yl)phenoxy)ethyl)-N5-(2-(2,6-dioxopiperidin-3-yl)-1-oxoisoindolin-4-yl)glutaramide;
N1-(2-(4-(1,2-bis(4-hydroxyphenyl)but-1-en-1-yl)phenoxy)ethyl)-N6-(2-(2,6-dioxopiperidin-3-yl)-1-oxoisoindolin-4-yl)adipamide;
N1-(2-(4-(1,2-bis(4-hydroxyphenyl)but-1-en-1-yl)phenoxy)ethyl)-N7-(2-(2,6-dioxopiperidin-3-yl)-1-oxoisoindolin-4-yl)heptanediamide;
N1-(2-(4-(1,2-bis(4-hydroxyphenyl)but-1-en-1-yl)phenoxy)ethyl)-N8-(2-(2,6-dioxopiperidin-3-yl)-1-oxoisoindolin-4-yl)octanediamide;
N-(2-(4-(1,2-bis(4-hydroxyphenyl)but-1-en-1-yl)phenoxy)ethyl)-3-((2-(2,6-dioxopiperidin-3-yl)-1-oxoisoindolin-4-yl)amino)propanamide;
N-(2-(4-(1,2-bis(4-hydroxyphenyl)but-1-en-1-yl)phenoxy)ethyl)-4-((2-(2,6-dioxopiperidin-3-yl)-1-oxoisoindolin-4-yl)amino)butanamide;
N-(2-(4-(1,2-bis(4-hydroxyphenyl)but-1-en-1-yl)phenoxy)ethyl)-5-((2-(2,6-dioxopiperidin-3-yl)-1-oxoisoindolin-4-yl)amino)pentanamide;
N-(2-(4-(1,2-bis(4-hydroxyphenyl)but-1-en-1-yl)phenoxy)ethyl)-6-((2-(2,6-dioxopiperidin-3-yl)-1-oxoisoindolin-4-yl)amino)hexanamide;
N-(2-(4-(1,2-bis(4-hydroxyphenyl)but-1-en-1-yl)phenoxy)ethyl)-7-((2-(2,6-dioxopiperidin-3-yl)-1-oxoisoindolin-4-yl)amino)heptanamide;
N-(2-(4-(1,2-bis(4-hydroxyphenyl)but-1-en-1-yl)phenoxy)ethyl)-8-((2-(2,6-dioxopiperidin-3-yl)-1-oxoisoindolin-4-yl)amino)octanamide;
(2S,4R)-1-((S)-14-(4-(1,2-bis(4-hydroxyphenyl)but-1-en-1-yl)phenoxy)-2-(tert-butyl)-4,11-dioxo-6,9-dioxa-3,12-diazatetradecanoyl)-4-hydroxy-N-(4-(4-methylthiazol-5-yl)benzyl)pyrrolidine-2-carboxamide;
(2S,4R)-1-((S)-16-(4-(1,2-bis(4-hydroxyphenyl)but-1-en-1-yl)phenoxy)-2-(tert-butyl)-4,13-dioxo-7,10-dioxa-3,14-diazahexadecanoyl)-4-hydroxy-N-(4-(4-methylthiazol-5-yl)benzyl)pyrrolidine-2-c arboxamide;
(2S,4R)-1-((S)-19-(4-(1,2-bis(4-hydroxyphenyl)but-1-en-1-yl)phenoxy)-2-(tert-butyl)-4,16-dioxo-7,10,13-trioxa-3,17-diazanonadec anoyl)-4-hydroxy-N-(4-(4-methylthiazol-5-yl)benzyl)pyrrolidine-2-c arboxamide;
N1-(2-(4-(1,2-bis(4-hydroxyphenyl)but-1-en-1-yl)phenoxy)ethyl)-N16-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)-4,7,10,13-tetraoxahexadecanediamide;
N1-(2-(4-(1,2-bis(4-hydroxyphenyl)but-1-en-1-yl)phenoxy)ethyl)-N19-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)-4,7,10,13,16-pentaoxanonadecanediamide;
N1-(2-(4-(1,2-bis(4-hydroxyphenyl)but-1-en-1-yl)phenoxy)ethyl)-N4-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)succinamide;
N1-(2-(4-(1,2-bis(4-hydroxyphenyl)but-1-en-1-yl)phenoxy)ethyl)-N5-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)glutaramide;
N1-(2-(4-(1,2-bis(4-hydroxyphenyl)but-1-en-1-yl)phenoxy)ethyl)-N6-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)adipamide;
N1-(2-(4-(1,2-bis(4-hydroxyphenyl)but-1-en-1-yl)phenoxy)ethyl)-N7-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)heptanediamide;
N1-(2-(4-(1,2-bis(4-hydroxyphenyl)but-1-en-1-yl)phenoxy)ethyl)-N8-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)octanediamide;
N1-(2-(4-(1,2-bis(4-hydroxyphenyl)but-1-en-1-yl)phenoxy)ethyl)-N9-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)nonanediamide;
N1-(2-(4-(1,2-bis(4-hydroxyphenyl)but-1-en-1-yl)phenoxy)ethyl)-N10-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)decanediamide;
N1-(2-(4-(1,2-bis(4-hydroxyphenyl)but-1-en-1-yl)phenoxy)ethyl)-N11-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)undecanediamide;
N1-(2-(4-(1,2-bis(4-hydroxyphenyl)but-1-en-1-yl)phenoxy)ethyl)-N14-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)tetradecanediamide;
N1-(2-(4-(1,2-bis(4-hydroxyphenyl)but-1-en-1-yl)phenoxy)ethyl)-N16-((S)-1-((2S,4R)-4-hydroxy-2-((4-(4-methylthiazol-5-yl)benzyl)carbamoyl)pyrrolidin-1-yl)-3,3-dimethyl-1-oxobutan-2-yl)hexadecanediamide;
(2S,4R)-1-((S)-2-(3-(4-(3-((2-(4-(1,2-bis(4-hydroxyphenyl)but-1-en-1-yl)phenoxy)ethyl)amino)-3-oxopropyl)piperazin-1-yl)propanamido)-3,3-dimethylbutanoyl)-4-hydroxy-N-(4-(4-methylthiazol-5-yl)benzyl)pyrrolidine-2-carboxamide;
(2S,4R)-1-((S)-2-(3-(4-(3-((2-(4-(1,2-bis(4-hydroxyphenyl)but-1-en-1-yl)phenoxy)ethyl)amino)-3-oxopropyl)phenyl)propanamido)-3,3-dimethylbutanoyl)-4-hydroxy-N-(4-(4-methylthiazol-5-yl)benzyl)pyrrolidine-2-carboxamide;
N-(2-(4-(1,2-bis(4-hydroxyphenyl)but-1-en-1-yl)phenoxy)ethyl)-3-(4-(2-((2-(2,6-ioxopiperidin-3-yl)-1,3-dioxoisoindolin-4-yl)amino)ethyl)piperazin-1-yl)propanamide;
N-(2-(4-(1,2-bis(4-hydroxyphenyl)but-1-en-1-yl)phenoxy)ethyl)-3-(4-(2-((2-(2,6-dioxopiperidin-3-yl)-1,3-dioxoisoindolin-4-yl)amino)ethyl)phenyl)propanamide;
(2S,4R)-1-((S)-2-(7-((2-(4-(1,2-bis(4-hydroxyphenyl)but-1-en-1-yl)phenoxy)ethyl)amino)heptanamido)-3,3-dimethylbutanoyl)-4-hydroxy-N-(4-(4-methylthiazol-5-yl)benzyl)pyrrolidine-2-carboxamide;
(2S,4R)-1-((S)-2-((7-((2-(4-(1,2-bis(4-hydroxyphenyl)but-1-en-1-yl)phenoxy)ethyl)amino)-7-oxoheptyl)amino)-3,3-dimethylbutanoyl)-4-hydroxy-N-(4-(4-methylthiazol-5-yl)benzyl)pyrrolidine-2-carboxamide;
(2S,4R)-1-((S)-2-((7-((2-(4-(1,2-bis(4-hydroxyphenyl)but-1-en-1-yl)phenoxy)ethyl)amino)heptyl)amino)-3,3-dimethylbutanoyl)-4-hydroxy-N-(4-(4-methylthiazol-5-yl)benzyl)pyrrolidine-2-carboxamide;
4-((2-(3-(4-(2-(4-(1,2-bis (4-hydroxyphenyl)but-1-en-1-yl)phenoxy)ethyl)piperazin-1-yl)-3-oxopropoxy)ethyl)amino)-2-(2,6-dioxopiperidin-3-yl)isoindoline-1,3-dione;
4-((2-(2-(3-(4-(2-(4-(1,2-bis(4-hydroxyphenyl)but-1-en-1-yl)phenoxy)ethyl)piperazin-1-yl)-3-oxopropoxy)ethoxy)ethyl)amino)-2-(2,6-dioxopiperidin-3-yl)isoindoline-1,3-dione;
4-((2-(2-(2-(3-(4-(2-(4-(1,2-bis(4-hydroxyphenyl)but-1-en-1-yl)phenoxy)ethyl)piperazin-1-yl)-3-oxopropoxy)ethoxy)ethoxy)ethyl)amino)-2-(2,6-dioxopiperidin-3-yl)isoindoline-1,3-dione;
4-((15-(4-(2-(4-(1,2-bis(4-hydroxyphenyl)but-1-en-1-yl)phenoxy)ethyl)piperazin-1-yl)-15-oxo-3 ,6,9,12-tetraoxapentadecyl)amino)-2-(2,6-dioxopiperidin-3-yl)isoindoline-1,3-dione;
4-((18-(4-(2-(4-(1,2-bis(4-hydroxyphenyl)but-1-en-1-yl)phenoxy)ethyl)piperazin-1-yl)-18-oxo-3 ,6,9,12,15-pentaoxaoctadecyl)amino)-2-(2,6-dioxopiperidin-3-yl)isoindoline-1,3-dione;
4-((2-(4-(2-(4-(1,2-bis(4-hydroxyphenyl)but-1-en-1-yl)phenoxy)ethyl)piperazin-1-yl)-2-oxoethyl)amino)-2-(2,6-dioxopiperidin-3-yl)isoindoline-1,3-dione;
4-((3-(4-(2-(4-(1,2-bis(4-hydroxyphenyl)but-1-en-1-yl)phenoxy)ethyl)piperazin-1-yl)-3-oxopropyl)amino)-2-(2,6-dioxopiperidin-3-yl)isoindoline-1,3-dione;
4-((4-(4-(2-(4-(1,2-bis(4-hydroxyphenyl)but-1-en-1-yl)phenoxy)ethyl)piperazin-1-yl)-4-oxobutyl)amino)-2-(2,6-dioxopiperidin-3-yl)isoindoline-1,3-dione;
4-((5-(4-(2-(4-(1,2-bis(4-hydroxyphenyl)but-1-en-1-yl)phenoxy)ethyl)piperazin-1-yl)-5-oxopentyl)amino)-2-(2,6-dioxopiperidin-3-yl)isoindoline-1,3-dione;
4-((6-(4-(2-(4-(1,2-bis(4-hydroxyphenyl)but-1-en-1-yl)phenoxy)ethyl)piperazin-1-yl)-6-oxohexyl)amino)-2-(2,6-dioxopiperidin-3-yl)isoindoline-1,3-dione;
4-((7-(4-(2-(4-(1,2-bis(4-hydroxyphenyl)but-1-en-1-yl)phenoxy)ethyl)piperazin-1-yl)-'7-oxoheptyl)amino)-2-(2,6-dioxopiperidin-3-yl)isoindoline-1,3-dione;
3-(4-((2-(3-(4-(2-(4-(1,2-bis(4-hydroxyphenyl)but-1-en-1-yl)phenoxy)ethyl)piperazin-1-yl)-3-oxopropoxy)ethyl)amino)-1-oxoisoindolin-2-yl)piperidine-2,6-dione;
3-(4-((2-(2-(3-(4-(2-(4-(1,2-bis(4-hydroxyphenyl)but-1-en-1-yl)phenoxy)ethyl)piperazin-1-yl)-3-oxopropoxy)ethoxy)ethyl)amino)-1-oxoisoindolin-2-yl)piperidine-2,6-dione;
3-(4-((2-(2-(2-(3-(4-(2-(4-(1,2-bis(4-hydroxyphenyl)but-1-en-1-yl)phenoxy)ethyl)piperazin-1-yl)-3-oxopropoxy)ethoxy)ethoxy)ethyl)amino)-1-oxoisoindolin-2-yl)piperidine-2,6-dione;
3-(4-((15-(4-(2-(4-(1,2-bis(4-hydroxyphenyl)but-1-en-1-yl)phenoxy)ethyl)piperazin-1-yl)-15-oxo-3 ,6,9,12-tetraoxapentadecyl)amino)-1-oxoisoindolin-2-yl)piperidine-2,6-dione;
3-(4-((18-(4-(2-(4-(1,2-bis(4-hydroxyphenyl)but-1-en-1-yl)phenoxy)ethyl)piperazin-1-yl)-18-oxo-3 ,6,9,12,15-pentaoxaoctadecyl)amino)-1-oxoisoindolin-2-yl)piperidine-2,6-dione;
3-(4-((2-(4-(2-(4-(1,2-bis(4-hydroxyphenyl)but-1-en-1-yl)phenoxy)ethyl)piperazin-1-yl)-2-oxoethyl)amino)-1-oxoisoindolin-2-yl)piperidine-2,6-dione;
3-(4-((3-(4-(2-(4-(1,2-bis(4-hydroxyphenyl)but-1-en-1-yl)phenoxy)ethyl)piperazin-1-yl)-3-oxopropyl)amino)-1-oxoisoindolin-2-yl)piperidine-2,6-dione;
3-(4-((4-(4-(2-(4-(1,2-bis(4-hydroxyphenyl)but-1-en-1-yl)phenoxy)ethyl)piperazin-1-yl)-4-oxobutyl)amino)-1-oxoisoindolin-2-yl)piperidine-2,6-dione;
3-(4-((5-(4-(2-(4-(1,2-bis(4-hydroxyphenyl)but-1-en-1-yl)phenoxy)ethyl)piperazin-1-yl)-5-oxopentyl)amino)-1-oxoisoindolin-2-yl)piperidine-2,6-dione;
3-(4-((6-(4-(2-(4-(1,2-bis(4-hydroxyphenyl)but-1-en-1-yl)phenoxy)ethyl)piperazin-1-yl)-6-oxohexyl)amino)-1-oxoisoindolin-2-yl)piperidine-2,6-dione;
3-(4-((7-(4-(2-(4-(1,2-bis(4-hydroxyphenyl)but-1-en-1-yl)phenoxy)ethyl)piperazin-1-yl)-7-oxoheptyl)amino)-1-oxoisoindolin-2-yl)piperidine-2,6-dione;
(2S,4R)-1-((S)-2-(2-(2-(2-(4-(2-(4-(1,2-bis(4-hydroxyphenyl)but-1-en-1-yl)phenoxy)ethyl)piperazin-1-yl)-2-oxoethoxy)ethoxy)acetamido)-3,3-dimethylbutanoyl)-4-hydroxy-N-(4-(4-methylthiazol-5-yl)benzyl)pyrrolidine-2-carboxamide;
(2S,4R)-1-((S)-2-(3-(2-(3-(4-(2-(4-(1,2-bis(4-hydroxyphenyl)but-1-en-1-yl)phenoxy)ethyl)piperazin-1-yl)-3-oxopropoxy)ethoxy)propanamido)-3,3-dimethylbutanoyl)-4-hydroxy-N-(4-(4-methylthiazol-5-yl)benzyl)pyrrolidine-2-carboxamide;
(2S,4R)-1-((S)-16-(4-(2-(4-(1,2-bis(4-hydroxyphenyl)but-1-en-1-yl)phenoxy)ethyl)piperazin-1-yl)-2-(tert-butyl)-4,16-dioxo-7,10,13-trioxa-3-azahexadecanoyl)-4-hydroxy-N-(4-(4-methylthiazol-5-yl)benzyl)pyrrolidine-2-carboxamide;
(2S,4R)-1-((S)-19-(4-(2-(4-(1,2-bis(4-hydroxyphenyl)but-1-en-1-yl)phenoxy)ethyl)piperazin-1-yl)-2-(tert-butyl)-4,19-dioxo-7,10,13,16-tetraoxa-3-azanonadecanoyl)-4-hydroxy-N-(4-(4-methylthiazol-5-yl)benzyl)pyrrolidine-2-carboxamide;
(2S,4R)-1-((S)-22-(4-(2-(4-(1,2-bis(4-hydroxyphenyl)but-1-en-1-yl)phenoxy)ethyl)piperazin-1-yl)-2-(tert-butyl)-4,22-dioxo-7,10,13,16,19-pentaoxa-3-azadoco sanoyl)-4-hydroxy-N-(4-(4-methylthiazol-5-yl)benzyl)pyrrolidine-2-carboxamide;
(2S,4R)-1-((S)-2-(4-(4-(2-(4-(1,2-bis(4-hydroxyphenyl)but-1-en-1-yl)phenoxy)ethyl)piperazin-1-yl)-4-oxobutanamido)-3,3-dimethylbutanoyl)-4-hydroxy-N-(4-(4-methylthiazol-5-yl)benzyl)pyrrolidine-2-carboxamide;
(2S,4R)-1-((S)-2-(5-(4-(2-(4-(1,2-bis(4-hydroxyphenyl)but-1-en-1-yl)phenoxy)ethyl)piperazin-1-yl)-5-oxopentanamido)-3,3-dimethylbutanoyl)-4-hydroxy-N-(4-(4-methylthiazol-5-yl)benzyl)pyrrolidine-2-carboxamide;
(2S,4R)-1-((S)-2-(6-(4-(2-(4-(1,2-bis(4-hydroxyphenyl)but-1-en-1-yl)phenoxy)ethyl)piperazin-1-yl)-6-oxohexanamido)-3,3-dimethylbutanoyl)-4-hydroxy-N-(4-(4-methylthiazol-5-yl)benzyl)pyrrolidine-2-carboxamide;
(2S,4R)-1-((S)-2-(7-(4-(2-(4-(1,2-bis(4-hydroxyphenyl)but-1-en-1-yl)phenoxy)ethyl)piperazin-1-yl)-7-oxoheptanamido)-3,3-dimethylbutanoyl)-4-hydroxy-N-(4-(4-methylthiazol-5-yl)benzyl)pyrrolidine-2-carboxamide;
(2S,4R)-1-((S)-2-(8-(4-(2-(4-(1,2-bis(4-hydroxyphenyl)but-1-en-1-yl)phenoxy)ethyl)piperazin-1-yl)-8-oxooctanamido)-3,3-dimethylbutanoyl)-4-hydroxy-N-(4-(4-methylthiazol-5-yl)benzyl)pyrrolidine-2-carboxamide;
(2S,4R)-1-((S)-2-(9-(4-(2-(4-(1,2-bis(4-hydroxyphenyl)but-1-en-1-yl)phenoxy)ethyl)piperazin-1-yl)-9-oxononanamido)-3,3-dimethylbutanoyl)-4-hydroxy-N-(4-(4-methylthiazol-5-yl)benzyl)pyrrolidine-2-carboxamide;
(2S,4R)-1-((S)-2-(10-(4-(2-(4-(1,2-bis(4-hydroxyphenyl)but-1-en-1-yl)phenoxy)ethyl)piperazin-1-yl)-10-oxodecanamido)-3,3-dimethylbutanoyl)-4-hydroxy-N-(4-(4-methylthiazol-5-yl)benzyl)pyrrolidine-2-carboxamide;
(2S,4R)-1-((S)-2-(7-(4-(2-(4-(1,2-bis(4-hydroxyphenyl)but-1-en-1-yl)phenoxy)ethyl)piperazin-1-yl)heptanamido)-3,3-dimethylbutanoyl)-4-hydroxy-N-(4-(4-methylthiazol-5-yl)benzyl)pyrrolidine-2-carboxamide;
(2S,4R)-1-((S)-2-((7-(4-(2-(4-(1,2-bis(4-hydroxyphenyl)but-1-en-1-yl)phenoxy)ethyl)piperazin-1-yl)-7-oxoheptyl)amino)-3,3-dimethylbutanoyl)-4-hydroxy-N-(4-(4-methylthiazol-5-yl)benzyl)pyrrolidine-2-carboxamide; and
(2S,4R)-1-((S)-2-((7-(4-(2-(4-(1,2-bis(4-hydroxyphenyl)but-1-en-1-yl)phenoxy)ethyl)piperazin-1-yl)heptyl)amino)-3,3-dimethylbutanoyl)-4-hydroxy-N-(4-(4-methylthiazol-5-yl)benzyl)pyrrolidine-2-carboxamide.

54. A pharmaceutical composition comprising the compound of formula (I) of claim 1 or a pharmaceutically acceptable salt thereof, and at least one pharmaceutically acceptable carrier.

55. The pharmaceutical composition of claim 54, further comprising at least one additional therapeutic agent.

56. The pharmaceutical composition according to claim 55, wherein the at least one additional therapeutic agent is used to treat or prevent cancer.

57. The compound of formula (I) of claim 1, or a pharmaceutically acceptable salt thereof, for use as a medicament.

58. The compound of formula (I) of claim 1, or a pharmaceutically acceptable salt thereof, for use in the prevention and/or treatment of diseases or disorders associated with estrogen receptor.

59. The compound of formula (I) according to claim 58, or a pharmaceutically acceptable salt thereof, wherein the diseases or disorders associated with estrogen receptor are selected from the group consisting of:
cancer, osteoporosis, atherosclerosis, atrophic vaginitis, proliferative diseases, tumor metastasis, bipolar disorder, depression, and inducing ovulation in anovulatory infertile subject.

60. The compound of formula (I) according to claim 59, or a pharmaceutically acceptable salt thereof, wherein the cancer is selected from the group consisting of: breast cancer, uterine cancer, ovarian tumor, and malignant melanoma.

61. The compound of formula (I) according to claim 60, or a pharmaceutically acceptable salt thereof, wherein the breast cancer is selected from the group consisting of: ER-positive breast cancer in menopausal women with CYP2D6 gene defect, lymph node-positive breast cancer, and ductal carcinoma in situ of the breast.

62.-65. (cnceled)

66. A method of treating or preventing diseases or disorders associated with estrogen receptor, comprising administering to a subject a therapeutically effective amount of a compound of formula (I), or a pharmaceutically acceptable salt thereof, or a pharmaceutical composition of claim 54,
wherein the compound of formula (I) is: 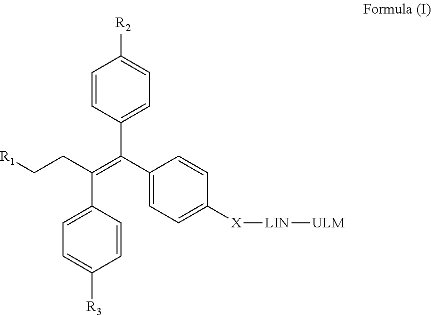
or salts, enantiomers, stereoisomers, solvates, or polymorphs thereof, wherein X is covalently bonded to ULM through a linking group LIN;
wherein R₁ represents halogen, R₂ represents H, halogen, or OH, and R₃ represents H, halogen, or OH; or R₁ represents H, and R₂ and R₃ are both halogen or OH;
X represents CH₂, O, or NH;
LIN is a linking group and represents -alkylene-,
wherein the alkylene group is a linear or branched alkylene group optionally interrupted one or more times by one or more groups selected from the group consisting of O, CO, CON(R₄), N(R₅)CO, N(R₆), alkynylene, alkenylene, cycloalkylene, arylene, heterocyclylene, heteroarylene, or any combination thereof, and
wherein the linear or branched alkylene group is optionally substituted with one or more substituents; and R₄, R₅, and R₆ are each independently selected from the group consisting of H and C₁₋₃ alkyl; and
ULM is a small-molecule ligand of VHL or CRBN protease having a ubiquitylation function.

67. The method of claim 66, wherein the diseases or disorders associated with estrogen receptor are selected from the group consisting of: cancer, osteoporosis, atherosclerosis, atrophic vaginitis, proliferative diseases, tumor metastasis, bipolar disorder, depression, and inducing ovulation in anovulatory infertile subject.

68. The method of claim 67, wherein the cancer is selected from the group consisting of: breast cancer, uterine cancer, ovarian tumor, and malignant melanoma.

69. The method of claim 68, wherein the breast cancer is selected from the group consisting of: ER-positive breast cancer in menopausal women with CYP2D6 gene defect, lymph node-positive breast cancer, and ductal carcinoma in situ of the breast.

70. The method of claim 66, wherein the administering to the subject is carried out through at least one route of administration selected from the group consisting of: nasal administration, inhalation administration, topical administration, oral administration, oral mucosal administration, rectal administration, intrapleural administration, intraperitoneal administration, vaginal administration, intramuscular administration, subcutaneous administration, transdermal administration, epidural administration, intrathecal administration and intravenous administration.
